(19) United States
(12) Patent Application Publication
(10) Pub. No.: US 2025/0059214 A1
(43) Pub. Date: Feb. 20, 2025

(54) AMIDE-CONTAINING LONP1 INHIBITOR COMPOUNDS, USES AND METHODS

(71) Applicant: Pretzel Therapeutics, Inc., Waltham, MA (US)

(72) Inventors: Jeremy Green, Waltham, MA (US); Paul Charifson, Carrabassett Valley, ME (US); Gabriel Martinez Botella, Wayland, MA (US); Andrew Griffin, L'Île Bizard, Quebec (CA)

(73) Assignee: Pretzel Therapeutics, Inc., Waltham, MA (US)

(21) Appl. No.: 18/716,899

(22) PCT Filed: Dec. 6, 2022
(86) PCT No.: PCT/US2022/051996

§ 371 Date: Jun. 5, 2024

Related U.S. Application Data
(60) Provisional application No. 63/286,441, filed on Dec. 6, 2021.
(60) Provisional application No. 63/290,841, filed on Dec. 17, 2021.
(60) Provisional application No. 63/319,596, filed on Mar. 14, 2022.
(60) Provisional application No. 63/358,741, filed on Jul. 6, 2022.

(51) Int. Cl. C07F 5/02; A61K 31/69

(52) U.S. Cl. CPC C07F 5/025; A61K 31/69

(57) ABSTRACT

Disclosed are compounds according to Formula (1) which inhibit LONP1, and pharmaceutical compositions comprising compounds of the disclosure. Compounds and pharmaceutical compositions of the disclosure may be useful for the treatment of diseases and disorders associated with LONP1, including oncologic diseases and disorders, such as cancer, and diseases and disorders related to mitochondrial dysfunction, such as neurodegenerative disorders, metabolic disorders, and diseases associated with the aging process. The disclosure also relates to methods of using such compounds and compositions for the treatment of such diseases and disorders.

### FIELD OF THE INVENTION

The present invention relates to novel LONP1 inhibitors, their pharmaceutically acceptable salts, and pharmaceutical compositions thereof. The present invention also relates to methods of using such compounds and compositions, including to inhibit LONP1 and to treat oncologic diseases and disorders, such as cancer, and various diseases and disorders related to mitochondrial dysfunction, such as neurodegenerative disorders, metabolic disorders, and diseases associated with the aging process.

### BACKGROUND OF THE INVENTION

The mitochondrial Lon serine protease, LONP1, is an enzyme that is a member of the AAA+ superfamily of proteases (i.e., ATP-dependent proteases (ATPases) associated with diverse cellular activities). Widely conserved across eukaryotic species, human LONP1 is a 959-amino acid protein that consists of three domains: the N-terminal domain involved in substrate binding, the AAA+ (ATPase) domain, and the C-terminal domain (named the P-domain) involved in proteolytic activity. The ATPase and protease domains are the most well-conserved across species, while the N-terminal domain is the most variable.

LONP1 performs at least four different functions: proteolysis of damaged and oxidized proteins of the mitochondrial matrix; chaperone activity, namely the correct folding of proteins imported into the mitochondria; regulation of mitochondrial protein levels, including mitochondrial transcription factor A (TFAM); and binding to mitochondrial DNA (“mtDNA”) and RNA. As for the proteolytic activity of LONP1, like all the other proteases in the AAA+ family, it binds its substrate, unfolds it using the ATPase domain, and then digests it from the N or C-terminus. Its chaperone activity, mediated by the ATP-binding domain and the N-terminal domain, is crucial for mitochondrial homeostasis, as it is involved in the assembly of mitochondrial membrane complexes.

LONP1 has multiple, natural substrates, one of which is the mtDNA binding and packaging protein TFAM. TFAM has a crucial role in transcription initiation and mtDNA replication. Inhibition of LONP1 reportedly leads to increased levels of the TFAM protein, which in turn may lead to higher levels of mtDNA.

TFAM and mtDNA have a mutual dependence for stability, whereby TFAM binds mtDNA and protects it from degradation, but when not bound to mtDNA, TFAM is rapidly degraded. LONP1 has been shown to regulate mtDNA copy number in Drosophila melanogaster by cleaving TFAM. In human cells with severe mtDNA deficits, depletion of LONP1 can increase levels of TFAM and upregulate mtDNA content.

Another natural substrate of LONP1 is POLγA, the catalytic subunit of DNA polymerase γ (POLγ). POLγ is the main protein responsible for mitochondrial DNA (mtDNA) replication.

The accessory POLγB subunit acts to stabilize POLγA and to prevent LONP1-dependent degradation. Disease causing mutations such as A467T weaken interactions between POLγA and POLγB, which in turn makes POLγA susceptible to degradation by LONP1.

LONP1 is also required during embryogenesis. A homozygous deletion of the LONP1 gene in a mouse causes embryonic lethality. In line with this observation, mutations that change LONP1 activity during embryogenesis can cause a congenital syndrome known as CODAS, characterized by Cerebral, Ocular, Dental, Auricular and Skeletal anomalies. Further supporting a role during embryogenesis, defective mitochondrial protease LONP1 has also been linked to a classical, congenital mitochondrial disease. The mutant (Tyr565His) protein displayed higher ATPase activity, but reduced protease activity (see Peter, B. et. al., “Defective Mitochondrial Protease LonP1 Can Cause Classical Mitochondrial Disease,” Hum. Mol. Genet., 27, 10, 1743-1750 (2018)).

Additionally, LONP1 has a central role in the regulation of mitochondrial function, impacting bioenergetics in various cells and often causing disease (see Gibellini L. et. al., “LonP1 Differently Modulates Mitochondrial Function and Bioenergetics of Primary Versus Metastatic Colon Cancer Cells,” Front. Oncol. 8, 254 (2018). LONP1 upregulation is a characteristic shared by various types of cancer cells. Higher expression of LONP1 is correlated with tumor progression and aggressiveness. For instance, LONP1 overproduction is functionally linked to colorectal cancer cells by inducing the epithelial mesenchymal transition, an early step in the formation of metastases (see id). Furthermore, LONP1 is a regulator of mitochondrial proteostasis, which is required for maintaining the respiratory chain and degrading misfolded, oxidatively damaged or unassembled proteins. As such, inhibition of LONP1 is believed to be a mechanism by which various oncogenic diseases, such as cancers may be treated.

Similarly, multiple myeloma is an exceedingly prevalent and incurable cancer in the elderly (see Maneix, L. et al., “The Mitochondrial Protease LonP1 Promotes Proteasome Inhibitor Resistance in Multiple Myeloma,” Cancers 13, 843, 14-19 (2021)). Proteasome inhibitors are a common treatment for myeloma, but for unknown reasons, over time, a resistance to treatment develops. Compounds that inhibit LONP1 may provide a means to more thoroughly understand the molecular mechanisms that lead to such drug resistance in the treatment of multiple myeloma (see id).

While aspects of LONP1 biochemistry are known, its full physiological role in mitochondrial gene expression and homeostasis, as well as its underlying impact in the etiology of various disease states, remains unclear. LONP1 inhibitors will provide insight into, for example, the relationship between LONP1, mtDNA copy number, and human diseases. Pharmacological inhibition of LONP1 is one means by which to gain a further understanding of the role of this protease in cell physiology and the development of disease. LONP1 inhibitors have been reported, for example, in Kingsley, L. J. et al., J. Med. Chem. 64, 8, 4857-4869 (2021). In view of the numerous and varied roles of LONP1, there is a need for additional, potent, and specific inhibitors of LONP1.

### SUMMARY OF THE INVENTION

Provided are compounds, pharmaceutically acceptable salts of the compounds, pharmaceutical compositions comprising the compounds or their salts, methods of using the compounds, salts of the compounds, or pharmaceutical compositions of the compounds or their salts, and therapeutic uses of the compounds, or pharmaceutical compositions of the compounds or their salts, for treating diseases related to oncologic diseases and disorders, such as cancer, and/or various diseases and disorders related to mitochondrial dysfunction, such as neurodegenerative disorders, metabolic disorders, and diseases associated with the aging process. The compounds and their pharmaceutically acceptable salts are particularly useful as inhibitors of LONP1.

In one aspect there is provided a compound of structural Formula 1:

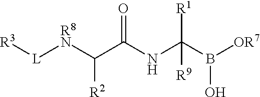

or a pharmaceutically acceptable salt, solvate, stereoisomer or mixture of stereoisomers, tautomer, isotopic form, pharmaceutically active metabolite thereof, or combinations thereof, wherein:
R¹ is selected from the group consisting of: deuterium, C1-C4 alkyl, C1-C4 alkoxyl, C1-C4 oxoalkyl, C1-C5 alkyl-alkoxyl, wherein each alkyl, oxoalkyl or alkoxyl is optionally substituted with C3-C6 cycloalkyl, phenyl, phenoxy, or a 5- or 6-membered heteroaryl, wherein said phenyl, phenoxy, or heteroaryl are each optionally substituted with one or more substituent selected from deuterium, halogen, hydroxyl, CN, CO2H, CO2R¹⁰, CONR¹⁰R¹¹, NR¹⁰R¹¹, SR¹⁰, SO2NR¹⁰R¹¹, C1-C4 alkyl, C1-C4 haloalkyl, C1-C4 alkoxy, phenyl, or a 5- or 6-membered heteroaryl;
R² is (C₁-C₄ alkyl)-C(O)NR⁴R⁵;
R⁴ and R⁵ are each independently selected from hydrogen, deuterium, C1-C4 alkyl; C1-C4 haloalkyl, C1-C5 alkyl-alkoxyl or R⁴ and R⁵ together with the N to which they are attached form 5 or 6 membered heterocyclic ring optionally having one or more additional heteroatoms selected from N, O and S, wherein the 5 or 6 membered heterocyclic ring is optionally substituted with one or more substituent selected from deuterium, halogen, hydroxyl, oxo, CN, C1-C4 alkyl, C1-C4 haloalkyl or C1-C4 alkoxyl;
L is C(O), C(O)O, C(O)NR⁶, S(O)₂, or a bond;
R³ is C₁-C₄ alkyl optionally substituted with one or more substituents each independently selected from the group consisting of deuterium, halogen, cyano, hydroxyl, C₁-C₄ alkoxyl, 5 or 6 membered aryl (e.g. phenyl) or 5 or 6 membered heteroaryl; or
R³ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, oxo, C₁-C₄ alkoxyl, or C₁-C₄ alkyl that is optionally substituted with one to three substituents selected from deuterium, halogen, cyano, hydroxyl, or C₁-C₄ alkoxyl; or
R³ is aryl or heteroaryl having one or more heteroatoms selected from N, O and S, wherein aryl or heteroaryl is optionally substituted with one or more substituents selected from deuterium, halogen, cyano, hydroxyl, OR, CO2H, CO2R¹⁰, CONR¹⁰R¹¹, NR¹⁰R¹¹, SR¹⁰, SO2NR¹⁰R¹¹, C₁-C₄ alkoxyl, or C₁-C₄ alkyl that is optionally substituted with one to three substituents selected from deuterium, halogen, cyano, hydroxyl, or C₁-C₄ alkoxyl;
R⁶ is hydrogen or C₁-C₄ alkyl optionally substituted with one or more of halogen, hydroxyl and phenyl, wherein phenyl is optionally substituted with one or more substituent selected from halogen, hydroxyl and C1-C2 alkyl;
R⁷ is hydrogen, or R⁷ and R¹, together with the boron atom to which R⁷ is attached form a 5-membered heteroalkyl ring;
R^{a} is selected from hydrogen, deuterium or C₁-C₂ alkyl optionally substituted with one or more substituents each independently selected from the group consisting of deuterium, halogen, hydroxyl, cyano, methoxyl and phenyl;
R⁹ is selected from hydrogen, deuterium or C₁-C₂ alkyl; and
R¹⁰ and R¹¹ are each independently selected from hydrogen, deuterium, C1-C4 alkyl; C1-C4 haloalkyl, C1-C5 alkyl-alkoxyl, C3-C7 cycloalkyl, or R¹⁰ and R¹¹ together with the N to which they are attached form 3 to 7 membered heterocyclic ring optionally having one or more additional heteroatoms selected from N, O and S, wherein the C3-C7 cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl.

Embodiments of the present disclosure include compounds of the disclosure (that is, compounds of Formula 1) or their pharmaceutically acceptable salts wherein one or more hydrogen atom is substituted with a deuterium atom.

Another aspect of the disclosure is directed to pharmaceutical compositions comprising a compound of the disclosure (that is, compounds of Formula 1) or a pharmaceutically acceptable salt thereof and a pharmaceutically acceptable excipient.

Other aspects of the disclosure are directed to methods of treating a disease or disorder, such as a disease or disorder characterized by mitochondrial dysfunction, such methods comprising administering to a subject in need thereof a therapeutically effective amount of a compound of the disclosure, a pharmaceutically acceptable salt thereof, or a composition comprising such as compound.

In various aspects and embodiments of the methods and therapeutic uses disclosed herein, the disease is selected from Alper's syndrome (Alpers-Huttenlocher syndrome), ataxia neuropathy syndrome (ANS), Mitochondrial DNA Depletion Syndrome (MDDS), Leigh Syndrome (Leigh Disease), Leber's Hereditary Optic Neuropathy (LHON), chronic progressive external ophthalmoplegia (CPEO), myoclonic epilepsy myopathy sensory ataxia (MEMSA), MELAS (Mitochondrial Encephalopathy, Lactic Acidosis, and Stroke-like episodes) syndrome, MERRF (myoclonus epilepsy with ragged-red fibers) syndrome, mitochondrial neurogastrointestinal encephalomyopathy (MNGIE), neuropathy, ataxia, and retinitis pigmentosa (NARP), Kearn's-Sayre Syndrome (KSS), and Pearson's Syndrome. In some aspects and embodiments the disease or disorder is selected from Alzheimer's disease, Parkinson's disease, obesity, diabetes, non-alcoholic steatohepatitis (NASH), and related metabolic syndromes such as non-alcoholic fatty liver disease (NAFLD).

Other aspects of the disclosure are directed to compounds or (pharmaceutical) compositions comprising compounds of the disclosure for use in methods for treating a disease or disorder, such as a disease or disorder characterized by mitochondrial dysfunction. These therapeutic uses may comprise administering to a subject in need thereof a therapeutically effective amount of a compound of the disclosure, a pharmaceutically acceptable salt thereof, or a composition comprising such a compound. Suitable diseases or disorders are those described above and herein below.

In some embodiments, the disease to be treated with a compound or composition of the disclosure is associated with mtDNA mutations or deletions, for example: m.3243A>G, m.11778G>A, m.14484T>C, m.3460G>A, m.8344A>G, m.3271T>C, m.3251A>G, m.8356T>C, m.4274T>C, m.14709T>C, m.12320A>G, m.4269A>G, m.12258C>A, m.1606G>A, m.10010T>C, m.7445A>G and m.1555A>G (see https://mitomap.org/MITOMAP).

Additional aspects and embodiments of the disclosure relate to methods of treating cancers and compounds or compositions for use in such methods: for example, those identified in Wong, K. S. et al. “Recent Advances in Targeting Human Mitochondrial AAA+ Proteases to Develop Novel Cancer Therapeutics,” Advances in Experimental Medicine and Biology, 1158,119-142 (2019), wherein the use or method comprising using a compound or composition of the disclosure or its pharmaceutically acceptable salt.

Further aspects and embodiments of the disclosure relate to methods of treating cancer, neurodegenerative disorders, metabolic disorders, and diseases associated with the aging process; and compounds and compositions of the disclosure for use in such methods.

Within the scope of this disclosure it is expressly intended that the various aspects, embodiments, examples and alternatives set out in the preceding paragraphs, in the claims and/or in the following description, and in particular the individual features thereof, may be taken independently or in any combination. That is, all embodiments and/or features of any aspect or embodiment can be combined in any way and/or combination, unless such features are incompatible. More particularly, it is specifically intended that any embodiment of any aspect may form an embodiment of any other aspect, and all such combinations are encompassed within the scope of the disclosure. The applicant reserves the right to change any originally filed claim or file any new claim accordingly, including the right to amend any originally filed claim to depend from and/or incorporate any feature of any other claim although not originally claimed in that manner.

### DETAILED DESCRIPTION OF THE INVENTION

Described herein are compounds and compositions (e.g. organic molecules, research tools, pharmaceutical formulations and therapeutics); uses for the compounds and compositions of the disclosure (in vitro and in vivo); as well as corresponding methods, whether diagnostic, therapeutic or for research applications. The chemical synthesis and biological testing of the compounds of the disclosure are also described. Beneficially, the compounds, compositions, uses and methods have utility in research towards and/or the treatment of diseases or disorders in animals, such as humans. Diseases or disorders which may benefit from LONP1 modulation include mitochondrial diseases, cancer and/or oncologic disease.

However, the compounds of the disclosure may also or alternatively be useful as lead molecules for the selection, screening and development of further derivatives that may have one or more improved beneficial drug property, as desired.

The disclosure also encompasses salts, solvates and functional derivatives of the compounds described herein. These compounds may be useful in the treatment of diseases or disorders characterized by mitochondrial disfunction; particularly those which may benefit from LONP1 inhibition.

Inhibitors of LONP1 are useful in compositions and methods suitable for treating many disorders, such as disorders characterized by mitochondrial dysfunction, including cancer. In some embodiments, the disease is selected from the group consisting of adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma colon cancer, colorectal cancer, cancer of connective tissue, esophageal cancer, embryonal carcinoma, fibrosarcoma, glioblastomas, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), liposarcoma, liver cancer, lung cancer, lymphoid cancers (e.g., Hodgkin's and non-Hodgkin's lymphomas, mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, and vulvar cancer.

### Definitions

Unless defined otherwise, all technical and scientific terms used herein have the same meaning as commonly understood by one of ordinary skill in the art (e.g. in organic, physical or theoretical chemistry; biochemistry and molecular biology).

Unless otherwise indicated, the practice of the present disclosure employs conventional techniques in chemistry and chemical methods, biochemistry, molecular biology, pharmaceutical formulation, and delivery and treatment regimens for patients, which are within the capabilities of a person of ordinary skill in the art. Such techniques are also described in the literature cited herein. All documents cited in this disclosure are herein incorporated by reference in their entirety.

Prior to setting forth the further detailed description and Examples of this disclosure, a number of definitions are provided that will assist in the understanding of the disclosure.

In accordance with this disclosure, the terms ‘molecule’ or ‘molecules’ are used interchangeably with the terms ‘compound’ or ‘compounds’, and sometimes the term ‘chemical structure’. The term ‘drug’ is typically used in the context of a pharmaceutical, pharmaceutical composition, medicament or the like, which has a known or predicted physiological or in vitro activity of medical significance; but such characteristics and qualities are not excluded in a molecule or compound of the disclosure. The term ‘drug’ is therefore used interchangeably with the alternative terms and phrases ‘therapeutic (agent)’, ‘pharmaceutical (agent)’, and ‘active (agent)’. Therapeutics according to the disclosure also encompass compositions and pharmaceutical formulations comprising the compounds of the disclosure.

Prodrugs and solvates of the compounds of the disclosure are also encompassed within the scope of the disclosure. The term ‘prodrug’ means a compound (e.g. a drug precursor) that is transformed in vivo to yield a compound of the disclosure or a pharmaceutically acceptable salt, solvate or ester of the compound. The transformation may occur by various mechanisms (e.g. by metabolic or chemical processes), such as by hydrolysis of a hydrolysable bond, e.g. in blood (see Higuchi & Stella (1987), “Pro-drugs as Novel Delivery Systems”, vol. 14 of the A. C. S. Symposium Series; (1987), “Bioreversible Carriers in Drug Design”, Roche, ed., American Pharmaceutical Association and Pergamon Press). The compositions and medicaments of the disclosure therefore may comprise prodrugs of the compounds of the disclosure. In some aspects and embodiments the compounds of the disclosure may be themselves prodrugs which may be metabolized in vivo to give the therapeutically effective compound.

The scope of this disclosure also includes various deuterated forms of the compounds of any of Formula 1 (inc. corresponding subgeneric formulas defined herein), respectively, or a pharmaceutically acceptable salt and/or a corresponding tautomer form thereof (including subgeneric formulas, as defined above). Each available hydrogen atom attached to a carbon atom may be independently replaced with a deuterium atom. A person of ordinary skill in the art will know how to synthesise deuterated forms of the compounds of Formula 1 disclosed herein (including subgeneric formulas, as defined above) or a pharmaceutically acceptable salt and/or a corresponding tautomer form thereof (including subgeneric formulas, as defined herein) of the present disclosure. For example, deuterated materials, such as alkyl groups may be prepared by conventional techniques (see for example: methyl-d3-amine available from Aldrich Chemical Co., Milwaukee, WI, Cat. No. 489,689-2).

The disclosure also includes isotopically-labelled compounds which are identical to those recited in Formula 1 disclosed herein (inc. corresponding subgeneric formulas defined herein), respectively, or a pharmaceutically acceptable salt and/or a corresponding tautomer form thereof (including subgeneric formulas, as defined above), but for the fact that one or more atoms are replaced by an atom having an atomic mass or mass number different from the atomic mass or mass number most commonly found in nature. Examples of isotopes that can be incorporated into compounds of this disclosure include isotopes of hydrogen, carbon, nitrogen, oxygen, fluorine, iodine and chlorine such as 3H, 11C, 14C, 18F, 123I or 125I. Compounds of the present disclosure and pharmaceutically acceptable salts of said compounds that contain the aforementioned isotopes and/or other isotopes of other atoms are within the scope of this disclosure. Isotopically labelled compounds of the present disclosure, for example those into which radioactive isotopes such as 3H or 14C have been incorporated, are useful in drug and/or substrate tissue distribution assays. Tritiated, i.e. 3H, and carbon-14, i.e. 14C, isotopes can be particularly beneficial for their ease of preparation and detectability. 11C and 18F isotopes are particularly useful in PET (positron emission tomography).

In the context of the present disclosure, the terms ‘individual’, ‘subject’, or ‘patient’ are used interchangeably to indicate an animal that may be suffering from a medical (pathological) condition and may be responsive to a compound/molecule, pharmaceutical drug, medical treatment or therapeutic treatment regimen of the disclosure. The animal is suitably a mammal, such as a human, cow, sheep, pig, dog, cat, bat, mouse or rat. In particular, the subject may be a human.

The term ‘alkyl’ refers to a monovalent, optionally substituted, saturated aliphatic hydrocarbon radical. Any number of carbon atoms may be present, but typically the number of carbon atoms in the alkyl group may be from 1 to about 20, from 1 to about 12, from 1 to about 6 or from 1 to about 4. Usefully, the number of carbon atoms is indicated, for example, a C1-C12 alkyl (or C1-C12 alkyl) refers to any alkyl group containing 1 to 12 carbon atoms in the chain. An alkyl group may be a straight chain (i.e. linear), branched chain, or cyclic. ‘Lower alkyl’ refers to an alkyl of 1 to 6 carbon atoms in the chain, and may have from 1 to 4 carbon atoms, or 1 to 2 carbon atoms. Thus, representative examples of lower alkyl radicals include methyl, ethyl, n-propyl, n-butyl, n-pentyl, n-hexyl, isopropyl, isobutyl, isopentyl, amyl (C₅H₁₁), sec-butyl, tert-butyl, sec-amyl, tert-pentyl, 2-ethylbutyl, 2,3-dimethylbutyl, and the like. ‘Higher alkyl’ refers to alkyls of 7 carbons and above, including n-heptyl, n-octyl, n-nonyl, n-decyl, n-dodecyl, n-tetradecyl, n-hexadecyl, n-octadecyl, n-eicosyl, and the like, along with branched variations thereof. A linear carbon chain of say 4 to 6 carbons would refer to the chain length not including any carbons residing on a branch, whereas in a branched chain it would refer to the total number. Optional substituents for alkyl and other groups are described herein.

The term ‘alkoxy’ or ‘alkoxyl’ as used herein refers to a monovalent radical of the formula RO—, where R is any alkyl, alkenyl or alkynyl as defined herein. Alkoxyl groups may be optionally substituted by any of the optional substituents described herein. ‘Lower alkoxyl’ has the formula RO—, where the R group is a lower alkyl, alkenyl or alkynyl. Representative alkoxy radicals include methoxy, ethoxy, n-propoxy, n-butoxy, n-pentyloxy, n-hexyloxy, isopropoxy, isobutoxy, isopentyloxy, amyloxy, sec-butoxy, tert-butoxy, tert-pentyloxy, and the like. Particularly exemplary alkoxyl groups are methoxyl and ethoxyl.

The term ‘cycloalkyl’ as used herein refers to a cyclized alkyl ring having the indicated number of carbon atoms in a specified range. Thus, for example, ‘C₃-C₆ cycloalkyl’ encompasses each of cyclopropyl, cyclobutyl, cyclopentyl, and cyclohexyl.

The term ‘aryl’ as used herein refers to a substituted or unsubstituted aromatic carbocyclic radical containing from 5 to about 15 carbon atoms (‘C6-C15 aryl’); and preferably 6 to 12 carbon atoms (‘C6-C12 aryl’). An aryl group may have only one individual carbon ring, or may comprise one or more fused rings in which at least one ring is aromatic in nature. A ‘phenyl’ is a radical formed by removal of a hydrogen atom from a benzene ring, and may be substituted or unsubstituted. A ‘phenoxyl’ group, therefore, is a radical of the formula RO—, wherein R is a phenyl radical. ‘Benzyl’ is a radical of the formula R—CH2-, wherein R is phenyl, and ‘benzyloxy’ is a radical of the formula RO—, wherein R is benzyl. The point of attachment to the base molecule on such fused aryl ring systems may be a C atom of the aromatic portion or a C or a N atom of the non-aromatic portion of the ring system. Non-limiting examples of aryl radicals include, phenyl, naphthyl, anthracenyl, benzyl, biphenyl, furanyl, pyridinyl, indanyl, anthraquinolyl, tetrahydronaphthyl, a benzoic acid radical, a furan-2-carboxylic acid radical, and the like.

The term ‘cycloaryl’ herein refers to a polycyclic group wherein an aryl group is fused to a 5- or 6-membered aliphatic ring. For example, C₆-C₁₂ cycloaryl means a C₆-C₁₂ aryl fused to a 5- or 6-membered aliphatic ring.

The term ‘heteroaryl’ as used herein refers to (i) a 5- or 6-membered ring having the characteristics of aromaticity containing at least one heteroatom selected from N, O and S, wherein each N is optionally in the form of an oxide, and (ii) a 9- or 10-membered bicyclic fused ring system, wherein the fused ring system of (ii) contains at least one heteroatom independently selected from N, O and S, wherein each ring in the fused ring system contains zero, one or more than one heteroatom, at least one ring is aromatic, each N is optionally in the form of an oxide, and each S in a ring which is not aromatic is optionally S(O) or S(O)₂. Typically, heteroaryl groups contain 5 to 14 ring atoms (‘5-14 membered heteroaryl’), and preferably 5 to 12 ring atoms (‘5-12 membered heteroaryl’). Heteroaryl rings are attached to the base molecule via a ring atom of the heteroaromatic ring, such that aromaticity is maintained. Suitable 5- and 6-membered heteroaromatic rings include, for example, pyridyl, 3-fluroropyridyl, 4-fluoropyridyl, 3-methoxypyridyl, 4-methoxypyridyl, pyrrolyl, pyrazinyl, pyrimidinyl, pyridazinyl, triazinyl, thienyl, furanyl, imidazolyl, pyrazolyl, triazolyl (i.e., 1,2,3-triazolyl or 1,2,4-triazolyl), tetrazolyl, oxazolyl, isooxazolyl, oxadiazolyl (i.e., the 1,2,3-, 1,2,4-, 1,2,5-(furazanyl), or 1,3,4-isomer), oxatriazolyl, thiazolyl, isothiazolyl, and thiadiazolyl. Suitable 9- and 10-membered heterobicyclic, fused ring systems include, for example, benzofuranyl, indolyl, indazolyl, naphthyridinyl, isobenzofuranyl, benzisoxazolyl, benzoxazolyl, benzothiazolyl, chromenyl, quinolinyl, isoquinolinyl, benzopiperidinyl, benzofuranyl, imidazo[1,2-a]pyridinyl, benzotriazolyl, indazolyl, indolinyl, and isoindolinyl.

The term ‘heteroaryloxy’ or ‘heteroaryloxyl’ as used herein refers to an —O— heteroaryl group.

The terms ‘heterocycle’ or ‘heterocyclic’ group or ‘heterocyclyl’ as used herein refer to a monovalent radical of from about 4- to about 15-ring atoms, and preferably 3-, 4-, 5-, 6-, 7-, 8-, 9- or 10-ring members. Generally, the heterocyclic group contains one, two or three heteroatoms, selected independently from nitrogen, oxygen and sulphur. A preferred heteroatom is N. A heterocyclic group may have only one individual ring or may comprise more than one fused rings in which at least one ring contains a heteroatom. It may be fully saturated or partially saturated and may be substituted or unsubstituted as in the case or aryl and heteroaryl groups.

Representative examples of unsaturated 5-membered heterocycles with only one heteroatom include 2- or 3-pyrrolyl, 2- or 3-furanyl, and 2- or 3-thiophenyl. Corresponding partially saturated or fully saturated radicals include 3-pyrrolin-2-yl, 2- or 3-pyrrolindinyl, 2- or 3-tetrahydrofuranyl, and 2- or 3-tetrahydrothiophenyl. Representative unsaturated 5-membered heterocyclic radicals having two heteroatoms include imidazolyl, oxazolyl, thiazolyl, pyrazolyl, and the like. The corresponding fully saturated and partially saturated radicals are also included. Representative examples of unsaturated 6-membered heterocycles with only one heteroatom include 2-, 3-, or 4-pyridinyl, 2H-pyranyl, and 4H-pyranyl. Corresponding partially saturated or fully saturated radicals include 2-, 3-, or 4-piperidinyl, 2-, 3-, or 4-tetrahydropyranyl and the like. Representative unsaturated 6-membered heterocyclic radicals having two heteroatoms include 3- or 4-pyridazinyl, 2-, 4-, or 5-pyrimidinyl, 2-pyrazinyl, morpholino, and the like. The corresponding fully saturated and partially saturated radicals are also included, e.g. 2-piperazine. The heterocyclic radical is bonded through an available carbon atom or heteroatom in the heterocyclic ring directly to the entity. In some embodiments, where indicated, the heterocyclic group may be bonded to the entity through a linker such as an alkylene such as methylene or ethylene.

The term ‘substituted’ means that one or more hydrogen atoms (attached to a carbon or heteroatom) is replaced with a selection from the indicated group of substituents, provided that the designated atom's normal valency under the existing circumstances is not exceeded. The group may be optionally substituted with particular substituents at positions that do not significantly interfere with the preparation of compounds falling within the scope of this invention and on the understanding that the substitution(s) does not significantly adversely affect the biological activity or structural stability of the compound. Combinations of substituents are permissible only if such combinations result in stable compounds. By ‘stable compound’ or ‘stable structure’, it is meant a compound that is sufficiently robust to survive isolation to a useful degree of purity from a reaction mixture and/or formulation into an efficacious therapeutic agent. The term ‘optionally substituted’ or ‘optional substituents’ as used herein means that the groups in question are either unsubstituted or substituted with one or more of the substituents specified. When the groups in question are substituted with more than one substituent, the substituents may be the same or different. Furthermore, the terms ‘independently’, ‘independently are’, and ‘independently selected from’ mean that the substituents in question may be the same or different.

The term ‘deuterium’ as used herein refers to an isotope of hydrogen that has one proton and one neutron in its nucleus and that has twice the mass of ordinary hydrogen. Deuterium herein is represented by the symbol ‘D’. The term ‘deuterated’ by itself or used to modify a compound or group as used herein refers to the presence of at least one deuterium atom attached to carbon. For example, the term ‘deuterated compound’ refers to a compound which contains one or more carbon-bound deuterium(s). In a deuterated compound of the present disclosure, when a particular position is designated as having deuterium, it is understood that the abundance of deuterium at that position is substantially greater than the natural abundance of deuterium, which is about 0.015%. The term ‘undeuterated’ or ‘non-deuterated’ as used herein refers to the ratio of deuterium atoms of which is not more than the natural isotopic deuterium content, which is about 0.015%; in other words, all hydrogen are present at their natural isotopic percentages. Unless otherwise stated, when a position is designated specifically as ‘H’ or ‘hydrogen’, the position is understood to have hydrogen at its natural abundance isotopic composition.

The term ‘isotopic enrichment factor’ as used herein refers to the ratio between the isotope abundance and the natural abundance of a specified isotope.

The term ‘isotopologue’ as used herein refers to a species in which the chemical structure differs from a specific compound of the invention only in the isotopic composition thereof.

The term ‘substantially free of other stereoisomers’ as used herein means less than 10% of other stereoisomers, preferably less than 5% of other stereoisomers, more preferably less than 2% of other stereoisomers and most preferably less than 1% of other stereoisomers are present.

The term ‘pharmaceutically acceptable salt’ as used herein refers to a salt that is not biologically or otherwise undesirable (e.g., not toxic or otherwise harmful). A salt of a compound of the invention is formed between an acid and a basic group of the compound, or a base and an acidic group of the compound. For example, when the compounds of the invention contain at least one basic group (i.e., groups that can be protonated), the invention includes the compounds in the form of their acid addition salts with organic or inorganic acids such as, for example, but not limited to salts with hydrogen chloride, hydrogen bromide, phosphoric acid, sulfuric acid, nitric acid, benzenesulfonic acid, acetic acid, citric acid, glutamic acid, lactic acid, and methanesulfonic acid. When compounds of the invention contain one or more acidic groups (e.g., a carboxylic acid), the invention includes the pharmaceutically acceptable salts of the compounds formed with but not limited to alkali metal salts, alkaline earth metal salts or ammonium salts. Examples of such salts include, but are not limited to, sodium salts, potassium salts, calcium salts, magnesium salts or salts with ammonia or organic amines such as, for example, ethylamine, ethanolamine, triethanolamine or amino acids. Additional examples of such salts can be found in Stahl, P. H. et al. Pharmaceutical Salts: Properties, Selection, and Use, 2nd Revised Edition, Wiley, 2011.

The terms ‘treatment’, ‘treating’ and ‘treat’ as used herein, include their generally accepted meanings, i.e., the management and care of a patient for the purpose of preventing, reducing the risk in incurring or developing a given condition or disease, prohibiting, restraining, alleviating, ameliorating, slowing, stopping, delaying, or reversing the progression or severity, and holding in check existing characteristics of a disease, disorder, or pathological condition, including the alleviation or relief of symptoms or complications, or the cure or elimination of the disease, disorder, or condition.

The term ‘therapeutically effective amount’ as used herein refers to that amount of compound of the invention that will elicit the biological or medical response of a tissue, system, animal, or human that is being sought by a researcher, veterinarian, medical doctor or other. As will be recognized by a person of ordinary skill in the art, a therapeutically effective amount of the compounds of the invention will vary and will depend on the disease treated, the severity of the disease, the route of administration, and the gender, age, and general health condition of the subject to whom the compound is being administered. The therapeutically effective amount may be administered as a single dose once a day, or as split doses administered multiple (e.g., two, three or four) times a day. The therapeutically effective amount may also be administered through continuous dosing, such as through infusion or with an implant.

Unless defined otherwise, ‘room temperature’ is intended to mean a temperature of from about 18 to 28° C., typically between about 18 and 25° C., and more typically between about 18 and 22° C. As used herein, the phrase ‘room temperature’ may be shortened to ‘rt’ or ‘RT’.

### Compounds:

Disclosed herein is a compound having the structural Formula 1:

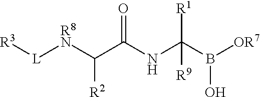

or a pharmaceutically acceptable salt, solvate, stereoisomer or mixture of stereoisomers, tautomer, isotopic form, pharmaceutically active metabolite thereof, or combinations thereof, wherein:
R¹ is selected from the group consisting of: deuterium, C1-C4 alkyl, C1-C4 alkoxyl, C1-C4 oxoalkyl, C1-C5 alkyl-alkoxyl, wherein each alkyl, oxoalkyl or alkoxyl is optionally substituted with C3-C6 cycloalkyl, phenyl, phenoxy, or a 5- or 6-membered heteroaryl, wherein said phenyl, phenoxy, or heteroaryl are each optionally substituted with one or more substituent selected from deuterium, halogen, hydroxyl, CN, CO2H, CO2R¹⁰, CONR¹⁰R¹¹, NR¹⁰R¹¹, SR¹⁰, SO2NR¹⁰R¹¹, C1-C4 alkyl, C1-C4 haloalkyl, C1-C4 alkoxy, phenyl, or a 5- or 6-membered heteroaryl;
R² is (C1-C4 alkyl)-C(O)NR⁴R⁵;
R⁴ and R⁵ are each independently selected from hydrogen, deuterium, C1-C4 alkyl; C1-C4 haloalkyl, C1-C5 alkyl-alkoxyl or R⁴ and R⁵ together with the N to which they are attached form 5 or 6 membered heterocyclic ring optionally having one or more additional heteroatoms selected from N, O and S, wherein the 5 or 6 membered heterocyclic ring is optionally substituted with one or more substituent selected from deuterium, halogen, hydroxyl, oxo, CN, C1-C4 alkyl, C1-C4 haloalkyl or C1-C4 alkoxyl;
L is C(O), C(O)O, C(O)NR⁶, S(O)₂, or a bond;
R³ is C₁-C₄ alkyl optionally substituted with one or more substituents each independently selected from the group consisting of deuterium, halogen, cyano, hydroxyl, C₁-C₄ alkoxyl, 5 or 6 membered aryl (e.g. phenyl) or 5 or 6 membered heteroaryl; or
R³ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, oxo, C₁-C₄ alkoxyl, or C₁-C₄ alkyl that is optionally substituted with one to three substituents selected from deuterium, halogen, cyano, hydroxyl, or C₁-C₄ alkoxyl; or
R³ is aryl or heteroaryl having one or more heteroatoms selected from N, O and S, wherein aryl or heteroaryl is optionally substituted with one or more substituents selected from deuterium, halogen, cyano, hydroxyl, OR, CO2H, CO2R¹⁰, CONR¹⁰R¹¹, NR¹⁰R¹¹, SR¹⁰, SO2NR¹⁰R¹¹, C₁-C₄ alkoxyl, or C₁-C₄ alkyl that is optionally substituted with one to three substituents selected from deuterium, halogen, cyano, hydroxyl, or C₁-C₄ alkoxyl;
R⁶ is hydrogen or C₁-C₄ alkyl optionally substituted with one or more of halogen, hydroxyl and phenyl, wherein phenyl is optionally substituted with one or more substituent selected from halogen, hydroxyl and C1-C2 alkyl;
R⁷ is hydrogen, or R⁷ and R¹, together with the boron atom to which R⁷ is attached form a 5-membered heteroalkyl ring;
R^{a} is selected from hydrogen, deuterium or C₁-C₂ alkyl optionally substituted with one or more substituents each independently selected from the group consisting of deuterium, halogen, hydroxyl, cyano, methoxyl and phenyl;
R⁹ is selected from hydrogen, deuterium or C₁-C₂ alkyl; and
R¹⁰ and R¹¹ are each independently selected from hydrogen, deuterium, C1-C4 alkyl; C1-C4 haloalkyl, C1-C5 alkyl-alkoxyl, C3-C7 cycloalkyl, or R¹⁰ and R¹¹ together with the N to which they are attached form 3 to 7 membered heterocyclic ring optionally having one or more additional heteroatoms selected from N, O and S, wherein the C3-C7 cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl.

In embodiments, R¹ is methyl, ethyl, n-propyl, i-propyl, n-butyl or tert-butyl, each optionally substituted with a phenyl ring. In some embodiments R¹ is suitably selected from methyl, n-propyl, n-butyl or tert-butyl. In various embodiments R¹ is selected from phenyl-(CH₂)— or phenyl-(CH₂)₃—. In certain embodiments, R¹ is methyl. In certain embodiments, R¹ is methyl substituted with a phenyl ring. In certain embodiments, R¹ is ethyl. In certain embodiments, R¹ is ethyl substituted with a phenyl ring. In certain embodiments, R¹ is n-propyl. In certain embodiments, R¹ is n-propyl substituted with a phenyl ring. In certain embodiments, R¹ is tert-butyl. In certain embodiments, R¹ is n-butyl. In certain embodiments, R¹ is methoxymethyl optionally substituted with a phenyl ring. In embodiments, R¹ is CO2H. In embodiments, R¹ is CO2R¹⁰. In embodiments, R¹ is CONR¹⁰R¹¹. In embodiments, R¹ is NR¹⁰R¹¹. In embodiments, R¹ is SR¹⁰. In embodiments, R¹ is SO2NR¹⁰R¹¹.

In embodiments, R² is —CH₂—C(O)NR⁴R⁵. In such embodiments, one of R⁴ and R⁵ may be C₁-C₂ alkyl, or in other embodiments both of R⁴ and R⁵ may be C₁-C₂ alkyl. In various embodiments, one of R⁴ and R⁵ is methyl, or both of R⁴ and R⁵ are methyl. In various embodiments, one of R⁴ and R⁵ is hydrogen; or both of R⁴ and R⁵ are hydrogen. In various embodiments, one of R⁴ and R⁵ is hydrogen and the other of R⁴ and R⁵ is ethyl.

In alternative embodiments, NR⁴R⁵ is selected from a 5- or 6-membered heterocycloalkyl group having one or two heteroatoms. In various such embodiments, NR⁴R⁵ may be selected from N-pyrrolidinyl, N-morpholinyl and N-piperidinyl.

In various embodiments, R⁴ and R⁵ together with the N to which they are attached form 5 or 6 membered heterocyclic ring optionally having one or more additional heteroatoms selected from N, O and S, wherein the 5 or 6 membered heterocyclic ring is optionally substituted and wherein two adjacent substituents join together to form a 5 or 6 membered aryl or heterocyclic ring optionally having one or more additional heteroatoms selected from N, O and S, wherein the 5 or 6 membered aryl heterocyclic ring is optionally substituted with one or more substituent selected from deuterium, halogen, hydroxyl, CN, C₁-C₄ alkyl, C₁-C₄ haloalkyl or C₁-C₄ alkoxyl.

In embodiments, NR⁴R⁵ is selected from a 5- or 6-membered heterocycloalkyl group having one or two heteroatoms. In embodiments, NR⁴R⁵ is a 5- or 6-membered heterocycloalkyl group, wherein two adjacent substituents join together to form a 5- or 6-membered aryl or heterocycloalkyl group. In embodiments, NR⁴R⁵ is selected from a 5-membered heterocycloalkyl group and wherein two adjacent substituents join together to form a 6-membered aryl group. N-pyrrolidinyl, N-morpholinyl and N-piperidinyl. In embodiments, the 5-membered heterocycloalkyl group is N-pyrrolidinyl. In embodiments, the 6-membered aryl group is phenyl. In embodiments, NR⁴R⁵ is N-isoindolinyl.

In embodiments, L is selected from C(O), C(O)O, C(O)NH, C(O)N(CH₃) or SO₂. Suitably, L may be selected from C(O), C(O)O and C(O)NH. In certain embodiments, L is C(O). In certain embodiments, L is a bond. In certain embodiments, L is C(O)O. In certain embodiments, L is C(O)NR⁶. In certain embodiments, L is SO₂.

In embodiments, R³ is selected from C₁-C₄ alkyl, a 5- or 6-membered heteroaryl, C₆ aryl, a 5- or 6-membered heterocycloalkyl and C6 cycloalkyl. R³ is optionally substituted. In some embodiments, R³ is selected from methyl, ethyl, n-propyl, i-propyl, n-butyl or tert-butyl, each optionally substituted with a phenyl ring. In various embodiments, R³ is selected from methyl, i-propyl and tert-butyl. In some suitable embodiments, R³ is selected from phenyl, phenyl-(CH₂)— and phenyl-(CH₂)₂—, wherein the phenyl group is optionally substituted. In particular embodiments, R³ may be selected from an aryl, heteroaryl, cycloalkyl or heterocycloalkyl selected from tetrahydropyranyl, pyrazinyl, tetrahydropyrrolyl, tetrahydrofuranyl, tetrahydropyranyl, cyclohexanyl, oxazolyl and morpholinyl, wherein said aryl, heteroaryl, cycloalkyl or heterocycloalkyl is optionally substituted. In such embodiments, R³ may be selected from n-tetrahydropyrrolyl, and n-morpholinyl. In any such embodiments, the substituent on said R³ group may be selected from one to three of halogen, hydroxyl and C1-C2 alkyl. In particular, the substituent may be selected from one or two of Cl, hydroxyl and methyl. In some embodiments, R³ is selected from 2-chlorophenyl, 3-chlorophenyl, 2,5-dichlorophenyl, 2,4-dimethyloxazolyl, and 3-hydroxy n-tetrahydropyrrolyl.

In certain embodiments, R³ may be C₁-C₄ alkyl optionally substituted with one or more substituents independently selected from the groups consisting of fluoro, chloro, cyano, or methoxyl; or R³ may be cycloalkyl, heterocyclyl, aryl, cycloaryl, or heteroaryl, any of which is optionally substituted with one or more fluoro, chloro, cyano, methoxyl, or C₁-C₄ alkyl that is optionally substituted with one to three fluoro, chloro, cyano, or methoxyl. In certain embodiments, R³ is C₁-C₄ alkyl optionally substituted with one or more substituents each independently selected from the groups consisting of fluoro, chloro, cyano, or methoxyl. In certain embodiments, R³ is cycloalkyl which is optionally substituted with one or more fluoro, chloro, cyano, methoxyl, or C₁—C₄ alkyl that is optionally substituted with one to three fluoro, chloro, cyano, or methoxyl. In certain embodiments, R³ is heterocyclyl which is optionally substituted with one or more fluoro, chloro, cyano, methoxyl, or C₁-C₄ alkyl that is optionally substituted with one to three fluoro, chloro, cyano, or methoxyl. In certain embodiments, R³ is aryl which is optionally substituted with one or more fluoro, chloro, cyano, methoxyl, or C₁-C₄ alkyl that is optionally substituted with one to three fluoro, chloro, cyano, or methoxyl. In certain embodiments, R³ is cycloaryl which is optionally substituted with one or more fluoro, chloro, cyano, methoxyl, or C₁-C₄ alkyl that is optionally substituted with one to three fluoro, chloro, cyano, or methoxylln certain embodiments, R³ is heteroaryl which is optionally substituted with one or more fluoro, chloro, cyano, methoxyl, or C₁-C₄ alkyl that is optionally substituted with one to three fluoro, chloro, cyano, or methoxyl. In certain embodiments, R³ is methyl, tert-butyl, trifluoromethyl, phenyl optionally substituted with one or more fluoro, chloro, cyano, methoxyl, or C₁-C₄ alkyl that is optionally substituted with one to three fluoro, chloro, cyano, or methoxyl; pyridinyl optionally substituted with one or more fluoro, chloro, cyano, methoxyl, or C₁-C₄ alkyl that is optionally substituted with one to three fluoro, chloro, cyano, or methoxyl; piperidinyl optionally substituted with one or more fluoro, chloro, cyano, methoxyl, or C₁-C₄ alkyl that is optionally substituted with one to three fluoro, chloro, cyano, or methoxyl; pyrrolidinyl optionally substituted with one or more fluoro, chloro, cyano, methoxyl, or C₁-C₄ alkyl that is optionally substituted with one to three fluoro, chloro, cyano, or methoxyl; imidazolyl optionally substituted with one or more fluoro, chloro, cyano, methoxyl, or C₁-C₄ alkyl that is optionally substituted with one to three fluoro, chloro, cyano, or methoxyl; pyrazolyl optionally substituted with one or more fluoro, chloro, cyano, methoxyl, or C₁-C₄ alkyl that is optionally substituted with one to three fluoro, chloro, cyano, or methoxyl; thiazolyl optionally substituted one or more fluoro, chloro, cyano, methoxyl, or C₁-C₄ alkyl that is optionally substituted with one to three fluoro, chloro, cyano, or methoxyl; pyrazinyl optionally substituted with one or more fluoro, chloro, cyano, methoxyl, or C₁-C₄ alkyl that is optionally substituted with one to three fluoro, chloro, cyano, or methoxyl; oxazolyl optionally substituted with one or more fluoro, chloro, cyano, methoxyl, or C₁-C₄ alkyl that is optionally substituted with one to three fluoro, chloro, cyano, or methoxyl; or morpholinyl optionally substituted with one or more fluoro, chloro, cyano, methoxyl, or C₁-C₄ alkyl that is optionally substituted with one to three fluoro, chloro, cyano, or methoxyl.

In certain embodiments, R³ is methyl. In certain embodiments, R³ is tert-butyl. In certain embodiments, R³ is trifluoromethyl. In certain embodiments, R³ is phenyl optionally substituted with one or more fluoro, chloro, cyano, methoxyl, or C₁-C₄ alkyl that is optionally substituted with one to three fluoro, chloro, cyano, or methoxyl. In certain embodiments, R³ is pyridinyl optionally substituted with one or more fluoro, chloro, cyano, methoxyl, or C₁-C₄ alkyl that is optionally substituted with one to three fluoro, chloro, cyano, or methoxyl. In certain embodiments, R³ is piperidinyl optionally substituted with one or more fluoro, chloro, cyano, methoxyl, or C₁-C₄ alkyl that is optionally substituted with one to three fluoro, chloro, cyano, or methoxyl. In certain embodiments, R³ is pyrrolidinyl optionally substituted with one or more fluoro, chloro, cyano, methoxyl, or C₁-C₄ alkyl that is optionally substituted with one to three fluoro, chloro, cyano, or methoxyl. In certain embodiments, R³ is imidazolyl optionally substituted with one or more fluoro, chloro, cyano, methoxyl, or C₁-C₄ alkyl that is optionally substituted with one to three fluoro, chloro, cyano, or methoxyl. In certain embodiments, R³ is pyrazolyl optionally substituted with one or more fluoro, chloro, cyano, methoxyl, or C₁-C₄ alkyl that is optionally substituted with one to three fluoro, chloro, cyano, or methoxyl. In certain embodiments, R³ is thiazolyl optionally substituted with one or more fluoro, chloro, cyano, methoxyl, or C₁-C₄ alkyl that is optionally substituted with one to three fluoro, chloro, cyano, or methoxyl. In certain embodiments, R³ is pyrazinyl optionally substituted with one or more fluoro, chloro, cyano, methoxyl, or C₁-C₄ alkyl that is optionally substituted with one to three fluoro, chloro, cyano, or methoxyl. In certain embodiments, R³ is oxazolyl optionally substituted with one or more fluoro, chloro, cyano, methoxyl, or C₁-C₄ alkyl that is optionally substituted with one to three fluoro, chloro, cyano, or methoxyl. In certain embodiments, R³ is morpholinyl optionally substituted with one or more fluoro, chloro, cyano, methoxyl, or C₁-C₄ alkyl that is optionally substituted with one to three fluoro, chloro, cyano, or methoxyl.

In embodiments, R³ is an 8, 9 or 10 membered bicyclic heteroaryl, aryl, saturated or unsaturated heterocycloalkyl or saturated or unsaturated cycloalkyl. In such embodiments, R³ is optionally substituted. In various embodiment, R³ may be selected from indolyl, isoindolyl, indolinyl, isoindolinyl, indolizinyl, benzimidazolyl, purinyl, quinolinyl, isoquinolinyl, quinazolinyl, or pteridinyl. In embodiments, R³ is isoindolinyl, such as N-isoindolinyl. Such may be optionally substituted with one or more fluoro, chloro, cyano, methoxyl, or C₁-C₄ alkyl that is optionally substituted with one to three fluoro, chloro, cyano, or methoxyl.

In embodiments, R³ is CO2H. In embodiments, R³ is CO2R¹⁰. In embodiments, R³ is CONR¹⁰R¹¹. In embodiments, R³ is NR¹⁰R¹¹. In embodiments, R³ is SR¹⁰. In embodiments, R³ is SO2NR¹⁰R¹¹.

In embodiments, R¹⁰ and R¹¹ are each independently selected from hydrogen, deuterium, C1-C4 alkyl; C1-C4 haloalkyl or C1-C5 alkyl-alkoxyl. In embodiments, R¹⁰ and R¹¹ are each independently selected from hydrogen, deuterium, C1-C4 alkyl; C1-C4 haloalkyl, C1-C5 alkyl-alkoxyl or C3-C7 cycloalkyl. In embodiments, R¹⁰ and R¹¹ are each independently selected from hydrogen, deuterium, C1-C2 alkyl; C1-C2 haloalkyl, C1-C2 alkyl-alkoxyl or C3-C5 cycloalkyl. In embodiments, R¹⁰ and R¹¹ together with the N to which they are attached form 3 to 7 membered heterocyclic ring optionally having one or more additional heteroatoms selected from N, O and S.

In any such embodiments, the C3-C7 cycloalkyl, C3-C5 cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl. In embodiments, the substituents may be selected from one, two or three of deuterium, F, C1, hydroxyl, oxo, CN, C1-C2 alkyl, C1-C2 haloalkyl or C1-C2 alkoxyl.

In embodiments, R⁶ is selected from hydrogen and methyl.

In embodiments, R⁷ is hydrogen.

In embodiments, R⁸ is selected from hydrogen, phenyl-(CH₂)— and phenyl-(CH₂)₂—.

In embodiments, R⁹ is selected from hydrogen or C1-C2 alkyl. In embodiments, R⁹ is hydrogen.

In embodiments, R⁹ is deuterium. In embodiments, R⁹ is C1-C2 alkyl. In other embodiments, In embodiments, R⁹ is methyl. In other embodiments, R⁹ is ethyl.

In any of the aspects and embodiments disclosed herein, halogen may suitably be selected from fluoro or chloro. In particular, in any such embodiments, halogen may be chloro.

In another embodiment, the present invention is directed to a compound, or a pharmaceutically acceptable salt thereof, represented by any one of the following structures:

TABLE 1
Chemical structures of compounds according to the disclosure. Any of these compounds may
also exist in the isomeric forms, particularly the oxaborolane isomer derivative, and such
isomers are explicitly intended to be encompassed within the scope of this disclosure.

| | |
|---|---|
| 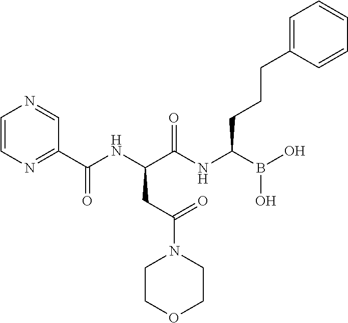 [SMILES: O=C(N[C@H](CC(=O)N1CCOCC1)C(=O)N[C@@H](CCCc1ccccc1)B(O)O)c1cnccn1] | Structure 1 |
| 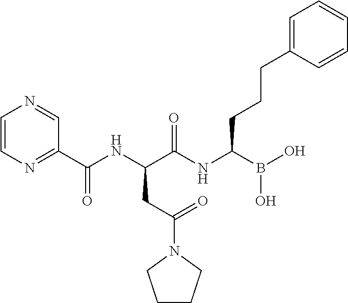 [SMILES: O=C(N[C@H](CC(=O)N1CCCC1)C(=O)N[C@@H](CCCc1ccccc1)B(O)O)c1cnccn1] | Structure 2 |
| 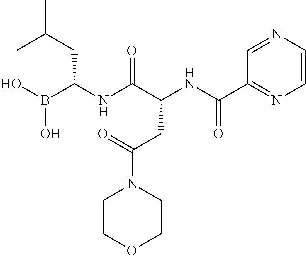 [SMILES: CC(C)C[C@H](NC(=O)[C@@H](CC(=O)N1CCOCC1)NC(=O)c1cnccn1)B(O)O] | Structure 3 |
| 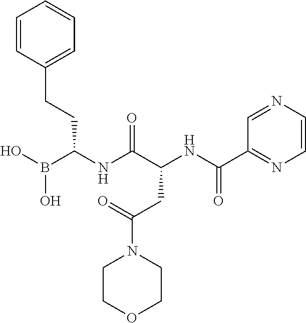 [SMILES: O=C(N[C@H](CC(=O)N1CCOCC1)C(=O)N[C@@H](CCc1ccccc1)B(O)O)c1cnccn1] | Structure 4 |
| 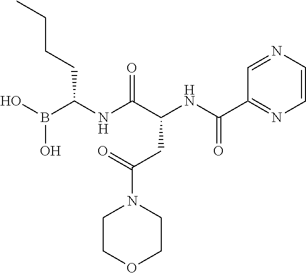 [SMILES: CCCC[C@H](NC(=O)[C@@H](CC(=O)N1CCOCC1)NC(=O)c1cnccn1)B(O)O] | Structure 5 |
| 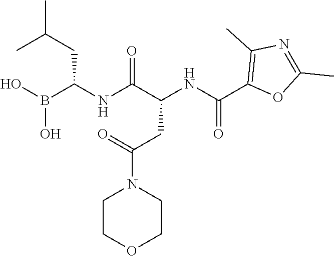 [SMILES: Cc1nc(C)c(C(=O)N[C@H](CC(=O)N2CCOCC2)C(=O)N[C@@H](CC(C)C)B(O)O)o1] | Structure 6 |
| 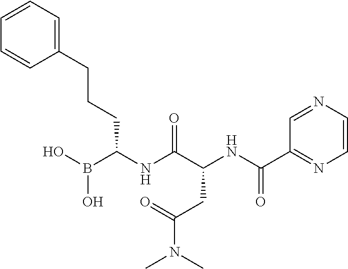 [SMILES: CN(C)C(=O)C[C@@H](NC(=O)c1cnccn1)C(=O)N[C@@H](CCCc1ccccc1)B(O)O] | Structure 7 |
| 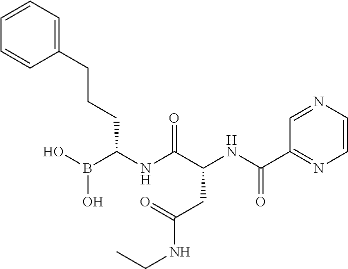 [SMILES: CCNC(=O)C[C@@H](NC(=O)c1cnccn1)C(=O)N[C@@H](CCCc1ccccc1)B(O)O] | Structure 8 |
| 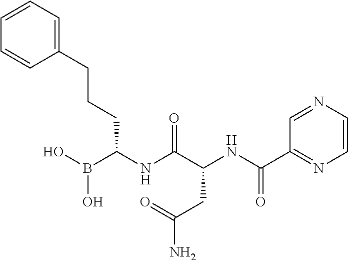 [SMILES: NC(=O)C[C@@H](NC(=O)c1cnccn1)C(=O)N[C@@H](CCCc1ccccc1)B(O)O] | Structure 9 |
| 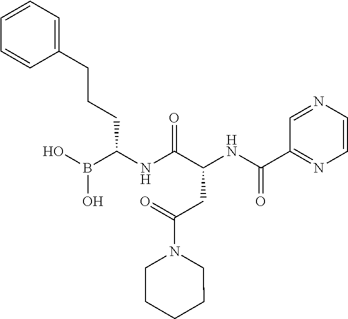 [SMILES: O=C(N[C@H](CC(=O)N1CCCCC1)C(=O)N[C@@H](CCCc1ccccc1)B(O)O)c1cnccn1] | Structure 10 |
| 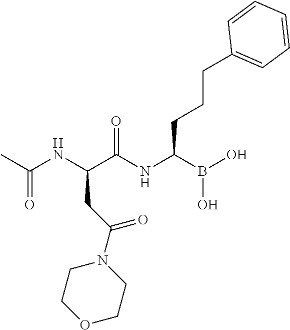 [SMILES: CC(=O)N[C@H](CC(=O)N1CCOCC1)C(=O)N[C@@H](CCCc1ccccc1)B(O)O] | Structure 11 |
| 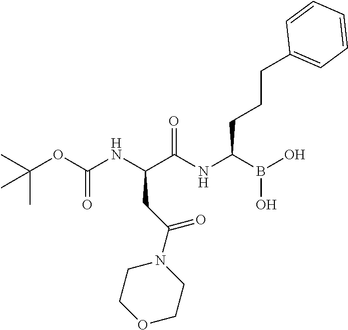 [SMILES: CC(C)(C)OC(=O)N[C@H](CC(=O)N1CCOCC1)C(=O)N[C@@H](CCCc1ccccc1)B(O)O] | Structure 12 |
| 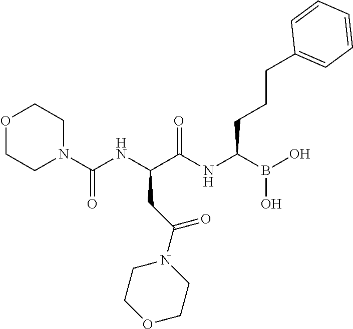 [SMILES: O=C(N[C@@H](CCCc1ccccc1)B(O)O)[C@@H](CC(=O)N1CCOCC1)NC(=O)N1CCOCC1] | Structure 13 |
| 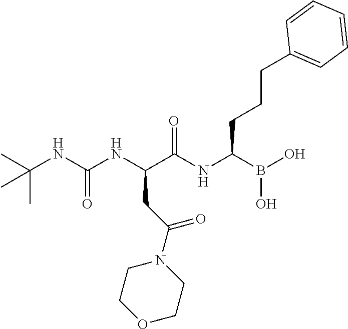 [SMILES: CC(C)(C)NC(=O)N[C@H](CC(=O)N1CCOCC1)C(=O)N[C@@H](CCCc1ccccc1)B(O)O] | Structure 14 |
| 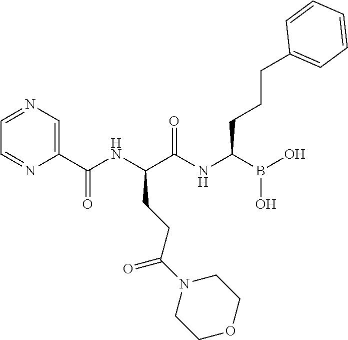 [SMILES: O=C(N[C@H](CCC(=O)N1CCOCC1)C(=O)N[C@@H](CCCc1ccccc1)B(O)O)c1cnccn1] | Structure 15 |
| 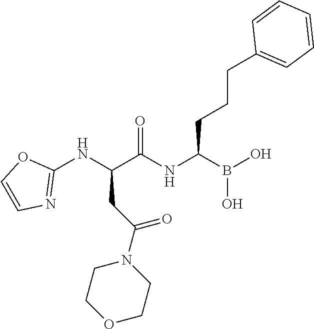 [SMILES: O=C(N[C@@H](CCCc1ccccc1)B(O)O)[C@@H](CC(=O)N1CCOCC1)Nc1ncco1] | Structure 16 |
| 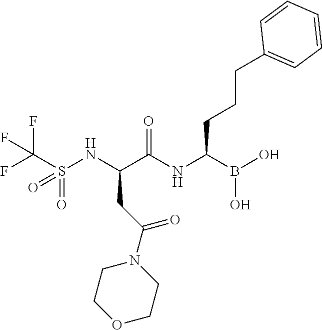 [SMILES: O=C(N[C@@H](CCCc1ccccc1)B(O)O)[C@@H](CC(=O)N1CCOCC1)NS(=O)(=O)C(F)(F)F] | Structure 17 |
| 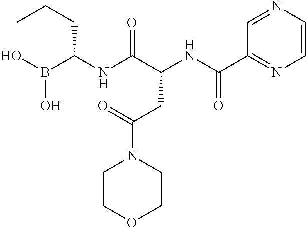 [SMILES: CCC[C@H](NC(=O)[C@@H](CC(=O)N1CCOCC1)NC(=O)c1cnccn1)B(O)O] | Structure 18 |
| 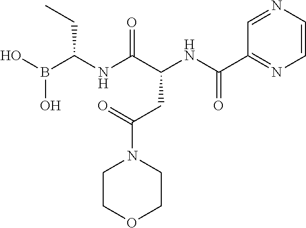 [SMILES: CC[C@H](NC(=O)[C@@H](CC(=O)N1CCOCC1)NC(=O)c1cnccn1)B(O)O] | Structure 19 |
| 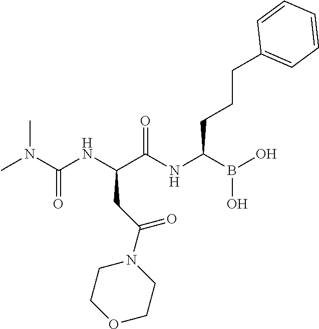 [SMILES: CN(C)C(=O)N[C@H](CC(=O)N1CCOCC1)C(=O)N[C@@H](CCCc1ccccc1)B(O)O] | Structure 20 |
| 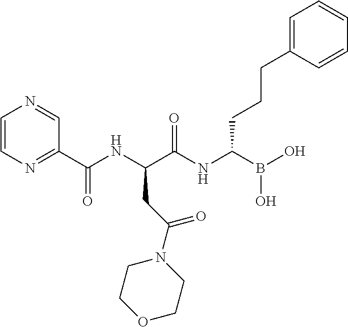 [SMILES: O=C(N[C@H](CC(=O)N1CCOCC1)C(=O)N[C@H](CCCc1ccccc1)B(O)O)c1cnccn1] | Structure 21 |
| 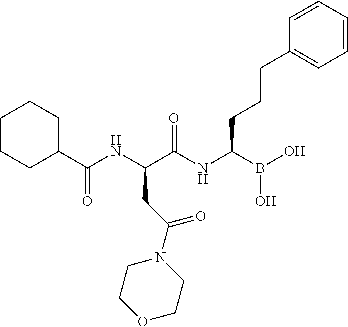 [SMILES: O=C(N[C@H](CC(=O)N1CCOCC1)C(=O)N[C@@H](CCCc1ccccc1)B(O)O)C1CCCCC1] | Structure 22 |
| 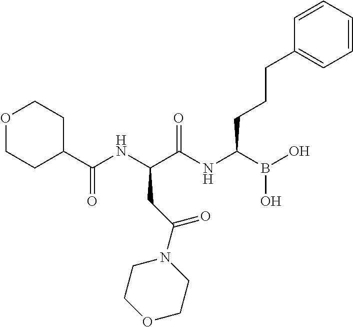 [SMILES: O=C(N[C@H](CC(=O)N1CCOCC1)C(=O)N[C@@H](CCCc1ccccc1)B(O)O)C1CCOCC1] | Structure 23 |
| 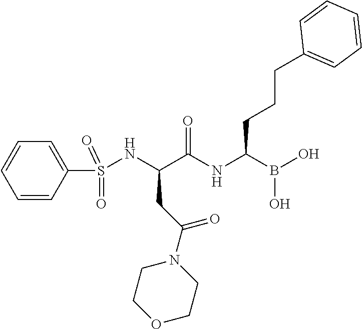 [SMILES: O=C(N[C@@H](CCCc1ccccc1)B(O)O)[C@@H](CC(=O)N1CCOCC1)NS(=O)(=O)c1ccccc1] | Structure 24 |
| 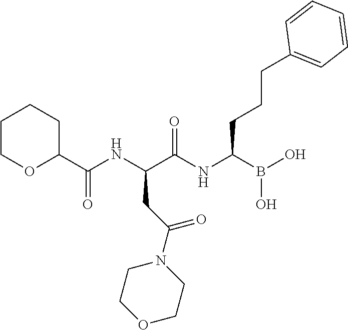 [SMILES: O=C(N[C@H](CC(=O)N1CCOCC1)C(=O)N[C@@H](CCCc1ccccc1)B(O)O)C1CCCCO1] | Structure 25 |
| 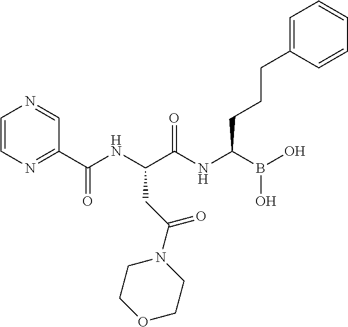 [SMILES: O=C(N[C@@H](CC(=O)N1CCOCC1)C(=O)N[C@@H](CCCc1ccccc1)B(O)O)c1cnccn1] | Structure 26 |
| 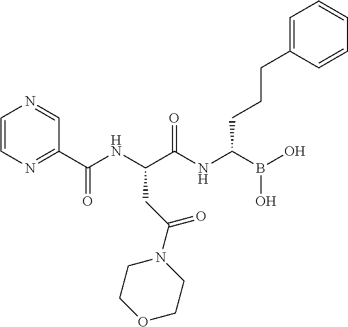 [SMILES: O=C(N[C@@H](CC(=O)N1CCOCC1)C(=O)N[C@H](CCCc1ccccc1)B(O)O)c1cnccn1] | Structure 27 |
| 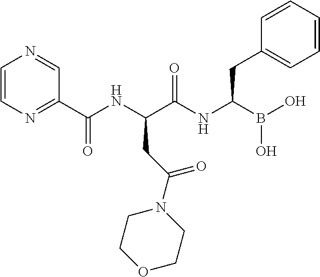 [SMILES: O=C(N[C@H](CC(=O)N1CCOCC1)C(=O)N[C@@H](Cc1ccccc1)B(O)O)c1cnccn1] | Structure 28 |
| 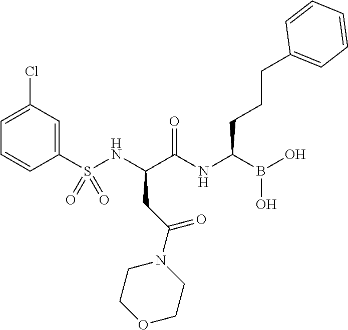 [SMILES: O=C(N[C@@H](CCCc1ccccc1)B(O)O)[C@@H](CC(=O)N1CCOCC1)NS(=O)(=O)c1cccc(Cl)c1] | Structure 29 |
| 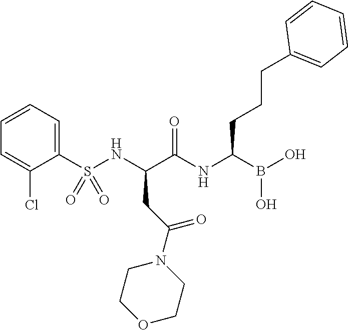 [SMILES: O=C(N[C@@H](CCCc1ccccc1)B(O)O)[C@@H](CC(=O)N1CCOCC1)NS(=O)(=O)c1ccccc1Cl] | Structure 30 |
| 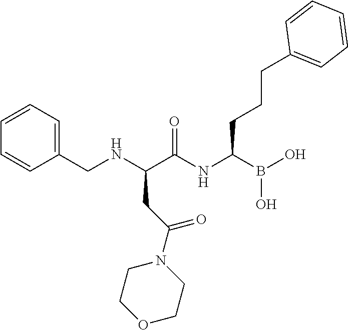 [SMILES: O=C(N[C@@H](CCCc1ccccc1)B(O)O)[C@@H](CC(=O)N1CCOCC1)NCc1ccccc1] | Structure 31 |
| 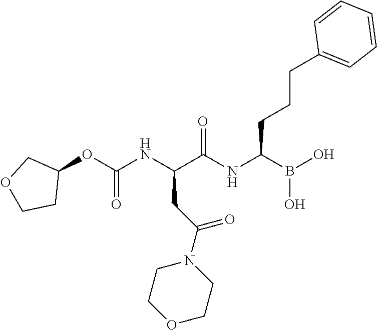 [SMILES: O=C(N[C@H](CC(=O)N1CCOCC1)C(=O)N[C@@H](CCCc1ccccc1)B(O)O)O[C@H]1CCOC1] | Structure 32 |
| 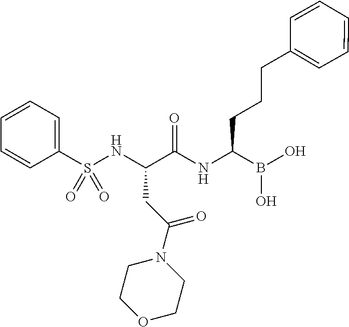 [SMILES: O=C(N[C@@H](CCCc1ccccc1)B(O)O)[C@H](CC(=O)N1CCOCC1)NS(=O)(=O)c1ccccc1] | Structure 33 |
| 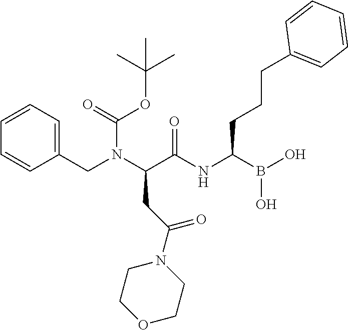 [SMILES: CC(C)(C)OC(=O)N(Cc1ccccc1)[C@H](CC(=O)N1CCOCC1)C(=O)N[C@@H](CCCc1ccccc1)B(O)O] | Structure 34 |
| 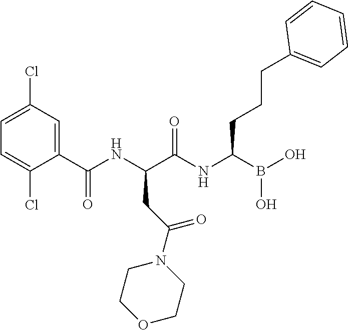 [SMILES: O=C(N[C@H](CC(=O)N1CCOCC1)C(=O)N[C@@H](CCCc1ccccc1)B(O)O)c1cc(Cl)ccc1Cl] | Structure 35 |
| 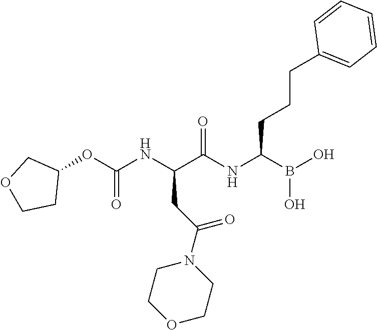 [SMILES: O=C(N[C@H](CC(=O)N1CCOCC1)C(=O)N[C@@H](CCCc1ccccc1)B(O)O)O[C@@H]1CCOC1] | Structure 36 |
| 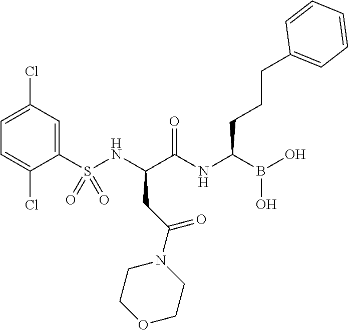 [SMILES: O=C(N[C@@H](CCCc1ccccc1)B(O)O)[C@@H](CC(=O)N1CCOCC1)NS(=O)(=O)c1cc(Cl)ccc1Cl] | Structure 37 |
| 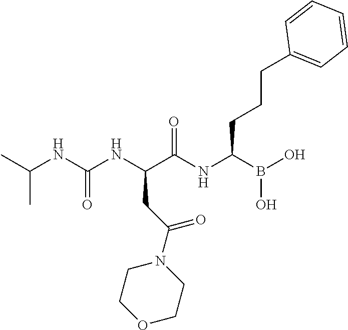 [SMILES: CC(C)NC(=O)N[C@H](CC(=O)N1CCOCC1)C(=O)N[C@@H](CCCc1ccccc1)B(O)O] | Structure 38 |
| 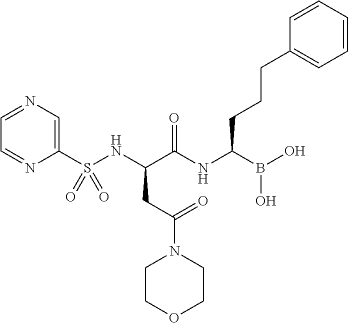 [SMILES: O=C(N[C@@H](CCCc1ccccc1)B(O)O)[C@@H](CC(=O)N1CCOCC1)NS(=O)(=O)c1cnccn1] | Structure 39 |
| 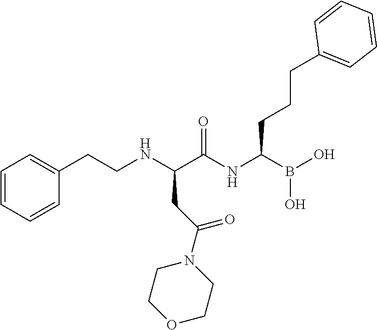 [SMILES: O=C(N[C@@H](CCCc1ccccc1)B(O)O)[C@@H](CC(=O)N1CCOCC1)NCCc1ccccc1] | Structure 40 |
| 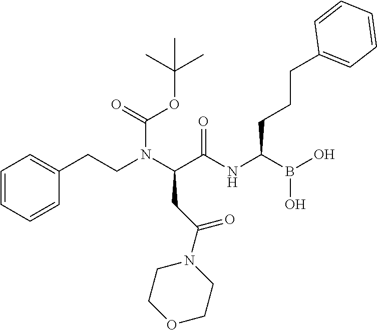 [SMILES: CC(C)(C)OC(=O)N(CCc1ccccc1)[C@H](CC(=O)N1CCOCC1)C(=O)N[C@@H](CCCc1ccccc1)B(O)O] | Structure 41 |
| 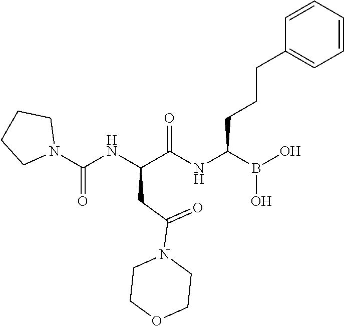 [SMILES: O=C(N[C@@H](CCCc1ccccc1)B(O)O)[C@@H](CC(=O)N1CCOCC1)NC(=O)N1CCCC1] | Structure 42 |
| 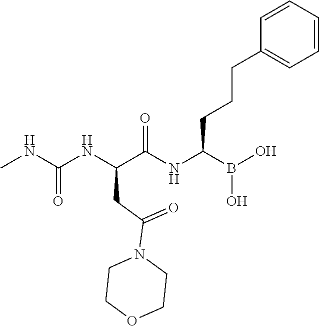 [SMILES: CNC(=O)N[C@H](CC(=O)N1CCOCC1)C(=O)N[C@@H](CCCc1ccccc1)B(O)O] | Structure 43 |
| 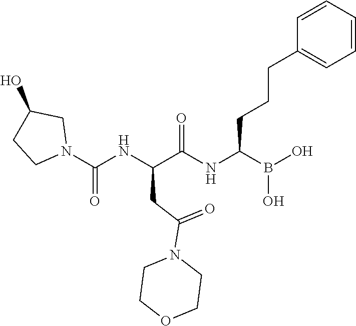 [SMILES: O=C(N[C@@H](CCCc1ccccc1)B(O)O)[C@@H](CC(=O)N1CCOCC1)NC(=O)N1CC[C@@H](O)C1] | Structure 44 |
| 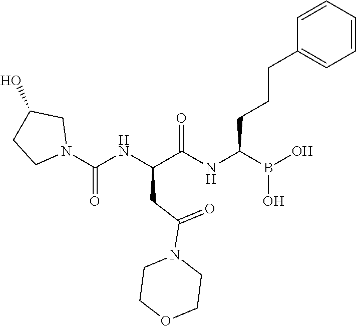 [SMILES: O=C(N[C@@H](CCCc1ccccc1)B(O)O)[C@@H](CC(=O)N1CCOCC1)NC(=O)N1CC[C@H](O)C1] | Structure 45 |
| 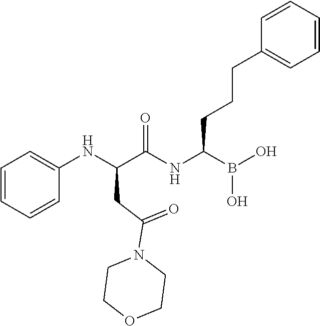 [SMILES: O=C(N[C@@H](CCCc1ccccc1)B(O)O)[C@@H](CC(=O)N1CCOCC1)Nc1ccccc1] | Structure 46 |
| 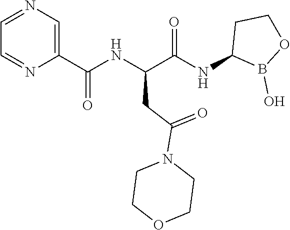 [SMILES: O=C(N[C@H](CC(=O)N1CCOCC1)C(=O)N[C@H]1CCOB1O)c1cnccn1] | Structure 47 |
| 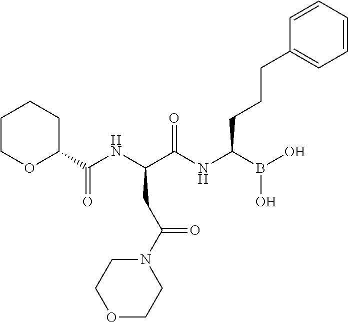 [SMILES: O=C(N[C@@H](CCCc1ccccc1)B(O)O)[C@@H](CC(=O)N1CCOCC1)NC(=O)[C@H]1CCCCO1] | Structure 48 |
| 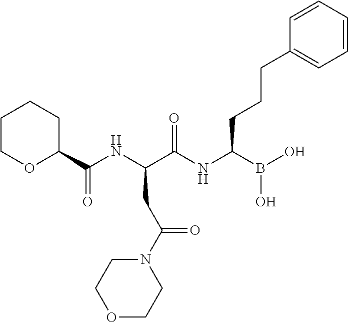 [SMILES: O=C(N[C@H](CC(=O)N1CCOCC1)C(=O)N[C@@H](CCCc1ccccc1)B(O)O)[C@@H]1CCCCO1] | Structure 49 |
| 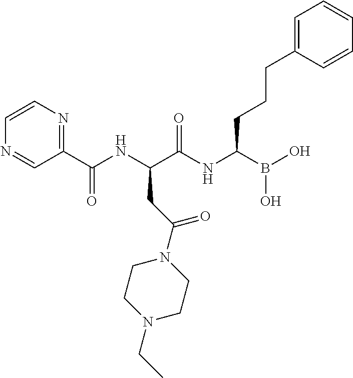 [SMILES: CCN1CCN(C(=O)C[C@@H](NC(=O)c2cnccn2)C(=O)N[C@@H](CCCc2ccccc2)B(O)O)CC1] | Structure 50 |
| 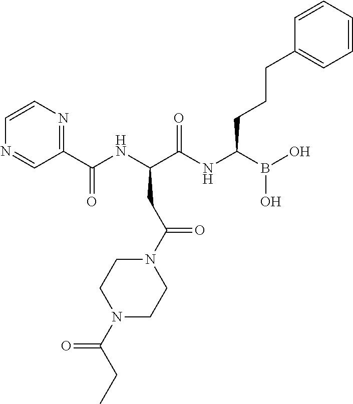 [SMILES: CCC(=O)N1CCN(C(=O)C[C@@H](NC(=O)c2cnccn2)C(=O)N[C@@H](CCCc2ccccc2)B(O)O)CC1] | Structure 51 |
| 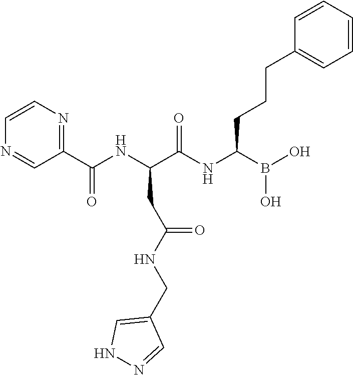 [SMILES: O=C(C[C@@H](NC(=O)c1cnccn1)C(=O)N[C@@H](CCCc1ccccc1)B(O)O)NCc1cn[nH]c1] | Structure 52 |
| 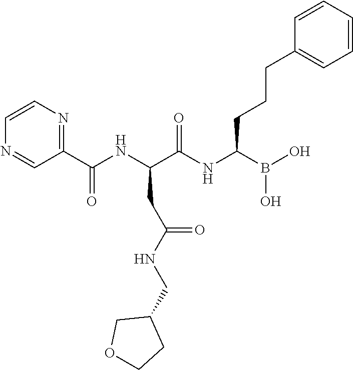 [SMILES: O=C(C[C@@H](NC(=O)c1cnccn1)C(=O)N[C@@H](CCCc1ccccc1)B(O)O)NC[C@@H]1CCOC1] | Structure 53 |
| 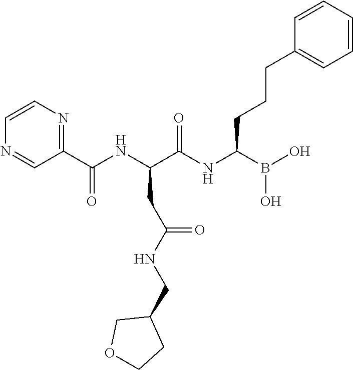 [SMILES: O=C(C[C@@H](NC(=O)c1cnccn1)C(=O)N[C@@H](CCCc1ccccc1)B(O)O)NC[C@H]1CCOC1] | Structure 54 |
| 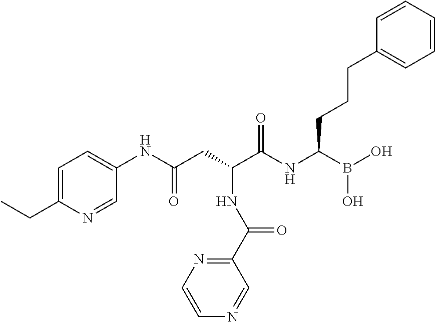 [SMILES: CCc1ccc(NC(=O)C[C@@H](NC(=O)c2cnccn2)C(=O)N[C@@H](CCCc2ccccc2)B(O)O)cn1] | Structure 55 |
| 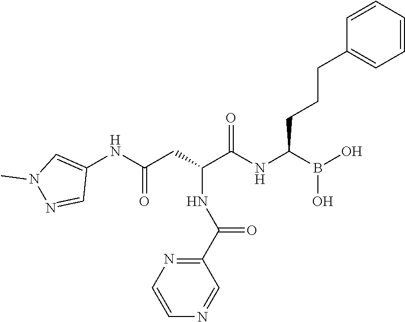 [SMILES: Cn1cc(NC(=O)C[C@@H](NC(=O)c2cnccn2)C(=O)N[C@@H](CCCc2ccccc2)B(O)O)cn1] | Structure 56 |
| 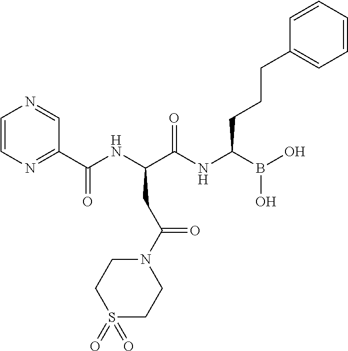 [SMILES: O=C(N[C@H](CC(=O)N1CCS(=O)(=O)CC1)C(=O)N[C@@H](CCCc1ccccc1)B(O)O)c1cnccn1] | Structure 57 |
| 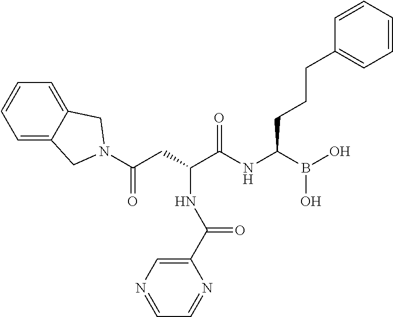 [SMILES: O=C(N[C@H](CC(=O)N1Cc2ccccc2C1)C(=O)N[C@@H](CCCc1ccccc1)B(O)O)c1cnccn1] | Structure 58 |
| 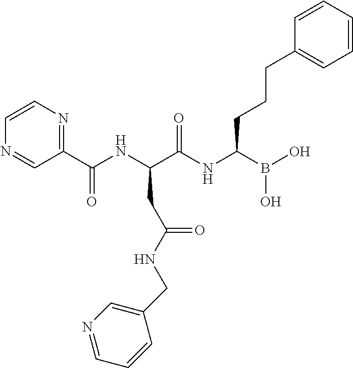 [SMILES: O=C(C[C@@H](NC(=O)c1cnccn1)C(=O)N[C@@H](CCCc1ccccc1)B(O)O)NCc1cccnc1] | Structure 59 |

The compounds of the present invention may contain asymmetric carbon atoms (sometimes as the result of a deuterium atom) and thereby can exist as either individual stereoisomers or mixtures of enantiomers or mixtures of diastereomers. Accordingly, a compound of the present invention may exist as either a racemic mixture, a mixture of diastereomers, or as individual stereoisomers that are substantially free of other stereoisomers. Synthetic, separation, or purification methods to be used to obtain an enantiomer of a given compound are known in the art and are applicable for obtaining the compounds identified herein.

Unless otherwise indicated, when a disclosed compound is named or depicted by a structure without specifying the stereochemistry and has one or more chiral centers, it is understood to represent all possible stereoisomers of the compound. In other words, chiral centers that lack solid wedged or hashed wedged bonds indicate a mixture of stereoisomers.

Certain compounds of the present invention may be able to exist as tautomers. All tautomeric forms of these compounds, whether isolated individually or in mixtures, are within the scope of the present invention. For example, in instances where an —OH substituent is permitted on a heteroaromatic ring and keto-enol tautomerism is possible, it is understood that the substituent might in fact be present, in whole or in part, in the oxo (═O) form.

Compounds of the present invention may exist in amorphous form and/or one or more crystalline forms. As such, all amorphous and crystalline forms and mixtures thereof of the compounds of the invention are intended to be included within the scope of the present invention. In addition, some of the compounds of the present invention may form solvates with water (i.e., a hydrate) or common organic solvents. Such solvates and hydrates, particularly the pharmaceutically acceptable solvates and hydrates, of the compounds of this invention are likewise encompassed within the scope of the compounds of the invention and the pharmaceutically acceptable salts thereof, along with un-solvated and anhydrous forms of such compounds.

In one embodiment, deuterium isotope content at the deuterium substituted position is greater than the natural isotopic deuterium content (0.015%), more preferably greater than 50%, more preferably greater than 60%, more preferably greater than 75%, more preferably greater than 90%, more preferably greater than 95%, more preferably greater than 97%, more preferably greater than 99%. It will be understood that some variation of natural isotopic abundance may occur in any compound depending upon the source of the reagents used in the synthesis. Thus, a preparation of undeuterated compounds may inherently contain small amounts of deuterated isotopologues, such amounts being insignificant as compared to the degree of stable isotopic substitution of the deuterated compounds of the invention (see, e.g., Gannes, L. Z. et al., Comp. Biochem. Physiol. Mol. Integr. Physiol, 119, 725 (1998)). Replacement of hydrogen with deuterium may affect the activity, toxicity, and pharmacokinetics (e.g., absorption, distribution, metabolism, and excretion (“ADME”)) of some drugs. For instance, such replacement may alter the chemical stability and biochemical reactivity of a compound through kinetic isotope effects. Because of the increased mass of deuterium relative to hydrogen, epimerization at stereogenic carbons may be slowed down when hydrogen is replaced with deuterium (see Pirali, T. et al, J. Med. Chem. 62, 5276-97 (2019)). Additionally, the presence of deuterium may affect how a molecule interacts with enzymes, thereby impacting enzyme kinetics. While in certain cases the increased mass of deuterium as compared to hydrogen can stabilize a compound and thereby improve activity, toxicity, or half-life, such impact is not predictable. In other instances deuteration may have little to no impact on these properties, or may affect them in an undesirable manner. Whether and/or how such replacement will impact drug properties can only be determined if the drug is synthesized, evaluated, and compared to its non-deuterated counterpart (see Fukuto, J. M., et al., J. Med. Chem. 34, 2871-76 (1991)). Because some drugs have multiple sites of metabolism or more than one active sites for binding to a target, it is unpredictable as to which sites may benefit by deuterium replacement or to what extent isotope enrichment is necessary to produce a beneficial effect.

A further embodiment of the present invention are compounds of the invention (that is, compounds of Compounds 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 and 49) or their pharmaceutically acceptable salts wherein one or more hydrogen is substituted with a deuterium atom.

Additional embodiments of the invention are pharmaceutical compositions comprising a compound of the disclosure (that is, compounds of Compounds 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 and 49) or a pharmaceutically acceptable salt thereof and a pharmaceutically acceptable excipient.

Further embodiments of the invention are methods of treating a disease characterized by mitochondrial dysfunction, such methods comprising administering to a subject in need thereof a therapeutically effective amount of a compound of the invention, or a pharmaceutically acceptable salt thereof. In some embodiments, the disease is selected from the group consisting of Alper's syndrome (Alpers-Huttenlocher syndrome), ataxia neuropathy syndrome (ANS), Mitochondrial DNA Depletion Syndrome (MDDS), Leigh Syndrome (Leigh Disease), Leber's Hereditary Optic Neuropathy (LHON), chronic progressive external ophthalmoplegia (CPEO), myoclonic epilepsy myopathy sensory ataxia (MEMSA), MELAS (Mitochondrial Encephalopathy, Lactic Acidosis, and Stroke-like episodes) syndrome, MERRF (myoclonus epilepsy with ragged-red fibers) syndrome, mitochondrial neurogastrointestinal encephalomyopathy (MNGIE), neuropathy, ataxia, and retinitis pigmentosa (NARP), Kearn's-Sayre Syndrome (KSS), and Pearson's Syndrome.

In some embodiments, the disease to be treated with a compounds of the invention or pharmaceutically acceptable salts thereof is associated with mtDNA mutations or deletions, for example m.3243A>G, m.11778G>A, m.14484T>C, m.3460G>A, m.8344A>G, m.3271T>C, m.3251A>G, m.8356T>C, m.4274T>C, m.14709T>C, m.12320A>G, m.4269A>G, m.12258C>A, m.1606G>A, m.10010T>C, m.7445A>G, and m.1555A>G (see https://mitomap.org/MITOMAP).

Additional embodiments of the invention are methods of treating cancers, such as those identified in Wong, K. S. et al. “Recent Advances in Targeting Human Mitochondrial AAA+ Proteases to Develop Novel Cancer Therapeutics,” Advances in Experimental Medicine and Biology, 1158,119-142 (2019), using a compound of the invention or its pharmaceutically acceptable salt.

For the treatment of cancer, the compounds described herein may be administered in combination with a chemotherapeutic agent. Therapeutically effective amounts of the additional chemotherapeutic agent(s) are well known to those skilled in the art. However, it is well within the attending physician to determine the amount of other chemotherapeutic agent(s) to be delivered.

Examples of these chemotherapeutic agents include, but are not limited to, Abitrexate (Methotrexate Injection), Abraxane (Paclitaxel Injection), Actemra (Tocilizumab), Adcetris (Brentuximab Vedotin Injection), Adriamycin (Doxorubicin), Adrucil Injection (5-FU (fluorouracil)), Afinitor (Everolimus), Afinitor Disperz (Everolimus), Aldara (Imiquimod), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Avelumab, Bexxar (Tositumomab), BiCNU (Carmustine), Blenoxane (Bleomycin), Blincyto (Blinatumomab), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Cyramza (Ramucirumab), Dacogen (Decitabine), Darzalex (Daratumumab), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Durvalumab, Eligard (Leuprolide), Ellence (Ellence (epirubicin)), Eloxatin (Eloxatin (oxaliplatin)), Elspar (Asparaginase), Emcyt (Estramustine), Empliciti (Elotuzumab), Enhertu (fam-trastuzumab deruxtecan-nxki), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gazyva (Obinutuzumab), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leflunomide (SU101), Lartruvo (Olaratumab), Leukeran (Chlorambucil), Leukine (Sargramostim), Leustatin (Cladribine), Libtayo (Cemiplimab), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Ninlaro (Ixazomib), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Portrazza (Necitumumab), Prednisone Intensol (Prednisone), Proleukin (Aldesleukin), Purinethol (Mercaptopurine), Reclast (Zoledronic acid), Revlimid (Lenalidomide), Removab (Catumaxomab), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Sarclisa (Isatuximab-irfc), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Tecentriq (Atezolizumab), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Tremelimumab, Trelstar (Triptorelin Injection), Trexall (Methotrexate), Trisenox (Arsenic trioxide), Tykerb (lapatinib), Unituxin (Dinutuximab), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (lbritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MED14736, MPDL3280A and MSB0010718C targeting.

Further embodiments of the invention are methods of treating neurodegenerative disorders, metabolic disorders, and diseases associated with the aging process.

### Dosage Forms, Medicaments and Pharmaceuticals:

The compounds, molecules or agents of the disclosure may be used to treat (e.g. cure, alleviate or prevent) one or more diseases, infections or disorders. Thus, in accordance with the disclosure, the compounds and molecules may be manufactured into medicaments or may be incorporated or formulated into pharmaceutical compositions.

The molecules, compounds and compositions of the disclosure may be administered by any convenient route, for example, methods of administration include intradermal, intramuscular, intraperitoneal, intravenous, subcutaneous, intranasal, epidural, oral, sublingual, intranasal, intravaginal, transdermal, rectally, by inhalation, or topically to the skin. Delivery systems are also known to include, for example, encapsulation in liposomes, microgels, microparticles, microcapsules, capsules, etc. Any other suitable delivery system known in the art is also envisioned in use. Administration can be systemic or local. The mode of administration may be left to the discretion of the practitioner.

The dosage administered will, of course, vary depending upon known factors, such as the pharmacodynamic properties of the particular active agent; the chosen mode and route of administration; the age, health and weight of the recipient; the nature of the disease or disorder to be treated; the extent of the symptoms; any simultaneous or concurrent treatments; the frequency of treatment; and the effect desired.

Depending on known factors, such as those noted above, the required dosage of the active agent may be administered in a single daily dose, or the total daily dosage may be administered in divided doses of e.g. two, three, or four times daily. Suitably, the therapeutic treatment regime according to the disclosure is devised for a single daily dose or for a divided daily dose of two doses.

The ‘effective amount’ or ‘therapeutically effective amount’ is meant to describe an amount of compound or a composition of the disclosure that is effective in curing, inhibiting, alleviating, reducing or preventing the adverse effects of the diseases or disorders to be treated, or the amount necessary to achieve a physiological or biochemically-detectable effect. Thus, at the effective amount, the compound or agent is able to produce the desired therapeutic, ameliorative, inhibitory or preventative effect in relation to disease or disorder. Beneficially, an effective amount of the compound or composition of the disclosure may have the effect of inhibiting LONP1. Diseases or disorders which may benefit from LONP1 inhibition include, for example, proliferative diseases or disorders and cancer.

When administered to a subject, a compound of the disclosure is suitably administered as a component of a composition that comprises a pharmaceutically acceptable carrier or vehicle. One or more additional pharmaceutical acceptable carrier (such as diluents, adjuvants, excipients or vehicles) may be combined with the compound of the disclosure in a pharmaceutical composition. Suitable pharmaceutical carriers are described in “Remington's Pharmaceutical Sciences” by E. W. Martin. Pharmaceutical formulations and compositions of the disclosure are formulated to conform to regulatory standards and according to the chosen route of administration.

Acceptable pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilising, thickening, lubricating and colouring agents may be used. When administered to a subject, the pharmaceutically acceptable vehicles are generally sterile. Water is a suitable vehicle when the compound is to be administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or buffering agents.

The medicaments and pharmaceutical compositions of the disclosure can take the form of solutions, suspensions, emulsion, tablets, pills, pellets, powders, gels, capsules (for example, capsules containing liquids or powders), modified-release formulations (such as slow or sustained-release formulations), suppositories, emulsions, aerosols, sprays, suspensions, or any other form suitable for use. Other examples of suitable pharmaceutical vehicles are described in Remington's Pharmaceutical Sciences, Alfonso R. Gennaro ed., Mack Publishing Co. Easton, Pa., 19th ed., 1995, see for example pages 1447-1676.

Suitably, the therapeutic compositions or medicaments of the disclosure are formulated in accordance with routine procedures as a pharmaceutical composition adapted for oral administration (more suitably for humans). Compositions for oral delivery may be in the form of tablets, lozenges, aqueous or oily suspensions, granules, powders, emulsions, capsules, syrups, or elixirs, for example. Thus, in one embodiment, the pharmaceutically acceptable vehicle is a capsule, tablet or pill.

Orally administered compositions may contain one or more agents, for example, sweetening agents such as fructose, aspartame or saccharin; flavouring agents such as peppermint, oil of wintergreen, or cherry; colouring agents; and preserving agents, to provide a pharmaceutically palatable preparation. When the composition is in the form of a tablet or pill, the compositions may be coated to delay disintegration and absorption in the gastrointestinal tract, so as to provide a sustained release of active agent over an extended period of time. Selectively permeable membranes surrounding an osmotically active driving compound are also suitable for orally administered compositions. In these dosage forms, fluid from the environment surrounding the capsule is imbibed by the driving compound, which swells to displace the agent or agent composition through an aperture. These dosage forms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade. For oral formulations, the location of release may be the stomach, the small intestine (the duodenum, the jejunum, or the ileum), or the large intestine. One skilled in the art is able to prepare formulations that will not dissolve in the stomach yet will release the material in the duodenum or elsewhere in the intestine. Suitably, the release will avoid the deleterious effects of the stomach environment, either by protection of the compound (or composition) or by release of the compound (or composition) beyond the stomach environment, such as in the intestine. To ensure full gastric resistance a coating impermeable to at least pH 5.0 would be essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac, which may be used as mixed films.

While it can be beneficial to provide therapeutic compositions and/or compounds of the disclosure in a form suitable for oral administration, for example, to improve patient compliance and for ease of administration, in some embodiments, compounds or compositions of the disclosure may cause undesirable side-effects, such as intestinal inflammation which may lead to premature termination of a therapeutic treatment regime. Thus, in some embodiments, the therapeutic treatment regime is adapted to accommodate ‘treatment holidays’, e.g. one or more days of non-administration. For example, treatment regimens and therapeutic methods of the disclosure may comprise a repetitive process comprising administration of the therapeutic composition or compound for a number of consecutive days, followed by a treatment holiday of one or more consecutive days. For example, a treatment regime of the disclosure may comprise a repetitive cycle of administration of the therapeutic composition or compound for between 1 and 49 consecutive days, between 2 and 42 days, between 3 and 35 days, between 4 and 28 days, between 5 and 21 days, between 6 and 14 days, or between 7 and 10 days; followed by a treatment holiday of between 1 and 14 consecutive days, between 1 and 12 days, between 1 and 10 days, or between 1 and 7 days (e.g. 1, 2, 3, 4, 5, 6 or 7 days).

To aid dissolution of the therapeutic agent into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. Potential nonionic detergents that could be included in the formulation as surfactants include: lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants, when used, could be present in the formulation of the compound or derivative either alone or as a mixture in different ratios.

Typically, compositions for intravenous administration comprise sterile isotonic aqueous buffer. Where necessary, the compositions may also include a solubilising agent.

Another suitable route of administration for the therapeutic compositions of the disclosure is via pulmonary or nasal delivery.

Additives may be included to enhance cellular uptake of the therapeutic agent of the disclosure, such as the fatty acids oleic acid, linoleic acid and linolenic acid.

The therapeutic agents of the disclosure may also be formulated into compositions for topical application to the skin of a subject.

Where the invention provides more than one active compound/agent for use in combination, generally, the agents may be formulated separately or in a single dosage form, depending on the prescribed most suitable administration regime for each of the agents concerned. When the therapeutic agents are formulated separately, the pharmaceutical compositions of the invention may be used in a treatment regime involving simultaneous, separate or sequential administration with the other one or more therapeutic agent. The other therapeutic agent(s) may comprise a compound of the disclosure or a therapeutic agent known in the art.

Specific and general embodiments of the disclosure will now be described by way of the following non-limiting examples.

### EXAMPLES

The examples and preparations provided below further illustrate and exemplify the compounds of the present invention and methods of preparing such compounds. It is to be understood that the scope of the present invention is not limited in any way by the scope of the following examples and preparations.

The structures of the compounds are confirmed by MS, elemental analysis and/or NMR, where peaks assigned to the characteristic protons in the title compound are presented where appropriate. 1H NMR shift (6) are given in parts per million (ppm) down field from an internal reference standard.

Preparative SFC method: Separation was performed on PIC-SOLUTION-175 instrument by using Reflect (R,R) WHELK-01 column (21.1 mm×250 mm), 5μ operating at 35° C., maintaining flow rate of 60 ml/min, using 65% CO2 in super critical state and 35% methanol as mobile phase, run for 12 minutes at 100 bar (detection at 230 nm).

The following abbreviations are used in the synthetic schemes above and the examples below. If an abbreviation used herein is not defined, it has its generally accepted meaning:
Abu 2-aminobutyric acid
ACN Acetonitrile
Bn Benzyl
BnBr Benzyl bromide
CuCl2 Copper chloride
D2O Deuterated water
DCM Dichloromethane
DIPEA Diisopropylethylamine
DMSO Dimethylsulfoxide
DMSO-d6 Deuterated dimethylsulfoxide
Et Ethyl
EtOAc Ethyl acetate
Et3N Triethylamine
EtOH Ethanol
Et2O Diethyl ether
h hour
H2O water
HBr Hydrobromic acid
HCl Hydrochloric acid
I2 Iodine
IBCF Isobutyl chloroformate
K2CO3 Potassium carbonate
KOAc Potassium acetate
KOtBu Potassium tert-butoxide
LAF Laminar Flow Box
LiOH Lithium hydroxide
min minutes
Me Methyl
MeCN Acetonitrile
MeI Methyl iodide
MeMgBr Methyl magnesium bromide
MeO or OMe Methoxy
MeOD Deuterated Methanol
MeOH Methanol
MgSO4 Magnesium sulfate
MS Mass spectrometry
N2 Nitrogen
NaH Sodium hydride
NMM N-Methylmorpholine
NaOAc Sodium acetate
NaOH Sodium hydroxide
Na2CO3 Sodium carbonate
Na2SO4 Sodium sulfate
NaH Sodium hydride
NaHCO₃Sodium bicarbonate
NH4Cl Ammonium chloride
Pd2(dba)₃ Tris(dibenzylideneacetone)dipalladium(0)
POCl3 Phosphoryl chloride
Prep-HPLC Preparative high performance liquid chromatography
Prep-TLC Preparative thin layer chromatography
PTSA p-Toluenesulfonic acid
Ph Phenyl
rt Room temperature (typically 18 to 22° C.)
SiO2 Silica gel
t-BuOH Tert-butanol
THF Tetrahydrofuran

### Preparation of the Compounds:

The starting materials and reagents used in each step in the preparation are known and can be readily prepared or purchased from commercial sources.

The compound obtained in each step can also be used for the next reaction as a reaction mixture thereof, or after obtaining a crude product thereof. Alternatively, the compound obtained in each step can be isolated and/or purified from the reaction mixture by a separation means such as concentration, crystallization, recrystallization, distillation, solvent extraction, fractionation, chromatography and the like according to a conventional method.

In each reaction step, while the reaction time varies depending on the reagents and solvents to be used, unless otherwise specified, it is generally 1 min to 48 hr, preferably 10 min to 8 hr.

In the reaction of each step, while the reaction temperature varies depending on the reagents and solvents to be used, unless otherwise specified, it is generally −78° C. to 300° C., preferably −78° C. to 150° C.

In the reaction of each step, unless otherwise specified, a reagent is used in 0.5 equivalent to 20 equivalents, preferably 0.8 equivalent to 5 equivalents, relative to the substrate. When a reagent is used as a catalyst, the reagent is used in 0.001 equivalent to 1 equivalent, preferably 0.01 equivalent to 0.2 equivalent, relative to the substrate. When the reagent is also a reaction solvent, the reagent is used in a solvent amount.

In the reaction of each step, unless otherwise specified, it is performed without solvent or by dissolving or suspending in a suitable solvent. Specific examples of the solvent include the following. Alcohols: methanol, ethanol, tert-butyl alcohol, 2-methoxyethanol and the like; ethers: diethyl ether, diphenyl ether, tetrahydrofuran, 1,2-dimethoxyethane and the like; aromatic hydrocarbons: chlorobenzene, toluene, xylene and the like; saturated hydrocarbons: cyclohexane, hexane and the like; amides: N,N-dimethylformamide, N-methylpyrrolidone and the like; halogenated hydrocarbons: dichloromethane, carbon tetrachloride and the like; nitriles: acetonitrile and the like; sulfoxides: dimethyl sulfoxide and the like; aromatic organic bases: pyridine and the like; acid anhydrides: acetic anhydride and the like; organic acids: formic acid, acetic acid, trifluoroacetic acid and the like; inorganic acids: hydrochloric acid, sulfuric acid and the like; esters: ethyl acetate and the like; ketones: acetone, methyl ethyl ketone and the like; and water. Two or more kinds of the above-mentioned solvents may be used by mixing at an appropriate ratio.

Unless otherwise specified, the reaction of each step is performed according to a known method, for example, the methods described in “Reactions and Syntheses: In the Organic Chemistry Laboratory 2nd Edition” (Lutz F. Tietze, Theophil Eicher, Ulf Diederichsen, Andreas Speicher, Nina Schutzenmeister) Wiley, 2015; “Organic Syntheses Collective Volumes 1-12” (John Wiley & Sons Inc); “Comprehensive Organic Transformations, Third Edition” (Richard C. Larock) Wiley, 2018 and the like.

In each step, protection or deprotection of a functional group is performed by a known method, for example, the methods described in “Protective Groups in Organic Synthesis, 4^{th} Ed.” (Theodora W. Greene, Peter G. M. Wuts) Wiley-Interscience, 2007; “Protecting Groups 3rd Ed.” (P. J. Kocienski) Thieme, 2004 and the like.

Deuterated LONP1 inhibitors of the present invention can be prepared using chemical reactions known to a person of ordinary skill in the art using deuterated starting materials or reagents. Deuterium-containing reagents are well known in the art and can be prepared using known procedures or purchased from commercial sources. The deuterated compounds obtained can be characterized by analytical techniques known to persons of ordinary skill in the art. For example, nuclear magnetic resonance (‘NMR’) can be used to determine a compound's structure while mass spectroscopy (‘MS’) can be used to determine the amount of deuterium atom in the compound by comparison to its non-deuterated form.

The Examples and preparations provided below further illustrate and exemplify the compounds of the present invention and methods of preparing such compounds. It is to be understood that the scope of the present invention is not limited in any way by the scope of the following examples and preparations.

The structures of the compounds are confirmed by either elemental analysis or NMR, where peaks assigned to the characteristic protons in the title compound are presented where appropriate. ¹H NMR shift (δH) are given in parts per million (ppm) down field from an internal reference standard.

### Example 1

### Synthesis of ((R)-1-((R)-4-morpholino-4-oxo-2-(pyrazine-2-carboxamido)butanamido)-4-phenylbutyl)boronic acid (Formula 2)

In certain embodiments, Compound 1/Structure 1 can be produced from compound (1-1) by the method shown in Scheme 1 (below). Amino acid (1-1) is coupled with morpholine (1-2) to produce amide (1-3), which is then deprotected to generate carboxylic acid (1-4). Subsequent coupling with the amine of a protected boronic acid compound (1-5) produces amide (1-6). Removal of the BOC protecting group yields the corresponding amine (1-7) as the hydrochloride salt, which is then coupled with carboxylic acid (1-8) to form amide (1-9). Deprotection of (1-9) with methylboronic acid (1-10) produces Compound 1.

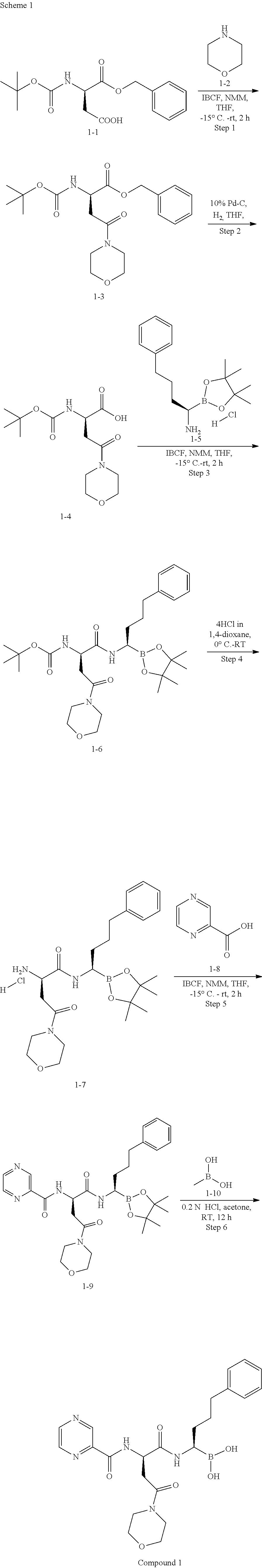

Synthesis of benzyl (R)-2-((tert-butoxycarbonyl)amino)-4-morpholino-4-oxobutanoate [Step 1]: To a stirred solution of (R)-4-(benzyloxy)-3-((tert-butoxycarbonyl)amino)-4-oxobutanoic acid (1-1, 2.0 g, 6.3 mmol) in tetrahydrofuran (25 mL) was added isobutyl chloroformate (IBCF) (0.8 mL, 6.3 mmol) and N-methylmorpholine (NMM) (0.7 mL, 6.3 mmol) at −15° C. The reaction mixture was stirred at the same temperature for 30 minutes. Then morpholine (1-2, 0.5 mL, 5.7 mmol) followed by NMM (0.6 mL, 5.7 mmol) was added to the reaction mixture at −15° C., which was gradually warmed to 0° C. and stirred for 2 hours. LCMS of the reaction mass confirmed the formation of the desired product. The reaction mixture was neutralized with aqueous 0.1 N HCl solution and extracted with ethyl acetate several times. The organic layers were combined and washed with a 5% potassium carbonate solution, water, brine, dried over sodium sulfate, filtered, and concentrated under reduced pressure to obtain the product. The product was purified by silica gel column chromatography to afford benzyl (R)-2-((tert-butoxycarbonyl)amino)-4-morpholino-4-oxobutanoate (1-3, 2.0 g). [M+H]⁺=392.9.

Synthesis of (R)-2-((tert-butoxycarbonyl)amino)-4-morpholino-4-oxobutanoic acid [Step 2]: To a stirred solution of benzyl (R)-2-((tert-butoxycarbonyl)amino)-4-morpholino-4-oxobutanoate (1-3, 2.0 g, 5.1 mmol) in tetrahydrofuran (50 mL) was added nitrogen gas for 10 minutes. Then 10% Pd—C (400 mg, 3.7 mmol) was added and the reaction mixture was hydrogenated under a hydrogen balloon for 3 hours. The reaction was monitored by TLC, and upon completion, the reaction mixture was filtered over celite using excess ethyl acetate. The solvent was removed by concentration under reduced pressure and provided (R)-2-((tert-butoxycarbonyl)amino)-4-morpholino-4-oxobutanoic acid (1-4, 1.5 g). [M−H]⁺=301.2. Compounds (3-1), (5-1), (6-1), (18-1), and (19-1) are the same as compound (1-4). Compound (27-1) is an isomer of compound (1-4).

Synthesis of tert-butyl ((R)-4-morpholino-1,4-dioxo-1-(((R)-4-phenyl-1-(4,4,5,5-tetramethyl-1,3,2-dioxaborolan-2-yl)butyl)amino)butan-2-yl)carbamate [Step 3]: To a stirred solution of (R)-2-((tert-butoxycarbonyl)amino)-4-morpholino-4-oxobutanoic acid (1-4, 450 mg, 1.5 mmol) in tetrahydrofuran (8 mL) was added isobutyl chloroformate (IBCF) (0.2 mL, 1.5 mmol) and N-methylmorpholine (NMM) (0.2 mL, 1.5 mmol) at −15° C. The reaction mixture was stirred at the same temperature for 30 minutes. Then (R)-4-phenyl-1-(4,4,5,5-tetramethyl-1,3,2-dioxaborolan-2-yl)butan-1-amine hydrochloride (1-5, 420 mg, 1.4 mmol) in dimethylformamide (1 mL) was added to the reaction mixture followed by NMM (0.15 mL, 1.4 mmol) at −15° C. The reaction mixture was gradually warmed to 0° C. and stirred for 2 hours. LCMS of the reaction mass confirmed the formation of the desired product, and the reaction mixture was neutralized with an aqueous solution of 0.1 N HCl and extracted with ethyl acetate. The organic layers were combined and washed with 5% potassium carbonate solution, water, brine, dried over sodium sulfate, filtered, and concentrated under reduced pressure to afford tert-butyl ((R)-4-morpholino-1,4-dioxo-1-(((R)-4-phenyl-1-(4,4,5,5-tetramethyl-1,3,2-dioxaborolan-2-yl)butyl)amino)butan-2-yl) carbamate (1-6, 635 mg). [M−H]: 558.4. Compound (13-1) is the same as compound (1-6).

Synthesis of (R)-2-amino-4-morpholino-4-oxo-N—((R)-4-phenyl-1-(4,4,5,5-tetramethyl-1,3,2-dioxaborolan-2-yl)butyl)butanamide hydrochloride [Step 4]: To a solution of tert-butyl ((R)-4-morpholino-1,4-dioxo-1-(((R)-4-phenyl-1-(4,4,5,5-tetramethyl-1,3,2-dioxaborolan-2-yl)butyl) amino)butan-2-yl)carbamate (1-6, 635 mg, 1.1 mmol) was added 4 M HCl in dioxane (3.0 mL, 11.0 mmol) at 0° C. The reaction mixture was gradually warmed to ambient temperature and stirred for 16 hours. TLC showed complete consumption of starting material and the reaction mixture was concentrated under reduced pressure to obtain (R)-2-amino-4-morpholino-4-oxo-N—((R)-4-phenyl-1-(4,4,5,5-tetramethyl-1,3,2-dioxaborolan-2-yl)butyl)butanamide hydrochloride (1-7, 560 mg). The product was used without purification. Compounds (11-1) and (12-1) are the same as compound (1-7).

Synthesis of N—((R)-4-morpholino-1,4-dioxo-1-(((R)-4-phenyl-1-(4,4,5,5-tetramethyl-1,3,2-dioxaborolan-2-yl)butyl)amino)butan-2-yl)pyrazine-2-carboxamide [Step 5]: To a stirred solution of pyrazine-2-carboxylic acid (1-8, 155 mg, 1.3 mmol) in tetrahydrofuran (8 mL) was added isobutyl chloroformate (IBCF) (0.2 mL, 1.3 mmol) and N-methylmorpholine (NMM) (0.15 mL, 1.3 mmol) at −15° C. The reaction mixture was stirred at the same temperature for 30 minutes. Then (R)-2-amino-4-morpholino-4-oxo-N—((R)-4-phenyl-1-(4,4,5,5-tetramethyl-1,3,2-dioxaborolan-2-yl)butyl)butanamide hydrochloride (1-7, 560 mg, 1.1 mmol) in dimethylformamide (1 mL) was added to the reaction mixture, followed by NMM (0.1 mL, 1.1 mmol). The reaction mixture was gradually warmed to 0° C. and stirred for 2 hours. LCMS of the reaction mixture confirmed formation of the product, and the reaction mixture was neutralized with aqueous 0.1 N HCl solution and extracted with ethyl acetate. The organic layers were combined and washed with 5% potassium carbonate solution, water, brine, dried over sodium sulfate, filtered and evaporated under reduced pressure. The product was purified by reverse-phase prep-HPLC and lyophilized to afford N—((R)-4-morpholino-1,4-dioxo-1-(((R)-4-phenyl-1-(4,4,5,5-tetramethyl-1,3,2-dioxaborolan-2-yl)butyl)amino)butan-2-yl)pyrazine-2-carboxamide (1-9, 45 mg). [M−H]⁺=564.4. ¹H NMR (400 MHz, MeOD) δ 9.26 (br s, 1H), 8.82-8.81 (m, 1H), 8.70 (br s, 1H), 7.21-7.11 (m, 5H), 5.30 (br s, 1H), 3.68-3.52 (m, 8H), 3.31-3.27 (m, 1H), 3.05-2.90 (m, 1H), 2.62-2.59 (m, 3H), 1.68-1.35 (m, 4H), 1.18-1.17 (m, 4H).

Synthesis of ((R)-1-((R)-4-morpholino-4-oxo-2-(pyrazine-2-carboxamido)butanamido)-4-phenylbutyl)boronic acid [Step 6]: To a stirred solution of N—((R)-4-morpholino-1,4-dioxo-1-(((R)-4-phenyl-1-(4,4,5,5-tetramethyl-1,3,2-dioxaborolan-2-yl)butyl) amino)butan-2-yl)pyrazine-2-carboxamide (1-9, 45 mg, 0.1 mmol) and methylboronic acid (1-10, 50 mg, 0.8 mmol) in acetone (2 mL) was added 0.2 N HCl (2 mL) and the reaction mixture was stirred at ambient temperature overnight. TLC and LCMS showed the complete disappearance of the starting material and the reaction mixture was concentrated under reduced pressure. The product was redissolved in a mixture of acetone and deionized water, and lyophilized to obtain ((R)-1-((R)-4-morpholino-4-oxo-2-(pyrazine-2-carboxamido)butanamido)-4-phenylbutyl)boronic acid (Compound 1, 38 mg). [M−H]⁺=482.2. ¹H NMR (400 MHz, DMSO-d₆, D₂O exchange) δ 9.15 (s, 1H), 8.84 (d, 1H), 8.71 (br s, 1H), 7.20-7.09 (m, 5H), 4.80 (t, 1H), 3.52-3.36 (m, 8H), 3.13-2.85 (m, 3H), 2.75-2.65 (m, 1H), 1.46-1.44 (m, 5H).

### Example 2

### Synthesis of ((R)-1-((R)-4-oxo-2-(pyrazine-2-carboxamido)-4-(pyrrolidin-1-yl)butanamido)-4-phenylbutyl)boronic acid

In other embodiments, Compound 2/Structure 2 can be produced from compound (2-1) by the method shown in Scheme 2. Amino acid (2-1) is coupled with pyrrolidone to form amide (2-2), which is then deprotected to generate the carboxylic acid compound (2-3). Coupling with the amine of a protected boronic acid compound (2-4) produces amide (2-5). Removal of the BOC protecting group yields the corresponding amine (2-6) as the hydrochloride salt, which is then coupled with carboxylic acid (2-7) to form amide (2-8). Deprotection of (2-8) with methylboronic acid (2-9) produces Compound 2.

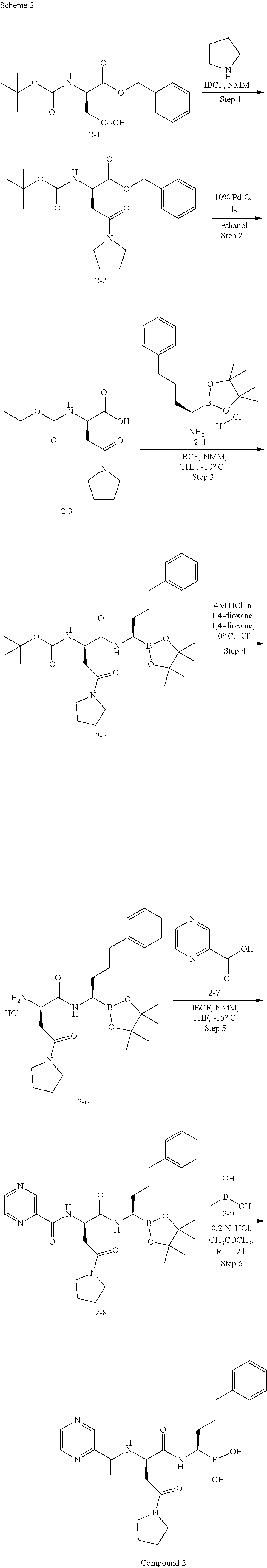

Synthesis of benzyl (R)-2-((tert-butoxycarbonyl)amino)-4-oxo-4-(pyrrolidin-1-yl)butanoate [Step 1]: To a solution of (R)-4-(benzyloxy)-3-((tert-butoxycarbonyl)amino)-4-oxobutanoic acid (2-1, 3.5 g, 11.0 mmol) in tetrahydrofuran (20 mL) was added isobutyl chloroformate (IBCF) (1.5 mL, 11.0 mmol) and N-methylmorpholine (NMM) (1.6 mL, 12.0 mmol) dropwise at −10° C. The reaction mixture was stirred at the same temperature for 30 minutes. Then pyrrolidine (0.8 mL, 9.8 mmol) and NMM (1.6 mL, 11.8 mmol) was added to the reaction mixture at −10° C., which was gradually warmed to ambient temperature and stirred over 2 hours. After the reaction was completed, as measure by LCMS, the reaction mixture was diluted with ethyl acetate and the organic layer was washed with aqueous 0.1 N HCl (twice), 10% aqueous potassium carbonate (twice), water (twice), brine (twice), dried over sodium sulfate, and concentrated under reduced pressure at ambient temperature. The product was purified by column chromatography using 20% ethyl acetate in hexane as eluent to afford benzyl (R)-2-((tert-butoxycarbonyl)amino)-4-oxo-4-(pyrrolidin-1-yl)butanoate (2-2, 3300 mg). [M+H]⁺=376.9.

Synthesis of (R)-2-((tert-butoxycarbonyl)amino)-4-oxo-4-(pyrrolidin-1-yl)butanoic acid [Step 2]: A 2-necked round-bottom flask was purged with nitrogen, and a solution of benzyl (R)-2-((tert-butoxycarbonyl)amino)-4-oxo-4-(pyrrolidin-1-yl)butanoate (2-2, 3.3 g, 8.8 mmol) in tetrahydrofuran (50 mL) was added to the flask, followed by 10% Pd—C (280 mg, 2.6 mmol). The atmosphere in the flask was exchanged with hydrogen gas and the reaction mixture was stirred overnight at ambient temperature under a hydrogen balloon. The reaction mixture was filtered through celite and washed with tetrahydrofuran. The combined filtrate was concentrated under reduced pressure to give the product that was purified by silica gel column chromatography using 20% ethyl acetate in hexanes as eluent to yield (R)-2-((tert-butoxycarbonyl)amino)-4-oxo-4-(pyrrolidin-1-yl)butanoic acid (2-3, 1.8 g). [M+H]⁺=287.1. ¹H NMR (400 MHz, DMSO-d₆) δ 12.51 (s, 1H), 6.76 (d, 1H), 4.32 (q, 1H), 3.40-3.32 (m, 2H), 3.26 (t, 2H), 2.71-2.62 (m, 2H), 1.89-1.82 (m, 2H), 1.79-1.72 (m, 2H), 1.37 (s, 9H).

Synthesis of tert-butyl ((R)-1,4-dioxo-1-(((R)-4-phenyl-1-(4,4,5,5-tetramethyl-1,3,2-dioxaborolan-2-yl)butyl)amino)-4-(pyrrolidin-1-yl)butan-2-yl)carbamate [Step 3]: To a solution of (R)-2-((tert-butoxycarbonyl)amino)-4-oxo-4-(pyrrolidin-1-yl) butanoic acid (2-6, 240 mg, 0.8 mmol) in tetrahydrofuran (7 mL) at −10° C., was added a mixture of isobutyl chloroformate (IBCF) (0.1 mL, 0.8 mmol) and N-methylmorpholine (NMM) (0.1 mL, 0.8 mmol) dropwise, and the reaction mixture was stirred for 30 minutes at −10° C. To this reaction mixture was added a mixture of (R)-4-phenyl-1-(4,4,5,5-tetramethyl-1,3,2-dioxaborolan-2-yl)butan-1-amine hydrochloride (2-4, 240 mg, 0.8 mmol) and NMM (0.1 mL, 0.8 mmol) in a mixture of tetrahydrofuran:dimethylformamide (3 mL:1 mL), the mixture was stirred for 2 hours, and gradually brought to ambient temperature. After the reaction was completed as monitored by LC-MS, the reaction mixture was diluted with ethyl acetate and the organic layer was washed with aqueous 0.1 N HCl (twice), 10% aqueous potassium carbonate (twice), water (twice), brine (twice), dried over sodium sulfate and concentrated under reduced pressure at ambient temperature to afford tert-butyl ((R)-1,4-dioxo-1-(((R)-4-phenyl-1-(4,4,5,5-tetramethyl-1,3,2-dioxaborolan-2-yl)butyl)amino)-4-(pyrrolidin-1-yl)butan-2-yl)carbamate (2-5, 400 mg). The product was used without purification. [M−H]⁻=5422.

Synthesis of (R)-2-amino-4-oxo-N—((R)-4-phenyl-1-(4,4,5,5-tetramethyl-1,3,2-dioxaborolan-2-yl)butyl)-4-(pyrrolidin-1-yl)butanamide hydrochloride [Step 4]: To a solution of tert-butyl ((R)-1,4-dioxo-1-(((R)-4-phenyl-1-(4,4,5,5-tetramethyl-1,3,2-dioxaborolan-2-yl)butyl)amino)-4-(pyrrolidin-1-yl)butan-2-yl)carbamate (2-5, 400 mg, 0.7 mmol) in 1,4-dioxane (4 mL) was added dropwise 4 M HCl in dioxane (1.8 mL, 7.4 mmol). The solution was stirred for 12 hours and concentrated under reduced pressure to yield the product, (R)-2-amino-4-oxo-N—((R)-4-phenyl-1-(4,4,5,5-tetramethyl-1,3,2-dioxaborolan-2-yl)butyl)-4-(pyrrolidin-1-yl)butanamide hydrochloride (2-6, 350 mg), which was used without purification. [M−H]⁻=442.5.

Synthesis of N—((R)-1,4-dioxo-1-(((R)-4-phenyl-1-(4,4,5,5-tetramethyl-1,3,2-dioxaborolan-2-yl)butyl) amino)-4-(pyrrolidin-1-yl)butan-2-yl)pyrazine-2-carboxamide [Step 5]: To a solution of pyrazine-2-carboxylic acid (2-7, 160 mg, 1.3 mmol) in tetrahydrofuran (7 mL) at −10° C. was added a mixture of isobutyl chloroformate (IBCF) (0.2 mL, 1.3 mmol) and N-methylmorpholine (NMM) (0.12 mL, 1.3 mmol) dropwise and the reaction mixture was stirred for 30 minutes at −10° C. To this reaction mixture was added a mixture of (R)-2-amino-4-oxo-N—((R)-4-phenyl-1-(4,4,5,5-tetramethyl-1,3,2-dioxaborolan-2-yl)butyl)-4-(pyrrolidin-1-yl)butanamide hydrochloride (2-6, 550 mg, 1.2 mmol) and NMM (0.17 mL, 1.3 mmol) in a mixture of tetrahydrofuran:dimethylformamide (3 mL:1 mL) and the reaction mixture was stirred for 2 hours and gradually brought to ambient temperature. The reaction was monitored by LC-MS, and when completed, the reaction mixture was diluted with ethyl acetate and the organic layer was washed with aqueous 0.1 N HCl (twice), 10% aqueous sodium carbonate (twice), water (twice), brine (twice), dried over sodium sulfate, and concentrated under reduced pressure to afford the product. The product was purified by reverse-phase prep-HPLC and lyophilized to yield N—((R)-1,4-dioxo-1-(((R)-4-phenyl-1-(4,4,5,5-tetramethyl-1,3,2-dioxaborolan-2-yl)butyl)amino)-4-(pyrrolidin-1-yl) butan-2-yl)pyrazine-2-carboxamide (2-8, 60 mg). [M−H]⁻=548.5.

Synthesis of ((R)-1-((R)-4-oxo-2-(pyrazine-2-carboxamido)-4-(pyrrolidin-1-yl)butanamido)-4-phenylbutyl)boronic acid [Step 6]: To a solution of N—((R)-1,4-dioxo-1-(((R)-4-phenyl-1-(4,4,5,5-tetramethyl-1,3,2-dioxaborolan-2-yl)butyl)amino)-4-(pyrrolidin-1-yl)butan-2-yl)pyrazine-2-carboxamide (2-8, 60 mg, 0.11 mmol) in acetone (2 mL) was added methylboronic acid (2-9, 65 mg, 1.1 mmol) followed by aqueous 0.2 M HCl (2 mL) dropwise. The reaction mixture was allowed to stir at ambient temperature overnight, and was concentrated under reduced pressure at ambient temperature. The product was redissolved in a mixture of acetonitrile and deionized water, and lyophilized to obtain ((R)-1-((R)-4-oxo-2-(pyrazine-2-carboxamido)-4-(pyrrolidin-1-yl)butanamido)-4-phenylbutyl)boronic acid (Compound 2, 45 mg). M−H]⁻=466.4. ¹H NMR (400 MHz, Methanol-d₄) δ 9.24 (d, 1H), 8.80 (d, 1H), 8.69 (d, 1H), 7.20 (t, 2H), 7.14 (d, 2H), 7.10 (d, 1H), 5.27 (s, 1H), 3.50 (q, 2H), 3.37 (q, 2H), 3.21 (d, 1H), 3.19 (d, 1H), 2.97 (d, 1H), 2.92 (d, 1H), 2.59 (t, 3H), 1.96 (q, 2H), 1.89-1.84 (m, 2H), 1.66-1.64 (m, 2H), 1.55-1.49 (m, 2H).

### Example 3

### Synthesis of ((R)-3-methyl-1-((R)-4-morpholino-4-oxo-2-(pyrazine-2-carboxamido) butanamido)butyl)boronic acid

In another embodiment, Compound 3 can be produced from compound (3-1) by the method shown in Scheme 3 (below). Carboxylic acid (3-1) is coupled with the amine of a protected boronic acid compound (3-2) to produce amide (3-3), which is then deprotected to generate amine (3-4) as the hydrochloride salt. Subsequent coupling with carboxylic acid (3-5) produces amide (3-6). Deprotection of (3-6) with methylboronic acid (3-7) produces Compound 3.

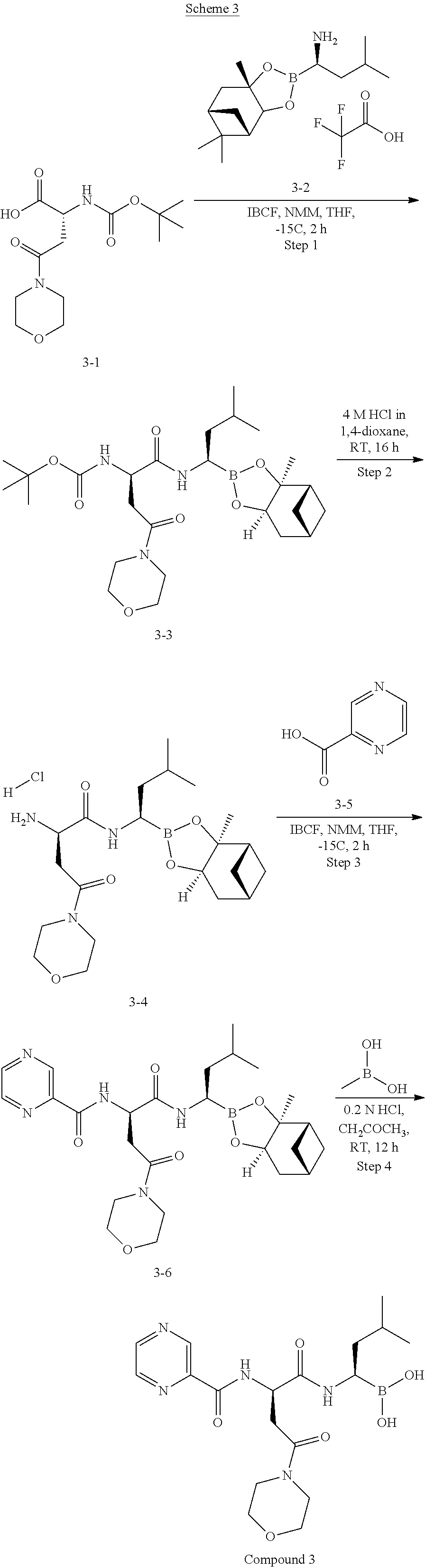

Synthesis of tert-butyl ((R)-1-(((R)-3-methyl-1-((3aS,4S,6S,7aR)-3a,5,5-trimethylhexahydro-4,6-methanobenzo[d][1,3,2]dioxaborol-2-yl)butyl)amino)-4-morpholino-1,4-dioxobutan-2-yl)carbamate [Step 1]: To a stirred solution of (R)-2-((tert-butoxycarbonyl)amino)-4-morpholino-4-oxobutanoic acid (3-1, 880 mg, 3 mmol) in THF (15 mL) was added NMM (0.4 mL, 3 mmol) followed by IBCF (0.4 mL, 3 mmol) at −15° C. and the reaction mixture was stirred at this temperature for 1 h. A mixture of (R)-3-methyl-1-((3aR,4R,6S,7aS)-3a,5,5-trimethylhexahydro-4,6-methanobenzo[d][1,3,2]dioxaborol-2-yl)butan-1-amine 2,2,2-trifluoroacetate (3-2, 1 g, 2.6 mmol) and NMM (0.4 mL, 3 mmol) were added and stirred at RT for 2 h. The reaction mixture was diluted with EtOAc and washed subsequently with 0.1 N HCl, 5% K₂CO₃, water and brine. The organic phase was dried over Na₂SO₄ and evaporated to get crude tert-butyl ((R)-1-(((R)-3-methyl-1-((3aR,4R,6R,7aS)-3a,5,5-trimethylhexahydro-4,6-methanobenzo[d][1,3,2]dioxaborol-2-yl)butyl)amino)-4-morpholino-1,4-dioxobutan-2-yl)carbamate (3-3, 1.4 g). This crude was used for forwarding step without further purification. [M+H]⁺=550.2.

Synthesis of (R)-2-amino-N—((R)-3-methyl-1-((3aS,4S,6S,7aR)-3a,5,5-trimethylhexahydro-4,6-methanobenzo[d][1,3,2]dioxaborol-2-yl)butyl)-4-morpholino-4-oxobutanamide hydrochloride [Step-2]: To a stirred solution of tert-butyl ((R)-1-(((R)-3-methyl-1-((3aR,4R,6R,7aS)-3a,5,5-trimethylhexahydro-4,6-methanobenzo[d][1,3,2]dioxaborol-2-yl)butyl) amino)-4-morpholino-1,4-dioxobutan-2-yl)carbamate (3-3, 1.4 g, 2.5 mmol) in 1,4-dioxane (6 mL) was added 4M HCl in 1,4-dioxane (6 mL, 25 mmol) in ice cold condition and stirred at RT for 2 h.

Volatiles were evaporated under reduced pressure below 35° C. and triturated with n-pentane to yield crude (R)-2-amino-N—((R)-3-methyl-1-((3aS,4S,6S,7aR)-3a,5,5-trimethylhexahydro-4,6-methanobenzo[d][1,3,2]dioxaborol-2-yl)butyl)-4-morpholino-4-oxobutanamide hydrochloride (3-4, 1.2 g). This crude was used for next step without further purification. [M+H]⁺=450.2.

Synthesis of N—((R)-1-(((R)-3-methyl-1-((3aS,4S,6S,7aR)-3a,5,5-trimethylhexahydro-4,6-methanobenzo[d][1,3,2]dioxaborol-2-yl)butyl)amino)-4-morpholino-1,4-dioxobutan-2-yl) pyrazine-2-carboxamide [Step 3]: To a stirred solution of pyrazine-2-carboxylic acid (3-5, 340 mg, 2.7 mmol) in THF (10 mL) was added NMM (0.4 mL, 2.7 mmol) followed by IBCF (0.4 mL, 2.7 mmol) at −15° C. and stirred at this temperature for 30 min. A mixture of (R)-2-amino-N—((R)-3-methyl-1-((3aS,4S,6S,7aR)-3a,5,5-trimethylhexahydro-4,6-methanobenzo[d][1,3,2]dioxaborol-2-yl)butyl)-4-morpholino-4-oxobutanamide hydrochloride (3-4, 1.2 g, 2.5 mmol) and NMM (0.4 mL, 2.7 mmol)) was added and the reaction mixture was stirred at RT for 2 h. The reaction mixture was diluted with EtOAc and washed subsequently with 0.1N HCl (2×15 mL), 5% K₂CO₃ (2×15 mL), water (2×15 mL) and brine. The organic phase was dried over Na₂SO₄ and evaporated.

The crude was purified via prep HPLC purification and lyophilized to afford N—((R)-1-(((R)-3-methyl-1-((3aS,4S,6S,7aR)-3a,5,5-trimethylhexahydro-4,6-methanobenzo [d][1,3,2]dioxaborol-2-yl)butyl)amino)-4-morpholino-1,4-dioxobutan-2-yl)pyrazine-2-carboxamide (3-6, 300 mg). [M−H]⁻=554.5 Synthesis of ((R)-3-methyl-1-((R)-4-morpholino-4-oxo-2-(pyrazine-2-carboxamido) butanamido)butyl)boronic acid [Step 4]: To a solution of N—((R)-1-(((R)-3-methyl-1-((3aS,4S,6S,7aR)-3a,5,5-trimethylhexahydro-4,6-methanobenzo[d][1,3,2]dioxaborol-2-yl)butyl) amino)-4-morpholino-1,4-dioxobutan-2-yl)pyrazine-2-carboxamide (3-6, 300 mg, 0.5 mmol) in acetone (4 mL) was added methylboronic acid (3-7, 320 mg, 5.4 mmol) followed by drop wise addition of 0.2 HCl (4 mL). The reaction mixture was stirred at room temperature for overnight. Volatiles were evaporated under reduced pressure and purified via prep HPLC purification and lyophilized to afford ((R)-3-methyl-1-((R)-4-morpholino-4-oxo-2-(pyrazine-2-carboxamido) butanamido)butyl) boronic acid (Compound 3, 90 mg). [M−H]⁻=420.3 ¹H NMR (400 MHz, MeOD) δ 9.25 (s, 1H), 8.80 (d, 1H), 8.69 (s, 1H), 5.30 (t, 1H), 3.68-3.61 (m, 4H), 3.55-3.51 (m, 4H), 3.28-3.27 (m, 1H), 3.02-2.97 (m, 1H), 2.70 (t, 1H), 1.67-1.66 (m, 1H), 1.35 (t, 2H), 0.80-0.87 (m, 6H).

### Example 4

### Synthesis of ((R)-1-((R)-4-morpholino-4-oxo-2-(pyrazine-2-carboxamido)butanamido)-3-phenylpropyl)boronic acid

In another embodiment, Compound 4 can be produced from compound (4-1) by the method shown in Scheme 4. Carboxylic acid (4-1) is coupled with the amine of a protected boronic acid compound (4-2) to produce amide (4-3), which is then deprotected to generate amine (4-4) as the hydrochloride salt. Subsequent coupling with carboxylic acid (4-5) produces amide (4-6). Deprotection of (4-6) with methylboronic acid (4-7) produces Compound 4.

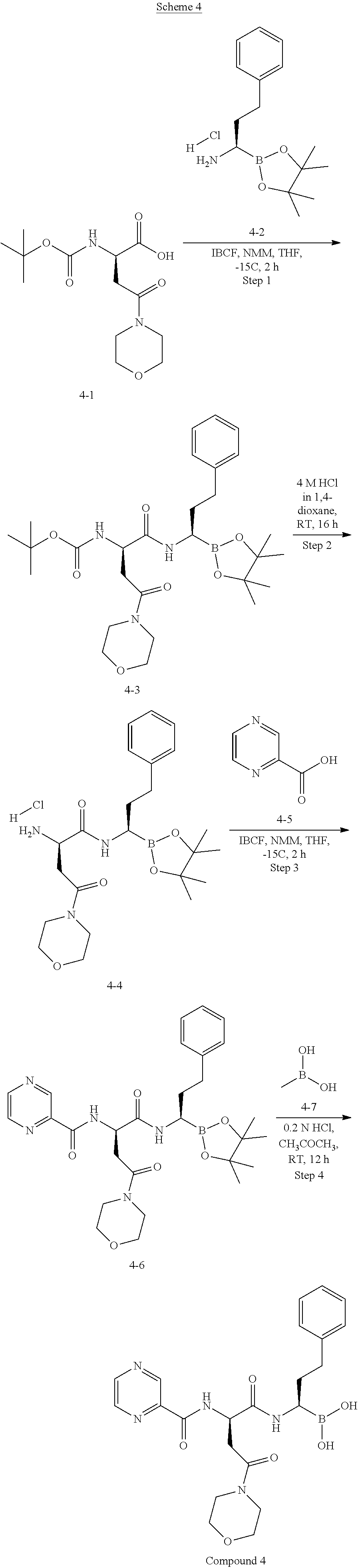

Synthesis of tert-butyl ((R)-4-morpholino-1,4-dioxo-1-(((R)-3-phenyl-1-(4,4,5,5-tetramethyl-1,3,2-dioxaborolan-2-yl)propyl)amino)butan-2-yl)carbamate [Step 1]: To a stirred solution of (R)-2-((tert-butoxycarbonyl)amino)-4-morpholino-4-oxobutanoic acid (4-1, 890 mg, 3 mmol) in THF (15 mL) was added NMM (0.40 mL, 3 mmol) followed by IBCF (0.40 mL, 3 mmol) at −15° C. and stirred at this temperature for 1 h. A mixture of (R)-3-phenyl-1-(4,4,5,5-tetramethyl-1,3,2-dioxaborolan-2-yl)propan-1-amine hydrochloride (4-2, 800 mg, 2.69 mmol) and NMM (0.40 mL, 2.96 mmol) was added and stirred at RT for 2 h. The reaction mixture was diluted with EtOAc and washed subsequently with 0.1N HCl, 5% K₂CO₃, water and brine. The organic phase was dried over Na₂SO₄ and evaporated to get crude tert-butyl ((R)-4-morpholino-1,4-dioxo-1-(((R)-3-phenyl-1-(4,4,5,5-tetramethyl-1,3,2-dioxaborolan-2-yl)propyl)amino) butan-2-yl)carbamate (4-3, 1.4 g). The crude was used for next step without further purification. [M−H]⁻=544.4.

Synthesis of (R)-2-amino-4-morpholino-4-oxo-N—((R)-3-phenyl-1-(4,4,5,5-tetramethyl-1,3,2-dioxaborolan-2-yl)propyl)butanamide hydrochloride [Step 2]: To a stirred solution of tert-butyl ((R)-4-morpholino-1,4-dioxo-1-(((R)-3-phenyl-1-(4,4,5,5-tetramethyl-1,3,2-dioxaborolan-2-yl) propyl)amino) butan-2-yl)carbamate (4-3, 1.4 mg, 2.6 mmol) in 1,4-dioxane (6 mL) was added 4N HCl in 1,4-dioxane (6 mL, 26 mmol) in ice cold condition and stirred at RT for 2 h. Volatiles were removed under reduced pressure and triturated with n-pentane to get crude (R)-2-amino-4-morpholino-4-oxo-N—((R)-3-phenyl-1-(4,4,5,5-tetramethyl-1,3,2-dioxaborolan-2-yl)propyl) butanamide hydrochloride (4-4, 1 g). This crude was used for next step without further purification. [M−H]⁻=444.4.

Synthesis of N—((R)-4-morpholino-1,4-dioxo-1-(((R)-3-phenyl-1-(4,4,5,5-tetramethyl-1,3,2-dioxaborolan-2-yl)propyl)amino)butan-2-yl)pyrazine-2-carboxamide [Step 3]: To a stirred solution of pyrazine-2-carboxylic acid (4-5, 280 mg, 2.3 mmol) in THF (10 mL) was added NMM (0.3 mL, 2.3 mmol) followed by IBCF (0.3 mL, 2.3 mmol) at −15° C. and stirred at this temperature for 30 min. A mixture of (R)-2-amino-4-morpholino-4-oxo-N—((R)-3-phenyl-1-(4,4,5,5-tetramethyl-1,3,2-dioxaborolan-2-yl)propyl) butanamide hydrochloride (4-4, 1 g, 2 mmol) and NMM (0.3 mL, 2.3 mmol) were added and stirred at RT for 2 h. The reaction mixture was diluted with EtOAc and washed subsequently with 0.1N HCl (2×15 mL), 5% K₂CO₃ (2×15 mL), water (2×15 mL) and brine, dried over Na₂SO₄ and evaporated. The crude was purified via prep HPLC purification and lyophilized to afford N—((R)-4-morpholino-1,4-dioxo-1-(((R)-3-phenyl-1-(4,4,5,5-tetramethyl-1,3,2-dioxaborolan-2-yl)propyl)amino)butan-2-yl)pyrazine-2-carboxamide (4-6, 150 mg). [M−H]⁻=550.4.

Synthesis of ((R)-1-((R)-4-morpholino-4-oxo-2-(pyrazine-2-carboxamido)butanamido)-3-phenylpropyl)boronic acid [step-4]: To a solution of N—((R)-4-morpholino-1,4-dioxo-1-(((R)-3-phenyl-1-(4,4,5,5-tetramethyl-1,3,2-dioxaborolan-2-yl)propyl)amino)butan-2-yl)pyrazine-2-carboxamide (4-6, 150 mg, 0.3 mmol) in acetone (2 mL) was added methylboronic acid (4-7, 160 mg, 2.7 mmol) followed by drop wise addition of 0.2N HCl (2 mL) and stirred at room temperature for overnight. Volatiles were removed under reduced pressure and lyophilized. The crude was purified via prep HPLC purification and lyophilized to afford ((R)-1-((R)-4-morpholino-4-oxo-2-(pyrazine-2-carboxamido)butanamido)-3-phenylpropyl)boronic acid (Compound 4, 50 mg). [M−H]⁻=468.3 ¹H NMR (400 MHz, MeOD) δ 9.27 (d, 1H), 8.81 (d, 1H), 8.70-8.69 (m, 1H), 7.22-7.16 (m, 4H), 7.09 (t, 1H), 5.31 (t, 1H), 3.65 (t, 2H), 3.63-3.6 (m, 2H), 3.57-3.51 (m, 5H), 3.04-2.98 (m, 1H), 2.63 (t, 3H), 1.84-1.71 (m, 2H).

### Example 5

### Synthesis of ((R)-1-((R)-4-morpholino-4-oxo-2-(pyrazine-2-carboxamido)butanamido) pentyl)boronic acid

In another embodiment, Compound 5 can be produced from compound (5-1) by the method shown in Scheme 5. Carboxylic acid (5-1) is coupled with the amine of a protected boronic acid compound (5-2) to produce amide (5-3), which is then deprotected to generate amine (5-4) as the hydrochloride salt. Subsequent coupling with carboxylic acid (5-5) produces amide (5-6). Deprotection of (5-6) with methylboronic acid (5-7) produces Compound 5.

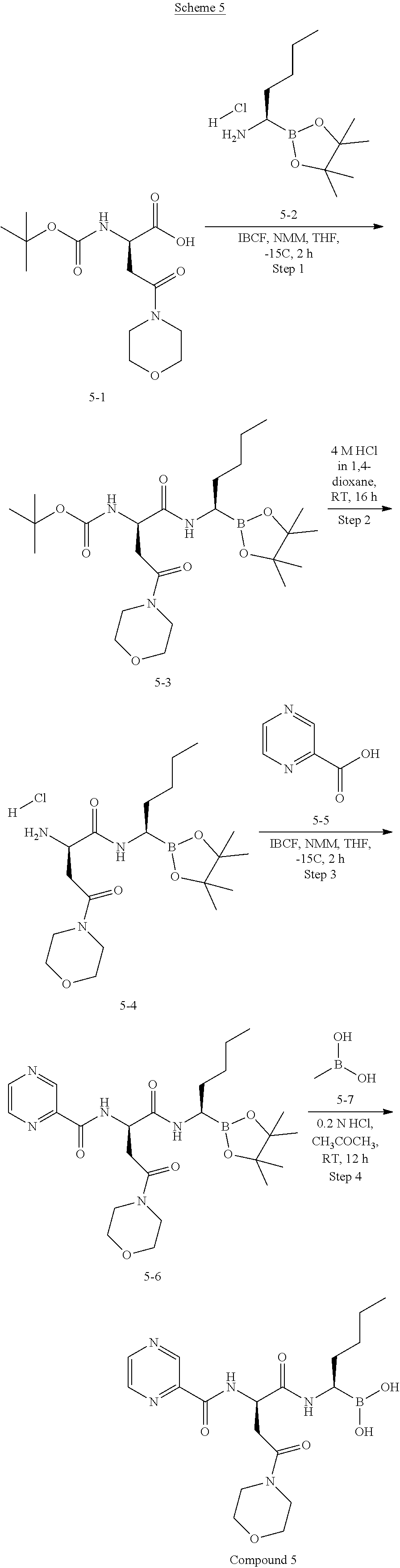

Synthesis of tert-butyl ((R)-4-morpholino-1,4-dioxo-1-(((R)-1-(4,4,5,5-tetramethyl-1,3,2-dioxaborolan-2-yl)pentyl)amino)butan-2-yl)carbamate [Step 1]: To a stirred solution of (R)-2-((tert-butoxycarbonyl)amino)-4-morpholino-4-oxobutanoic acid (5-1, 1.1 g, 3.5 mmol) in THF (15 mL), NMM (0.4 mL, 3.5 mmol) and IBCF (0.5 mL, 3.5 mmol) were added at −15° C. and the reaction mixture was stirred at this temperature for 1 h. To the above solution, (R)-1-(4,4,5,5-tetramethyl-1,3,2-dioxaborolan-2-yl)pentan-1-amine hydrochloride (5-2, 800 mg, 3.2 mmol) and NMM (0.4 mL, 3.5 mmol) were added and the reaction mixture was stirred at RT for 2 h. After completion of the reaction (monitored by TLC and LC-MS), the reaction mixture was diluted with EtOAc and washed subsequently with 0.1 M HCl, 5% K₂CO₃, water and brine. The organic phase was dried over Na₂SO₄, filtered and evaporated to yield tert-butyl ((R)-4-morpholino-1,4-dioxo-1-(((R)-1-(4,4,5,5-tetramethyl-1,3,2-dioxaborolan-2-yl)pentyl)amino)butan-2-yl)carbamate (5-3, 1500 mg). The crude was used for next step without further purification. [M+H]⁺=498.1.

Synthesis of (R)-2-amino-4-morpholino-4-oxo-N—((R)-1-(4,4,5,5-tetramethyl-1,3,2-dioxaborolan-2-yl)pentyl)butanamide hydrochloride [Step 2]: To a stirred solution of tert-butyl ((R)-4-morpholino-1,4-dioxo-1-(((R)-1-(4,4,5,5-tetramethyl-1,3,2-dioxaborolan-2-yl)pentyl) amino) butan-2-yl)carbamate (5-3, 1500 mg, 3 mmol) in 1,4-Dioxane (7 mL) was added 4 M HCl-dioxane (7 mL, 30 mmol) in ice cold condition and the reaction mixture was stirred at RT for 2 h. After completion of the reaction (monitored by LCMS) the solvent was evaporated from reaction mixture. The crude was triturated with n-pentane to yield (R)-2-amino-4-morpholino-4-oxo-N—((R)-1-(4,4,5,5-tetramethyl-1,3,2-dioxaborolan-2-yl)pentyl)butanamide hydrochloride (5-4, 1000 mg). This crude was used for next step without further purification. [M−H]⁻=396.4.

Synthesis of N—((R)-4-morpholino-1,4-dioxo-1-(((R)-1-(4,4,5,5-tetramethyl-1,3,2-dioxaborolan-2-yl)pentyl) amino)butan-2-yl)pyrazine-2-carboxamide [Step 3]: To a stirred solution of pyrazine-2-carboxylic acid (5-5, 190 mg, 1.5 mmol) in THF (8 mL), NMM (0.2 mL, 1.5 mmol) and IBCF (0.2 mL, 1.5 mmol) were added at −15° C. and the reaction mixture was stirred at this temperature for 30 min. Then ((R)-2-amino-4-morpholino-4-oxo-N—((R)-1-(4,4,5,5-tetramethyl-1,3,2-dioxaborolan-2-yl)pentyl)butanamide hydrochloride (5-4, 600 mg, 1.4 mmol) and NMM (0.2 mL, 1.5 mmol) were added and the reaction mixture was stirred at RT for 2 h. After completion of the reaction (monitored by TLC and LC-MS), the reaction mixture was diluted with EtOAc and washed subsequently with 0.1 M HCl, 5% K₂CO₃, water and brine. The organic phase was dried over Na₂SO₄, filtered and evaporated to yield a brown gum. The crude was purified by RP prep HPLC purification and the eluent was lyophilized to afford N—((R)-4-morpholino-1,4-dioxo-1-(((R)-1-(4,4,5,5-tetramethyl-1,3,2-dioxaborolan-2-yl)pentyl) amino)butan-2-yl)pyrazine-2-carboxamide (5-6, 60 mg). [M−H]⁻=502.4.

Synthesis of ((R)-1-((R)-4-morpholino-4-oxo-2-(pyrazine-2-carboxamido)butanamido) pentyl)boronic acid [Step 4]: To a solution of N—((R)-4-morpholino-1,4-dioxo-1-(((R)-1-(4,4,5,5-tetramethyl-1,3,2-dioxaborolan-2-yl)pentyl) amino)butan-2-yl)pyrazine-2-carboxamide (5-6, 60 mg, 0.1 mmol) in acetone (2 mL) was added methylboronic acid (5-7, 70 mg, 1 mmol), followed by drop wise addition of 0.2 HCl (2 mL). The reaction mixture was allowed to stirring at room temperature overnight. All the volatiles were evaporated at room temperature, and reaction mixture was re-dissolved in acetonitrile and deionized water and freeze-dried. The obtained crude was purified through RP prep HPLC purification and lyophilized to afford ((R)-1-((R)-4-morpholino-4-oxo-2-(pyrazine-2-carboxamido)butanamido) pentyl)boronic acid (Compound 5, 40 mg). [M−H]⁻=420.2. ¹H NMR (400 MHz, MeOD) δ 9.26 (s, 1H), 8.80 (d, 1H), 8.69 (s, 1H), 5.30 (d, 1H), 3.68-3.65 (m, 2H), 3.64-3.62 (m, 2H), 3.54-3.47 (m, 4H), 3.27 (s, 1H), 2.99 (dd, 1H), 2.57 (t, 1H), 1.51-1.48 (m, 1H), 1.46-1.44 (m, 1H), 1.31-1.28 (m, 4H), 0.88 (s, 3H).

### Example 6

### Synthesis of ((R)-1-((R)-2-(2,4-dimethyloxazole-5-carboxamido)-4-morpholino-4-oxobutanamido)-3-methylbutyl)boronic acid

In another embodiment, Compound 6 can be produced from compound (6-1) by the method shown in Scheme 8. Carboxylic acid (6-1) is coupled with the amine of a protected boronic acid compound as the trifluoroacetic acid salt (6-2) to produce amide (6-3). Removal of the BOC protecting group yields the corresponding amine (6-4) as the hydrochloride salt. Subsequent coupling with carboxylic acid (6-5) produces amide (6-6). Deprotection of (6-6) with methylboronic acid (6-7) produces Compound 6.

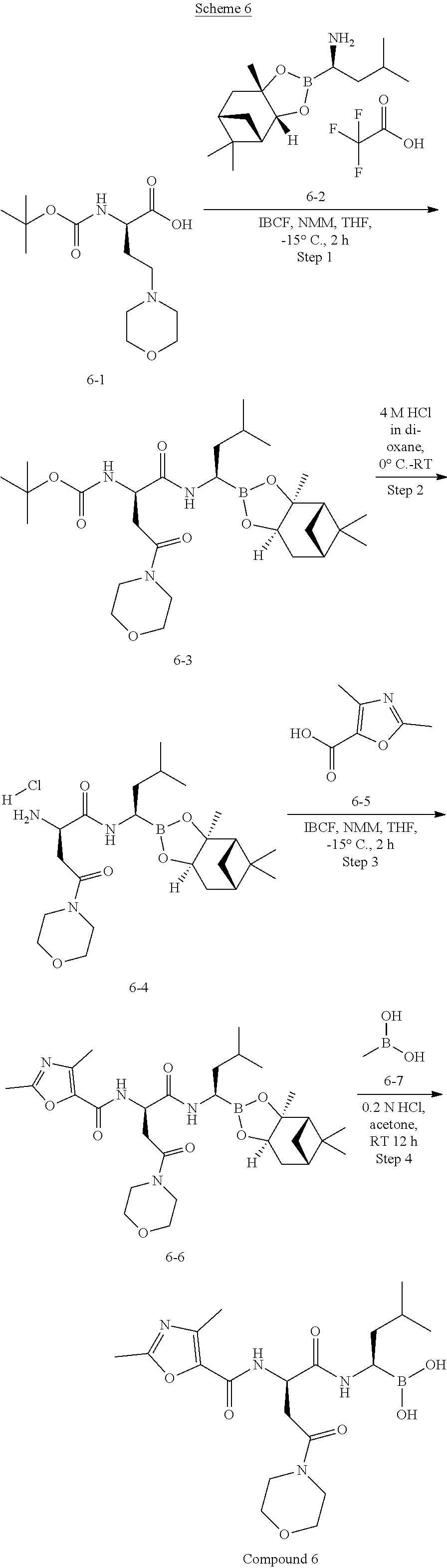

Synthesis of tert-butyl ((R)-1-(((R)-3-methyl-1-((3aS,4S,6S,7aR)-3a,5,5-trimethylhexahydro-4,6-methanobenzo[d][1,3,2]dioxaborol-2-yn)butyl)amino)-4-morpholino-1,4-dioxobutan-2-yl)carbamate [Step 1]: To a stirred solution of (R)-2-((tert-butoxycarbonyl)amino)-4-morpholino-4-oxobutanoic acid (6-1, 530 mg, 1.8 mmol) in THF (8 mL) was added IBCF (0.3 mL, 1.8 mmol) and NMM (0.3 mL, 2 mmol) at −15° C. The reaction mixture was stirred at same temperature for 30 min. Then (R)-3-methyl-1-((3aR,4S,6S,7aS)-5,5,7a-trimethyl hexahydro-4,6-methanobenzo[d][1,3,2]dioxaborol-2-yl)butan-1-amine TFA salt (6-2, 600 mg, 1.6 mmol) in DMF (1 mL) followed by NMM (0.3 mL, 1.8 mmol) was added to the reaction mixture at −15° C. It was gradually warmed to 0° C. and stirred for 2 h. The reaction mixture was neutralized with saturated aqueous 0.1 N HCl solution and extracted with ethyl acetate. The combined organic layer was washed with 5% K₂CO₃ solution, water and brine. It was dried over Na₂SO₄, filtered and evaporated under reduced pressure to afford tert-butyl ((R)-1-(((R)-3-methyl-1-((3aS,4S,6S,7aR)-3a,5,5-trimethylhexahydro-4,6-methanobenzo[d][1,3,2]dioxaborol-2-yl)butyl)amino)-4-morpholino-1,4-dioxobutan-2-yl)carbamate (6-3, 800 mg). [M−H]⁺: 548.

Synthesis of (R)-2-amino-N—((R)-3-methyl-1-((3aS,4S,6S,7aR)-3a,5,5-trimethylhexahydro-4,6-methanobenzo[d][1,3,2]dioxaborol-2-yl)butyl)-4-morpholino-4-oxobutanamide [Step 2]: To a solution of tert-butyl ((R)-1-(((R)-3-methyl-1-((3aS,4S,6S,7aR)-3a,5,5-trimethylhexahydro-4,6-methanobenzo[d][1,3,2]dioxaborol-2-yl)butyl)amino)-4-morpholino-1,4-dioxobutan-2-yl) carbamate (6-3, 800 mg, 1.5 mmol) in 1,4-dioxane (6 mL) was added 4 M HCl in 1,4 dioxane (5 mL, 22 mmol) at 0° C. It was gradually warmed to 25° C. and stirred for 16 h. The volatiles were removed under reduced pressure to get (R)-2-amino-N—((R)-3-methyl-1-((3aS,4S,6S,7aR)-3a,5,5-trimethylhexahydro-4,6-methanobenzo[d][1,3,2]dioxaborol-2-yl)butyl)-4-morpholino-4-oxobutanamide (6-4, 500 mg). [M−H]⁺=448.6.

Synthesis of 2,4-dimethyl-N—((R)-1-(((R)-3-methyl-1-((3aS,4S,6S,7aR)-3a,5,5-trimethyl hexahydro-4,6-methanobenzo[d][1,3,2]dioxaborol-2-yl)butyl)amino)-4-morpholino-1,4-dioxobutan-2-yl)oxazole-5-carboxamide [Step 3]: To a stirred solution of 2,4-dimethyloxazole-5-carboxylic acid (6-5, 175 mg, 1.2 mmol) in THF (8 mL) was added IBCF (0.3 mL, 1.8 mmol) and NMM (0.3 mL, 1.8 mmol) at −15° C. The reaction mixture was stirred at same temperature for 30 min. Then (R)-2-amino-N—((R)-3-methyl-1-((3aS,4S,6S,7aR)-3a,5,5-trimethylhexahydro-4,6-methanobenzo[d][1,3,2]dioxaborol-2-yl)butyl)-4-morpholino-4-oxobutanamide (6-4, 500 mg, 1.1 mmol) in DMF (1 mL) followed by NMM (0.3 mL, 1.8 mmol) were added to the reaction mixture at −15° C. It was gradually warmed to 0° C. and stirred for 2 h. It was neutralized with saturated aqueous 0.1 N HCl solution and extracted with ethyl acetate. The combined organic layer was washed with 5% K₂CO₃ solution, water and brine. It was dried over Na₂SO₄, filtered and evaporated under reduced pressure to afford 2,4-dimethyl-N—((R)-1-(((R)-3-methyl-1-((3aS,4S,6S,7aR)-3a,5,5-trimethylhexahydro-4,6-methanobenzo[d][1,3,2]dioxaborol-2-yl)butyl)amino)-4-morpholino-1,4-dioxobutan-2-yl)oxazole-5-carboxamide (6-6, 200 mg). [M−H]⁺=571.4.

Synthesis of ((R)-1-((R)-2-(2,4-dimethyloxazole-5-carboxamido)-4-morpholino-4-oxobutanamido)-3-methylbutyl)boronic acid [Step 4]: To a stirred solution of 2,4-dimethyl-N—((R)-1-(((R)-3-methyl-1-((3aS,4S,6S,7aR)-3a,5,5-trimethylhexahydro-4,6-methanobenzo[d][1,3,2]dioxaborol-2-yl)butyl)amino)-4-morpholino-1,4-dioxobutan-2-yl)oxazole-5-carboxamide (6-6, 100 mg, 0.2 mmol) and methyl boronic acid (6-7, 155 mg, 2.6 mmol) in acetone (2 mL) was added 0.2 N HCl (2.0 mL) and the reaction mixture was stirred at RT overnight. The volatiles were evaporated and the residue was redissolved in acetone and deionized water and freeze-dried to obtain crude. The crude material was purified by RP prep HPLC purification and lyophilized to afford ((R)-1-((R)-2-(2,4-dimethyloxazole-5-carboxamido)-4-morpholino-4-oxobutanamido)-3-methylbutyl)boronic acid (Compound 6, 40 mg). [M−H]⁺=437. ¹H NMR (400 MHz, MeOD) δ 5.20 (t, 1H), 3.68-3.66 (m, 2H), 3.62 (d, 2H), 3.55-3.51 (m, 4H), 3.17-3.11 (m, 1H), 3.01-2.96 (m, 1H), 2.71 (t, 1H), 2.48 (s, 3H), 2.39 (s, 3H), 1.98 (brs, 1H), 1.70-1.63 (m, 1H), 1.35 (t, 2H), 0.90 (d, 6H).

### Example 7

### Synthesis of ((R)-1-((R)-4-(dimethylamino)-4-oxo-2-(pyrazine-2-carboxamido)butanamido)-4-phenylbutyl)boronic acid

In another embodiment, Compound 7 can be produced from compound (7-1) by the method shown in Scheme 7. Carboxylic acid (7-1) is coupled with the dimethylamine (7-2) to produce amide (7-3), which is then deprotected to generate carboxylic acid (7-4). Subsequent coupling with amine of a protected boronic acid compound (7-5) produces amide (7-6). Removal of the BOC protecting group yields the corresponding amine (7-7) as the hydrochloride salt, which is then coupled with carboxylic acid (7-8) to form amide (7-9). Deprotection of (7-9) with methylboronic acid (7-10) produces Compound 7.

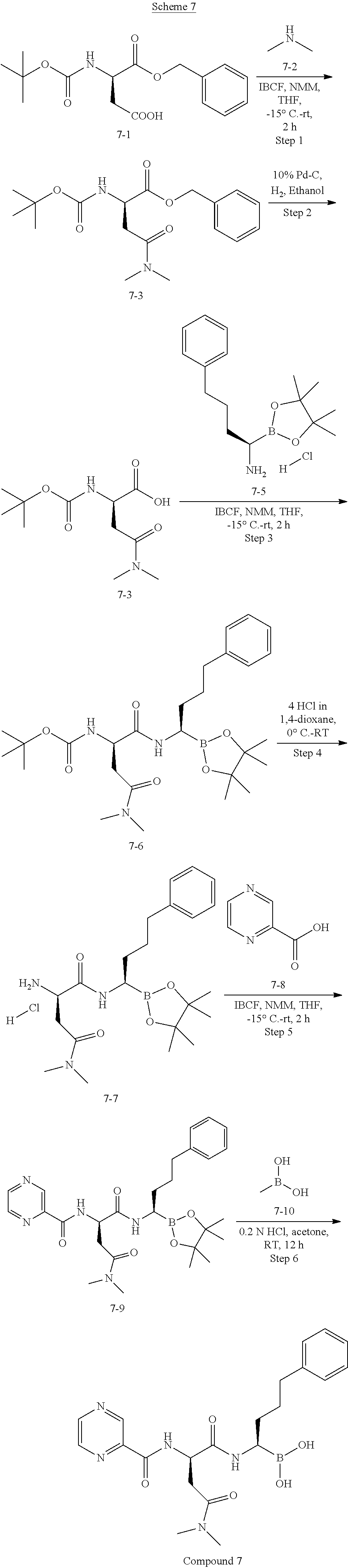

Synthesis of benzyl N2-(tert-butoxycarbonyl)-N⁴,N⁴-dimethyl-D-asparaginate [Step 1]: To a stirred solution of (R)-4-(benzyloxy)-3-((tert-butoxycarbonyl)amino)-4-oxobutanoic acid (7-1, 1 g, 3.2 mmol) in THF (20 mL) was added IBCF (0.4 mL, 3.2 mmol) and NMM (0.4 mL, 3.2 mmol) at −15° C. Reaction mixture was stirred at same temperature for 30 min. Then dimethylamine (7-2, 2 M in THF) (1.4 ml, 2.9 mmol) followed by NMM (0.3 mL, 2.9 mmol) was added to reaction mixture at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. The combined organic layer was washed with 5% K₂CO₃ solution, water and brine. It was dried over Na₂SO₄, filtered and evaporated under reduced pressure to get crude. The crude was purified by combi-flash column chromatography using 0-50% EtOAc in hexanes as eluent to afford benzyl N²-(tert-butoxycarbonyl)-N⁴,N⁴-dimethyl-D-asparaginate (7-3, 600 mg). [M+H]⁺: 350.8. ¹H NMR (400 MHz, DMSO-d₆) (400 MHz, DMSO-d₆) δ 7.52-7.17 (m, 5H), 5.10 (s, 2H), 4.56-4.36 (m, 1H), 2.91 (s, 3H), 2.80 (s, 3H), 2.79-2.64 (m, 2H), 1.36 (s, 9H), 1.32-1.26 (m, 1H).

Synthesis of N2-(tert-butoxycarbonyl)-N⁴,N⁴-dimethyl-D-asparagine [Step 2]: To a stirred solution of benzyl N²-(tert-butoxycarbonyl)-N⁴,N⁴-dimethyl-D-asparaginate (7-3, 1.0 g, 2.8 mmol) dissolved in THF (25 mL) was bubbled with nitrogen gas for 10 min. Then 10% Pd—C (400 mg) was added and the reaction mixture was hydrogenated under balloon pressure for 3 h. The progress of the reaction was monitored using TLC. Upon completion, the reaction mixture was filtered over celite bed and washed the bed with excess ethyl acetate to get crude N²-(tert-butoxycarbonyl)-N⁴,N⁴-dimethyl-D-asparagine (7-4, 700 mg). It was used in next step without further purification. [M+H]: 260.8. ¹H NMR (400 MHz, DMSO-d₆) δ 12.48 (s, 1H), 6.70 (d, 1H), 4.31 (s, 1H), 3.97-3.69 (m, 2H), 2.93 (s, 3H), 2.80 (s, 3H), 1.38 (s, 9H).

Synthesis of tert-butyl ((R)-4-(dimethylamino)-1,4-dioxo-1-(((R)-4-phenyl-1-(4,4,5,5-tetramethyl-1,3,2-dioxaborolan-2-yl)butyl)amino)butan-2-yl)carbamate [Step 3]: To a stirred solution of N²-(tert-butoxycarbonyl)-N⁴,N⁴-dimethyl-D-asparaginate (7-4, 200 mg, 0.8 mmol) in THF (8 mL) was added IBCF (0.1 mL, 0.8 mmol) and NMM (85 μmL, 0.8 mmol) at −15° C. The reaction mixture was stirred at same temperature for 30 min. Then (R)-4-phenyl-1-(4,4,5,5-tetramethyl-1,3,2-dioxaborolan-2-yl)butan-1-amine hydrochloride (7-5, 220 mg, 0.7 mmol) in DMF (1 mL) followed by NMM (0.08 mL, 0.7 mmol) was added to reaction mixture at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. The combined organic layer was washed with 5% K₂CO₃ solution, water, brine, dried over Na₂SO₄, filtered and evaporated under reduced pressure to afford tert-butyl ((R)-4-(dimethylamino)-1,4-dioxo-1-(((R)-4-phenyl-1-(4,4,5,5-tetramethyl-1,3,2-dioxaborolan-2-yl)butyl)amino)butan-2-yl)carbamate (7-6, 250 mg). The crude was used directly without further purification. [M+H]: 518.1.

Synthesis of (R)-2-amino-N⁴,N⁴-dimethyl-N¹—((R)-4-phenyl-1-(4,4,5,5-tetramethyl-1,3,2-dioxaborolan-2-yl)butyl)succinamide hydrochloride [Step 4]: To a solution of tert-butyl ((R)-4-(dimethylamino)-1,4-dioxo-1-(((R)-4-phenyl-1-(4,4,5,5-tetramethyl-1,3,2-dioxaborolan-2-yl) butyl)amino)butan-2-yl)carbamate (7-6, 650 mg, 1.2 mmol) in 1,4-dioxane (6 mL) was added 4 M HCl in 1,4 dioxane (6.0 mL, 25 mmol) at 0° C. It was gradually warmed to 25° C. and stirred for 16 h. TLC showed complete consumption of starting material to form new polar spot. Volatiles were removed under reduced pressure to get crude (R)-2-amino-N⁴,N⁴-dimethyl-N¹—((R)-4-phenyl-1-(4,4,5,5-tetramethyl-1,3,2-dioxaborolan-2-yl)butyl)succinamide hydrochloride (7-7, 550 mg, 1.2 mmol). The crude was directly used for next step without purification.

Synthesis of (R)—N⁴,N⁴-dimethyl-N¹—((R)-4-phenyl-1-(4,4,5,5-tetramethyl-1,3,2-dioxaborolan-2-yl)butyl)-2-(pyrazine-2-carboxamido)succinamide [Step 5]: To a stirred solution of pyrazine-2-carboxylic acid (7-8, 165 mg, 1.3 mmol) in THF (8 mL) was added IBCF (0.2 mL, 1.3 mmol) and NMM (0.2 mL, 1.33 mmol) at −15° C. The reaction mixture was stirred at same temperature for 30 min. Then (R)-2-amino-N⁴,N⁴-dimethyl-N¹—((R)-4-phenyl-1-(4,4,5,5-tetramethyl-1,3,2-dioxaborolan-2-yl)butyl)succinamide hydrochloride (7-7, 550 mg, 1.2 mmol) in DMF (1 mL) followed by NMM (0.13 mL, 1.2 mmol) was added to reaction mixture under same condition. It was gradually warmed to 0° C. and stirred for 2 h. LCMS of crude reaction mass confirmed the formation of desired product. It was neutralized with saturated aqueous (0.1 N) HCl solution and extracted with ethyl acetate. The combined organic layer was washed with 5% K₂CO₃ solution, water, brine, dried over Na₂SO₄, filtered and evaporated under reduced pressure. The crude material was purified by RP prep HPLC purification and lyophilized to afford (R)—N⁴,N⁴-dimethyl-N¹—((R)-4-phenyl-1-(4,4,5,5-tetramethyl-1,3,2-dioxaborolan-2-yl)butyl)-2-(pyrazine-2-carboxamido)succinamide (7-9, 30 mg). [M−H]⁺=522.5; [M-84+H]⁺=440.6.

Synthesis of ((R)-1-((R)-4-(dimethylamino)-4-oxo-2-(pyrazine-2-carboxamido) butanamido)-4-phenylbutyl)boronic acid [Step 6]: To a stirred solution of (R)—N⁴,N⁴-dimethyl-N¹—((R)-4-phenyl-1-(4,4,5,5-tetramethyl-1,3,2-dioxaborolan-2-yl)butyl)-2-(pyrazine-2-carboxamido) succinamide (7-9, 30 mg, 0.06 mmol) and methyl boronic acid (7-10, 34 mg, 0.6 mmol) in acetone (2 mL) was added 0.2 N HCl (2 mL) and the reaction mixture was stirred at RT overnight. TLC and LCMS showed full conversion of starting material with formation of new polar spot. The volatiles were evaporated and the residue was purified by RP prep HPLC purification to obtain ((R)-1-((R)-4-(dimethylamino)-4-oxo-2-(pyrazine-2-carboxamido) butanamido)-4-phenylbutyl)boronic acid (Compound 7, 7.0 mg). [M−H]⁺=440.2. ¹H NMR (400 MHz, Methanol-d₄) δ 9.24 (s, 1H), 8.80 (d, 1H), 8.71-8.65 (m, 1H), 7.24-7.05 (m, 5H), 5.26 (s, 1H), 3.05 (s, 3H), 2.90 (s, 3H), 2.58 (d, 3H), 1.77-1.41 (m, 6H).

### Example 8

### Synthesis of ((R)-1-((R)-4-(ethylamino)-4-oxo-2-(pyrazine-2-carboxamido)butanamido)-4-phenylbutyl)boronic acid

In another embodiment, Compound 8 can be produced from compound (8-1) by the method shown in Scheme 8. Carboxylic acid (8-1) is coupled with the ethylamine (8-2) to produce amide (8-3), which is deprotected to generate carboxylic acid (8-4). Subsequent coupling with the amine of a protected boronic acid compound (8-5) produces amide (8-6). Removal of the BOC protecting group yields the corresponding amine (8-7) as the hydrochloride salt, which is then coupled with carboxylic acid (8-8) to form amide (8-9). Deprotection of (8-9) with methylboronic acid (8-10) produces Compound 8.

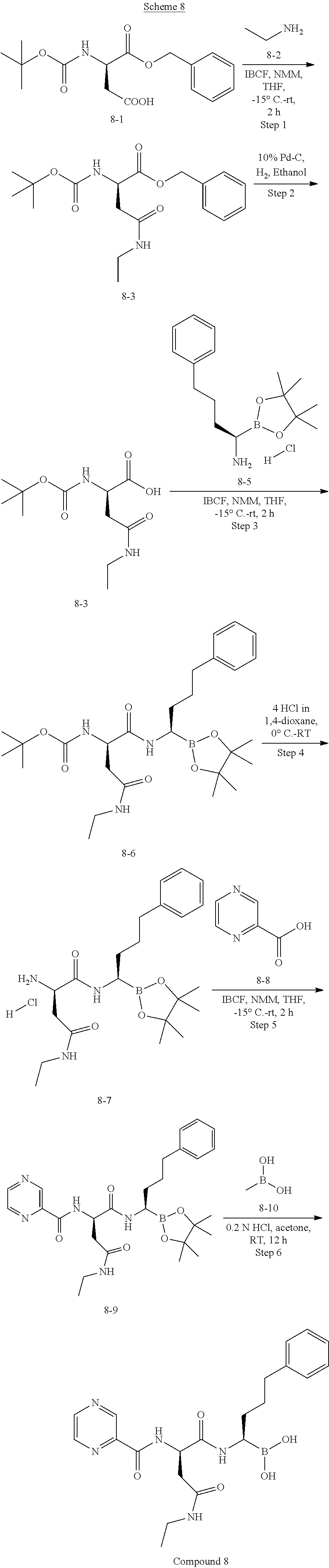

Synthesis of benzyl N²-(tert-butoxycarbonyl)-N⁴-ethyl-D-asparaginate [Step 1]: To a solution of (R)-4-(benzyloxy)-3-((tert-butoxycarbonyl)amino)-4-oxobutanoic acid (8-1, 1.0 g, 3.3 mmol) in THF (10 mL), IBCF (0.5 mL, 3.3 mmol) and NMM (0.4 mL, 3.3 mmol) were added and placed under N₂ and cooled to −10° C. The reaction mixture gradually became opaque with a fine white precipitate. Then ethyl amine in 2 M THF (8-2, 1.5 ml, 3.0 mmol) and NMM (0.3 mL, 3.0 mmol) were added at 0° C. The reaction mixture was stirred at room temperature for 2 h. After 2 h, starting material was consumed and desired mass was formed according to LCMS. The reaction mixture was diluted with ethyl acetate and was washed with 0.1N HCl solution (2 times), aq. 5% K₂CO₃ solution (2 times), water and brine. The organic phase was dried over Na₂SO₄, filtered, and concentrated under reduced pressure to get the crude compound. The crude compound was purified by combi-flash column chromatography using 0-50% EtOAc in hexanes as eluent to get benzyl N²-(tert-butoxycarbonyl)-N⁴-ethyl-D-asparaginate (8-3, 750 mg). [M+H]⁺: 350.8. ¹H NMR (400 MHz, DMSO-d₆) δ 7.86 (s, 1H), 7.34 (s, 5H), 7.14 (d, 1H), 5.09 (s, 2H), 4.40 (d, 1H), 3.15-2.92 (m, 2H), 2.65-2.51 (m, 1H), 2.48-2.34 (m, 1H), 1.45-1.22 (m, 9H), 0.97 (t, 3H).

Synthesis of N²-(tert-butoxycarbonyl)-N⁴-ethyl-D-asparagine [Step 2]: To a stirred solution of benzyl N²-(tert-butoxycarbonyl)-N⁴-ethyl-D-asparaginate (8-3, 1.1 g, 3.1 mmol) dissolved in THF (5 mL) was bubbled with nitrogen gas for 10 min. Then 10% Pd—C (400 mg) was added and the reaction mixture was hydrogenated under balloon pressure for 3 h. The reaction was monitored by TLC. Upon completion, the reaction was filtered over celite with excess ethyl acetate to get crude N²-(tert-butoxycarbonyl)-N⁴-ethyl-D-asparagine (8-4, 800 mg). The crude compound was used in next step without further purification. [M+H]: 260.8; ¹H NMR (400 MHz, DMSO-d₆) δ 12.52 (s, 1H), 7.89-7.76 (m, 1H), 6.89 (d, 1H), 4.26 (q, 1H), 3.10-2.98 (m, 2H), 2.50-2.35 (m, 2H), 1.37 (s, 9H), 0.99 (t, 3H).

Synthesis of tert-butyl ((R)-4-(ethylamino)-1,4-dioxo-1-(((R)-4-phenyl-1-(4,4,5,5-tetramethyl-1,3,2-dioxaborolan-2-yl)butyl)amino)butan-2-yl)carbamate [Step 3]: To a stirred solution of N²-(tert-butoxycarbonyl)-N⁴-ethyl-D-asparagine (8-4, 200 mg, 0.8 mmol) in THF (8 mL) was added IBCF (0.1 mL, 0.8 mmol) and NMM (0.08 mL, 0.8 mmol) at −10° C. The reaction mixture was stirred at same temperature for 30 min. Then (R)-4-phenyl-1-(4,4,5,5-tetramethyl-1,3,2-dioxaborolan-2-yl)butan-1-amine hydrochloride (8-5, 220 mg, 0.7 mmol) in DMF (1 mL) followed by NMM (0.08 mL, 0.7 mmol) was added to the reaction mixture at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. The combined organic layer was washed with 5% K₂CO₃ solution, water, and brine, dried over Na₂SO₄, filtered and evaporated under reduced pressure to tert-butyl ((R)-4-(ethylamino)-1,4-dioxo-1-(((R)-4-phenyl-1-(4,4,5,5-tetramethyl-1,3,2-dioxaborolan-2-yl) butyl)amino)butan-2-yl)carbamate (8-6, 250 mg). The crude was used directly without further purification. [M+H]: 518.1.

Synthesis of (R)-2-amino-N⁴-ethyl-N¹—((R)-4-phenyl-1-(4,4,5,5-tetramethyl-1,3,2-dioxaborolan-2-yl)butyl)succinamide hydrochloride [Step 4]: To a stirred solution of tert-butyl ((R)-4-(ethylamino)-1,4-dioxo-1-(((R)-4-phenyl-1-(4,4,5,5-tetramethyl-1,3,2-dioxaborolan-2-yl) butyl)amino)butan-2-yl)carbamate (8-6, 650 mg, 1.3 mmol) in 1,4-dioxane (6 mL) was added 4 M HCl in 1,4 dioxane (6.0 mL, 25.1 mmol) at 0° C. It was gradually warmed to 25° C. and stirred for 16 h. TLC showed complete consumption of starting material to form new polar spot. The volatiles were removed under reduced pressure to get the crude (R)-2-amino-N⁴-ethyl-N¹—((R)-4-phenyl-1-(4,4,5,5-tetramethyl-1,3,2-dioxaborolan-2-yl)butyl)succinamide hydrochloride (8-7, 550 mg). The crude was directly used for next step without purification.

Synthesis of (R)—N⁴-ethyl-N¹—((R)-4-phenyl-1-(4,4,5,5-tetramethyl-1,3,2-dioxaborolan-2-yl)butyl)-2-(pyrazine-2-carboxamido)succinamide [Step 5]: To a stirred solution of pyrazine-2-carboxylic acid (8-8, 165 mg, 1.3 mmol) in THF (2 mL) was added IBCF (0.2 mL, 1.3 mmol) and NMM (0.15 mL, 1.3 mmol) at −15° C. The reaction mixture was stirred at same temperature for 30 min. Then (R)-2-amino-N⁴-ethyl-N¹—((R)-4-phenyl-1-(4,4,5,5-tetramethyl-1,3,2-dioxaborolan-2-yl)butyl)succinamide hydrochloride (8-7, 550 mg, 1.2 mmol) in DMF (1 mL) followed by NMM (0.13 mL, 1.2 mmol) was added to the reaction mixture at same condition. It was gradually warmed to 0° C. and stirred for 2 h. LCMS of crude reaction mass confirmed the formation of desired product. It was neutralized with saturated aqueous 0.1 N HCl solution and extracted with ethyl acetate. The combined organic layer was washed with 5% K₂CO₃ solution, water, and brine, dried over Na₂SO₄, filtered and evaporated under reduced pressure. The crude material was purified by RP prep HPLC purification and lyophilized to afford (R)—N⁴-ethyl-N¹—((R)-4-phenyl-1-(4,4,5,5-tetramethyl-1,3,2-dioxaborolan-2-yl)butyl)-2-(pyrazine-2-carboxamido) succinamide (8-9, 30 mg). [M−H]⁺=522.6, [M-84+H]⁺=440.5.

Synthesis of ((R)-1-((R)-4-(ethylamino)-4-oxo-2-(pyrazine-2-carboxamido)butanamido)-4-phenylbutyl)boronic acid [Step 6]: To a stirred solution of (R)—N⁴-ethyl-N¹—((R)-4-phenyl-1-(4,4,5,5-tetramethyl-1,3,2-dioxaborolan-2-yl)butyl)-2-(pyrazine-2-carboxamido)succinamide (8-9, 30 mg, 0.06 mmol) and methylboronic acid (8-10, 35 mg, 0.6 mmol) in acetone (2 mL) was added 0.2 N HCl (2 mL) and the reaction mixture was stirred at RT overnight. TLC and LCMS showed full conversion of starting material with formation of new polar spot. The volatiles were evaporated and the residue was purified by RP prep HPLC purification and lyophilized to yield ((R)-1-((R)-4-(ethylamino)-4-oxo-2-(pyrazine-2-carboxamido)butanamido)-4-phenylbutyl)boronic acid (Compound 8.13 mg). [M−H]⁺=440.1 ¹H NMR δ ¹H NMR (400 MHz, Methanol-d₄) δ 9.23 (s, 1H), 8.80 (d, 1H), 8.72-8.66 (m, 1H), 7.24-7.05 (m, 5H), 5.23-5.12 (m, 1H), 3.23-3.06 (m, 2H), 2.93 (dd, 1H), 2.83 (dd, 1H), 2.63-2.54 (m, 3H), 1.81-1.36 (m, 4H), 1.07 (t, 3H).

### Example 9

### Synthesis of ((R)-1-((R)-4-amino-4-oxo-2-(pyrazine-2-carboxamido)butanamido)-4-phenylbutyl)boronic acid

In another embodiment, Compound 9 can be produced from compound (9-1) by the method shown in Scheme 9. Carboxylic acid (9-1) is coupled with ammonium bicarbonate (9-2) to produce amide (9-3). Removal of the BOC protecting group yields the corresponding amine (9-4) as the hydrochloride salt. Subsequent coupling with carboxylic acid (9-5) produces amide (9-6). Hydrolysis of amide (9-6) yields carboxylic acid (9-7). Subsequent coupling with the amine of a protected boronic acid compound (9-8) produces amide (9-9). Deprotection of (9-9) with methylboronic acid produces Compound 9.

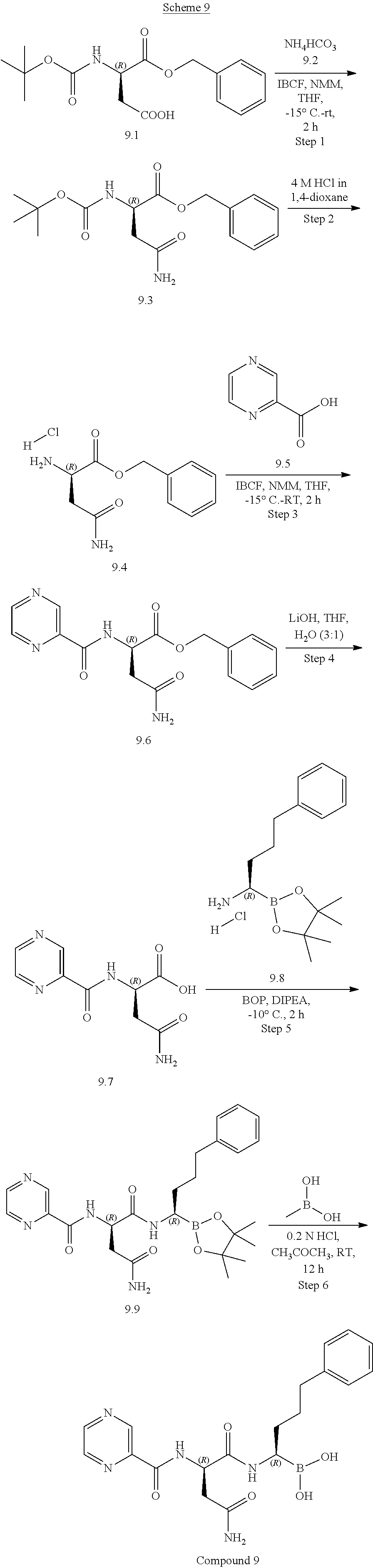

Synthesis of benzyl (tert-butoxycarbonyl)-D-asparaginate [Step 1]: To a stirred solution of (R)-4-(benzyloxy)-3-((tert-butoxycarbonyl)amino)-4-oxobutanoic acid (9-1, 1.80 g, 5.57 mmol) in THF (20 mL) was added NMM (0.61 mL, 5.57 mmol) and IBCF (0.73 mL, 5.57 mmol) at −15° C. The reaction mixture was stirred at same temperature for 30 min. Then NH₄HCO₃ (9-2, 400 mg, 5.06 mmol) and NMM (0.55 mL, 5.06 mmol) were added to the reaction. It was gradually warmed to 0° C. and stirred for 2 h (monitored by LCMS). It was neutralized with saturated aqueous 0.1 N HCl solution and extracted with ethyl acetate. Combined organic layer was washed with 5% K₂CO₃ solution, water, brine, dried over Na₂SO₄, filtered and evaporated under reduced pressure to get crude. Crude was purified through combi-flash column chromatography to afford benzyl (tert-butoxycarbonyl)-D-asparaginate (9-3, 700 mg). [M+H]⁺=323.1

Synthesis of benzyl D-asparaginate, hydrochloride [Step 2]: To a solution of benzyl (tert-butoxycarbonyl)-D-asparaginate (9-3, 650 mg, 2.02 mmol) in 1,4-Dioxane (5 mL), HCl 4.0 M in dioxane (5.0 mL, 20.2 mmol) was added drop wise to the solution at 0° C. The reaction mixture was stirred at room temperature for 16 h. The reaction mixture was monitored by LCMS. Then it was concentrated under reduced pressure to get crude benzyl D-asparaginate, hydrochloride (9-4, 500 mg). This crude compound was used for the next step without further purification. [M+H]⁺=222.9

Synthesis of benzyl (pyrazine-2-carbonyl)-D-asparaginate [Step 3]: To a stirred solution of (pyrazine-2-carboxylic acid (9-5, 246 mg, 1.98 mmol) in THF (5 mL) was added IBCF (0.29 mL, 2.18 mmol) and NMM (0.30 mL, 2.18 mmol) at −15° C. Reaction mixture was stirred at same temperature for 30 min. benzyl D-asparaginate hydrochloride (9-4, 441 mg, 1.98 mmol) in DMF (1 mL) followed by NMM (0.27 mL, 1.98 mmol) was added to reaction mixture at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. Combined organic layer was washed with 5% K₂CO₃ solution, water, brine, dried over Na₂SO₄, filtered and evaporated under reduced pressure. Crude was purified by combi-flash column chromatography using 0-5% MeOH in DCM as a eluent to afford desired benzyl (pyrazine-2-carbonyl)-D-asparaginate (9-6, 300 mg). [M+H]⁺=328.9

Synthesis of (pyrazine-2-carbonyl)-D-asparagine [Step 4]: To a stirred solution of benzyl (pyrazine-2-carbonyl)-D-asparaginate (9-6, 300 mg, 0.914 mmol) in THF (3 mL), LiOH (42 mg, 1.01 mmol) dissolved in water (1 mL) was added to the reaction mixture. Then reaction mixture was stirred for overnight at 25° C. The progress of the reaction was monitored by LCMS. After completion the reaction mixture was concentrated and diluted with water. The aqueous part was washed with EtOAc. The aqueous part was acidified with 1N HCl (pH=2) and lyophilized to get (pyrazine-2-carbonyl)-D-asparagine (9-7, 200 mg). ¹H NMR (400 MHz, DMSO-d₄) δ 9.18 (s, 1H), 8.91-8.87 (m, 2H), 8.74 (s, 1H), 7.90 (brs, 1H), 6.80 (s, 1H), 4.32 (brs, 1H), 2.57 (d, 2H).

Synthesis of (R)—N1-((R)-4-phenyl-1-(4,4,5,5-tetramethyl-1,3,2-dioxaborolan-2-yl)butyl)-2-(pyrazine-2-carboxamido)succinamide [Step 5]: To a stirred solution of (pyrazine-2-carbonyl)-D-asparagine (9-7, 100 mg, 0.420 mmol) in DMF (2 mL), DIPEA (0.17 mL, 1.26 mmol) and (1R)-4-phenyl-1-(4,4,5,5-tetramethyl-1,3,2-dioxaborolan-2-yl)butan-1-amine hydrochloride (9-8, 144 mg, 0.46 mmol), BOP (755 mg, 1.71 mmol) were added to the reaction mixture at −30° C. It was stirred at same condition for 2 h (monitored by LCMS). It was quenched with water and extracted with ethyl acetate. Organic part was washed with 5% K₂CO₃ solution, brine. Then it was dried with Na2SO4, filtered, concentrated under reduced pressure to get 200 mg of crude compound. This crude compound was purified by Prep-HPLC to get 17 mg of (R)—N1-((R)-4-phenyl-1-(4,4,5,5-tetramethyl-1,3,2-dioxaborolan-2-yl)butyl)-2-(pyrazine-2-carboxamido)succinamide (9-9) with corresponding boronic acid. [M−H]⁻=494.4

Synthesis of ((R)-1-((R)-4-amino-4-oxo-2-(pyrazine-2-carboxamido)butanamido)-4-phenylbutyl)boronic acid [Step 6]: To a stirred solution of (R)—N1-((R)-4-phenyl-1-(4,4,5,5-tetramethyl-1,3,2-dioxaborolan-2-yl)butyl)-2-(pyrazine-2-carboxamido)succinamide (9-9, 34 mg, 0.07 mmol) in Acetone (2 mL) was added 0.2 N HCl (2 mL) and the reaction mixture was stirred at RT overnight. TLC and LCMS showed full conversion of starting material with formation of new polar spot. The volatiles were evaporated and the residue was redissolved in acetone and deionized water and freeze-dried to obtain crude compound. This crude compound was purified by prep-HPLC (RP) to afford desired ((R)-1-((R)-4-amino-4-oxo-2-(pyrazine-2-carboxamido)butanamido)-4-phenylbutyl)boronic acid (Compound 9, 25 mg). [M−H]⁻=412.3; ¹H NMR (400 MHz, Methanol-d₄) δ 9.27-9.21 (m, 1H), 8.79 (d, 1H), 8.72-8.66 (m, 1H), 7.29-7.02 (m, 5H), 5.19 (d, 1H), 3.88 (s, 1H), 3.08 (brs, 1H), 2.72-2.66 (m, 1H), 2.62-2.49 (m, 2H), 1.72-1.36 (m, 4H).

### Example 10

### Synthesis of ((R)-1-((R)-4-amino-4-oxo-2-(pyrazine-2-carboxamido)butanamido)-4-phenylbutyl)boronic acid

In another embodiment, Compound 10 can be produced from compound (10-1) by the method shown in Scheme 10. Carboxylic acid (10-1) is coupled with piperidine (10-2) to produce amide (10-3), which is then deprotected to generate carboxylic acid (10-4). Subsequent coupling with the amine of a protected boronic acid compound (10-5) produces amide (10-6). Removal of the BOC protecting group yields the corresponding amine (10-7) as the hydrochloride salt. Subsequent coupling with carboxylic acid (10-8) produces amide (10-9). Deprotection of (10-9) with methylboronic acid (10-10) produces Compound 10.

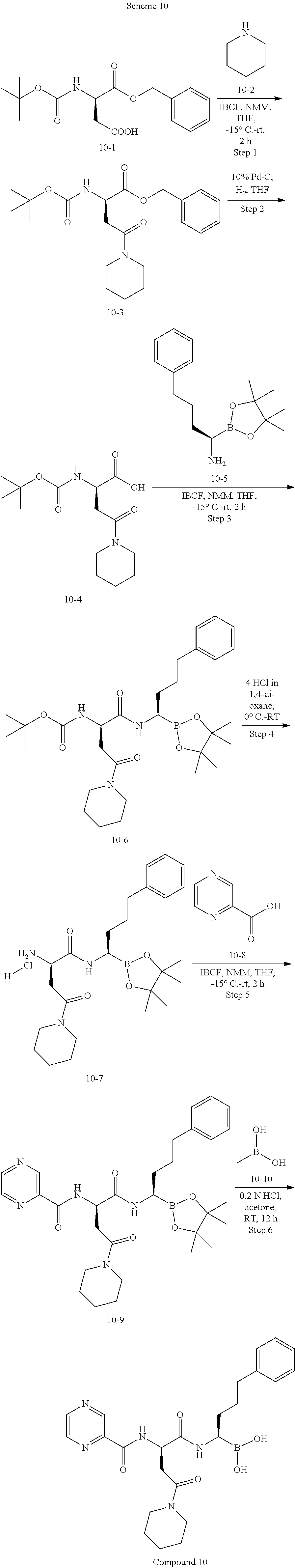

Synthesis of benzyl (R)-2-((tert-butoxycarbonyl)amino)-4-oxo-4-(piperidin-1-yl)butanoate [Step 1]: To a stirred solution of (R)-4-(benzyloxy)-3-((tert-butoxycarbonyl)amino)-4-oxobutanoic acid (10-1, 1.0 g, 3.2 mmol) in THF (25 mL) was added IBCF (0.4 mL, 3.2 mmol) and NMM (0.4 mL, 3.3 mmol) at −15° C. The reaction mixture was stirred at same temperature for 30 min. Then piperidine (10-2, 0.3 mL, 2.9 mmol) followed by NMM (0.3 mL, 2.9 mmol) was added to the reaction mixture at −15° C. It was gradually warmed to 0° C. and stirred for 2 h. LCMS of the crude reaction mass confirmed the formation of desired product. It was neutralized with saturated aqueous 0.1 N HCl solution and extracted with ethyl acetate. The combined organic layer was washed with 5% K₂CO₃ solution, water and brine. It was dried over Na₂SO₄, filtered and evaporated under reduced pressure to get crude residue. The crude was purified by silica gel column chromatography to afford benzyl (R)-2-((tert-butoxycarbonyl)amino)-4-oxo-4-(piperidin-1-yl)butanoate (10-3, 1.0 g). [M+H]⁺: 390.8.

Synthesis of (R)-2-((tert-butoxycarbonyl)amino)-4-oxo-4-(piperidin-1-yl)butanoic acid [Step 2]: To a stirred solution of benzyl (R)-2-((tert-butoxycarbonyl)amino)-4-oxo-4-(piperidin-1-yl)butanoate (10-3, 750 mg, 1.9 mmol) dissolved in THF (20 mL) was bubbled with nitrogen gas for 10 min. Then 10% Pd—C (300 mg, 2.6 mmol) was added and the reaction mixture was hydrogenated under balloon pressure for 16 h. The reaction was monitored by TLC. Upon completion, the reaction was filtered over celite with excess ethyl acetate. Removal of solvent under reduced pressure provided (R)-2-((tert-butoxycarbonyl)amino)-4-oxo-4-(piperidin-1-yl)butanoic acid (10-4, 550 mg). [M+H]: 301.3.

Synthesis of tert-butyl ((R)-1,4-dioxo-1-(((R)-4-phenyl-1-(4,4,5,5-tetramethyl-1,3,2-dioxaborolan-2-yl)butyl)amino)-4-(piperidin-1-yl)butan-2-yl)carbamate [Step 3]: To a stirred solution of (R)-2-((tert-butoxycarbonyl)amino)-4-oxo-4-(piperidin-1-yl)butanoic acid (10-4, 740 mg, 2.5 mmol) in THF (10 mL) was added IBCF (0.3 mL, 2.5 mmol) and NMM (0.3 mL, 2.5 mmol) at −15° C. The reaction mixture was stirred at same temperature for 30 min. Then (R)-4-phenyl-1-(4,4,5,5-tetramethyl-1,3,2-dioxaborolan-2-yl)butan-1-amine hydrochloride (10-5, 700 mg, 2.2 mmol) in DMF (1 mL) followed by NMM (0.2 mL, 2.2 mmol) was added to the reaction mixture at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. The combined organic layer was washed with 5% K₂CO₃ solution, water and brine, dried over Na₂SO₄, filtered and evaporated under reduced pressure to afford tert-butyl ((R)-1,4-dioxo-1-(((R)-4-phenyl-1-(4,4,5,5-tetramethyl-1,3,2-dioxaborolan-2-yl)butyl)amino)-4-(piperidin-1-yl)butan-2-yl)carbamate (10-6, 700 mg, crude). [M+H]⁺: 558.0.

Synthesis of (R)-2-amino-4-oxo-N—((R)-4-phenyl-1-(4,4,5,5-tetramethyl-1,3,2-dioxaborolan-2-yl)butyl)-4-(piperidin-1-yl)butanamide hydrochloride [Step 4]: To a solution of tert-butyl ((R)-1,4-dioxo-1-(((R)-4-phenyl-1-(4,4,5,5-tetramethyl-1,3,2-dioxaborolan-2-yl)butyl)amino)-4-(piperidin-1-yl)butan-2-yl)carbamate (10-6, 700 mg, 1.2 mmol) in 1,4 dioxane (6 mL) was added 4 M HCl in dioxane (6.0 mL, 24.0 mmol) at 0° C. It was gradually warmed to 25° C. and stirred for 16 h. TLC showed complete consumption of starting material to form new polar spot. The volatiles were removed under reduced pressure to get (R)-2-amino-4-oxo-N—((R)-4-phenyl-1-(4,4,5,5-tetramethyl-1,3,2-dioxaborolan-2-yl)butyl)-4-(piperidin-1-yl)butanamide hydrochloride (10-7, 560 mg, crude). The crude was directly used for next step without purification.

Synthesis of N—((R)-1,4-dioxo-1-(((R)-4-phenyl-1-(4,4,5,5-tetramethyl-1,3,2-dioxaborolan-2-yl)butyl)amino)-4-(piperidin-1-yl)butan-2-yl)pyrazine-2-carboxamide [Step 5]: To a stirred solution of pyrazine-2-carboxylic acid (10-7, 135 mg, 1.1 mmol) in THF (8 mL) was added IBCF (0.13 mL, 1.1 mmol) and NMM (0.11 mL, 1.1 mmol) at −15° C. The reaction mixture was stirred at same temperature for 30 min. Then (R)-2-amino-4-oxo-N—((R)-4-phenyl-1-(4,4,5,5-tetramethyl-1,3,2-dioxaborolan-2-yl)butyl)-4-(piperidin-1-yl)butanamide hydrochloride (10-8, 480 mg, 1.0 mmol) in DMF (1 mL) followed by NMM (0.1 mL, 1.0 mmol) was added to the reaction mixture under same condition. It was gradually warmed to 0° C. and stirred for 2 h. LCMS of the crude reaction mass confirmed the formation of desired product. It was neutralized with saturated aqueous 0.1 N HCl solution and extracted with ethyl acetate. The combined organic layer was washed with 5% K₂CO₃ solution, water and brine. It was dried over Na₂SO₄, filtered and evaporated under reduced pressure. The crude material was purified by RP prep HPLC purification and lyophilized to afford N—((R)-1,4-dioxo-1-(((R)-4-phenyl-1-(4,4,5,5-tetramethyl-1,3,2-dioxaborolan-2-yl)butyl)amino)-4-(piperidin-1-yl)butan-2-yl)pyrazine-2-carboxamide (10-9, 45 mg). [M−H]⁺=562.6 [M-84+H]⁺=480.5.

Synthesis of ((R)-1-((R)-4-oxo-4-(piperidin-1-yl)-2-(pyrazine-2-carboxamido)butanamido)-4-phenylbutyl)boronic acid [Step 6]: To a stirred solution of N—((R)-1,4-dioxo-1-(((R)-4-phenyl-1-(4,4,5,5-tetramethyl-1,3,2-dioxaborolan-2-yl)butyl)amino)-4-(piperidin-1-yl)butan-2-yl)pyrazine-2-carboxamide (10-9, 45 mg, 0.08 mmol) and methylboronic acid (10-10, 45 mg, 0.8 mmol) in acetone (2 mL) was added 0.2 N HCl (2 mL) and the reaction mixture was stirred at RT overnight.

TLC and LCMS showed full conversion of starting material with formation of new polar spot. The volatiles were evaporated and the residue was redissolved in acetone and deionized water and freeze-dried to obtain ((R)-1-((R)-4-oxo-4-(piperidin-1-yl)-2-(pyrazine-2-carboxamido) butanamido)-4-phenylbutyl)boronic acid (Compound 10, 22 mg). [M−H]⁺: 480.2; ¹H NMR (400 MHz, MeOD) δ 9.24 (s, 1H), 8.79 (d, 1H), 8.68 (s, 1H), 7.21-7.08 (m, 5H), 5.27 (t, 1H), 3.51-3.45 (m, 4H), 2.99-2.98 (m, 2H), 2.60-2.56 (m, 3H), 1.65-1.48 (m, 10H).

### Example 11

### Synthesis of (R)-1-((R)-2-acetamido-4-morpholino-4-oxobutanamido)-4-phenylbutyl) boronic acid

In another embodiment, Compound 11 can be produced from compound (11-1) by the method shown in Scheme 11. Amine (11-1) is coupled with acetic anhydride (11-2) to afford amide (11-3). Subsequent deprotection of (11-3) with methylboronic acid (11-4) produces Compound 11.

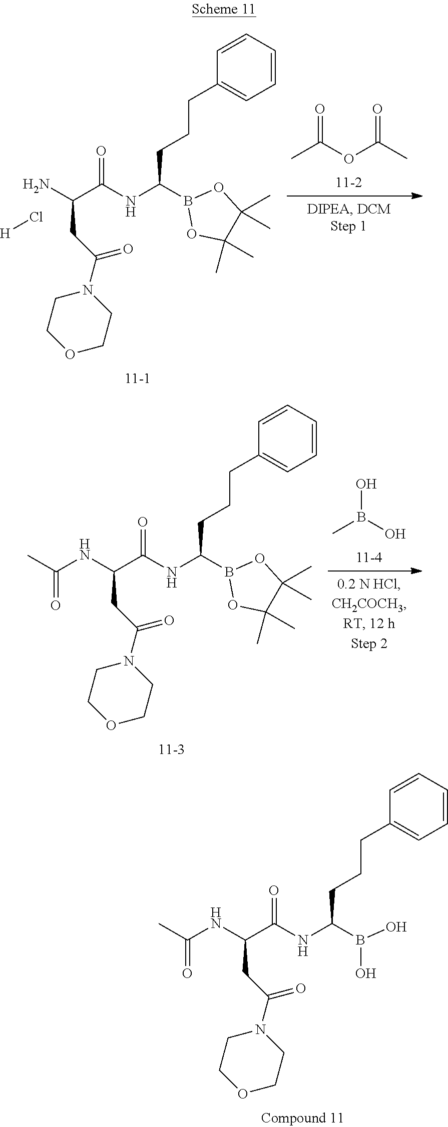

Synthesis of (R)-2-acetamido-4-morpholino-4-oxo-N—((R)-4-phenyl-1-(4,4,5,5-tetramethyl-1,3,2-dioxaborolan-2-yl)butyl)butanamide [Step 1]: To a stirred solution of (R)-2-amino-4-morpholino-4-oxo-N—((R)-4-phenyl-1-(4,4,5,5-tetramethyl-1,3,2-dioxaborolan-2-yl)butyl) butanamide hydrochloride (11-1, 300 mg, 0.6 mmol) and acetic anhydride (11-2, 0.06 mL, 0.7 mmol) in DCM (4 mL), DIPEA (0.5 mL, 3 mmol) was added in ice cold condition and the reaction mixture was stirred at RT for 2 h. TLC and LCMS showed full conversion of starting material with formation of new spot. The reaction was diluted with DCM and washed with water and brine solution. The organic phase was dried over Na₂SO₄, filtered and evaporated. The crude material was purified by RP prep HPLC purification and lyophilized to afford (R)-2-acetamido-4-morpholino-4-oxo-N—((R)-4-phenyl-1-(4,4,5,5-tetramethyl-1,3,2-dioxaborolan-2-yl) butyl)butanamide (11-3, 50 mg). [M−H]⁺=500.1; [M-84+H]⁺=418.3.

Synthesis of (R)-1-((R)-2-acetamido-4-morpholino-4-oxobutanamido)-4-phenylbutyl) boronic acid [Step 2]: To a stirred solution of (R)-2-acetamido-4-morpholino-4-oxo-N—((R)-4-phenyl-1-(4,4,5,5-tetramethyl-1,3,2-dioxaborolan-2-yl)butyl)butanamide (11-3, 50 mg, 0.1 mmol) and methylboronic acid (11-4, 60 mg, 1.0 mmol) in acetone (3 mL) was added 0.2 N HCl (3.0 mL) and the reaction mixture was stirred at RT overnight. TLC and LCMS showed full conversion of starting material with formation of new polar spot. The volatiles were evaporated and the crude was purified via RP prep HPLC purification and lyophilized to afford (R)-1-((R)-2-acetamido-4-morpholino-4-oxobutanamido)-4-phenylbutyl)boronic acid (Formula 11, 25 mg). [M−H]⁺: 418.3; ¹H NMR (400 MHz, MeOD) δ 7.24-7.10 (m, 5H), 5.00 (t, 1H), 3.66-3.59 (m, 4H), 3.52-3.48 (m, 4H), 3.0-2.86 (m, 2H), 2.64-2.57 (m, 3H), 1.97 (s, 3H), 1.71-1.47 (m, 4H).

### Example 12

### Synthesis of ((R)-1-((R)-2-((tert-butoxycarbonyl)amino)-4-morpholino-4-oxobutanamido)-4-phenylbutyl)boronic acid

In another embodiment, Compound 12 can be produced from compound (12-1) by the method shown in Scheme 12. Deprotection of (12-1) with a mixture comprising sodium periodate and ammonium acetate produces Compound 12.

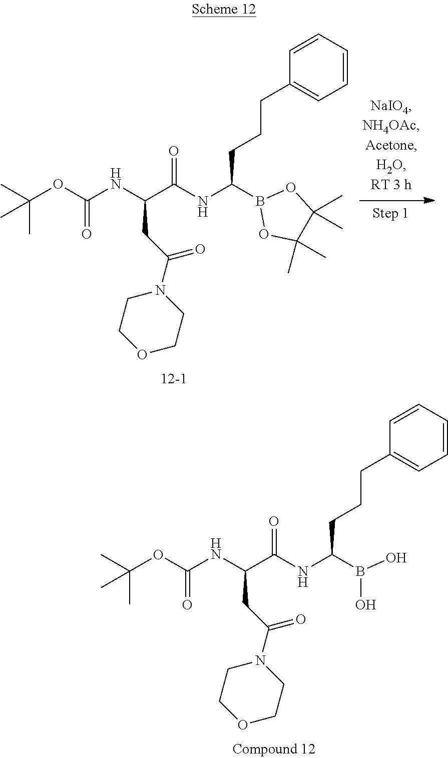 [SMILES: CC(C)(C)OC(=O)N[C@H](CC(=O)N1CCOCC1)C(=O)N[C@@H](CCCc1ccccc1)B1OC(C)(C)C(C)(C)O1 ; CC(C)(C)OC(=O)N[C@H](CC(=O)N1CCOCC1)C(=O)N[C@@H](CCCc1ccccc1)B(O)O]

Synthesis of ((R)-1-((R)-2-((tert-butoxycarbonyl)amino)-4-morpholino-4-oxobutanamido)-4-phenylbutyl)boronic acid [Step 1]: To a stirred solution tert-butyl ((R)-4-morpholino-1,4-dioxo-1-(((R)-4-phenyl-1-(4,4,5,5-tetramethyl-1,3,2-dioxaborolan-2-yl)butyl)amino)butan-2-yl) carbamate (12-1, 300 mg, 0.5 mmol) in acetone (6 mL) and water (6 mL) was added NH₄OAc (40 mg, 0.5 mmol) and stirred for 5 min. NaIO₄ (115 mg, 0.5 mmol) was added to it portion wise and stirred for 3 h. Volatiles were removed under reduced pressure and partitioned between ethyl acetate and water. The organic layer was collected. The aqueous layer was further extracted with EtOAc (twice). The combined organic layer was dried over anhydrous Na₂SO₄ and evaporated under reduced pressure. The crude was purified through RP prep HPLC purification to afford ((R)-1-((R)-2-((tert-butoxycarbonyl)amino)-4-morpholino-4-oxobutanamido)-4-phenylbutyl)boronic acid (Compound 12, 50 mg). [M−H]⁺=476.5; ¹H NMR (400 MHz, MeOD) b 7.26-7.13 (m, 5H), 4.74 (t, 1H), 3.72-3.60 (m, 4H), 3.52-2.49 (m, 4H), 2.92 (t, 2H), 2.65-2.55 (m, 3H), 1.71-1.50 (m, 4H), 1.45 (s, 9H).

### Example 13

### Synthesis of ((R)-1-((R)-2-(morpholine-4-carboxamido)-4-morpholino-4-oxobutanamido)-4-phenylbutyl)boronic acid

In another embodiment, Compound 13 can be produced from compound (13-1) by the method shown in Scheme 13. Amine (13-1) is coupled with compound (13-2) to afford amide (13-3). Subsequent deprotection of (13-3) with methylboronic acid (13-4) produces Compound 13.

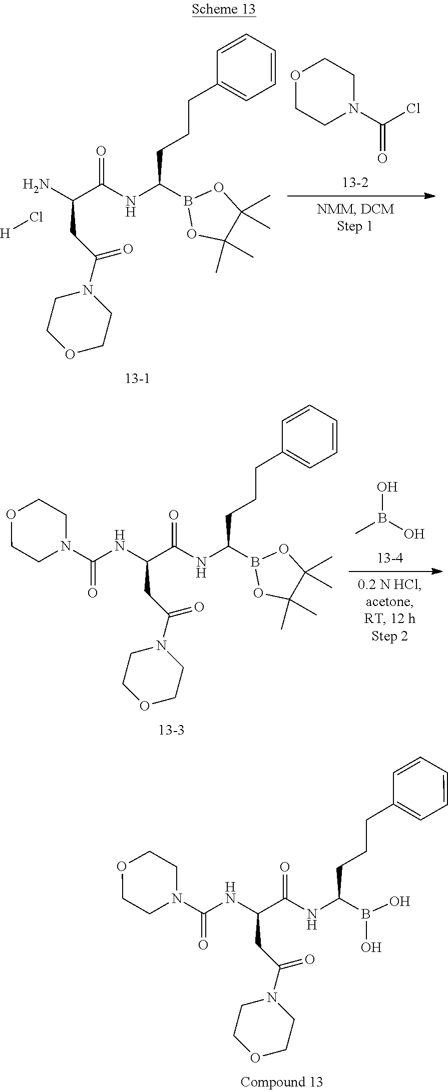

Synthesis of N—((R)-4-morpholino-1,4-dioxo-1-(((R)-4-phenyl-1-(4,4,5,5-tetramethyl-1,3,2-dioxaborolan-2-yl)butyl)amino)butan-2-yl)morpholine-4-carboxamide [Step 1]: To a stirred solution of (R)-2-amino-4-morpholino-4-oxo-N—((R)-4-phenyl-1-(4,4,5,5-tetramethyl-1,3,2-dioxaborolan-2-yl)butyl)butanamide hydrochloride (13-1, 250 mg, 0.5 mmol) and morpholine-4-carbonyl chloride (13-2, 0.07 mL, 0.6 mmol) in DCM (4 mL), NMM (0.1 mL, 1 mmol) was added in ice cold condition and the reaction mixture was stirred at RT for 2 h. TLC and LCMS showed full conversion of starting material with formation of new polar spot. The reaction was diluted with DCM and washed with water and brine solution. The organic phase was dried over Na₂SO₄, filtered and evaporated. The crude material was purified by RP prep HPLC purification and lyophilized to afford N—((R)-4-morpholino-1,4-dioxo-1-(((R)-4-phenyl-1-(4,4,5,5-tetramethyl-1,3,2-dioxaborolan-2-yl)butyl)amino)butan-2-yl)morpholine-4-carboxamide (13-3, 52 mg). [M−H]⁺=571.5; [M-84+H]⁺=489.4; ¹H NMR (400 MHz, MeOD) δ 7.24-7.09 (m, 5H), 4.96-4.92 (m, 1H), 3.65-3.59 (m, 8H), 3.51-3.49 (m, 4H), 3.40-3.32 (m, 4H), 3.01-2.87 (m, 2H), 2.62-2.57 (m, 3H), 1.70-1.48 (m, 4H), 1.19-1.14 (m, 5H).

Synthesis of ((R)-1-((R)-2-(morpholine-4-carboxamido)-4-morpholino-4-oxobutanamido)-4-phenylbutyl)boronic acid [Step 2]: To a stirred solution of N—((R)-4-morpholino-1,4-dioxo-1-(((R)-4-phenyl-1-(4,4,5,5-tetramethyl-1,3,2-dioxaborolan-2-yl)butyl)amino)butan-2-yl) morpholine-4-carboxamide (13-3, 50 mg, 0.09 mmol) and methylboronic acid (13-4, 52 mg, 0.9 mmol) in acetone (3 mL) was added 0.2 N HCl (3.0 mL) and the reaction mixture was stirred at RT overnight. TLC and LCMS showed full conversion of starting material with formation of new polar spot. The volatiles were evaporated and the residue was redissolved in acetonitrile and deionized water and freeze-dried to obtain crude. The crude material was purified by RP prep HPLC purification and lyophilized to afford ((R)-1-((R)-2-(morpholine-4-carboxamido)-4-morpholino-4-oxobutanamido)-4-phenylbutyl)boronic acid (Compound 13, 16 mg). [M−H]⁺=489.4; ¹H NMR (400 MHz, MeOD) δ 7.22-7.11 (m, 5H), 4.93 (t, 1H), 3.65-3.59 (m, 8H), 3.51-3.48 (m, 4H), 3.37-3.33 (m, 4H), 2.99-2.91 (m, 2H), 2.62-2.59 (m, 3H), 1.75-1.40 (m, 4H).

### Example 14

### Synthesis of ((R)-1-((R)-2-(3-(tert-butyl)ureido)-4-morpholino-4-oxobutanamido)-4-phenylbutyl)boronic acid

In another embodiment, Compound 14 can be produced from compound (14-1; 1-7) by the method shown in Scheme 14. Amine (14-1) is coupled with compound (14-2) to afford amide (14-3). Subsequent deprotection of (14-3) with methylboronic acid produces Compound 14.

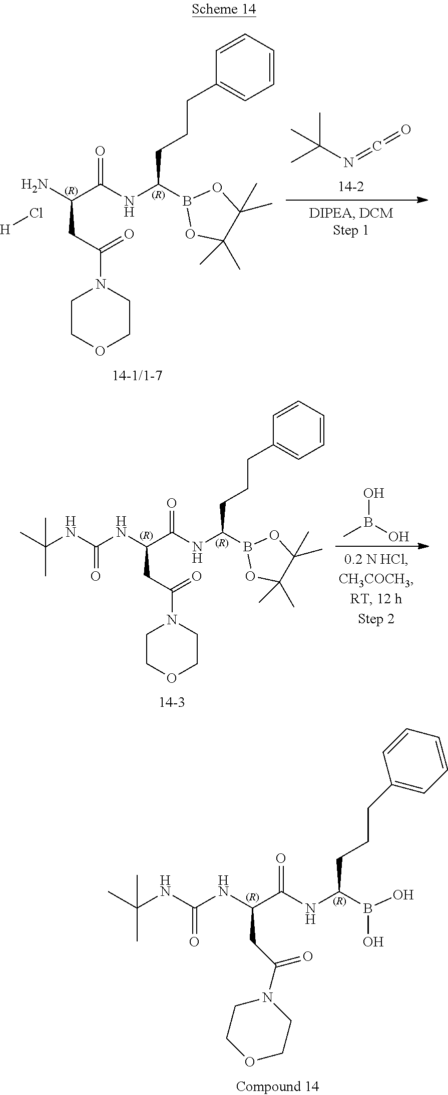

Synthesis of (R)-2-(3-(tert-butyl)ureido)-4-morpholino-4-oxo-N—((R)-4-phenyl-1-(4,4,5,5-tetramethyl-1,3,2-dioxaborolan-2-yl)butyl)butanamide [Step 1]: To a stirred solution of (R)-2-amino-4-morpholino-4-oxo-N—((R)-4-phenyl-1-(4,4,5,5-tetramethyl-1,3,2-dioxaborolan-2-yl)butyl) butanamide hydrochloride (14-1/1-7, 300 mg, 0.6 mmol) and 2-isocyanato-2-methylpropane (14-2, 0.08 mL, 0.7 mmol) in DCM (3 mL) was added DIPEA (0.5 mL, 3 mmol) at ice cold condition and stirred at RT for 2 h. The reaction was diluted with DCM and washed with water and brine, dried over Na₂SO₄ and evaporated. The crude material was purified by prep HPLC purification and lyophilized to afford (R)-2-(3-(tert-butyl) ureido)-4-morpholino-4-oxo-N—((R)-4-phenyl-1-(4,4,5,5-tetramethyl-1,3,2-dioxaborolan-2-yl) butyl) butanamide (14-3, 42 mg). [M−H]⁻=557.5

Synthesis of ((R)-1-((R)-2-(3-(tert-butyl)ureido)-4-morpholino-4-oxobutanamido)-4-phenylbutyl)boronic acid [Step 2]: To a stirred solution of (R)-2-(3-(tert-butyl)ureido)-4-morpholino-4-oxo-N—((R)-4-phenyl-1-(4,4,5,5-tetramethyl-1,3,2-dioxaborolan-2-yl)butyl) butanamide (14-3, 47 mg, 0.08 mmol) and methylboronic acid (47 mg, 0.8 mmol) in Acetone (3 mL) was added 0.2 N HCl (3 mL) and stirred at RT for overnight. TLC and LCMS showed conversion of starting material with formation of new polar spot. The volatiles were evaporated and the residue was purified by prep HPLC purification and lyophilized to afford ((R)-1-((R)-2-(3-(tert-butyl) ureido)-4-morpholino-4-oxobutanamido)-4-phenylbutyl) boronic acid (Compound 14, 31 mg). [M−H]⁻475.3; ¹H NMR (400 MHz, MeOD) δ 7.24-7.11 (m, 5H), 4.85 (brs, 1H), 3.65-3.59 (m, 4H), 3.51-3.47 (m, 4H), 3.04 (dd, 1H), 5.60 (dd, 1H), 2.59 (q, 3H), 1.72-1.66 (m, 2H) 1.56-1.49 (m, 2H), 1.28 (s, 9H).

### Example 15

### Synthesis of ((R)-1-((R)-5-morpholino-5-oxo-2-(pyrazine-2-carboxamido)pentanamido)-4-phenylbutyl)boronic acid

In another embodiment, Compound 15 can be produced from compound (15-1) by the method shown in Scheme 15. Carboxylic acid (15-1) is coupled morpholine (15-2) to produce amide (15-3), which is then deprotected to generate carboxylic acid (15-4). Subsequent coupling with the amine of a protected boronic acid compound (15-5) produces amide (15-6). Removal of the BOC protecting group yields the corresponding amine (15-7) as the hydrochloride salt. Subsequent coupling with carboxylic acid (15-8) produces amide (15-9). Deprotection of (15-9) with methylboronic acid (15-10) produces Compound 15.

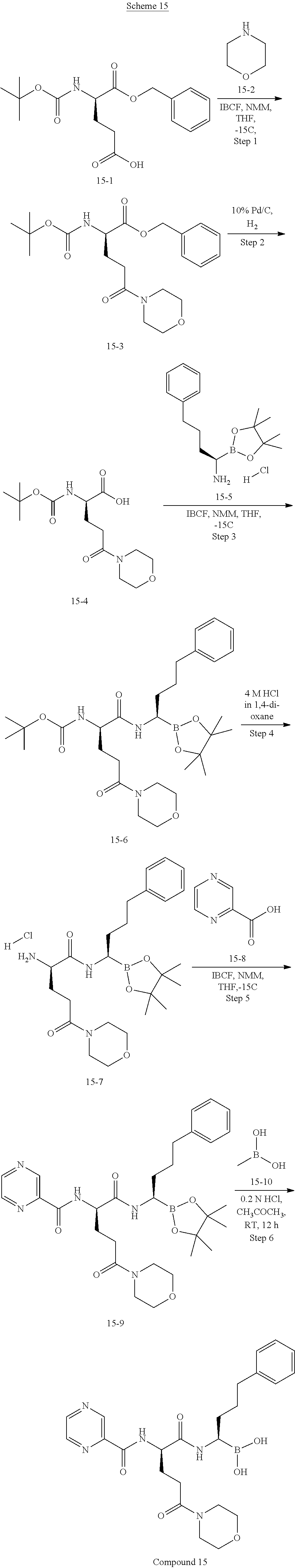

Synthesis of benzyl (R)-2-((tert-butoxycarbonyl)amino)-5-morpholino-5-oxopentanoate [Step 1]: To a stirred solution of (R)-5-(benzyloxy)-4-((tert-butoxycarbonyl)amino)-5-oxopentanoic acid (15-1, 1.3 g, 3.8 mmol) in THF (50 mL) was added IBCF (0.5 mL, 3.8 mmol) and NMM (0.4 mL, 3.8 mmol) at −15° C. Reaction mixture was stirred at same temperature for 30 min. Then morpholine (15-2, 0.3 mL, 3.5 mmol) followed by NMM (0.4 mL, 3.5 mmol) was added to reaction mixture at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. Combined organic layer was washed with 5% K₂CO₃ solution, water, brine, dried over Na₂SO₄, filtered and evaporated under reduced pressure to get crude. Crude was purified by silica gel column chromatography to afford benzyl (R)-2-((tert-butoxycarbonyl)amino)-5-morpholino-5-oxopentanoate (15-3, 1.10 g). ¹H NMR (400 MHz, DMSO-D₆) δ 7.36-7.31 (m, 6H), 5.17-5.05 (m, 2H), 4.06-4.01 (m, 1H), 3.51-3.50 (m, 4H), 3.41-3.39 (m, 2H), 2.39-2.28 (m, 2H), 1.95-1.89 (m, 1H), 1.84-1.77 (m, 1H), 1.37 (s, 9H).

Synthesis of (R)-2-((tert-butoxycarbonyl)amino)-5-morpholino-5-oxopentanoic acid [Step 2]: benzyl (R)-2-((tert-butoxycarbonyl)amino)-5-morpholino-5-oxopentanoate (15-3, 1.10 g, 2.7 mmol)) was dissolved in THF (20 mL), 10% Pd—C (425 mg, 4 mmol) was added and hydrogenated under balloon pressure for 3 h. After completion, the reaction was filtered over Celite and washed with excess ethyl acetate. Removal of solvents in vacuo provided the product as (R)-2-((tert-butoxycarbonyl)amino)-5-morpholino-5-oxopentanoic acid (15-4, 800 mg). ¹H NMR (400 MHz, DMSO-D₆) δ 12.5 (brs, 1H), 7.08 (d, 1H), 3.93-3.88 (m, 1H), 3.55-3.51 (m, 4H), 3.41 (d, 4H), 2.40-2.28 (m, 2H), 1.99-1.88 (m, 1H), 1.80-1.73 (m, 1H), 1.38 (s, 9H).

Synthesis of tert-butyl ((R)-5-morpholino-1,5-dioxo-1-(((R)-4-phenyl-1-(4,4,5,5-tetramethyl-1,3,2-dioxaborolan-2-yl)butyl)amino)pentan-2-yl)carbamate [Step 3]: To a stirred solution of (R)-2-((tert-butoxycarbonyl)amino)-5-morpholino-5-oxopentanoic acid (15-4, 730 mg, 2.3 mmol) in THF (8 mL) was added IBCF (0.30 mL, 2.3 mmol) and NMM (0.3 mL, 2.3 mmol) at −15° C. Reaction mixture was stirred at same temperature for 30 min. (R)-4-phenyl-1-(4,4,5,5-tetramethyl-1,3,2-dioxaborolan-2-yl)butan-1-amine:hydrochloride (15-5, 650 mg, 2.1 mmol) in DMF (1 mL) followed by NMM (0.2 mL, 2.1 mmol) was added to reaction mixture at −15° C. It was gradually warmed to 0° C. and stirred for 2 h. It was neutralized with saturated aqueous 0.1 N HCl solution and extracted with ethyl acetate. Combined organic layer was washed with 5% K₂CO solution, water, brine, dried over Na₂SO₄, filtered and evaporated under reduced pressure to afford tert-butyl ((R)-5-morpholino-1,5-dioxo-1-(((R)-4-phenyl-1-(4,4,5,5-tetramethyl-1,3,2-dioxaborolan-2-yl)butyl)amino)pentan-2-yl)carbamate (15-6, 1.10 g). Crude was used directly without further purification. [M−H]⁺=572.

Synthesis of (R)-2-amino-5-morpholino-5-oxo-N—((R)-4-phenyl-1-(4,4,5,5-tetramethyl-1,3,2-dioxaborolan-2-yl)butyl)pentanamide [Step 4]: To a solution of tert-butyl ((R)-5-morpholino-1,5-dioxo-1-(((R)-4-phenyl-1-(4,4,5,5-tetramethyl-1,3,2-dioxaborolan-2-yl)butyl)amino)pentan-2-yl)carbamate (15-6, 1.00 g, 1.7 mmol) in 1,4-Dioxane (5 mL) was added 4 M HCl in 1,4 Dioxane (6.5 mL, 26 mmol) at 0° C. It was gradually warmed to 25° C. and stirred for 16 h. Volatiles were removed under reduced pressure to get (R)-2-amino-5-morpholino-5-oxo-N—((R)-4-phenyl-1-(4,4,5,5-tetramethyl-1,3,2-dioxaborolan-2-yl)butyl)pentanamide (15-7, 800 mg), which was directly used in next step. [M−H]⁺=472.

Synthesis of N—((R)-5-morpholino-1,5-dioxo-1-(((R)-4-phenyl-1-(4,4,5,5-tetramethyl-1,3,2-dioxaborolan-2-yl)butyl)amino)pentan-2-yl)pyrazine-2-carboxamide [Step 5]: To a solution of pyrazine-2-carboxylic acid (15-8, 231 mg, 1.9 mmol) in THF (15 mL) under argon atmosphere was added IBCF (0.3 mL, 1.9 mmol) followed by NMM (0.3 mL, 1.9 mmol) at −15° C. and stirred for 45 min. (R)-2-amino-5-morpholino-5-oxo-N—((R)-4-phenyl-1-(4,4,5,5-tetramethyl-1,3,2-dioxaborolan-2-yl)butyl)pentanamide (15-7, 800 mg, 1.7 mmol) followed by NMM (0.2 mL, 1.9 mmol) was added to it and stirred for 2 h. The reaction was quenched with water and diluted with EtOAc. Organic layer was collected and washed successively with 0.1 M aqueous HCl, 5% aqueous K₂CO₃ solution, water and brine, dried over anhydrous Na₂SO₄ and concentrated under reduced pressure to get crude. Crude material was purified by prep HPLC purification and lyophilized to afford N—((R)-5-morpholino-1,5-dioxo-1-(((R)-4-phenyl-1-(4,4,5,5-tetramethyl-1,3,2-dioxaborolan-2-yl)butyl)amino)pentan-2-yl)pyrazine-2-carboxamide (15-9, 900 mg). [M−H]⁺=578.

Synthesis of ((R)-1-((R)-5-morpholino-5-oxo-2-(pyrazine-2-carboxamido)pentanamido)-4-phenylbutyl)boronic acid [Step 6]: To a stirred solution of N—((R)-5-morpholino-1,5-dioxo-1-(((R)-4-phenyl-1-(4,4,5,5-tetramethyl-1,3,2-dioxaborolan-2-yl)butyl)amino)pentan-2-yl)pyrazine-2-carboxamide (15-9, 75 mg, 0.2 mmol) and methyl boronic acid (15-10, 116 mg, 1.9 mmol) in Acetone (4 mL) was added 0.2 N HCl (4.0 mL) and the reaction mixture was stirred at RT overnight. TLC and LCMS showed full conversion of starting material with formation of new polar spot. The volatiles were evaporated and the residue was redissolved in acetone and deionized water and freeze-dried to obtain crude. Crude material was purified by prep HPLC purification and lyophilized to afford ((R)-1-((R)-5-morpholino-5-oxo-2-(pyrazine-2-carboxamido) pentanamido)-4-phenylbutyl)boronic acid (Compound 15, 40 mg). HPLC: tR=7.14 min (99.28%). [M−H]⁺=496. ¹H NMR (400 MHz, MeOD) δ 9.21 (s, 1H), 8.80 (d, 1H), 8.70 (t, 1H), 7.22-7.19 (m, 2H), 7.16-7.10 (m, 3H), 3.60-3.54 (m, 6H), 3.47-3.45 (m, 2H), 2.65-2.53 (m, 5H), 2.34-2.28 (m, 1H), 2.24-2.17 (m, 1H), 1.69-1.62 (m, 2H) 1.59-1.49 (m, 3H).

### Example 16

### Synthesis of ((R)-1-((R)-4-morpholino-2-(oxazol-2-ylamino)-4-oxobutanamido)-4-phenyl butyl)boronic acid

In another embodiment, the present invention is directed to a compound, or a pharmaceutically acceptable salt thereof, represented by Structure 16:

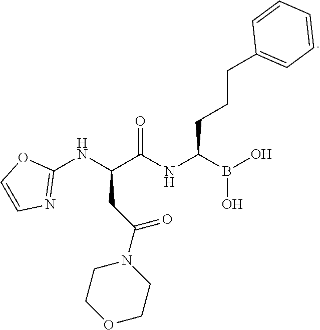 [SMILES: O=C(N[C@@H](CCCc1ccccc1)B(O)O)[C@@H](CC(=O)N1CCOCC1)Nc1ncco1]

In certain embodiments, the compound is ((R)-1-((R)-4-morpholino-2-(oxazol-2-ylamino)-4-oxobutanamido)-4-phenylbutyl)boronic acid (Compound 16) or a pharmaceutically acceptable salt thereof.

### Example 17

Synthesis of ((R)-1-((R)-4-morpholino-4-oxo-2-(trifluoromethylsulfonamido)butanamido)-4-phenylbutyl)boronic acid In another embodiment, the present invention is directed to a compound, or a pharmaceutically acceptable salt thereof, represented by Structure 17:

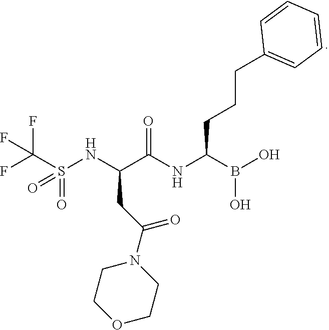 [SMILES: O=C(N[C@@H](CCCc1ccccc1)B(O)O)[C@@H](CC(=O)N1CCOCC1)NS(=O)(=O)C(F)(F)F]

In certain embodiments, the compound is ((R)-1-((R)-4-morpholino-4-oxo-2-(trifluoromethylsulfonamido)butanamido)-4-phenylbutyl)boronic acid (Compound 17) or a pharmaceutically acceptable salt thereof.

### Example 18

### Synthesis of ((R)-1-((R)-4-morpholino-4-oxo-2-(pyrazine-2-carboxamido)butanamido) butyl)boronic acid

In another embodiment, Compound 18 can be produced from compound (18-1) by the method shown in Scheme 18. Carboxylic acid (18-1) is coupled with the amine of a protected boronic acid compound (18-2) to produce amide (18-3), which is then deprotected to generate amine (18-4) as the hydrochloride salt. Subsequent coupling with carboxylic acid (18-5) produces amide (18-6). Deprotection of (18-6) with methylboronic acid (18-7) produces Compound 18.

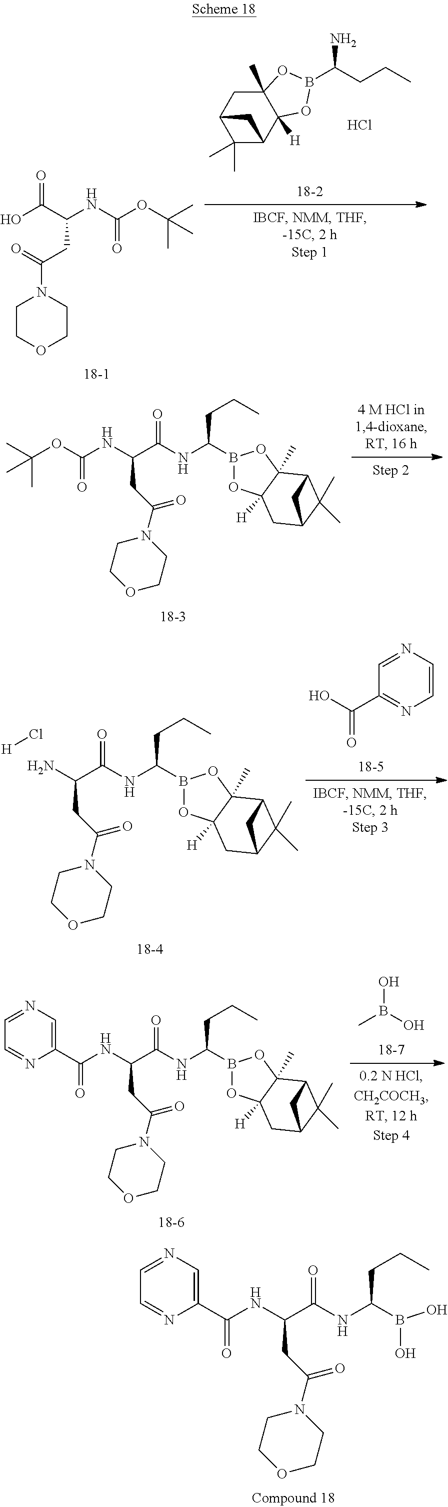

Synthesis of tert-butyl ((R)-4-morpholino-1,4-dioxo-1-(((R)-1-((3aS,4S,6S,7aR)-3a,5,5-trimethylhexa hydro-4,6-methanobenzo[d][1,3,2]dioxaborol-2-yl)butyl)amino)butan-2-yl) carbamate [Step1]: To a stirred solution of (R)-2-((tert-butoxycarbonyl)amino)-4-morpholino-4-oxobutanoic acid (18-1, 690 mg, 2.3 mmol) in THF (10 mL), NMM (0.3 mL, 2.3 mmol) and IBCF (0.3 mL, 2.3 mmol) were added at −15° C. and the reaction mixture was stirred at this temperature for 1 h. To the above solution, (R)-BoroNva-(+)-Pinanediol hydrochloride (18-2, 600 mg, 2.1 mmol) and NMM (0.3 mL, 2.3 mmol) were added and the reaction mixture was stirred at RT for 2 h. After completion of the reaction (monitored by TLC and LC-MS), the reaction mixture was diluted with EtOAc and washed subsequently with 0.1 M HCl, 5% K₂CO₃, water and brine. The organic phase was dried over Na₂SO₄, filtered and evaporated to yield tert-butyl ((R)-4-morpholino-1,4-dioxo-1-(((R)-1-((3aS,4S,6S,7aR)-3a,5,5-trimethylhexahydro-4,6-methanobenzo [d][1,3,2]dioxaborol-2-yl)butyl)amino) butan-2-yl)carbamate (18-3, 1100 mg). The crude was used for next step without further purification. [M−H]⁻=534.5.

Synthesis of (R)-2-amino-4-morpholino-4-oxo-N—((R)-1-((3aS,4S,6S,7aR)-3a,5,5-trimethyl hexahydro-4,6-methanobenzo[d][1,3,2]dioxaborol-2-yl)butyl)butanamide hydrochloride [Step 2]: To a stirred solution of tert-butyl ((R)-4-morpholino-1,4-dioxo-1-(((R)-1-((3aS,4S,6S,7aR)-3a,5,5-trimethylhexa hydro-4,6-methanobenzo[d][1,3,2]dioxaborol-2-yl)butyl) amino)butan-2-yl)carbamate (18-3, 1100 mg, 2 mmol) in 1,4-Dioxane (5 mL) was added 4 M HCl-dioxane (5 mL, 20 mmol) in ice cold condition and the reaction mixture was stirred at RT for 2 h. After completion of the reaction (monitored by LCMS) the solvent was evaporated from reaction mixture. The crude was triturated with n-pentane to yield (R)-2-amino-4-morpholino-4-oxo-N—((R)-1-((3aS,4S,6S,7aR)-3a,5,5-trimethylhexahydro-4,6-methanobenzo[d][1,3,2]dioxaborol-2-yl) butyl)butanamide hydrochloride (18-4, 960 mg). This crude was used for next step without further purification. [M−H]⁻=434.3.

Synthesis of N—((R)-4-morpholino-1,4-dioxo-1-(((R)-1-((3aS,4S,6S,7aR)-3a,5,5-trimethyl hexahydro-4,6-methanobenzo[d][1,3,2]dioxaborol-2-yl)butyl)amino)butan-2-yl)pyrazine-2-carboxamide [Step 3]: To a stirred solution of pyrazine-2-carboxylic acid (18-5, 280 mg, 2.2 mmol) in THF (10 mL), NMM (0.25 mL, 2.2 mmol) and IBCF (0.30 mL, 2.2 mmol) were added at −15° C. and the reaction mixture was stirred at this temperature for 30 min. Then (R)-2-amino-4-morpholino-4-oxo-N—((R)-1-((3aS,4S,6S,7aR)-3a,5,5-trimethylhexahydro-4,6-methanobenzo[d][1,3,2]dioxaborol-2-yl)butyl)butanamide hydrochloride (18-4, 960 mg, 2 mmol) and NMM (0.25 mL, 2.2 mmol) were added and the reaction mixture was stirred at RT for 2 h. After completion of the reaction (monitored by TLC and LC-MS), the reaction mixture was diluted with EtOAc and washed with 0.1 M HCl, 5% K₂CO₃, water and brine consecutively. The organic phase was dried over Na₂SO₄, filtered and evaporated to yield a brown gum. The crude was purified by RP prep HPLC purification and the eluent was lyophilized to afford N—((R)-4-morpholino-1,4-dioxo-1-(((R)-1-((3aS,4S,6S,7aR)-3a,5,5-trimethylhexahydro-4,6-methanobenzo[d][1,3,2]dioxaborol-2-yl) butyl)amino) butan-2-yl)pyrazine-2-carboxamide (18-6, 200 mg). [M−H]⁻=540.4.

Synthesis of ((R)-1-((R)-4-morpholino-4-oxo-2-(pyrazine-2-carboxamido)butanamido) butyl)boronic acid [Step 4]: To a solution of N—((R)-4-morpholino-1,4-dioxo-1-(((R)-1-((3aS,4S,6S,7aR)-3a,5,5-trimethylhexahydro-4,6-methanobenzo[d][1,3,2]dioxaborol-2-yl)butyl)amino) butan-2-yl)pyrazine-2-carboxamide (18-6, 200 mg, 0.37 mmol) in acetone (3 mL) was added methylboronic acid (18-7, 220 mg, 3.7 mmol), followed by drop wise addition of 0.2 N HCl (3 mL). The reaction mixture was allowed to stirring at room temperature overnight. All the volatiles were evaporated at room temperature. The crude was redissolved in acetonitrile and deionized water and freeze-dried to obtain solid. The crude solid was purified through RP prep HPLC purification and lyophilized to afford ((R)-1-((R)-4-morpholino-4-oxo-2-(pyrazine-2-carboxamido)butanamido)butyl)boronic acid (Compound 18, 60 mg). [M−H]⁻=406.1. ¹H NMR (400 MHz, MeOD) δ 9.26 (d, 1H), 8.80 (d, 1H), 8.70-8.69 (m, 1H), 5.29 (t, 1H), 3.68-3.61 (m, 4H), 3.58-3.49 (m, 4H), 3.32-3.27 (m, 1H), 2.99 (dd, 1H), 2.59 (t, 1H), 1.51-1.28 (m, 4H), 0.90 (t, 3H).

### Example 19

### Synthesis of ((R)-1-((R)-4-morpholino-4-oxo-2-(pyrazine-2-carboxamido)butanamido) propyl)boronic acid

In another embodiment, Compound 19 can be produced from compound (19-1) by the method shown in Scheme 19. Carboxylic acid (19-1) is coupled with the amine of a protected boronic acid compound (19-2) to produce amide (19-3), which is then deprotected to generate amine (19-4) as the hydrochloride salt. Subsequent coupling with carboxylic acid (19-5) produces amide (19-6). Deprotection of (19-6) with methylboronic acid (19-7) produces Compound 19.

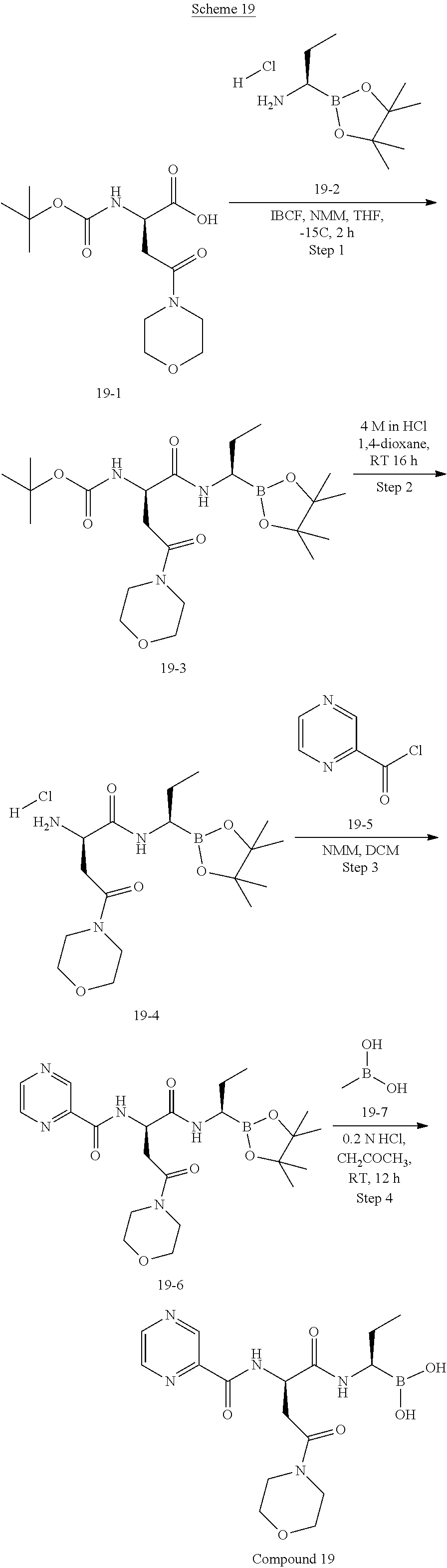

Synthesis of tert-butyl ((R)-4-morpholino-1,4-dioxo-1-(((R)-1-(4,4,5,5-tetramethyl-1,3,2-dioxaborolan-2-yl)propyl)amino)butan-2-yl)carbamate [Step1]: To a stirred solution of (R)-2-((tert-butoxycarbonyl)amino)-4-morpholino-4-oxobutanoic acid (19-1, 900 mg, 3 mmol) in THF (10 mL), NMM (0.4 mL, 3 mmol) and IBCF (0.4 mL, 3 mmol) were added at −15° C. and the reaction mixture was stirred at this temperature for 1 h. To the above solution, (R)-1-(4,4,5,5-tetramethyl-1,3,2-dioxaborolan-2-yl)propan-1-amine hydrochloride (19-2, 600 mg, 2.7 mmol) and NMM (0.4 mL, 3 mmol) were added and the reaction mixture was stirred at RT for 2 h. After completion of the reaction (monitored by TLC and LC-MS), the reaction mixture was diluted with EtOAc and washed subsequently with 0.1 M HCl, 5% K₂CO₃, water and brine. The organic phase was dried over Na₂SO₄, filtered and evaporated to yield tert-butyl ((R)-4-morpholino-1,4-dioxo-1-(((R)-1-(4,4,5,5-tetramethyl-1,3,2-dioxaborolan-2-yl)propyl)amino)butan-2-yl)carbamate (19-3, 1250 mg). The crude was used for next step without further purification. [M−H]⁻=468.4.

Synthesis of (R)-2-amino-4-morpholino-4-oxo-N—((R)-1-(4,4,5,5-tetramethyl-1,3,2-dioxaborolan-2-yl)propyl)butanamide hydrochloride [Step 2]: To a stirred solution of tert-butyl ((R)-4-morpholino-1,4-dioxo-1-(((R)-1-(4,4,5,5-tetramethyl-1,3,2-dioxaborolan-2-yl)propyl)amino) butan-2-yl)carbamate (19-3, 1250 mg, 2.7 mmol) in 1,4-Dioxane (7 mL), add 4 M HCl-dioxane (7 mL, 27 mmol) was added in ice cold condition and the reaction mixture was stirred at RT for 2 h. After completion of the reaction (monitored by LCMS) the solvent was evaporated from reaction mixture. The crude was triturated with n-pentane to yield (R)-2-amino-4-morpholino-4-oxo-N—((R)-1-(4,4,5,5-tetramethyl-1,3,2-dioxaborolan-2-yl)propyl)butanamide hydrochloride (19-4, 1000 mg). This crude was used for next step without further purification. [M+H]⁺=370.3.

Synthesis of N—((R)-4-morpholino-1,4-dioxo-1-(((R)-1-(4,4,5,5-tetramethyl-1,3,2-dioxaborolan-2-yl)propyl)amino)butan-2-yl)pyrazine-2-carboxamide [Step 3]: To a stirred solution of (R)-2-amino-4-morpholino-4-oxo-N—((R)-1-(4,4,5,5-tetramethyl-1,3,2-dioxaborolan-2-yl)propyl)butanamide hydrochloride (19-4, 250 mg, 0.6 mmol) in DCM (7 mL), NMM (0.2 mL, 1.2) was added at −15° C. and the reaction mixture was stirred at this temperature for 20 min. To the above solution, pyrazine-2-carbonyl chloride (19-5, 90 mg, 0.6 mmol) added and the reaction mixture was stirred at RT for 2 h. After completion of the reaction (monitored by TLC and LC-MS), the reaction mixture was diluted with DCM and washed subsequently with 0.1 M HCl, 5% K₂CO₃, water and brine. The organic phase was dried over Na₂SO₄, filtered and evaporated to obtain crude. The crude was purified by PREP-RP HPLC to afford N—((R)-4-morpholino-1,4-dioxo-1-(((R)-1-(4,4,5,5-tetramethyl-1,3,2-dioxaborolan-2-yl)propyl)amino)butan-2-yl)pyrazine-2-carboxamide (19-6, 30 mg). [M−H]⁻=474.3.

Synthesis of ((R)-1-((R)-4-morpholino-4-oxo-2-(pyrazine-2-carboxamido)butanamido) propyl)boronic acid [Step 4]: To a solution of N—((R)-4-morpholino-1,4-dioxo-1-(((R)-1-(4,4,5,5-tetramethyl-1,3,2-dioxaborolan-2-yl)propyl)amino)butan-2-yl)pyrazine-2-carboxamide (19-6, 30 mg, 0.06 mmol) in acetone (1.5 mL) was added methylboronic acid (19-7, 38 mg, 0.6 mmol), followed by drop wise addition of 0.2M HCl (1.5 mL). The reaction mixture was allowed to stirring at room temperature overnight. All the volatiles were evaporated at room temperature, and reaction mixture was re-dissolved in acetonitrile and deionized water and freeze-dried. The obtained crude was purified through RP prep HPLC purification and lyophilized to afford ((R)-1-((R)-4-morpholino-4-oxo-2-(pyrazine-2-carboxamido)butanamido)propyl) boronic acid (Compound 19, 20 mg). [M−H]⁻=392.2. ¹H NMR (400 MHz, MeOD) δ 9.26 (d, 1H), 8.80 (d, 1H), 8.69 (t, 1H), 5.30 (t, 1H), 3.63-3.61 (m, 2H), 3.58 (d, 2H), 3.55-3.51 (m, 4H), 3.02-2.97 (dd, 1H), 2.50 (t, 1H), 1.59-1.52 (m, 1H), 1.50-1.48 (m, 1H), 1.28 (s, 1H), 0.92 (t, 3H).

### Example 20

In another embodiment, Compound 20 can be produced from compound (20-1) by the method shown in Scheme 20. Amine (20-1) is coupled with compound (20-2) to afford amide (20-3). Subsequent deprotection of (20-3) with methylboronic acid (20-4) produces Compound 20.

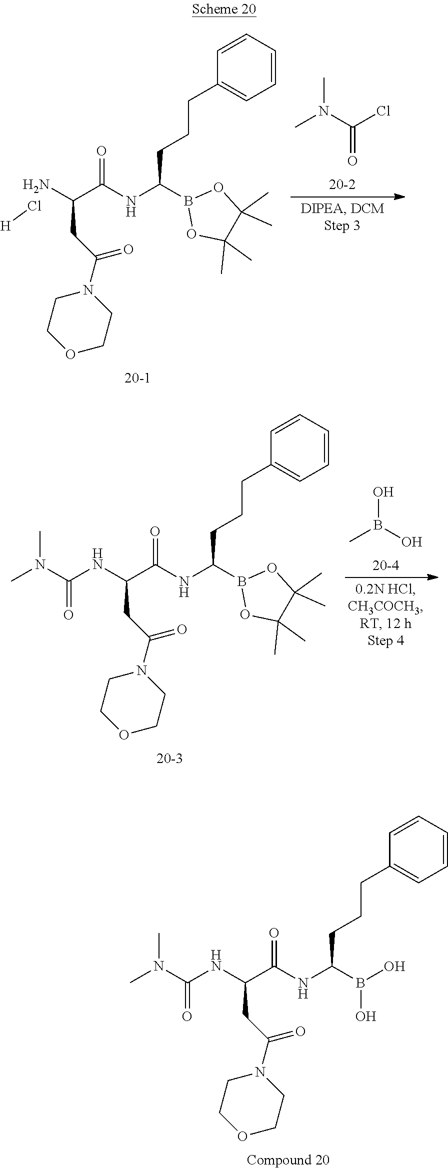

Synthesis of (R)-2-(3,3-dimethylureido)-4-morpholino-4-oxo-N—((R)-4-phenyl-1-(4,4,5,5-tetramethyl-1,3,2-dioxaborolan-2-yl)butyl)butanamide [Step 3]: To a stirred solution of (R)-2-amino-4-morpholino-4-oxo-N—((R)-4-phenyl-1-(4,4,5,5-tetramethyl-1,3,2-dioxaborolan-2-yl) butyl)butanamide hydrochloride (20-1, 300 mg, 0.6 mmol) and N,N-dimethylcarbamoyl chloride (20-2, 0.06 mL, 0.7 mmol) in DCM (3 mL), DIPEA (0.5 mL, 3.0 mmol) was added in ice cold condition and the reaction mixture was stirred at RT for 2 h. TLC and LCMS showed full conversion of starting material with formation of new polar spot. The reaction mixture was diluted with DCM and washed with water and brine solution. The organic phase was dried over Na₂SO₄, filtered and evaporated. The crude material was purified by RP prep HPLC purification and lyophilized to afford (R)-2-(3,3-dimethylureido)-4-morpholino-4-oxo-N—((R)-4-phenyl-1-(4,4,5,5-tetramethyl-1,3,2-dioxaborolan-2-yl)butyl)butanamide (20-3, 46 mg). [M−H]⁺=529.5. [M-84+H]⁺=447.3.

Synthesis of (R)-1-((R)-2-(3,3-dimethylureido)-4-morpholino-4-oxobutanamido)-4-phenyl butyl)boronic acid [Step 4]: To a stirred solution of (R)-2-(3,3-dimethylureido)-4-morpholino-4-oxo-N—((R)-4-phenyl-1-(4,4,5,5-tetramethyl-1,3,2-dioxaborolan-2-yl)butyl) butanamide (20-3, 46 mg, 0.1 mmol) and methylboronic acid (20-4, 52 mg, 1.0 mmol) in acetone (3 mL) was added 0.2 N HCl (3.0 mL) and the reaction mixture was stirred at RT overnight. TLC and LCMS showed full conversion of starting material with formation of new polar spot. The volatiles were evaporated and the residue was purified via RP prep HPLC purification and lyophilized to afford (R)-1-((R)-2-(3,3-dimethylureido)-4-morpholino-4-oxobutanamido)-4-phenylbutyl)boronic acid (Compound 20.22 mg). [M−H]⁺: 447.2. ¹H NMR (400 MHz, MeOD) δ 7.24-7.11 (m, 5H), 4.92 (t, 1H), 3.65-3.59 (m, 4H), 3.51-3.48 (m, 4H), 3.03-3.01 (m, 1H), 2.91-2.86 (m, 7H), 2.62-2.56 (m, 3H), 1.70-1.48 (m, 4H).

### Example 21

### Synthesis of ((S)-1-((R)-4-morpholino-4-oxo-2-(pyrazine-2-carboxamido)butanamido)-4-phenylbutyl)boronic acid

In another embodiment, Compound 21 can be produced from compound (21-1) by the method shown in Scheme 21. Amino acid (21-1) is coupled with the amine of a protected boronic acid compound (21-2) to produce amide (21-3), which is then deprotected to generate amine (21-4) as the hydrochloride salt. Subsequent coupling with carboxylic acid (21-5) produces amide (21-6). Deprotection of (21-6) with methylboronic acid (21-7) produces Compound 21.

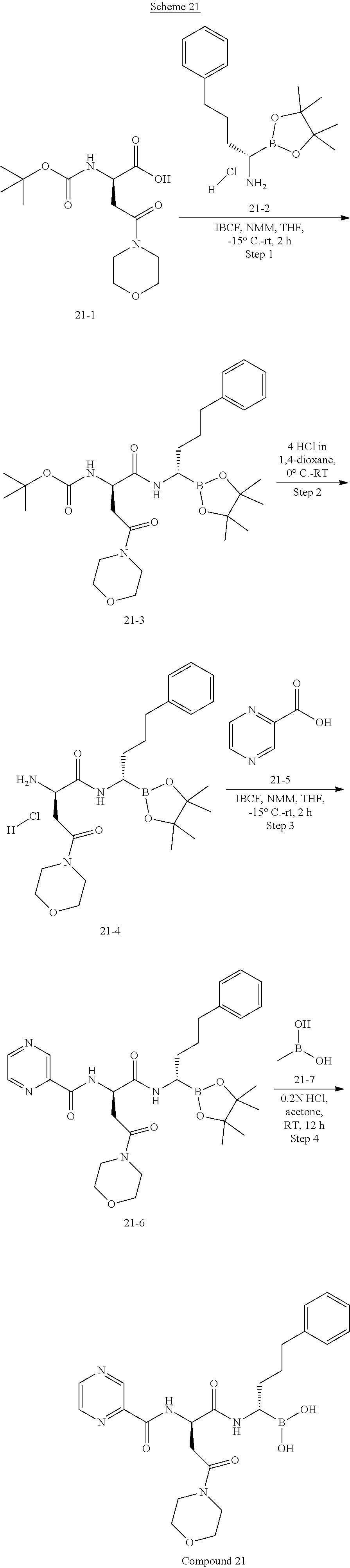

Synthesis of tert-butyl ((R)-4-morpholino-1,4-dioxo-1-(((S)-4-phenyl-1-(4,4,5,5-tetramethyl-1,3,2-dioxaborolan-2-yl)butyl)amino)butan-2-yl)carbamate [Step 1]: To a stirred solution of (R)-2-((tert-butoxycarbonyl)amino)-4-morpholino-4-oxobutanoic acid (21-1, 50 mg, 2.4 mmol) in THF (8 mL) was added IBCF (0.3 mL, 2.4 mmol) and NMM (0.3 mL, 2.4 mmol) at −15° C. Reaction mixture was stirred at same temperature for 30 min. Then (S)-4-phenyl-1-(4,4,5,5-tetramethyl-1,3,2-dioxaborolan-2-yl)butan-1-amine hydrochloride (21-2, 700 mg, 2.2 mmol) in DMF (1 mL) followed by NMM (0.2 mL, 2.2 mmol) was added to the reaction mixture at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. Combined organic layer was washed with 5% K₂CO₃ solution, water, brine, dried over Na₂SO₄, filtered and evaporated under reduced pressure to afford tert-butyl ((R)-4-morpholino-1,4-dioxo-1-(((S)-4-phenyl-1-(4,4,5,5-tetramethyl-1,3,2-dioxaborolan-2-yl)butyl) amino)butan-2-yl)carbamate (21-3, 700 mg). [M+H]⁺: 560.1.

Synthesis of (R)-2-amino-4-morpholino-4-oxo-N—((S)-4-phenyl-1-(4,4,5,5-tetramethyl-1,3,2-dioxaborolan-2-yl)butyl)butanamide hydrochloride [Step 2]: To a solution of tert-butyl ((R)-4-morpholino-1,4-dioxo-1-(((S)-4-phenyl-1-(4,4,5,5-tetramethyl-1,3,2-dioxaborolan-2-yl)butyl) amino)butan-2-yl)carbamate (21-3, 700 mg, 1.2 mmol) in 1,4 dioxane (6 mL) was added 4 M HCl in dioxane (6.0 mL, 25.0 mmol) at 0° C. It was gradually warmed to 25° C. and stirred for 16 h. TLC showed complete consumption of starting material to form new polar spot. Volatiles were removed under reduced pressure to get (R)-2-amino-4-morpholino-4-oxo-N—((S)-4-phenyl-1-(4,4,5,5-tetramethyl-1,3,2-dioxaborolan-2-yl)butyl)butanamide hydrochloride (21-4, 600 mg). The crude was directly used for next step without purification.

Synthesis of N—((R)-4-morpholino-1,4-dioxo-1-(((S)-4-phenyl-1-(4,4,5,5-tetramethyl-1,3,2-dioxaborolan-2-yl)butyl)amino)butan-2-yl)pyrazine-2-carboxamide [Step 3]: To a stirred solution of pyrazine-2-carboxylic acid (21-5, 165 mg, 1.3 mmol) in THF (8 mL) was added IBCF (0.2 mL, 1.3 mmol) and NMM (0.15 mL, 1.3 mmol) at −15° C. The reaction mixture was stirred at same temperature for 30 min. Then (R)-2-amino-4-morpholino-4-oxo-N—((S)-4-phenyl-1-(4,4,5,5-tetramethyl-1,3,2-dioxaborolan-2-yl)butyl)butanamide hydrochloride (21-4, 600 mg, 1.2 mmol) in DMF (1 mL) followed by NMM (0.1 mL, 1.2 mmol) was added to the reaction mixture under same condition. It was gradually warmed to 0° C. and stirred for 2 h. LCMS of the crude reaction mass confirmed the formation of desired product. It was neutralized with saturated aqueous 0.1 N HCl solution and extracted with ethyl acetate. The combined organic layer was washed with 5% K₂CO₃ solution, water, brine, dried over Na₂SO₄, filtered and evaporated under reduced pressure. The crude material was purified by RP prep HPLC purification and lyophilized to afford N—((R)-4-morpholino-1,4-dioxo-1-(((S)-4-phenyl-1-(4,4,5,5-tetramethyl-1,3,2-dioxaborolan-2-yl)butyl) amino)butan-2-yl)pyrazine-2-carboxamide (21-6, 80 mg). [M−H]⁺=564.4.

Synthesis of ((S)-1-((R)-4-morpholino-4-oxo-2-(pyrazine-2-carboxamido)butanamido)-4-phenylbutyl)boronic acid [Step 4]: To a stirred solution of N—((R)-4-morpholino-1,4-dioxo-1-(((S)-4-phenyl-1-(4,4,5,5-tetramethyl-1,3,2-dioxaborolan-2-yl)butyl)amino)butan-2-yl)pyrazine-2-carboxamide (21-6, 70 mg, 0.1 mmol) and methylboronic acid (21-7, 74 mg, 1.0 mmol) in acetone (4 mL) was added 0.2 N HCl (4 mL) and the reaction mixture was stirred at RT overnight. TLC and LCMS showed full conversion of starting material with formation of new polar spot. The volatiles were evaporated and the crude material was purified by RP prep HPLC purification and lyophilized to afford ((S)-1-((R)-4-morpholino-4-oxo-2-(pyrazine-2-carboxamido)butanamido)-4-phenylbutyl) boronic acid (Compound 21, 16 mg). [M−H]⁺: 482.4. ¹H NMR (400 MHz, MeOD) δ 9.25 (s, 1H), 8.79 (d, 1H), 8.68 (s, 1H), 7.21-7.07 (m, 5H), 5.28 (t, 1H), 3.66 (t, 2H), 3.61-3.47 (m, 6H), 2.98-2.93 (m, 1H), 2.65 (t, 1H), 2.58-2.57 (m, 2H), 1.65-1.39 (m, 5H).

### Example 22

### Synthesis of ((R)-1-((R)-2-(cyclohexanecarboxamido)-4-morpholino-4-oxobutanamido)-4-phenylbutyl)boronic acid

In another embodiment, Compound 22 can be produced from compound (22-1) by the method shown in Scheme 22. Amine (22-1) is coupled with compound (22-2) to afford amide (22-3). Subsequent deprotection of (22-3) with methylboronic acid (22-4) produces Compound 22.

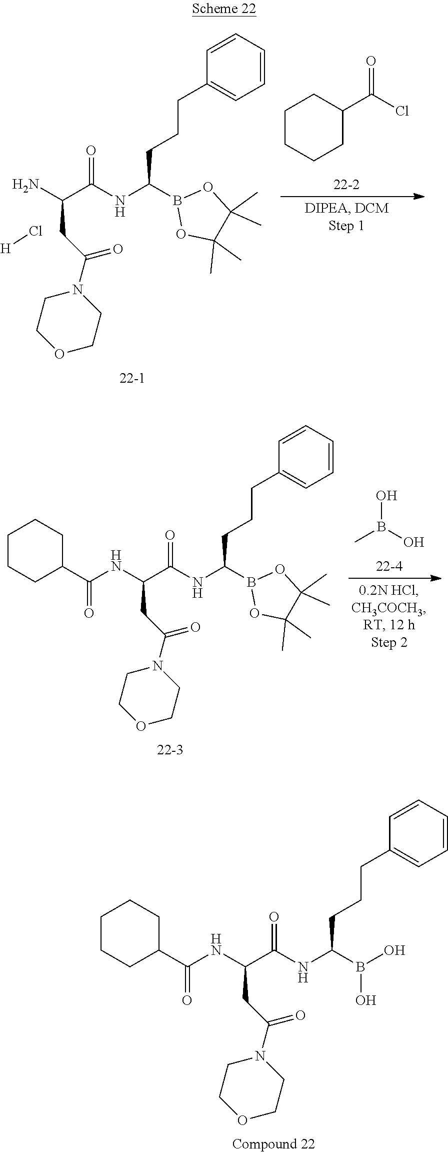

Synthesis of N—((R)-4-morpholino-1,4-dioxo-1-(((R)-4-phenyl-1-(4,4,5,5-tetramethyl-1,3,2-dioxaborolan-2-yl)butyl)amino)butan-2-yl)cyclohexanecarboxamide [Step 1]: To a stirred solution of (R)-2-amino-4-morpholino-4-oxo-N—((R)-4-phenyl-1-(4,4,5,5-tetramethyl-1,3,2-dioxaborolan-2-yl)butyl)butanamide hydrochloride (22-1, 300 mg, 0.6 mmol) and cyclohexane carbonyl chloride (22-2, 0.1 mL, 0.7 mmol) in DCM (4 mL), DIPEA (0.5 mL, 3. mmol) was added in ice cold condition and the reaction mixture was stirred at RT for 2 h. TLC and LCMS showed full conversion of starting material with formation of new polar spot. The reaction was diluted with DCM and washed with water and brine solution. The organic phase was dried over Na₂SO₄, filtered and evaporated. The crude material was purified by RP prep HPLC purification and lyophilized to afford N—((R)-4-morpholino-1,4-dioxo-1-(((R)-4-phenyl-1-(4,4,5,5-tetramethyl-1,3,2-dioxaborolan-2-yl)butyl)amino)butan-2-yl)cyclohexanecarboxamide (22-3, 45 mg). [M−H]⁺=568.7. [M-84+H]⁺=486.4.

Synthesis of ((R)-1-((R)-2-(cyclohexanecarboxamido)-4-morpholino-4-oxobutanamido)-4-phenylbutyl)boronic acid [Step 2]: To a stirred solution of N—((R)-4-morpholino-1,4-dioxo-1-(((R)-4-phenyl-1-(4,4,5,5-tetramethyl-1,3,2-dioxaborolan-2-yl)butyl)amino)butan-2-yl)cyclo hexanecarboxamide (22-3, 45 mg, 0.1 mmol) and methylboronic acid (22-4, 45 mg, 0.8 mmol) in acetone (3 mL) was added 0.2 N HCl (3.0 mL) and the reaction mixture was stirred at RT overnight. TLC and LCMS showed full conversion of starting material with formation of new polar spot. The volatiles were evaporated and the residue was redissolved in acetone and deionized water and freeze-dried to obtain crude. The crude material was purified by RP prep HPLC purification and lyophilized to afford ((R)-1-((R)-2-(cyclohexanecarboxamido)-4-morpholino-4-oxobutanamido)-4-phenylbutyl)boronic acid (Compound 22.17 mg). [M−H]⁺: 486.3. ¹H NMR (400 MHz, MeOD) δ 7.24-7.10 (m, 5H), 4.97 (brs, 1H), 3.65-3.59 (m, 4H), 3.50-3.48 (m, 4H), 2.92-2.90 (m, 2H), 2.61-2.59 (m, 3H), 2.19 (t, 1H), 1.78-1.67 (m, 7H), 1.46-1.28 (m, 7H).

### Example 23

### Synthesis of ((R)-1-((R)-4-morpholino-4-oxo-2-(tetrahydro-2H-pyran-4-carboxamido) butanamido)-4-phenylbutyl)boronic acid

In another embodiment, Compound 23 can be produced from compound (23-1) by the method shown in Scheme 23. Amine (23-1) is coupled with compound (23-2) to afford amide (23-3). Subsequent deprotection of (23-3) with methylboronic acid (23-4) produces Compound 23.

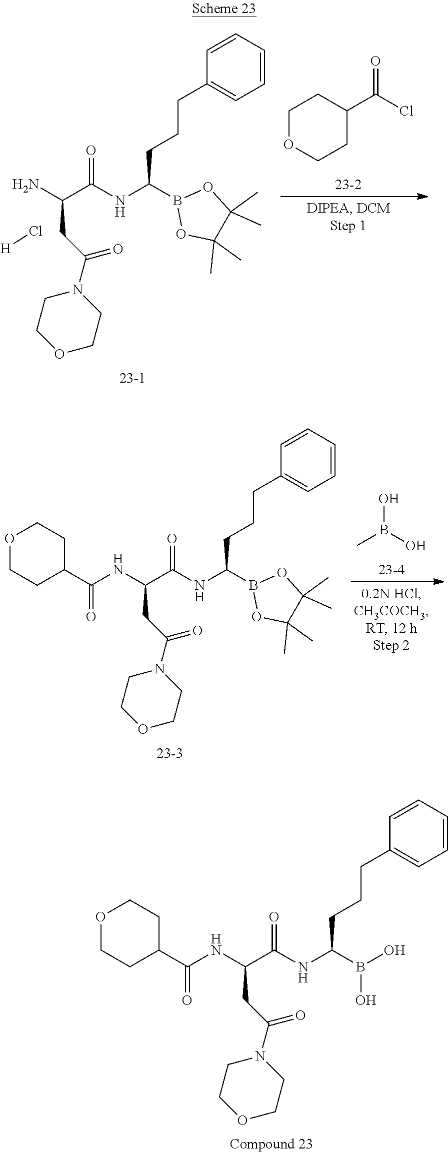

Synthesis of N—((R)-4-morpholino-1,4-dioxo-1-(((R)-4-phenyl-1-(4,4,5,5-tetramethyl-1,3,2-dioxaborolan-2-yl)butyl)amino)butan-2-yl)tetrahydro-2H-pyran-4-carboxamide [Step 1]: To a stirred solution of (R)-2-amino-4-morpholino-4-oxo-N—((R)-4-phenyl-1-(4,4,5,5-tetramethyl-1,3,2-dioxaborolan-2-yl)butyl)butanamide hydrochloride (23-1, 300 mg, 0.6 mmol) and tetrahydro-2H-pyran-4-carbonyl chloride (23-2, 0.08 mL, 0.7 mmol) in DCM (4 mL), DIPEA (0.5 mL, 3 mmol) was added in ice cold condition and the reaction mixture was stirred at RT for 2 h. TLC and LCMS showed full conversion of starting material with formation of new polar spot. The reaction was diluted with DCM and washed with water and brine solution. The organic phase was dried over Na₂SO₄, filtered and evaporated. The crude material was purified by RP prep HPLC purification and lyophilized to afford N—((R)-4-morpholino-1,4-dioxo-1-(((R)-4-phenyl-1-(4,4,5,5-tetramethyl-1,3,2-dioxaborolan-2-yl)butyl)amino)butan-2-yl)tetrahydro-2H-pyran-4-carboxamide (23-3, 75 mg). [M−H]⁺=570.4. [M-84+H]⁺=488.5.

Synthesis of ((R)-1-((R)-4-morpholino-4-oxo-2-(tetrahydro-2H-pyran-4-carboxamido) butanamido)-4-phenylbutyl)boronic acid [Step 2]: To a stirred solution of N—((R)-4-morpholino-1,4-dioxo-1-(((R)-4-phenyl-1-(4,4,5,5-tetramethyl-1,3,2-dioxaborolan-2-yl)butyl) amino)butan-2-yl)tetrahydro-2H-pyran-4-carboxamide (23-3, 75 mg, 0.13 mmol) and methylboronic acid (23-4, 80 mg, 1.3 mmol) in acetone (4 mL) was added 0.2 N HCl (4 mL) and the reaction mixture was stirred at RT overnight. TLC and LCMS showed full conversion of starting material with formation of new polar spot. The volatiles were evaporated and the residue was redissolved in acetone and deionized water and freeze-dried to obtain crude. The crude material was purified by RP prep HPLC purification and lyophilized to afford ((R)-1-((R)-4-morpholino-4-oxo-2-(tetrahydro-2H-pyran-4-carboxamido)butanamido)-4-phenylbutyl)boronic acid (Compound 23, 49 mg). [M−H]⁺: 488.2. ¹H NMR (400 MHz, MeOD) δ 7.24-7.12 (m, 5H), 4.97 (t, 1H), 3.95-3.92 (m, 2H), 3.67-3.59 (m, 4H), 3.51-3.37 (m, 6H), 2.92 (d, 2H), 2.60-2.45 (m, 4H), 1.72-1.47 (m, 8H).

### Example 24

### Synthesis of ((R)-1-((R)-4-morpholino-4-oxo-2-(phenylsulfonamido)butanamido)-4-phenyl butyl)boronic acid

In another embodiment, Compound 24 can be produced from compound (24-1) by the method shown in Scheme 24. Amine (24-1) is coupled with compound (24-2) to afford sulfonamide (24-3). Subsequent deprotection of (24-3) with methylboronic acid (24-4) produces Compound 24.

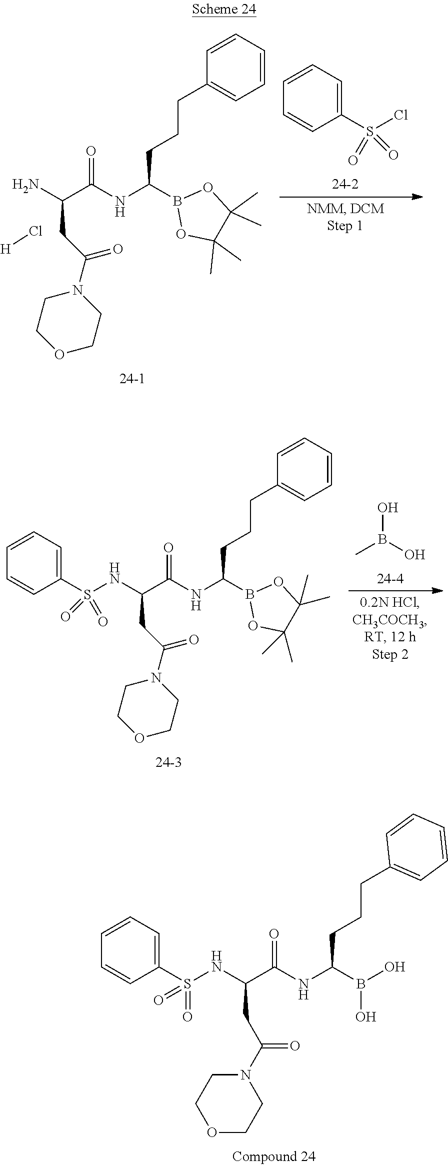

Synthesis of (R)-4-morpholino-4-oxo-N—((R)-4-phenyl-1-(4,4,5,5-tetramethyl-1,3,2-dioxaborolan-2-yl)butyl)-2-(phenylsulfonamido)butanamide [Step 1]: To a stirred solution of (R)-2-amino-4-morpholino-4-oxo-N—((R)-4-phenyl-1-(4,4,5,5-tetramethyl-1,3,2-dioxaborolan-2-yl)butyl)butanamide hydrochloride (24-1, 300 mg, 0.6 mmol) and benzenesulfonyl chloride (24-2, 0.08 mL, 0.67 mmol) in DCM (4 mL) was added NMM (0.33 mL, 3 mmol) in ice cold condition and stirred at RT for 2 h. TLC and LCMS showed complete conversion of starting material to desired product. The reaction was diluted with DCM and washed with water and brine, dried over Na₂SO₄ and evaporated. The crude was purified via prep HPLC purification and lyophilized to afford mixture of (R)-4-morpholino-4-oxo-N—((R)-4-phenyl-1-(4,4,5,5-tetramethyl-1,3,2-dioxaborolan-2-yl)butyl)-2-(phenylsulfonamido)butanamide and ((R)-1-((R)-4-morpholino-4-oxo-2-(phenylsulfonamido) butanamido)-4-phenylbutyl)boronic acid (24-3, 47 mg). [M−H]⁺=598.3. [M-84+H]⁺=516.3.

Synthesis of ((R)-1-((R)-4-morpholino-4-oxo-2-(phenylsulfonamido)butanamido)-4-phenyl butyl)boronic acid [Step 2]: To a stirred solution of (R)-4-morpholino-4-oxo-N—((R)-4-phenyl-1-(4,4,5,5-tetramethyl-1,3,2-dioxaborolan-2-yl)butyl)-2-(phenylsulfonamido)butanamide [mixture with ((R)-1-((R)-4-morpholino-4-oxo-2-(phenylsulfonamido)butanamido)-4-phenylbutyl)boronic acid (24-3, 47 mg, 0.08 mmol) and methylboronic acid (24-4, 47 mg, 0.8 mmol) in Acetone (3 mL) was added 0.2 N HCl (3.0 mL) and stirred at RT for overnight. TLC and LCMS showed full conversion of starting material to desired product. Volatiles were evaporated under reduced pressure and lyophilized. The crude was purified via prep HPLC purification and lyophilized to afford ((R)-1-((R)-4-morpholino-4-oxo-2-(phenylsulfonamido)butanamido)-4-phenylbutyl)boronic acid (Compound 24, 31 mg). [M−H]⁺: 516.3. ¹H NMR (400 MHz, MeOD) δ 7.87-7.85 (m, 2H), 7.60-7.51 (m, 3H), 7.26-7.13 (m, 5H), 4.45 (t, 1H), 3.58-3.53 (m, 4H), 3.43-3.33 (m, 4H), 2.76-2.69 (m, 2H), 2.61-2.56 (m, 3H), 1.61-1.59 (m, 2H), 1.50-1.20 (m, 2H).

### Example 25

### Synthesis of ((1R)-1-((2R)-4-morpholino-4-oxo-2-(tetrahydro-2H-pyran-2-carboxamido) butanamido)-4-phenylbutyl)boronic acid

In another embodiment, Compound 25 can be produced from compound (25-1) by the method shown in Scheme 25. Amine (25-1) is coupled with compound (25-2) to afford amide (25-3). Subsequent deprotection of (25-3) with methylboronic acid (25-4) produces Compound 25.

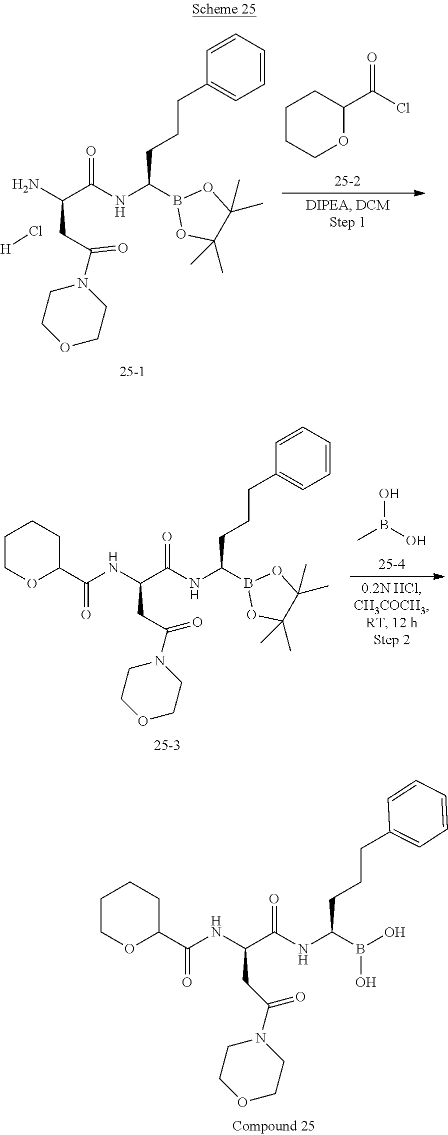

Synthesis of N—((R)-4-morpholino-1,4-dioxo-1-(((R)-4-phenyl-1-(4,4,5,5-tetramethyl-1,3,2-dioxaborolan-2-yl)butyl)amino)butan-2-yl)tetrahydro-2H-pyran-2-carboxamide [Step 1]: To a stirred solution of (R)-2-amino-4-morpholino-4-oxo-N—((R)-4-phenyl-1-(4,4,5,5-tetramethyl-1,3,2-dioxaborolan-2-yl)butyl)butanamide hydrochloride (25-1, 250 mg, 0.5 mmol) and tetrahydropyran-2-carbonyl chloride (25-2, 82 mg, 0.5 mmol) in DCM (4 mL), DIPEA (0.4 mL, 2.5 mmol) was added in ice cold condition and the reaction mixture was stirred at RT for 2 h. The reaction was diluted with DCM and washed with water and brine solution. The organic phase was dried over Na₂SO₄, filtered and evaporated. Crude material was purified by prep HPLC purification and lyophilized to afford N—((R)-4-morpholino-1,4-dioxo-1-(((R)-4-phenyl-1-(4,4,5,5-tetramethyl-1,3,2-dioxaborolan-2-yl)butyl)amino)butan-2-yl)tetrahydro-2H-pyran-2-carboxamide (25-3, 60 mg). [M−H]⁺=570.5. [M-84+H]⁺=488.4.

Synthesis of ((1R)-1-((2R)-4-morpholino-4-oxo-2-(tetrahydro-2H-pyran-2-carboxamido) butanamido)-4-phenylbutyl)boronic acid [Step 2]: To a stirred solution of N—((R)-4-morpholino-1,4-dioxo-1-(((R)-4-phenyl-1-(4,4,5,5-tetramethyl-1,3,2-dioxaborolan-2-yl)butyl) amino)butan-2-yl)tetrahydro-2H-pyran-2-carboxamide (25-3, 60 mg, 0.1 mmol) and methylboronic acid (25-4, 63 mg, 1 mmol) in acetone (4 mL) was added 0.2 N HCl (4.0 mL) and the reaction mixture was stirred at RT overnight. The volatiles were evaporated and the residue was redissolved in acetone and deionized water and freeze-dried to obtain crude. Crude material was purified by prep HPLC purification and lyophilized to afford ((1R)-1-((2R)-4-morpholino-4-oxo-2-(tetrahydro-2H-pyran-2-carboxamido)butanamido)-4-phenylbutyl)boronic acid (Compound 25, 25 mg). [M−H]⁺: 488.5. ¹H NMR (400 MHz, MeOD) δ 7.23-7.12 (m, 5H), 5.05 (brs, 1H), 4.05 (d, 1H), 3.78-3.88 (m, 1H), 3.66-3.59 (m, 4H), 3.51-3.48 (m, 6H), 3.28-3.05 (m, 2H), 2.94-2.84 (m, 1H), 2.63-2.58 (m, 3H), 1.97-1.88 (m.3H) 1.67-1.50 (m, 10H).

### Example 26

### Synthesis of ((R)-1-((S)-4-morpholino-4-oxo-2-(pyrazine-2-carboxamido)butanamido)-4-phenylbutyl)boronic acid

In another embodiment, Compound 26 can be produced from compound (26-1) by the method shown in Scheme 26. Amine (26-1) is coupled with morpholine to produce amide (26-3), which is deprotected to produce carboxylic acid (26-4), and then coupled with the amine of a protected boronic acid compound (26-5) to produce amide (26-6). Amide (26-6) is deprotected to generate amine (26-7) as the hydrochloride salt. Subsequent coupling with carboxylic acid (26-8) produces amide (26-9). Deprotection of (26-9) with methylboronic acid (26-10) produces Compound 26.

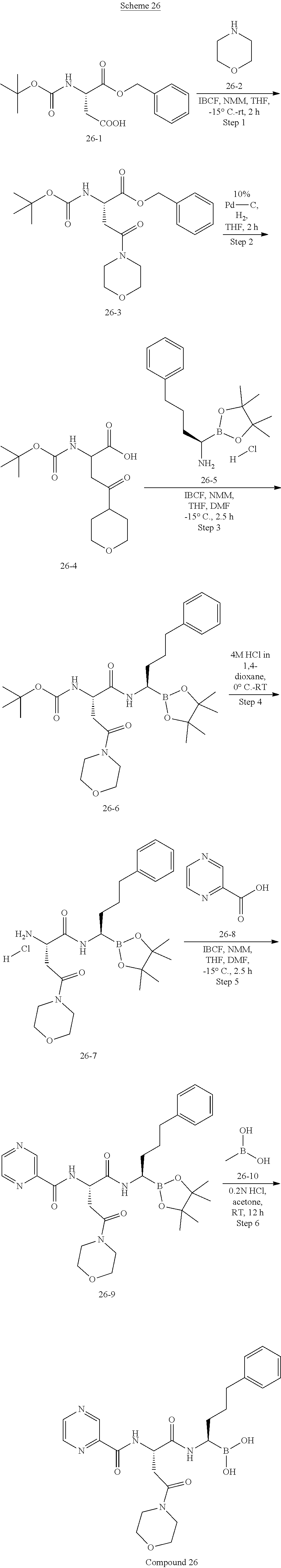

Synthesis of benzyl (S)-2-((tert-butoxycarbonyl)amino)-4-morpholino-4-oxobutanoate [Step 1]: To a stirred solution of (S)-4-(benzyloxy)-3-((tert-butoxycarbonyl)amino)-4-oxobutanoic acid (26-1, 3.0 g, 9.4 mmol) in THF (25 mL) was added IBCF (1.2 mL, 9.4 mmol) and NMM (1.0 mL, 9.4 mmol) at −15° C. The reaction mixture was stirred at same temperature for 30 min. Then morpholine (26-2, 0.75 mL, 8.6 mmol) followed by NMM (0.9 mL, 8.6 mmol) was added to the reaction mixture at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. Combined organic layer was washed with 5% K₂CO solution, water, brine, dried over Na₂SO₄, filtered and evaporated under reduced pressure to get crude residue. The crude was purified by silica gel column chromatography to afford benzyl (S)-2-((tert-butoxycarbonyl)amino)-4-morpholino-4-oxobutanoate (26-3, 3.2 g). [M+H]⁺: 393.0.

Synthesis of (S)-2-((tert-butoxycarbonyl)amino)-4-morpholino-4-oxobutanoic acid [Step 2]: To a stirred solution of benzyl (S)-2-((tert-butoxycarbonyl)amino)-4-morpholino-4-oxobutanoate (26-3, 3.4 g, 8.6 mmol) dissolved in THF (30 mL) was bubbled with nitrogen gas for 10 min. Then 10% Pd—C (1.3 g, 11.9 mmol) was added and the reaction mixture was hydrogenated under balloon pressure for 16 h. The reaction was monitored by TLC. Upon completion, the reaction was filtered over celite with excess ethyl acetate. Removal of solvent under reduced pressure provided (S)-2-((tert-butoxycarbonyl)amino)-4-morpholino-4-oxobutanoic acid (26-4, 2.4 mg). [M+H]: 301.2.

Synthesis of tert-butyl ((S)-4-morpholino-1,4-dioxo-1-(((R)-4-phenyl-1-(4,4,5,5-tetramethyl-1,3,2-dioxaborolan-2-yl)butyl)amino)butan-2-yl)carbamate [Step 3]: To a stirred solution of (S)-2-((tert-butoxycarbonyl)amino)-4-morpholino-4-oxobutanoic acid (26-4, 695 mg, 2.3 mmol) in THF (10 mL) was added IBCF (0.3 mL, 2.3 mmol) and NMM (0.3 mL, 2.3 mmol) at −15° C.

Reaction mixture was stirred at same temperature for 30 min. Then (R)-4-phenyl-1-(4,4,5,5-tetramethyl-1,3,2-dioxaborolan-2-yl)butan-1-amine hydrochloride (26-5, 650 mg, 2.1 mmol) in DMF (1 mL) followed by NMM (0.2 mL, 2.1 mmol) was added to the reaction mixture at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. Combined organic layer was washed with 5% K₂CO₃ solution, water, brine, dried over Na₂SO₄, filtered and evaporated under reduced pressure to afford tert-butyl ((S)-4-morpholino-1,4-dioxo-1-(((R)-4-phenyl-1-(4,4,5,5-tetramethyl-1,3,2-dioxaborolan-2-yl)butyl) amino)butan-2-yl)carbamate (26-6, 700 mg, crude), which is directly used in next step. [M+H]: 558.4.

Synthesis of (S)-2-amino-4-morpholino-4-oxo-N—((R)-4-phenyl-1-(4,4,5,5-tetramethyl-1,3,2-dioxaborolan-2-yl)butyl)butanamide hydrochloride [Step 4]: To a solution of tert-butyl ((S)-4-morpholino-1,4-dioxo-1-(((R)-4-phenyl-1-(4,4,5,5-tetramethyl-1,3,2-dioxaborolan-2-yl)butyl) amino)butan-2-yl)carbamate (26-6, 700 mg, 1.2 mmol) in 6 mL 1,4 dioxane was added 4 M HCl in dioxane (6.0 mL, 24.0 mmol) at 0° C. It was gradually warmed to 25° C. and stirred for 16 h. TLC showed complete consumption of starting material to form new polar spot. Volatiles were removed under reduced pressure to get (S)-2-amino-4-morpholino-4-oxo-N—((R)-4-phenyl-1-(4,4,5,5-tetramethyl-1,3,2-dioxaborolan-2-yl)butyl)butanamide hydrochloride (26-7, 560 mg, crude). Crude was directly used for next step without purification. [M+H]: 458.4.

Synthesis of N—((S)-4-morpholino-1,4-dioxo-1-(((R)-4-phenyl-1-(4,4,5,5-tetramethyl-1,3,2-dioxaborolan-2-yl)butyl)amino)butan-2-yl)pyrazine-2-carboxamide [Step 5]: To a stirred solution of pyrazine-2-carboxylic acid (26-8, 165 mg, 1.3 mmol) in THF (8 mL) was added IBCF (0.17 mL, 1.3 mmol) and NMM (0.17 mL, 1.3 mmol) at −15° C. The reaction mixture was stirred at same temperature for 30 min. Then (S)-2-amino-4-morpholino-4-oxo-N—((R)-4-phenyl-1-(4,4,5,5-tetramethyl-1,3,2-dioxaborolan-2-yl)butyl)butanamide hydrochloride (26-7, 600 mg, 1.2 mmol) in DMF (1 mL) followed by NMM (0.14 mL, 1.2 mmol) was added to the reaction mixture under same condition. It was gradually warmed to 0° C. and stirred for 2 h. LCMS of the crude reaction mass confirmed the formation of desired product. It was neutralized with saturated aqueous 0.1 N HCl solution and extracted with ethyl acetate. The combined organic layer was washed with 5% K₂CO₃ solution, water, brine, dried over Na₂SO₄, filtered and evaporated under reduced pressure. The crude material was purified by RP prep HPLC purification and lyophilized to afford N—((S)-4-morpholino-1,4-dioxo-1-(((R)-4-phenyl-1-(4,4,5,5-tetramethyl-1,3,2-dioxaborolan-2-yl)butyl) amino)butan-2-yl)pyrazine-2-carboxamide (26-9, 105 mg). [M−H]⁺=564.4. [M-84+H]⁺=482.1.

Synthesis of ((R)-1-((S)-4-morpholino-4-oxo-2-(pyrazine-2-carboxamido)butanamido)-4-phenylbutyl)boronic acid [Step 6]: To a stirred solution of N—((S)-4-morpholino-1,4-dioxo-1-(((R)-4-phenyl-1-(4,4,5,5-tetramethyl-1,3,2-dioxaborolan-2-yl)butyl)amino)butan-2-yl)pyrazine-2-carboxamide (26-9, 100 mg, 0.17 mmol) and methylboronic acid (26-10, 105 mg, 1.7 mmol) in acetone (4 mL) was added 0.2 N HCl (4 mL) and the reaction mixture was stirred at RT overnight. TLC and LCMS showed full conversion of starting material with formation of new polar spot. The volatiles were evaporated and the residue was redissolved in acetone and deionized water and freeze-dried to obtain the crude. The crude material was purified by RP prep HPLC purification and lyophilized to afford ((R)-1-((S)-4-morpholino-4-oxo-2-(pyrazine-2-carboxamido) butanamido)-4-phenylbutyl) boronic acid (Compound 26, 62 mg). [M−H]⁺: 482.5. ¹H NMR (400 MHz, MeOD) δ 9.25 (s, 1H), 8.79 (d, 1H), 8.68 (s, 1H), 7.21-7.07 (m, 5H), 5.28 (t, 1H), 3.66 (t, 2H), 3.61-3.47 (m, 6H), 2.98-2.93 (m, 1H), 2.64 (t, 1H), 2.58-2.55 (m, 2H), 1.65-1.28 (m, 5H).

### Example 27

### Synthesis of ((S)-1-((S)-4-morpholino-4-oxo-2-(pyrazine-2-carboxamido)butanamido)-4-phenylbutyl)boronic acid

In another embodiment, Compound 27 can be produced from compound (27-1) by the method shown in Scheme 27. Amino acid (27-1) is coupled with the amine of a protected boronic acid compound (27-2) to produce amide (27-3), which is then deprotected to generate amine (27-4) as the hydrochloride salt. Subsequent coupling with carboxylic acid (27-5) produces amide (27-6). Deprotection of (27-6) with methylboronic acid (27-7) produces Compound 27.

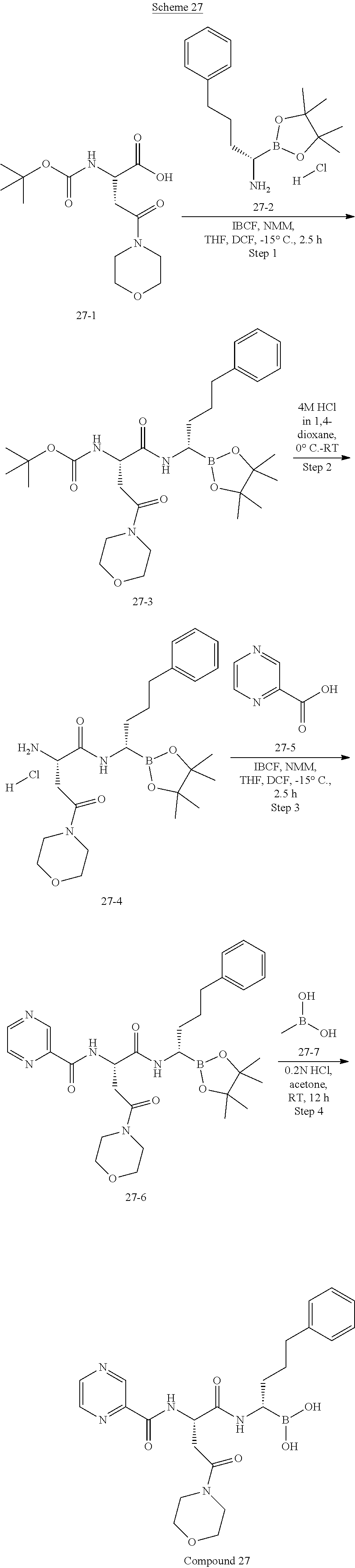

Synthesis of tert-butyl ((S)-4-morpholino-1,4-dioxo-1-(((S)-4-phenyl-1-(4,4,5,5-tetramethyl-1,3,2-dioxaborolan-2-yl)butyl)amino)butan-2-yl)carbamate [Step 1]: To a stirred solution of (S)-2-((tert-butoxycarbonyl)amino)-4-morpholino-4-oxobutanoic acid (27-1, 610 mg, 2.0 mmol) in THF (10 mL) was added IBCF (0.25 mL, 2.0 mmol) and NMM (0.26 mL, 2.0 mmol) at −15° C. Reaction mixture was stirred at same temperature for 30 min. Then (S)-4-phenyl-1-(4,4,5,5-tetramethyl-1,3,2-dioxaborolan-2-yl)butan-1-amine hydrochloride (27-2, 570 mg, 1.8 mmol) in DMF (1 mL) followed by NMM (0.2 mL, 1.8 mmol) was added to the reaction mixture at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. Combined organic layer was washed with 5% K₂CO solution, water, brine, dried over Na₂SO₄, filtered and evaporated under reduced pressure to afford tert-butyl ((S)-4-morpholino-1,4-dioxo-1-(((S)-4-phenyl-1-(4,4,5,5-tetramethyl-1,3,2-dioxaborolan-2-yl)butyl) amino)butan-2-yl)carbamate (27-3, 700 mg, crude), which is directly used in next step. [M−H]: 558.5.

Synthesis of (S)-2-amino-4-morpholino-4-oxo-N—((S)-4-phenyl-1-(4,4,5,5-tetramethyl-1,3,2-dioxaborolan-2-yl)butyl)butanamide hydrochloride [Step 2]: To a solution of tert-butyl ((S)-4-morpholino-1,4-dioxo-1-(((S)-4-phenyl-1-(4,4,5,5-tetramethyl-1,3,2-dioxaborolan-2-yl)butyl) amino)butan-2-yl)carbamate (27-3, 700 mg, 1.2 mmol) in 6 mL 1,4 dioxane was added 4 M HCl in dioxane (6.0 mL, 24.0 mmol) at 0° C. It was gradually warmed to 25° C. and stirred for 16 h.

TLC showed complete consumption of starting material to form new polar spot. Volatiles were removed under reduced pressure to get (S)-2-amino-4-morpholino-4-oxo-N—((S)-4-phenyl-1-(4,4,5,5-tetramethyl-1,3,2-dioxaborolan-2-yl)butyl)butanamide hydrochloride (27-4, 560 mg, crude). Crude was directly used for next step without purification. [M+H]: 458.4.

Synthesis of N—((S)-4-morpholino-1,4-dioxo-1-(((S)-4-phenyl-1-(4,4,5,5-tetramethyl-1,3,2-dioxaborolan-2-yl)butyl)amino)butan-2-yl)pyrazine-2-carboxamide [Step 3]: To a stirred solution of pyrazine-2-carboxylic acid (27-5, 165 mg, 1.3 mmol) in THF (8 mL) was added IBCF (0.17 mL, 1.3 mmol) and NMM (0.17 mL, 1.3 mmol) at −15° C. The reaction mixture was stirred at same temperature for 30 min. Then (S)-2-amino-4-morpholino-4-oxo-N—((S)-4-phenyl-1-(4,4,5,5-tetramethyl-1,3,2-dioxaborolan-2-yl)butyl)butanamide hydrochloride (27-4, 600 mg, 1.2 mmol) in DMF (1 mL) followed by NMM (0.14 mL, 1.2 mmol) was added to the reaction mixture under same condition. It was gradually warmed to 0° C. and stirred for 2 h. LCMS of the crude reaction mass confirmed the formation of desired product. It was neutralized with saturated aqueous 0.1 N HCl solution and extracted with ethyl acetate. The combined organic layer was washed with 5% K₂CO₃ solution, water, brine, dried over Na₂SO₄, filtered and evaporated under reduced pressure. The crude material was purified by RP prep HPLC purification and lyophilized to afford N—((S)-4-morpholino-1,4-dioxo-1-(((S)-4-phenyl-1-(4,4,5,5-tetramethyl-1,3,2-dioxaborolan-2-yl)butyl) amino)butan-2-yl)pyrazine-2-carboxamide (27-6, 120 mg). [M−H]⁺=564.3. [M-84+H]⁺=482.3.

Synthesis of ((S)-1-((S)-4-morpholino-4-oxo-2-(pyrazine-2-carboxamido)butanamido)-4-phenylbutyl)boronic acid [Step 4]: To a stirred solution of N—((S)-4-morpholino-1,4-dioxo-1-(((S)-4-phenyl-1-(4,4,5,5-tetramethyl-1,3,2-dioxaborolan-2-yl)butyl)amino)butan-2-yl)pyrazine-2-carboxamide (27-6, 120 mg, 0.21 mmol) and methylboronic acid (27-7, 127 mg, 2.1 mmol) in acetone (4 mL) was added 0.2 N HCl (4 mL) and the reaction mixture was stirred at RT overnight. TLC and LCMS showed full conversion of starting material with formation of new polar spot. The volatiles were evaporated and the residue was redissolved in acetone and deionized water and freeze-dried to obtain to get crude. The crude material was purified by RP prep HPLC purification and lyophilized to afford ((S)-1-((S)-4-morpholino-4-oxo-2-(pyrazine-2-carboxamido) butanamido)-4-phenylbutyl) boronic acid (Compound 27, 45 mg). [M−H]⁻: 482.3, ¹H NMR (400 MHz, MeOD) δ 9.25 (s, 1H), 8.79 (d, 1H), 8.68 (s, 1H), 7.19-7.09 (m, 5H), 5.28 (t, 1H), 3.66-3.61 (m, 4H), 3.59-3.51 (m, 4H), 3.01-3.00 (m, 1H), 2.60-2.56 (m, 4H), 1.65-1.63 (m, 4H).

### Example 28

### Synthesis of ((R)-1-((R)-4-morpholino-4-oxo-2-(pyrazine-2-carboxamido)butanamido)-2-phenylethyl) boronic acid

In another embodiment, Compound 28 can be produced from compound (28-1) by the method shown in Scheme 28. Carboxylic acid (28-1) is coupled with the amine of a protected boronic acid compound (28-2) to produce amide (28-3), which is then deprotected to generate amine (28-4) as the hydrochloride salt. Subsequent coupling with carboxylic acid (28-5) produces amide (28-6). Deprotection of (28-6) with methylboronic acid produces Compound 28.

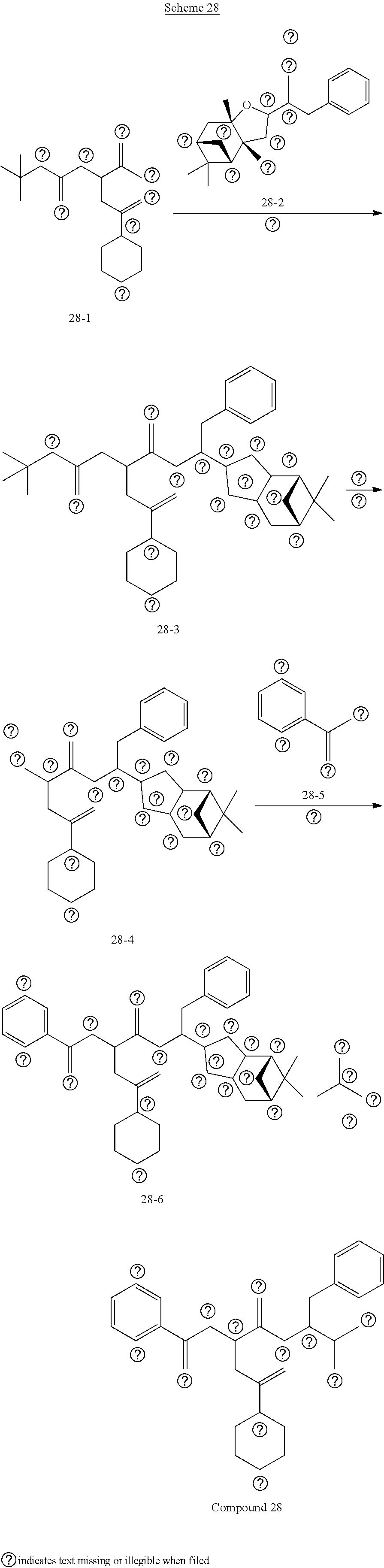

Synthesis of tert-butyl ((R)-4-morpholino-1,4-dioxo-1-(((R)-2-phenyl-1-((3aS,4S,6S,7aR)-3a,5,5-trimethylhexahydro-4,6-methanobenzo[d][1,3,2]dioxaborol-2-yl)ethyl)amino)butan-2-yl)carbamate [Step 1]: To a stirred solution of (R)-2-((tert-butoxycarbonyl)amino)-4-morpholino-4-oxobutanoic acid (28-1, 590 mg, 2 mmol) in tetrahydrofuran (10 mL) was added NMM (0.3 mL, 2 mmol) followed by IBCF (0.3 mL, 2 mmol) at −15° C. and stirred for 1 h. A solution of (1R)-2-phenyl-1-((4S,6S,7aS)-5,5,7a-trimethylhexahydro-4,6-methanobenzo[d][1,3,2]dioxaborol-2-yl)ethan-1-amine hydrochloride (28-2, 600 mg, 1.8 mmol) in dimethylformamide (1.5 mL) and NMM (0.3 mL, 2 mmol) were added and stirred at ambient temperature for 2 h. The reaction mixture was diluted with ethyl acetate and washed with 0.1M HCl, 5% K₂CO₃, water and brine, dried over anhydrous Na₂SO₄ and evaporated to give tert-butyl ((R)-4-morpholino-1,4-dioxo-1-(((R)-2-phenyl-1-((3aS,4S,6S,7aR)-3a,5,5-trimethylhexahydro-4,6-methanobenzo[d][1,3,2]dioxaborol-2-yl)ethyl)amino)butan-2-yl)carbamate (28-3, 800 mg). [M−H]⁻=582.5.

Synthesis of (R)-2-amino-4-morpholino-4-oxo-N—((R)-2-phenyl-1-((3aS,4S,6S,7aR)-3a,5,5-trimethylhexahydro-4,6-methanobenzo[d][1,3,2]dioxaborol-2-yl)ethyl)butanamide hydrochloride [Step 2]: To a stirred solution of tert-butyl ((R)-4-morpholino-1,4-dioxo-1-(((R)-2-phenyl-1-((3aS,4S,6S,7aR)-3a,5,5-trimethylhexahydro-4,6-methanobenzo[d][1,3,2]dioxaborol-2-yl)ethyl)amino) butan-2-yl)carbamate (28-3, 800 mg, 1.4 mmol) in 1,4-dioxane (4 mL) was added 4M HCl in 1,4-dioxane (4 mL, 14 mmol) in ice cold condition and stirred at ambient temperature for 2 h. Volatiles were removed under reduced pressure and triturated with n-pentane to give (R)-2-amino-4-morpholino-4-oxo-N—((R)-2-phenyl-1-((3aS,4S,6S,7aR)-3a,5,5-trimethylhexahydro-4,6-methanobenzo[d][1,3,2]dioxaborol-2-yl)ethyl)butanamide hydrochloride (28-4, 700 mg). [M−H]⁺=482.4.

Synthesis of N—((R)-4-morpholino-1,4-dioxo-1-(((R)-2-phenyl-1-((3aS,4S,6S,7aR)-3a,5,5-trimethyl hexahydro-4,6-methanobenzo[d][1,3,2]dioxaborol-2-yl)ethyl)amino)butan-2-yl) pyrazine-2-carboxamide [Step 3]: To a stirred solution of pyrazine-2-carboxylic acid (28-5, 220 mg, 1.7 mmol) in tetrahydrofuran (10 mL) was added NMM (0.2 mL, 1.5 mmol) followed by IBCF (0.2 mL, 1.4 mmol) at −15° C. and stirred for 30 min. A solution of (R)-2-amino-4-morpholino-4-oxo-N—((R)-2-phenyl-1-((3aS,4S,6S,7aR)-3a,5,5-trimethylhexahydro-4,6-methanobenzo[d][1,3,2]dioxaborol-2-yl)ethyl) butanamide hydrochloride (28-4, 700 mg, 1.4 mmol) in dimethylformamide (1 mL) and NMM (0.2 mL, 1.5 mmol) were added and stirred at RT for 2 h. The reaction mixture was diluted with ethyl acetate and washed with 0.1M HCl, 5% K₂CO₃, water and brine, dried over anhydrous Na₂SO₄ and concentrated under reduced pressure. The compound was purified by prep HPLC purification and lyophilized to afford N—((R)-4-morpholino-1,4-dioxo-1-(((R)-2-phenyl-1-((3aS,4S,6S,7aR)-3a,5,5-trimethyl hexahydro-4,6-methanobenzo [d][1,3,2]dioxaborol-2-yl)ethyl)amino)butan-2-yl)pyrazine-2-carboxamide (28-6, 100 mg). [M−H]⁻=588.3.

Synthesis of ((R)-1-((R)-4-morpholino-4-oxo-2-(pyrazine-2-carboxamido)butanamido)-2-phenylethyl) boronic acid [Step 4]: To a solution of N—((R)-4-morpholino-1,4-dioxo-1-(((R)-2-phenyl-1-((3aS,4S,6S,7aR)-3a,5,5-trimethyl hexahydro-4,6-methanobenzo[d][1,3,2]dioxaborol-2-yl)ethyl)amino)butan-2-yl) pyrazine-2-carboxamide (28-6, 100 mg, 0.2 mmol) in acetone (4 mL) was added methylboronic acid (200 mg, 3.4 mmol), followed by drop wise addition of 0.2N HCl (4 mL) and stirred at ambient temperature for overnight. Volatiles were removed under reduced pressure and purified by prep HPLC purification and lyophilized to afford ((R)-1-((R)-4-morpholino-4-oxo-2-(pyrazine-2-carboxamido)butanamido)-2-phenylethyl) boronic acid (Compound 28, 75 mg). [M−H]⁻=454.4. 1H NMR (400 MHz, CD3OD): δ_{H} 9.23 (d, 1H), 9.80 (d, 1H), 8.71 (q, 1H), 7.24-7.17 (m, 4H), 7.10 (d, 1H), 5.25 (t, 1H), 3.68-3.63 (m, 4H), 3.58-3.56 (m, 2H), 3.53 (t, 2H), 3.25 (d, 1H), 3.00 (dd, 1H), 2.87-2.81 (m, 2H), 2.66-2.63 (1H).

### Example 29

### Synthesis of ((R)-1-((R)-2-((3-chlorophenyl)sulfonamido)-4-morpholino-4-oxobutanamido)-4-phenylbutyl)boronic acid

In another embodiment, Compound 29 can be produced from compound (29-1) by the method shown in Scheme 29. Amine (29-1) is coupled with compound (29-2) to afford sulfonamide (29-3). Subsequent deprotection of (29-3) with methylboronic acid (29-4) produces Compound 29.

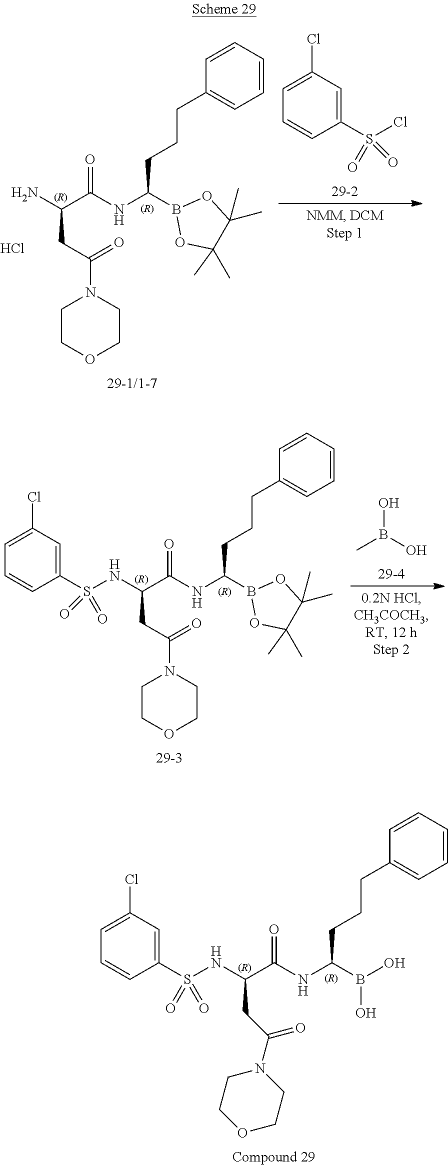

Synthesis of (R)-2-((3-chlorophenyl)sulfonamido)-4-morpholino-4-oxo-N—((R)-4-phenyl-1-(4,4,5,5-tetramethyl-1,3,2-dioxaborolan-2-yl)butyl)butanamide [Step 1]: To a stirred solution of (R)-2-amino-4-morpholino-4-oxo-N—((R)-4-phenyl-1-(4,4,5,5-tetramethyl-1,3,2-dioxaborolan-2-yl)butyl)butanamide hydrochloride (29-1, 200 mg, 0.4 mmol) and 3-chlorobenzenesulfonyl chloride (29-2, 0.06 mL, 0.4 mmol) in dichloromethane (2 mL) was added NMM (0.2 mL, 2 mmol) in ice cold condition and stirred at ambient temperature for 2 h. The reaction mixture was diluted with dichloromethane and washed with water and brine, dried over anhydrous Na₂SO₄ and evaporated under reduced pressure to afford (R)-2-((3-chlorophenyl)sulfonamido)-4-morpholino-4-oxo-N—((R)-4-phenyl-1-(4,4,5,5-tetramethyl-1,3,2-dioxaborolan-2-yl)butyl)butanamide (29-3, 200 mg). [M−H]⁺=632.

Synthesis of ((R)-1-((R)-2-((3-chlorophenyl)sulfonamido)-4-morpholino-4-oxobutanamido)-4-phenylbutyl)boronic acid [Step 2]: To a stirred solution of (R)-2-((3-chlorophenyl)sulfonamido)-4-morpholino-4-oxo-N—((R)-4-phenyl-1-(4,4,5,5-tetramethyl-1,3,2-dioxaborolan-2-yl)butyl)butanamide (29-3, 100 mg, 0.2 mmol) and methyl boronic acid (29-4, 113 mg, 2 mmol) in acetone (4 mL) was added 0.2N HCl (4 mL) and stirred at ambient temperature for overnight. The volatiles were removed under reduced pressure and purified by prep HPLC purification and lyophilized to afford ((R)-1-((R)-2-((3-chlorophenyl)sulfonamido)-4-morpholino-4-oxobutanamido)-4-phenylbutyl)boronic acid (Compound 29, 32 mg). [M−H]⁻: 550.3. ¹H NMR (400 MHz, CD₃OD): δ_{H} 7.87 (s, 1H), 7.77 (d, 1H), 7.60 (d, 1H), 7.50 (t, 1H), 7.26-7.17 (m, 4H), 7.15-7.11 (m, 1H), 4.48 (t, 1H), 3.60-3.55 (m, 4H), 3.44-3.38 (m, 4H), 2.84-2.70 (m, 2H), 2.61-2.55 (m, 3H), 1.61-1.58 (m, 2H), 1.43-1.26 (m, 3H).

### Example 30

### Synthesis of ((R)-1-((R)-2-((2-chlorophenyl)sulfonamido)-4-morpholino-4-oxobutanamido)-4-phenylbutyl)boronic acid

In another embodiment, Compound 30 can be produced from compound (30-1) by the method shown in Scheme 30. Amine (30-1) is coupled with compound (30-2) to afford sulfonamide (30-3). Subsequent deprotection of (30-3) with methylboronic acid (30-4) produces Compound 30.

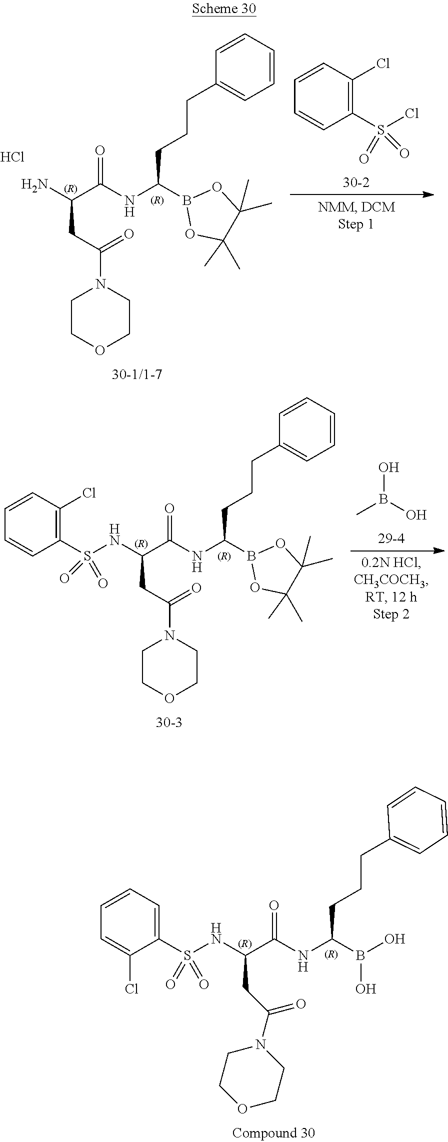

Synthesis of (R)-2-((2-chlorophenyl)sulfonamido)-4-morpholino-4-oxo-N—((R)-4-phenyl-1-(4,4,5,5-tetramethyl-1,3,2-dioxaborolan-2-yl)butyl)butanamide [Step 1]: To a stirred solution of (R)-2-amino-4-morpholino-4-oxo-N—((R)-4-phenyl-1-(4,4,5,5-tetramethyl-1,3,2-dioxaborolan-2-yl)butyl)butanamide hydrochloride (30-1, 300 mg, 0.6 mmol) in DCM (7 mL) was added NMM (0.33 mL, 3.0 mmol) in ice cold condition and stirred for 5 min. 2-chlorobenzenesulfonyl chloride (30-2, 140 mg, 0.7 mmol) was added to it and stirred at the same temperature and then gradually warmed to RT for 2 h. The reaction was monitored by LCMS. The reaction mixture was diluted with DCM and washed with water and brine, dried over Na₂SO₄ and evaporated under reduced pressure. The crude was purified via prep HPLC purification and lyophilized to afford (R)-2-((2-chlorophenyl)sulfonamido)-4-morpholino-4-oxo-N—((R)-4-phenyl-1-(4,4,5,5-tetramethyl-1,3,2-dioxaborolan-2-yl)butyl)butanamide (30-3, 45 mg). [M−H]⁻: 632.4 and [M-84-H]⁻: 551.

Synthesis of ((R)-1-((R)-2-((2-chlorophenyl)sulfonamido)-4-morpholino-4-oxobutanamido)-4-phenylbutyl)boronic acid [Step 2]: To a stirred solution of (R)-2-((2-chlorophenyl) sulfonamido)-4-morpholino-4-oxo-N—((R)-4-phenyl-1-(4,4,5,5-tetramethyl-1,3,2-dioxaborolan-2-yl)butyl)butanamide (30-3, 25 mg, 0.04 mmol) and methylboronic acid (24 mg, 0.4 mmol) in acetone (1 mL) was added 0.2 (N) HCl (1 mL) drop wise at ice cold condition and stirred at RT for overnight. The reaction was monitored by LCMS. The volatiles were evaporated and the residue was purified by prep HPLC purification and lyophilized to afford ((R)-1-((R)-2-((2-chlorophenyl) sulfonamido)-4-morpholino-4-oxobutanamido)-4-phenylbutyl) boronic acid (Compound 30, 14 mg). [M−H]⁻=550.3. ¹H NMR (400 MHz, MeOD) δ 8.06 (d, 1H), 7.58-7.57 (d, 2H), 7.47-7.43 (m, 1H), 7.26-7.13 (m, 5H), 4.48 (t, 1H), 3.61-3.55 (m, 4H), 3.42-3.35 (m, 4H), 2.91-2.57 (m, 5H), 1.60-1.58 (m, 2H), 1.43-1.28 (m, 2H).

### Example 31

### Synthesis of (R)-1-((R)-2-(benzylamino)-4-morpholino-4-oxobutanamido)-4-phenylbutyl) boronic acid

In another embodiment, Compound 31 can be produced from compound (31-1) by the method shown in Scheme 31.

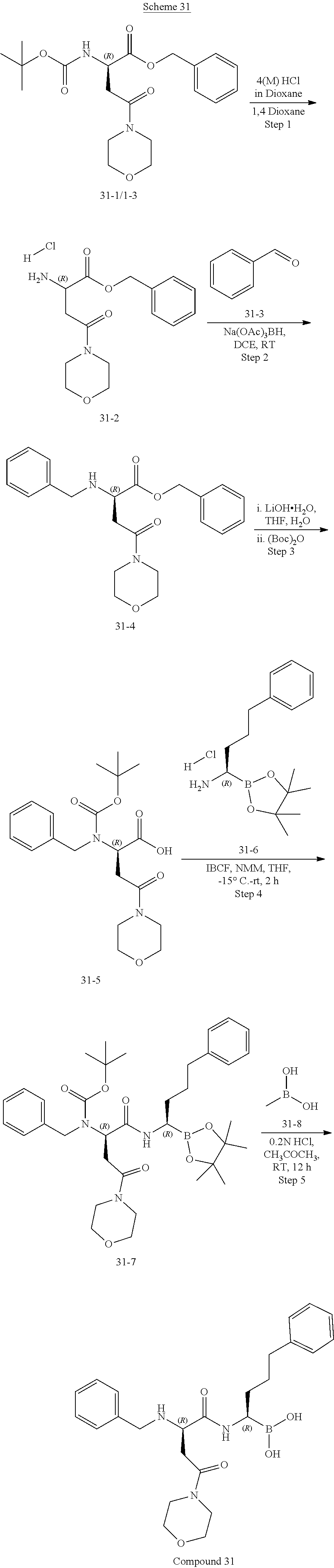

Synthesis of benzyl (R)-2-amino-4-morpholino-4-oxobutanoate hydrochloride [Step 1]: To a solution of benzyl (R)-2-((tert-butoxycarbonyl)amino)-4-morpholino-4-oxobutanoate (31-1, 3 g, 7.64 mmol) in 1,4-dioxane (30 mL) was added 4 (M) HCl in 1,4-Dioxane (19 mL) at 0° C. and stirred at 25° C. for 16 h. TLC showed complete consumption of starting material to new polar spot. Volatiles were removed under reduced pressure to get crude benzyl (R)-2-amino-4-morpholino-4-oxobutanoate hydrochloride (31-2, 2.4 g). ¹H NMR (400 MHz, DMSO-d₆) δ 8.50 (s, 3H), 7.39-7.34 (m, 5H), 5.19 (q, 2H), 4.36 (t, 1H), 3.61-3.37 (m, 8H), 3.17-3.08 (m, 2H).

Synthesis of benzyl (R)-2-(benzylamino)-4-morpholino-4-oxobutanoate [Step 2]: Benzyl (R)-2-amino-4-morpholino-4-oxobutanoate hydrochloride (31-2, 500 mg, 1.7 mmol) and benzaldehyde (31-3, 0.2 mL, 1.7 mmol) were dissolved in DCE (10 mL) under argon atmosphere. Acetic acid (0.02 mL, 0.3 mmol) was added to it and stirred at ambient temperature for 1 h. It was cooled to 0° C. NaBH(OAc)₃ (725 mg, 3.4 mmol) was added portion wise and stirred at room temperature for 16 h. It was poured into crashed ice and extracted with dichloromethane (twice). Combined organic layer was washed with water, dried over Na2SO4 and evaporated. The crude was purified by flash chromatography to get benzyl (R)-2-(benzyl amino)-4-morpholino-4-oxobutanoate (31-4, 320 mg). [M+H]⁺: 383.2.

Synthesis of (R)-2-(benzyl(tert-butoxycarbonyl)amino)-4-morpholino-4-oxobutanoic acid [Step 3]: To a stirred solution of benzyl (R)-2-(benzyl amino)-4-morpholino-4-oxobutanoate (31-4, 240 mg, 0.6 mmol) in THF (3 mL) was added a solution of LiOH·H2O (80 mg, 1.8 mmol) in Water (1 mL) and stirred for 2 h at room temperature. The reaction was monitored by LCMS showed acid mass peak. Di-tert-butyl pyrocarbonate (0.1 mL, 0.5 mmol) was added to it and stirred at RT for 16 h. Reaction was monitored by LCMS. Volatiles were evaporated under reduced pressure. It was partitioned between ethyl acetate and water. Aqueous part was acidified with 2(M) NaHSO4 solution (pH: 2-3) and extracted with ethyl acetate (thrice). Combined organic phase was dried over Na₂SO₄ and evaporated under reduced pressure to get crude (R)-2-(benzyl (tert-butoxycarbonyl) amino)-4-morpholino-4-oxobutanoic acid (31-5, 140 mg). [M−H]⁻: 391.4.

Synthesis of tert-butyl benzyl((R)-4-morpholino-1,4-dioxo-1-(((R)-4-phenyl-1-(4,4,5,5-tetramethyl-1,3,2-dioxaborolan-2-yl)butyl)amino)butan-2-yl)carbamate [Step 4]: To a stirred solution of (R)-2-(benzyl(tert-butoxycarbonyl)amino)-4-morpholino-4-oxobutanoic acid (31-5, 130 mg, 0.3 mmol) in THF (2 mL) was added IBCF (0.03 mL, 0.2 mmol) followed by NMM (0.03 mL, 0.3 mmol) drop wise at −15° C. and stirred for 1 h. (R)-4-phenyl-1-(4,4,5,5-tetramethyl-1,3,2-dioxaborolan-2-yl)butan-1-amine hydrochloride (31-6, 85 mg, 0.3 mmol) was added to it followed by NMM (0.03 mL, 0.3 mmol). It was gradually warmed to 0° C. and stirred for 2 h. LCMS of crude reaction mass showed mass of the desired product. The reaction mixture was neutralized with saturated aqueous 0.1 (N) HCl solutions and extracted with ethyl acetate (thrice). Combined organic layer was washed with 5% K2CO3 solution, water, brine, dried over Na₂SO₄ and evaporated under reduced pressure to get crude tert-butyl benzyl((R)-4-morpholino-1,4-dioxo-1—(((R)-4-phenyl-1-(4,4,5,5-tetramethyl-1,3,2-dioxaborolan-2-yl)butyl)amino)butan-2-yl)carbamate (31-7, 130 mg). [M−H]⁻=648.5

Synthesis of (R)-1-((R)-2-(benzylamino)-4-morpholino-4-oxobutanamido)-4-phenylbutyl) boronic acid [Step 5]: To a stirred solution of tert-butyl benzyl((R)-4-morpholino-1,4-dioxo-1-(((R)-4-phenyl-1-(4,4,5,5-tetramethyl-1,3,2-dioxaborolan-2-yl)butyl)amino)butan-2-yl)carbamate (31-7, 130 mg, 0.2 mmol) and methyl boronic acid (31-8, 120 mg, 2 mmol) in Acetone (4 mL) was added 0.2 (N) HCl (4 mL) and stirred at RT for overnight. The reaction was monitored by LCMS. Volatiles were evaporated and the residue was purified by prep HPLC purification and lyophilized to afford (R)-1-((R)-2-(benzylamino)-4-morpholino-4-oxobutanamido)-4-phenylbutyl) boronic acid (Compound 31, 9 mg). [M−H]⁻=466.4. ¹H NMR (400 MHz, Methanol-d₄) δ 7.38-7.12 (m, 10H), 4.00-3.89 (m, 3H), 3.61-3.58 (m, 4H), 3.55-3.43 (m, 4H), 2.93-2.76 (m, 3H), 2.66-2.58 (m, 2H), 1.67-1.50 (m, 4H).

### Example 32

### Synthesis of ((R)-1-((R)-4-morpholino-4-oxo-2-(((((S)tetrahydrofuran-3-yl)oxy)carbonyl) amino)butanamido)-4-phenylbutyl)boronic acid

In another embodiment, Compound 32 can be produced from compound (32-1) by the method shown in Scheme 32.

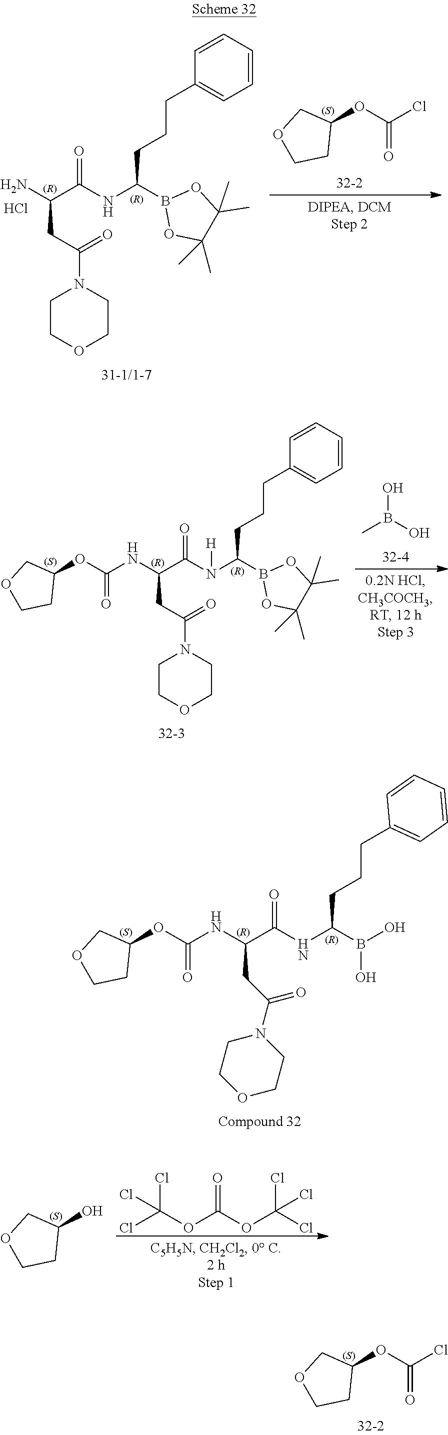

Synthesis of (S)-tetrahydrofuran-3yl-carbonochloride [Step 1]: To a stirred solution of (3S)-tetrahydrofuran-3-ol (200 mg, 2.27 mmol) in anhydrous DCM (5 mL) was added dropwise a solution triphosgene (269 mg, 0.9 mmol) and Pyridine (0.18 mL, 2.27 mmol) in anhydrous CH₂Cl₂ (1 mL) at ice cold condition. The mixture was allowed to warm to room temperature and stirred for 2 h. Volatiles were removed under reduced pressure at room temperature and the residue re-suspended in EtOAc (10 mL) and stirred for 30 minutes. The suspension was filtered and the solvent removed at 30° C. to get (S)-tetrahydrofuran-3yl-carbonochloride (32-2, 250 mg, crude), which is used in next step without any purification.

Synthesis of (S)-tetrahydrofuran-3-yl((R)-4-morpholino-1,4diooxo-1-(((R)-4-phenyl-1-(4,4,5,5-tetramethyl-1,3,2-dioxaborolan-2-yl)butyl)amino)butan-2yl)carbamate [Step 2]: To a stirred solution of (2R)-2-amino-4-morpholino-4-oxo-N-[(1R)-4-phenyl-1-(4,4,5,5-tetramethyl-1,3,2-dioxaborolan-2-yl)butyl]butanamide hydrochloride (32-1, 100 mg, 0.2 mmol) in DCM (5 mL) was added DIPEA (0.14 mL, 1 mmol) and stirred for 5 min. under argon atmosphere. To this mixture [(3S)-tetrahydrofuran-3-yl]carbonochloridate (32-2, 36 mg, 0.24 mmol) was added and continued stirring for 2 h at 20° C. After completion, reaction mixture was diluted with DCM and washed with water and 0.1 N HCl and brine. Organic layer was dried over Na₂SO₄ and the crude was purified by prep HPLC purification to afford (S)-tetrahydrofuran-3-yl((R)-4-morpholino-1,4diooxo-1-(((R)-4-phenyl-1-(4,4,5,5-tetramethyl-1,3,2-dioxaborolan-2-yl)butyl)amino)butan-2yl) carbamate (32-3, 12 mg). [M−H]⁻: 572.4.

Synthesis of ((R)-1-((R)-4-morpholino-4-oxo-2-(((((S)tetrahydrofuran-3-yl)oxy)carbonyl) amino)butanamido)-4-phenylbutyl)boronic acid [Step 3]: To a stirred solution of [(3S)-tetrahydrofuran-3-yl]N-[(1R)-3-morpholino-3-oxo-1-[[(1R)-4-phenyl-1-(4,4,5,5-tetramethyl-1,3,2-dioxaborolan-2-yl)butyl)carbamoyl)propyl)carbamate (32-3, 35 mg, 0.06 mmol) in acetone (4 mL) was added methyl boronic acid (32-4, 37 mg, 0.6 mmol), followed by drop wise addition of 0.2 HCl (2 mL). The reaction mixture was allowed to stir at room temperature overnight. All the volatiles were evaporated at room temperature and lyophilized. The crude was purified through prep HPLC purification and lyophilized to afford ((R)-1-((R)-4-morpholino-4-oxo-2-(((((S)tetrahydrofuran-3-yl)oxy) carbonyl)amino) butanamido)-4-phenylbutyl) boronic acid (Compound 32, 12 mg). [M−H]⁻: 590.3. ¹H NMR (400 MHz, MeOD) δ 7.09-7.24 (m, 5H), 5.18 (d, 1H), 4.78 (t, 1H), 3.80-3.87 (m, 4H), 3.59-3.66 (m, 4H), 3.46-3.51 (m, 4H), 2.91-2.96 (m, 2H), 2.57-2.63 (m, 3H), 2.13-2.14 (m, 1H), 2.00-2.02 (m, 1H), 1.65-1.69 (m, 2H), 1.47-1.54 (m, 2H).

### Example 33

### Synthesis of ((R)-1-((S)-4-morpholino-4-oxo-2-(phenylsulfonamido)butanamido)-4-phenyl butyl)boronic acid

In another embodiment, Compound 33 can be produced from compound (33-1) by the method shown in Scheme 33.

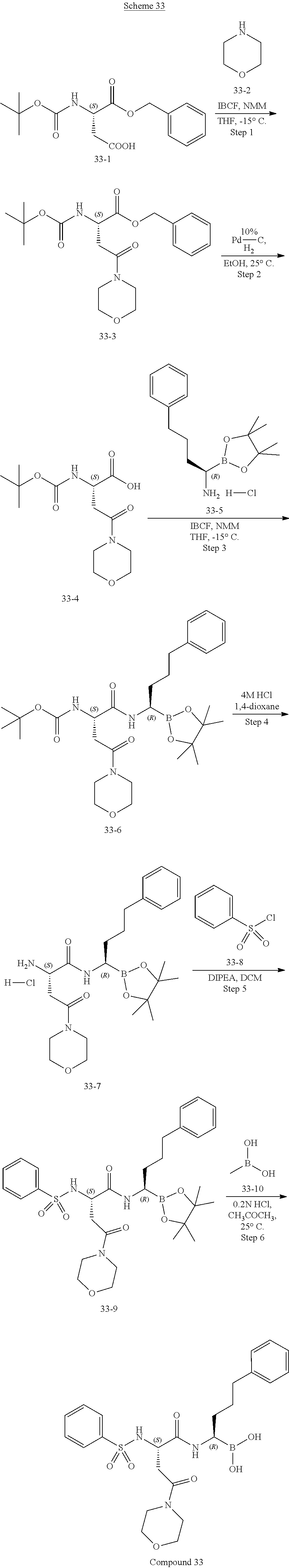

Synthesis of benzyl (S)-2-((tert-butoxycarbonyl)amino)-4-morpholino-4-oxobutanoate [Step 1]: To a solution of (S)-4-(benzyloxy)-3-((tert-butoxycarbonyl)amino)-4-oxobutanoic acid (33-1, 6.1 g, 18.9 mmol) in THF (30 ml) was added IBCF (2.6 mL, 18.9 mmol) followed by NMM (2.6 mL, 18.9 mmol) at −15° C. After 45 min, a solution of morpholine (33-2, 1.5 mL, 17.1 mmol) in DMF (2 mL) was added dropwise followed by NMM (2.3 mL, 17.1 mmol). After stirring for 1 h at the same temperature, the reaction was diluted with EtOAc and washed successively with 0.1 N aq. HCl (×2), 5% aq. K₂CO₃ (×2), water (×2) and brine (×2). The organic phase was dried over anhy. Na₂SO₄, filtered and concentrated under reduced pressure. The crude was purified on a silica gel column using 0-50% EtOAc in hexanes to afford benzyl (S)-2-((tert-butoxycarbonyl)amino)-4-morpholino-4-oxobutanoate (33-3, 5 g). [M+H]⁺=392.9.

Synthesis of (S)-2-((tert-butoxycarbonyl)amino)-4-morpholino-4-oxobutanoic acid [Step 2]: To a solution of benzyl (S)-2-((tert-butoxycarbonyl)amino)-4-morpholino-4-oxobutanoate (33-3, 1.50 g, 3.8 mmol) in ethanol (20 mL) was added Pd/C (150 mg, 10 wt %) under N₂. The reaction vessel was evacuated and backfilled with H₂ (×2) and then kept under a H₂ balloon. After stirring for 2 h at 25° C., the reaction mixture was filtered through a pad of celite. The filtrate was concentrated to dryness to afford crude (S)-2-((tert-butoxycarbonyl)amino)-4-morpholino-4-oxobutanoic acid (33-4, 1.0 g), which was used in the next step without further purification. [M+H]⁺=302.8.

Synthesis of tert-butyl ((S)-4-morpholino-1,4-dioxo-1-(((R)-4-phenyl-1-(4,4,5,5-tetramethyl-1,3,2-dioxaborolan-2-yl)butyl)amino)butan-2-yl)carbamate [Step 3]: To a solution of (S)-2-((tert-butoxycarbonyl)amino)-4-morpholino-4-oxobutanoic acid (33-4, 508 mg, 1.7 mmol) in THF (5 mL) was added IBCF (0.2 mL, 1.7 mmol) followed by NMM (0.2 mL, 1.7 mmol) at −15° C. After 45 min, a solution of (R)-4-phenyl-1-(4,4,5,5-tetramethyl-1,3,2-dioxaborolan-2-yl)butan-1-amine hydrochloride (33-5, 476 mg, 1.5 mmol) in DMF (1 mL) was added dropwise followed by NMM (0.2 mL, 1.5 mmol). After stirring for 1 h at the same temperature, the reaction mixture was diluted with EtOAc, and washed successively with 0.1 N aq. HCl (×2), 5% aq. K2CO3 (×2), water (×2) and brine (×2). The organic phase was dried over anhydrous Na2SO4, filtered and concentrated under reduced pressure to get crude tert-butyl ((S)-4-morpholino-1,4-dioxo-1-(((R)-4-phenyl-1-(4,4,5,5-tetramethyl-1,3,2-dioxaborolan-2-yl)butyl)amino)butan-2-yl)carbamate (33-6, 700 mg). The crude was used for next step without further purification. [M−H]⁻=558.4.

Synthesis of (S)-2-amino-4-morpholino-4-oxo-N—((R)-4-phenyl-1-(4,4,5,5-tetramethyl-1,3,2-dioxaborolan-2-yl)butyl)butanamide hydrochloride [Step 4]: To a solution of crude tert-butyl ((S)-4-morpholino-1,4-dioxo-1-(((R)-4-phenyl-1-(4,4,5,5-tetramethyl-1,3,2-dioxaborolan-2-yl) butyl)amino)butan-2-yl)carbamate (33-6, 700 mg, 1.2 mmol) in 1,4-dioxane (5 mL) was added HCl (3.0 mL, 4M in 1,4-dioxane, 12.5 mmol) at 0° C., and the mixture was stirred at 25° C. for 16 h. The reaction mixture was concentrated under reduced pressure to afford crude (S)-2-amino-4-morpholino-4-oxo-N—((R)-4-phenyl-1-(4,4,5,5-tetramethyl-1,3,2-dioxaborolan-2-yl)butyl) butanamide hydrochloride (33-7, 500 mg). The crude was used in the next step without further purification. [M−H]⁻=458.3.

Synthesis of (S)-4-morpholino-4-oxo-N—((R)-4-phenyl-1-(4,4,5,5-tetramethyl-1,3,2-dioxaborolan-2-yl)butyl)-2-(phenylsulfonamido)butanamide [Step 5]: To a solution of crude (S)-2-amino-4-morpholino-4-oxo-N—((R)-4-phenyl-1-(4,4,5,5-tetramethyl-1,3,2-dioxaborolan-2-yl)butyl)butanamide hydrochloride (33-7, 500 mg, 1.1 mmol) in DCM (5 mL) were added DIPEA (0.4 mL, 3.3 mmol) and benzenesulfonyl chloride (33-8, 0.2 mL, 1.3 mmol) at 0° C. After stirring for 16 h at 25° C., the reaction mixture was diluted with water and extracted with DCM (×2). Combined organic phase was washed with brine, dried over anhydrous Na2SO4, filtered and concentrated under reduced pressure to afford crude (S)-4-morpholino-4-oxo-N—((R)-4-phenyl-1-(4,4,5,5-tetramethyl-1,3,2-dioxaborolan-2-yl)butyl)-2-(phenylsulfonamido)butanamide (33-9, 550 mg), which was directly used to the next step without further purification. [M−H]⁻=598.4.

Synthesis of ((R)-1-((S)-4-morpholino-4-oxo-2-(phenylsulfonamido)butanamido)-4-phenyl butyl)boronic acid [Step 6]: To a solution of (S)-4-morpholino-4-oxo-N—((R)-4-phenyl-1-(4,4,5,5-tetramethyl-1,3,2-dioxaborolan-2-yl)butyl)-2-(phenylsulfonamido)butanamide (33-9, 550 mg, 0.9 mmol) and methylboronic acid (33-10, 824 mg, 13.8 mmol) in acetone (20 mL) was added 0.2N HCl (20 mL, 4.0 mmol) at 25° C. The reaction mixture was stirred for 16 h (monitored by LCMS) at 25° C. The reaction mixture was concentrated under reduced pressure and lyophilized. The material was purified by prep HPLC (RP) and lyophilized again to afford ((R)-1-((S)-4-morpholino-4-oxo-2-(phenylsulfonamido)butanamido)-4-phenylbutyl)boronic acid (Compound 33, 110 mg). [M−H]⁻=516.2. ¹H NMR (400 MHz, CD3OD): δ 7.88-7.86 (d, 2H), 7.63-7.52 (m, 3H), 7.25-7.10 (m, 5H), 4.47-4.44 (t, 1H), 3.56-3.29 (m, 8H), 2.85-2.48 (m, 5H), 1.62-1.42 (m, 4H).

### Example 34

### Synthesis of ((R)-1-((R)-2-(benzyl(tert-butoxycarbonyl)amino)-4-morpholino-4-oxobutanamido)-4-phenylbutyl)boronic acid

In another embodiment, Compound 34 can be produced from compound (34-1/31-7) by the method shown in Scheme 34.

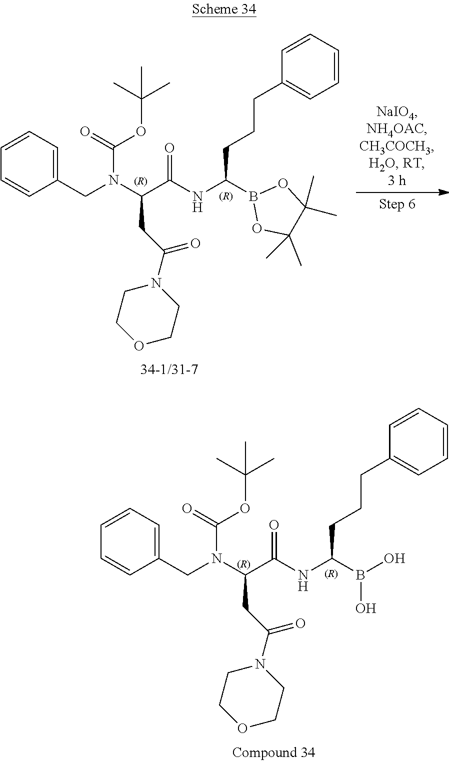

Synthesis of ((R)-1-((R)-2-(benzyl(tert-butoxycarbonyl)amino)-4-morpholino-4-oxobutanamido)-4-phenylbutyl)boronic acid [Step 1]: To a stirred solution of tert-butyl N-benzyl-N-[(1R)-3-morpholino-3-oxo-1-[[(1R)-4-phenyl-1-(4,4,5,5-tetramethyl-1,3,2-dioxaborolan-2-yl)butyl]carbamoyl]propyl]carbamate (34-1/31-7, 290 mg, 0.45 mmol) in acetone (2 mL) and water (2 mL) were added NaIO4 (95 mg, 0.45 mmol) followed by NH4OAc (34 mg, 0.45 mmol) and stirred at RT for overnight. The reaction was monitored by LCMS. Volatiles were evaporated. It was partitioned between ethyl acetate and water. The organic layer was washed with water, brine, dried over Na2SO4 and concentrated under reduced pressure. The crude was purified by prep HPLC purification and lyophilized to afford ((R)-1-((R)-2-(benzyl(tert-butoxycarbonyl)amino)-4-morpholino-4-oxobutanamido)-4-phenylbutyl)boronic acid (Compound 34, 56 mg). [M−H]⁻=566. ¹H NMR (400 MHz, CD3OD): δ 7.29-7.1 (m, 10H), 5.09 (brs, 1H), 4.58-4.46 (m, 3H), 3.54-3.39 (m, 7H), 3.12 (m, 1H), 2.65-2.52 (m, 4H), 1.61 (m, 4H), 1.41-1.28 (m, 9H).

### Example 35

### Synthesis of ((R)-1-((R)-2-(2,5-dichlorobenzamido)-4-morpholino-4-oxobutanamido)-4-phenylbutyl)boronic acid

In another embodiment, Compound 35 can be produced from compound (35-1/1-7) by the method shown in Scheme 35.

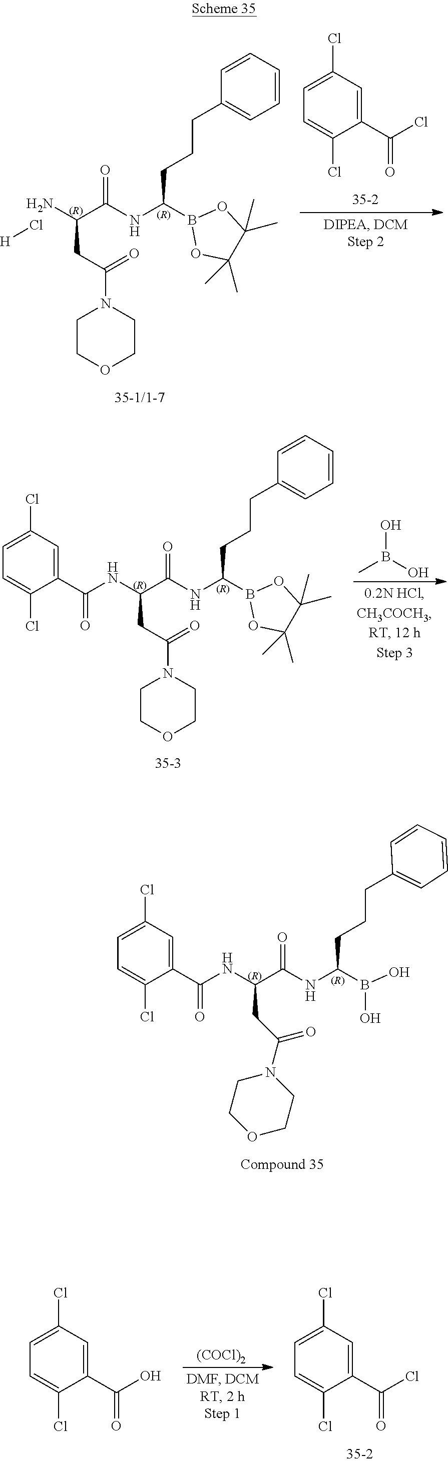

Synthesis of 2,5-dichlorobenzoyl chloride [Step 1]: To a stirred solution of 2,5-dichlorobenzoic acid (200 mg, 1.0 mmol) in DCM (5 mL) under inert atmosphere, Oxalyl chloride (0.1 mL, 1.1 mmol) was added dropwise at room temperature followed by catalytic amount of DMF. The reaction mixture was stirred for 2 h at room temperature. After completion, reaction mixture was mixture was evaporated under reduced pressure in complete argon atmosphere to afford 2,5-dichlorobenzoyl chloride (35-2, 200 mg, crude) and directly used for next step.

Synthesis of 2,5-dichloro-N—((R)-4-morpholino-1,4-dioxo-1-(((R)-4-phenyl-1-(4,4,5,5-tetramethyl-1,3,2-dioxaborolan-2-yl)butyl)amino)butan-2-yl)benzamide [Step 2]: To a stirred solution of (R)-2-amino-4-morpholino-4-oxo-N—((R)-4-phenyl-1-(4,4,5,5-tetramethyl-1,3,2-dioxaborolan-2-yl)butyl)butanamide hydrochloride (35-1/1-7, 150 mg, 0.3 mmol) and 2,5-dichlorobenzoyl chloride (35-2, 70 mg, 0.33 mmol) in DCM (4 mL), NMM (0.17 mL, 1.5 mmol) was added in ice cold condition and the reaction mixture was stirred at RT for 2 h. The reaction was diluted with DCM and washed with water and brine solution. The organic phase was dried over Na₂SO₄, filtered and evaporated to yield crude 2,5-dichloro-N—((R)-4-morpholino-1,4-dioxo-1-(((R)-4-phenyl-1-(4,4,5,5-tetramethyl-1,3,2-dioxaborolan-2-yl)butyl)amino)butan-2-yl)benzamide (35-3, 190 mg, 0.3 mmol). Crude material was directly used for next step without further purification. [M−H]⁻=630.5 and [M-83]⁻=548.3

Synthesis of ((R)-1-((R)-2-(2,5-dichlorobenzamido)-4-morpholino-4-oxobutanamido)-4-phenylbutyl)boronic acid [Step 3]: To a stirred solution of 2,5-dichloro-N—((R)-4-morpholino-1,4-dioxo-1-(((R)-4-phenyl-1-(4,4,5,5-tetramethyl-1,3,2-dioxaborolan-2-yl)butyl)amino)butan-2-yl)benzamide (35-3, 190 mg, 0.3 mmol) and methylboronic acid (180 mg, 3.0 mmol) in acetone (5 mL) was added 0.2 N HCl (5.0 mL) and the reaction mixture was stirred at RT overnight. The volatiles were evaporated and the residue was lyophilized to obtain crude. Crude material was purified by prep HPLC purification and lyophilized to afford ((R)-1-((R)-2-(2,5-dichlorobenzamido)-4-morpholino-4-oxobutanamido)-4-phenylbutyl)boronic acid (Compound 35, 38 mg). [M−H]⁻: 548.3. ¹H NMR (400 MHz, MeOD) δ 7.55 (brs, 1H), 7.46 (d, 2H), 7.23-7.10 (m, 5H), 5.12 (t, 1H), 3.67-3.61 (m, 4H), 3.54-3.50 (m, 4H), 3.04-3.02 (m, 2H), 2.70-2.60 (m, 3H), 1.71-1.65 (m, 2H), 1.56-1.50 (m, 2H).

### Example 36

### Synthesis of ((R)-1-((R)-4-morpholino-4-oxo-2-(((((R)-tetrahydrofuran-3-yl)oxy)carbonyl) amino)butanamido)-4-phenylbutyl)boronic acid

In another embodiment, Compound 36 can be produced from compound (36-1/1-7) by the method shown in Scheme 36.

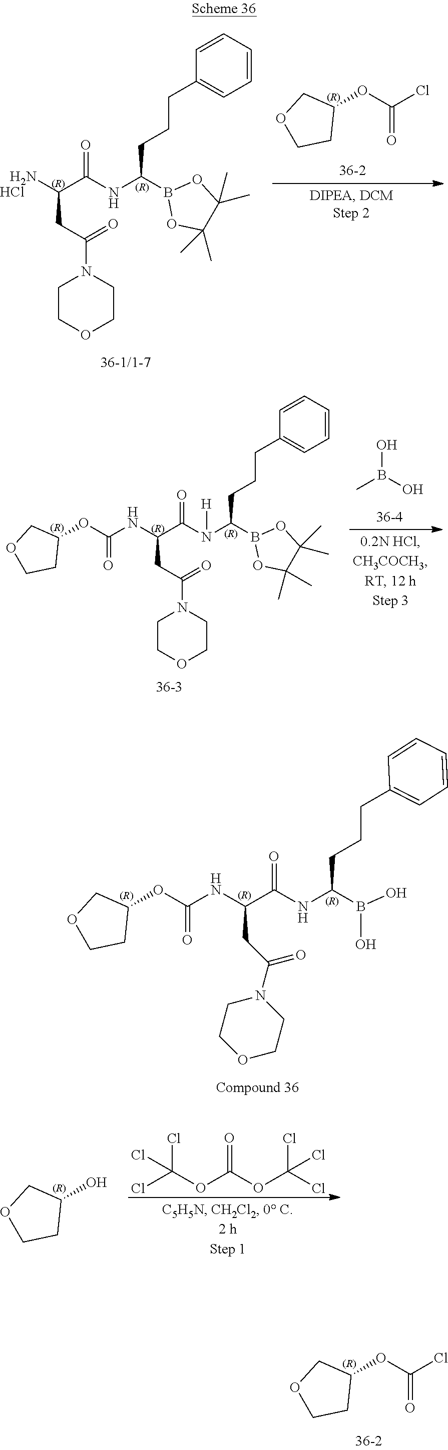

Synthesis of (R)-tetrahydrofuran-3-yl carbonochloridate [Step 1]: To a stirred solution of (R)-tetrahydrofuran-3-ol (400 mg, 4.54 mmol) in anhydrous CH2Cl2 (8 mL) cooled at 0° C., was added dropwise a solution of triphosgene (269 mg, 0.9 mmol) and Pyridine (0.18 mL, 2.27 mmol) in anhydrous CH₂Cl₂ (2 mL). The mixture was allowed to stir at 25° C. and stirred for 2 h. Reaction was monitored by LCMS (0-15% EtOAc-Hexane) by quenching few drops of reaction mixture by benzyl alcohol. The solvent was then evaporated at room temperature and the residue re-suspended in EtOAc (20 mL) and stirred for 30 minutes. The suspension was filtered and the solvent removed at 30° C. to get (R)-tetrahydrofuran-3-yl carbonochloridate (36-2, 190 mg, crude), which was used for next step without any purification.

Synthesis of (R)-tetrahydrofuran-3-yl ((R)-4-morpholino-1,4-dioxo-1-(((R)-4-phenyl-1-(4,4,5,5-tetramethyl-1,3,2-dioxaborolan-2-yl)butyl)amino)butan-2-yl)carbamate [Step 2]: To a stirred solution of (R)-2-amino-4-morpholino-4-oxo-N—((R)-4-phenyl-1-(4,4,5,5-tetramethyl-1,3,2-dioxaborolan-2-yl)butyl)butanamide hydrochloride (36-1/1-7, 400 mg, 0.8 mmol) in DCM (10 mL) was added DIPEA (0.55 mL, 4 mmol) and stirred for 5 minute under argon atmosphere. To this mixture (R)-tetrahydrofuran-3-yl carbonochloridate (36-3, 182 mg, 1.2 mmol) was added and continued stirring for 2 h at 20° C. Reaction mixture was diluted with DCM (100 ml) and washed with water and 0.1 N HCl and brine. The organic layer was dried over Na₂SO₄ and evaporated under reduced pressure to afford crude (R)-tetrahydrofuran-3-yl ((R)-4-morpholino-1,4-dioxo-1-(((R)-4-phenyl-1-(4,4,5,5-tetramethyl-1,3,2-dioxaborolan-2-yl)butyl)amino)butan-2-yl)carbamate (36-4, 650 mg). [M−H]⁻: 572.4.

Synthesis of ((R)-1-((R)-4-morpholino-4-oxo-2-(((((R)-tetrahydrofuran-3-yl)oxy)carbonyl) amino)butanamido)-4-phenylbutyl)boronic acid [Step 3]: To a stirred solution of (R)-tetrahydrofuran-3-yl ((R)-4-morpholino-1,4-dioxo-1-(((R)-4-phenyl-1-(4,4,5,5-tetramethyl-1,3,2-dioxaborolan-2-yl)butyl)amino)butan-2-yl)carbamate (36-4, 650 mg, 1 mmol) in acetone (5 mL) was added methyl boronic acid (36-5, 678 mg, 11.3 mmol), followed by drop wise addition of 0.2 HCl (5 mL). The reaction mixture was allowed to stirring at 25° C. for 16 h. All the volatiles were evaporated at 25° C. under reduced pressure, and reaction mixture was re-dissolved in acetonitrile and deionized water and freeze-dried. The obtained crude was purified through RP prep HPLC purification and lyophilized to afford ((R)-1-((R)-4-morpholino-4-oxo-2-(((((R)-tetrahydrofuran-3-yl)oxy)carbonyl)amino)butanamido)-4-phenylbutyl)boronic acid (Compound 36, 36.5 mg). [M−H]⁻: 490.3. ¹H NMR (400 MHz, MeOD) δ 7.09-7.24 (m, 5H), 5.18 (bs, 1H), 4.77 (t, 1H), 3.77-3.87 (m, 4H), 3.59-3.66 (m, 4H), 3.46-3.51 (m, 4H), 2.88-2.96 (m, 2H), 2.57-2.63 (m, 2H), 2.12-2.17 (m, 1H), 1.94-2.03 (m, 1H), 1.65-1.70 (m, 2H), 1.53-1.63 (m, 2H).

### Example 37

### Synthesis of ((R)-1-((R)-2-((2,5-dichlorophenyl)sulfonamido)-4-morpholino-4-oxobutanamido)-4-phenylbutyl)boronic acid

In another embodiment, Compound 37 can be produced from compound (37-1/1-7) by the method shown in Scheme 37.

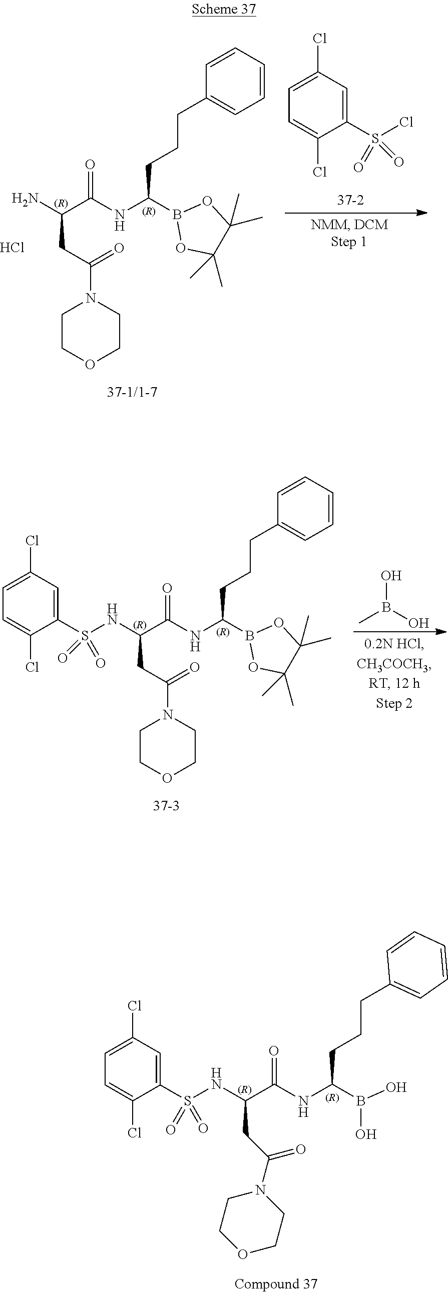

Synthesis of (R)-2-((2,5-dichlorophenyl)sulfonamido)-4-morpholino-4-oxo-N—((R)-4-phenyl-1-(4,4,5,5-tetramethyl-1,3,2-dioxaborolan-2-yl)butyl)butanamide [Step 1]: To a stirred solution of (R)-2-amino-4-morpholino-4-oxo-N—((R)-4-phenyl-1-(4,4,5,5-tetramethyl-1,3,2-dioxaborolan-2-yl)butyl)butanamide (37-1/1-7, 300 mg, 0.6 mmol) in DCM (7 mL) was added NMM (0.3 mL, 3 mmol) at ice cold condition and stirred for 5 minutes then 2,5-dichlorobenzenesulfonyl chloride (37-2, 165 mg, 0.7 mmol) was slowly added to this reaction mixture at the same temperature. The reaction mixture was warmed up to RT over 2 h. The reaction mixture was diluted with DCM and washed with water and brine, dried over Na₂SO₄ and evaporated under reduced pressure to give the crude. Crude was purified via prep HPLC development and lyophilized to afford a mixture of (R)-2-((2,5-dichlorophenyl)sulfonamido)-4-morpholino-4-oxo-N—((R)-4-phenyl-1-(4,4,5,5-tetramethyl-1,3,2-dioxaborolan-2-yl)butyl) butanamide (37-3, 120 mg) and its corresponding boronic acid. [M−H]⁻=666.4 and [M-83]⁻=584.3

Synthesis of ((R)-1-((R)-2-((2,5-dichlorophenyl)sulfonamido)-4-morpholino-4-oxobutanamido)-4-phenylbutyl)boronic acid [Step 2]: To a stirred solution of (R)-2-((2,5-dichlorophenyl)sulfonamido)-4-morpholino-4-oxo-N—((R)-4-phenyl-1-(4,4,5,5-tetramethyl-1,3,2-dioxaborolan-2-yl)butyl)butanamide (37-3, 125 mg, 0.2 mmol) and methylboronic acid (112 mg, 1.9 mmol) in acetone (5 mL) was added 0.2 (N)HCl (5 mL) and the reaction mixture was stirred at RT for 16 h. The volatiles were evaporated and the residue was lyophilized to obtain crude. Crude material was purified by prep HPLC purification and lyophilized to afford ((R)-1-((R)-2-((2,5-dichlorophenyl)sulfonamido)-4-morpholino-4-oxobutanamido)-4-phenylbutyl)boronic acid (Compound 37, 90 mg). [M−H]⁻: 584.4. ¹H NMR (400 MHz, MeOD) δ 8.05 (s, 1H), 7.60-7.54 (m, 2H), 7.26-7.13 (m, 5H), 4.45 (t, 1H), 3.58-3.53 (m, 4H), 3.43-3.33 (m, 4H), 2.93-2.88 (m, 1H), 2.77-2.71 (m, 1H), 2.61-2.56 (m, 3H), 1.61-1.59 (m, 2H), 1.50-1.20 (m, 2H).

### Example 38

### Synthesis of ((R)-1-((R)-2-(3-isopropylureido)-4-morpholino-4-oxobutanamido)-4-phenyl butyl)boronic acid

In another embodiment, Compound 38 can be produced from compound (38-1/1-7) by the method shown in Scheme 38.

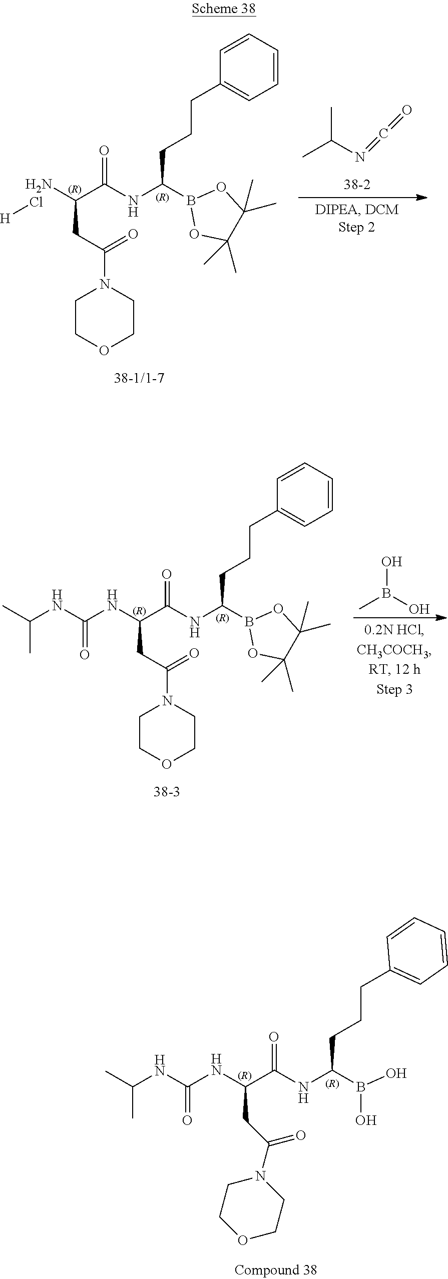

Synthesis of (R)-2-(3-isopropylureido)-4-morpholino-4-oxo-N—((R)-4-phenyl-1-(4,4,5,5-tetramethyl-1,3,2-dioxaborolan-2-yl)butyl)butanamide [Step 1]: To a stirred solution of (R)-2-amino-4-morpholino-4-oxo-N—((R)-4-phenyl-1-(4,4,5,5-tetramethyl-1,3,2-dioxaborolan-2-yl)butyl) butanamide hydrochloride (38-1/1-7, 250 mg, 0.5 mmol) and 2-isocyanatopropane (38-2, 0.05 mL, 0.6 mmol) in DCM (4 mL) was added DIPEA (0.4 mL, 2.5 mmol) at ice cold condition and stirred at RT for 2 h. The reaction was diluted with DCM and washed with water and brine, dried over Na₂SO₄ and evaporated to yield (R)-2-(3-isopropylureido)-4-morpholino-4-oxo-N—((R)-4-phenyl-1-(4,4,5,5-tetramethyl-1,3,2-dioxaborolan-2-yl)butyl)butanamide (38-3, 270 mg). [M−H]⁻: 543.5 and [M-83]⁻=461.4.

Synthesis of ((R)-1-((R)-2-(3-isopropylureido)-4-morpholino-4-oxobutanamido)-4-phenyl butyl)boronic acid [Step 2]: To a stirred solution of (R)-2-(3-isopropylureido)-4-morpholino-4-oxo-N—((R)-4-phenyl-1-(4,4,5,5-tetramethyl-1,3,2-dioxaborolan-2-yl)butyl)butanamide (38-3, 47 mg, 0.1 mmol) and methylboronic acid (47 mg, 0.8 mmol) in Acetone (6 mL) was added 0.2 N HCl (6 mL) and stirred at RT for overnight. The reaction was monitored by LCMS. The volatiles were evaporated and the residue was purified by prep HPLC purification and lyophilized to afford ((R)-1-((R)-2-(3-isopropylureido)-4-morpholino-4-oxobutanamido)-4-phenylbutyl)boronic acid (Compound 38, 31 mg). [M−H]⁻461.4. ¹H NMR (400 MHz, MeOD) δ 7.24-7.11 (m, 5H), 4.88-4.87 (m, 1H), 3.79-3.76 (m, 1H), 3.64-3.59 (m, 4H), 3.49-3.48 (m, 4H), 3.06 (dd, 1H), 2.83-2.80 (m, 1H), 2.62-2.56 (m, 3H), 1.63 (brs, 2H), 1.54-1.50 (m, 2H), 1.12-1.10 (m, 6H).

### Example 39

### Synthesis of ((R)-1-((R)-4-morpholino-4-oxo-2-(pyrazine-2-sulfonamido)butanamido)-4-phenylbutyl)boronic acid

In another embodiment, Compound 39 can be produced from compound (39-1/1-7) by the method shown in Scheme 39.

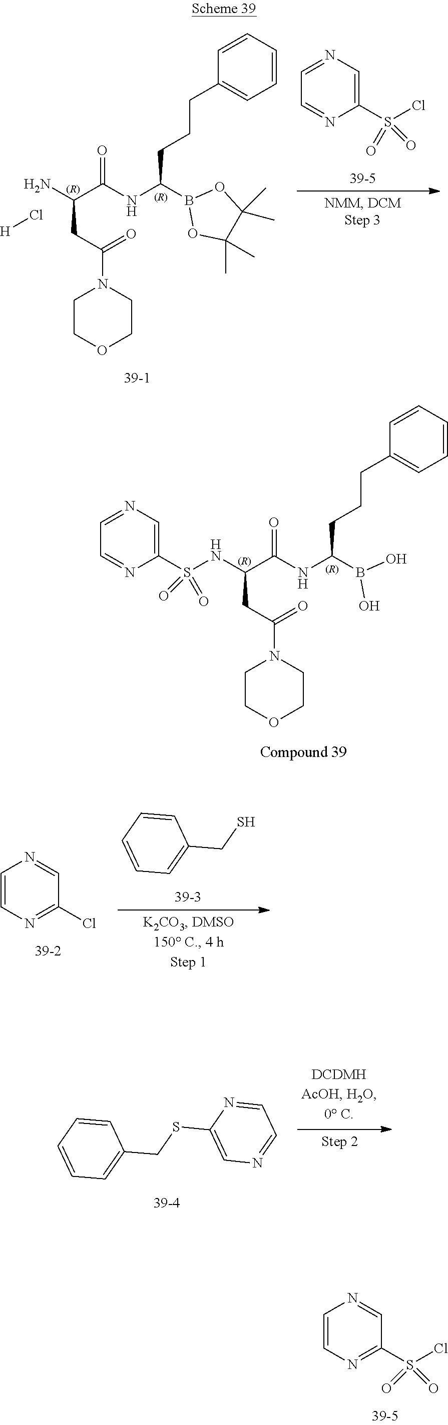

Synthesis of 2-(benzylthio)pyrazine [Step-1]: To a stirred solution of 2-chloropyrazine (39-2, 2.0 g, 17.5 mmol) and phenylmethanethiol (39-3, 3.1 mL, 26.0 mmol) in DMSO (16 mL) was added K₂CO₃ (3.6 g, 26.0 mmol) and the reaction mixture was stirred at 150° C. for 4 h. The reaction mixture was diluted with ethyl acetate and washed with water (thrice) and brine, dried over anhydrous Na₂SO₄ and concentrated under reduced pressure. The product was purified by flash chromatography to afford 2-(benzylthio)pyrazine (39-4, 2.1 g). [M+H]⁺=203. ¹H NMR (400 MHz, DMSO-d₆) δ 8.59 (s, 1H), 8.51 (brs, 1H), 8.34-8.33 (m, 1H), 7.41 (d, 2H), 7.33-7.22 (m, 3H), 4.45 (s, 2H).

Synthesis of pyrazine-2-sulfonyl chloride [Step-2]: To a stirred solution of 2-(benzylthio)pyrazine (39-4, 400 mg, 2.0 mmol) in DCM (2 mL) was added AcOH (0.04 mL, 0.66 mmol) and H2O (0.08 mL) at −20° C. 1,3-Dichloro-5,5-dimethylhydantoin (779 mg, 4.0 mmol) was added portion wise and the reaction mixture was stirred at same temperature for 1 h to obtain compound 39-5. The reaction mixture was diluted DCM and washed with water and brine, dried over anhydrous Na2SO4.

Synthesis of ((R)-1-((R)-4-morpholino-4-oxo-2-(pyrazine-2-sulfonamido)butanamido)-4-phenylbutyl)boronic acid [Step-3]: To a stirred solution of (R)-2-amino-4-morpholino-4-oxo-N—((R)-4-phenyl-1-(4,4,5,5-tetramethyl-1,3,2-dioxaborolan-2-yl)butyl) butanamide hydrochloride (39-1, 200 mg, 0.4 mmol) in DCM (2 mL) was added NMM (0.22 mL, 2.0 mmol) at 0° C. A solution of pyrazine-2-sulfonyl chloride (39-6, 264 mg, 0.44 mmol) in DCM (3 mL) was added to it and stirred at ambient temperature for 2 h. The reaction mixture was partitioned between DCM and water. Organic layer was washed with brine, dried over anhydrous Na₂SO₄ and concentrated under reduced pressure. The product was purified by prep HPLC purification and lyophilized to afford directly ((R)-1-((R)-4-morpholino-4-oxo-2-(pyrazine-2-sulfonamido)butanamido)-4-phenylbutyl)boronic acid (Compound 39.22 mg). [M−H]⁻=518.4. ¹H NMR (400 MHz, CD3OD) δ 9.13 (s, 1H), 8.80 (s, 1H), 8.62 (s, 1H), 7.24-7.13 (m, 5H), 4.73 (t, 1H), 3.63-3.53 (m, 4H), 3.43 (brs, 4H), 3.01-2.85 (m, 2H), 2.62 (brs, 3H), 1.65-1.39 (m, 4H).

### Example 40

### Synthesis of ((R)-1-((R)-4-morpholino-4-oxo-2-(phenethylamino)butanamido)-4-phenyl butyl)boronic acid

In another embodiment, Compound 40 can be produced from compound (40-1/31-2) by the method shown in Scheme 40.

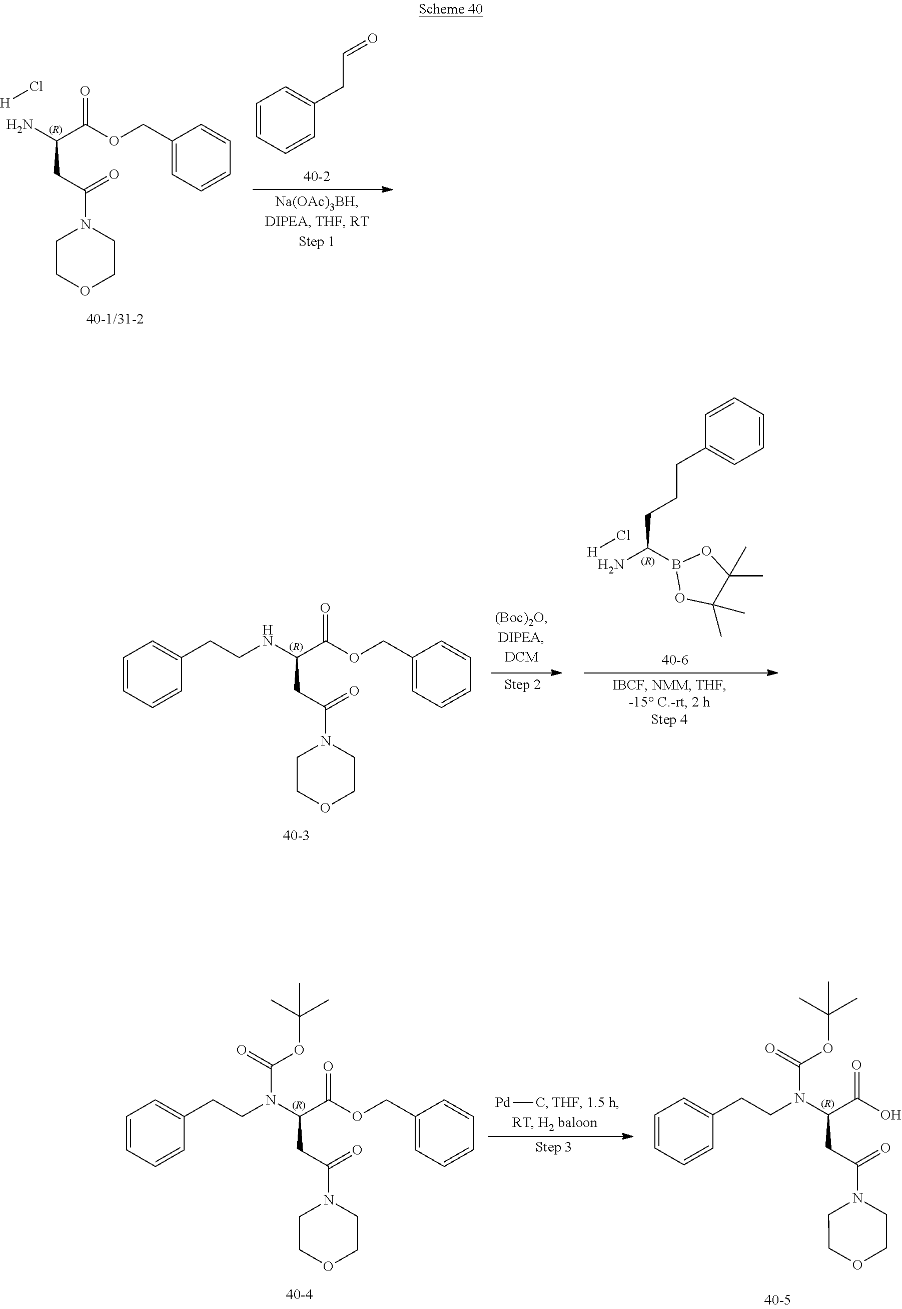

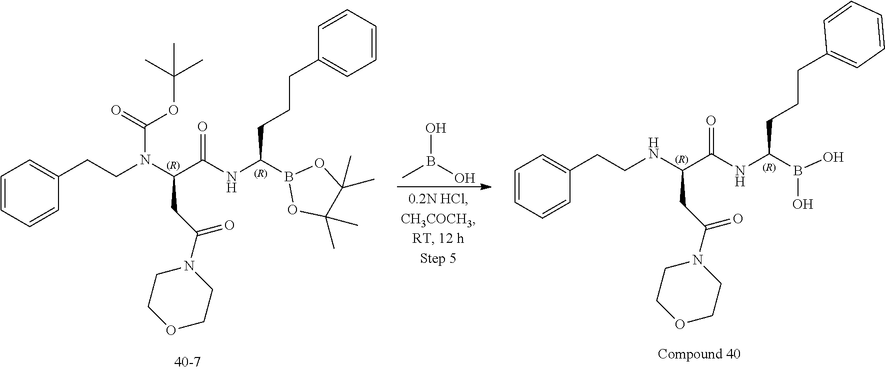

Synthesis of benzyl (R)-4-morpholino-4-oxo-2-(phenethylamino)butanoate [Step 1]: To a stirred solution of benzyl (R)-2-amino-4-morpholino-4-oxobutanoate hydrochloride (40-1/31-2, 800 mg, 2.4 mmol) in THF (12 mL) was added 2-phenylacetaldehyde (40-2, 0.3 mL, 2.4 mmol) under argon atmosphere. DIPEA (1.6 mL, 9.0 mmol) followed by NaBH(OAc)₃ (1 g, 4.9 mmol) was added at 0° C. and the reaction mixture was stirred at ambient temperature for 16 h. The reaction mixture was poured into crushed ice and partitioned between ethyl acetate and water. Organic layer was washed with water and brine, dried over anhydrous Na₂SO₄ and concentrated under reduced pressure. The product was purified by flash chromatography to afford benzyl (R)-4-morpholino-4-oxo-2-(phenethylamino)butanoate (40-3, 750 mg). ¹H NMR (400 MHz, DMSO-d₆) δ 7.35-7.12 (m, 10H), 5.09 (q, 2H), 3.63-3.36 (m, 10H), 2.83-2.61 (m, 6H).

Synthesis of benzyl (R)-2-((tert-butoxycarbonyl)(phenethyl)amino)-4-morpholino-4-oxobutanoate [Step 2]: To a stirred solution of benzyl (R)-4-morpholino-4-oxo-2-(phenethylamino)butanoate (40-3, 750 mg, 1.9 mmol) in DCM (5 mL) was added di-tert-butyl dicarbonate (0.5 mL, 2.3 mmol) followed by DIPEA (0.5 mL, 2.8 mmol) at ice cold condition and the reaction mixture was stirred at ambient temperature for 16 h. The reaction mixture was partitioned between DCM and water. Organic phase was washed with water and brine, dried over anhydrous Na₂SO₄ and concentrated under reduced pressure. The product was purified by flash chromatography to afford benzyl (R)-2-((tert-butoxycarbonyl)(phenethyl)amino)-4-morpholino-4-oxobutanoate (40-4, 800 mg). [M+H]⁺=497.4.

Synthesis of (R)-2-((tert-butoxycarbonyl)(phenethyl)amino)-4-morpholino-4-oxobutanoic acid [Step 3]: To a stirred solution of benzyl (R)-2-((tert-butoxycarbonyl)(phenethyl)amino)-4-morpholino-4-oxobutanoate (40-4, 750 mg, 1.5 mmol) in THF (10 mL) was added 10% Pd—C (160 mg, 1.5 mmol) and the reaction mixture was hydrogenated under hydrogen balloon pressure at ambient temperature for 3 h. The reaction mixture was filtered through a celite bed and the filtrate was concentrated under reduced pressure to afford (R)-2-((tert-butoxycarbonyl) (phenethyl)amino)-4-morpholino-4-oxobutanoic acid (40-5, 550 mg). [M+H]⁺=407.2.

Synthesis of tert-butyl ((R)-4-morpholino-1,4-dioxo-1-(((R)-4-phenyl-1-(4,4,5,5-tetramethyl-1,3,2-dioxaborolan-2-yl)butyl)amino)butan-2-yl)(phenethyl)carbamate [Step 4]: To a stirred of (R)-2-((tert-butoxycarbonyl)(phenethyl)amino)-4-morpholino-4-oxobutanoic acid (40-5, 360 mg, 0.9 mmol) in THF (8 mL) was added IBCF (1 mL, 0.7 mmol) followed by NMM (1 mL, 0.9 mmol) drop wise at −15° C. The reaction mixture was stirred at same temperature for 1 h. A solution of (R)-4-phenyl-1-(4,4,5,5-tetramethyl-1,3,2-dioxaborolan-2-yl)butan-1-amine (40-6, 250 mg, 0.8 mmol) in DMF (1 mL) followed by NMM (0.09 mL, 0.8 mmol) was added to the reaction mixture under same temperature and gradually warmed to 0° C. and stirred for 2 h. The reaction mixture was neutralized with 0.1(N) HCl in water and extracted with ethyl acetate (thrice). Combined organic layer was washed with 5% aqueous K₂CO₃ solution (twice), water and brine, dried over anhydrous Na₂SO₄ and concentrated under reduced pressure. The product was purified by prep HPLC purification and lyophilized to afford tert-butyl ((R)-4-morpholino-1,4-dioxo-1-(((R)-4-phenyl-1-(4,4,5,5-tetramethyl-1,3,2-dioxaborolan-2-yl)butyl)amino)butan-2-yl) (phenethyl)carbamate (40-7, 130 mg). [M−H]⁻=662.5 and [M-83]⁻=580.5.

Synthesis of ((R)-1-((R)-4-morpholino-4-oxo-2-(phenethylamino)butanamido)-4-phenyl butyl)boronic acid [Step 5]: To a stirred solution of tert-butyl ((R)-4-morpholino-1,4-dioxo-1-(((R)-4-phenyl-1-(4,4,5,5-tetramethyl-1,3,2-dioxaborolan-2-yl)butyl)amino)butan-2-yl)(phenethyl) carbamate (40-7, 60 mg, 0.09 mmol) and methyl boronic acid (54 mg, 0.90 mmol) in Acetone (2.5 mL) was added 1N HCl in water (2.5 mL) and the reaction mixture was stirred at ambient temperature for 16 h. The volatiles were evaporated and the product was purified by prep HPLC purification and lyophilized to afford ((R)-1-((R)-4-morpholino-4-oxo-2-(phenethylamino) butanamido)-4-phenylbutyl)boronic acid (Compound 40, 22 mg). [M−H]⁻=480.5. ¹H NMR (400 MHz, CD3OD): δ 7.26-7.12 (m, 10H), 3.76 (t, 1H), 3.63-3.47 (m, 8H), 2.87-2.53 (m, 9H), 1.65-1.42 (m, 4H).

### Example 41

### Synthesis of ((R)-1-((R)-2-((tert-butoxycarbonyl)(phenethyl)amino)-4-morpholino-4-oxo butanamido)-4-phenylbutyl)boronic acid

In another embodiment, Compound 41 can be produced from compound (40-7) by the method shown in Scheme 41.

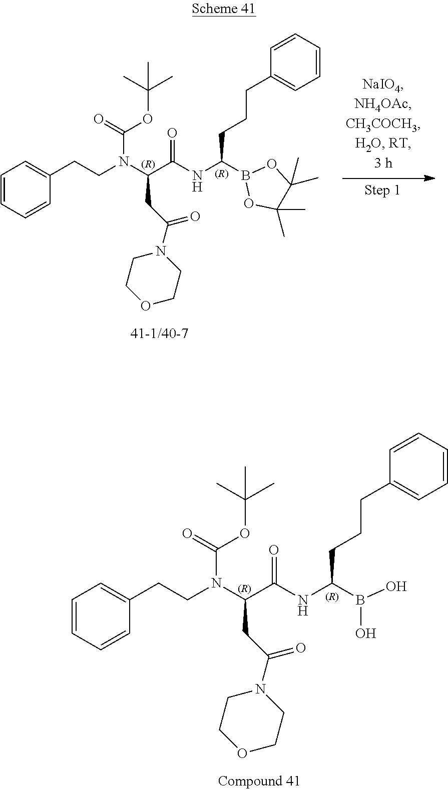

Synthesis of ((R)-1-((R)-2-((tert-butoxycarbonyl)(phenethyl)amino)-4-morpholino-4-oxo butanamido)-4-phenylbutyl)boronic acid [Step 1]: To a stirred solution of tert-butyl ((R)-4-morpholino-1,4-dioxo-1-(((R)-4-phenyl-1-(4,4,5,5-tetramethyl-1,3,2-dioxaborolan-2-yl)butyl) amino)butan-2-yl)(phenethyl)carbamate (41-1/40-7, 60 mg, 0.09 mmol) in acetone (1.5 mL) and water (1.5 mL) was added NaIO₄ (21 mg, 0.09 mmol) followed by NH₄OAc (8.4 mg, 0.10 mmol) and the reaction mixture was stirred at ambient temperature for 16 h. Volatiles was evaporated under reduced pressure and the residue was partitioned between ethyl acetate and water. Organic layer was washed with water and brine, dried over anhydrous Na₂SO₄ and concentrated under reduced pressure. The product was purified by prep HPLC purification and lyophilized to afford ((R)-1-((R)-2-((tert-butoxycarbonyl)(phenethyl)amino)-4-morpholino-4-oxobutanamido)-4-phenylbutyl)boronic acid (Compound 41, 9.0 mg). [M−H]⁻=580.5. ¹H NMR (400 MHz, CD₃OD): δ 7.29-7.10 (m, 10H), 4.94 (brs, 1H), 3.66-3.45 (m, 10H), 3.17-3.11 (m, 1H), 2.86 (brs, 2H), 2.59 (brs, 4H), 1.64-1.53 (m, 4H), 1.46 (s, 9H).

### Example 42

### Synthesis of ((R)-1-((R)-4-morpholino-4-oxo-2-(pyrrolidine-1-carboxamido)butanamido)-4-phenylbutyl)boronic acid

In another embodiment, Compound 42 can be produced from compound (42-1/1-7) by the method shown in Scheme 42.

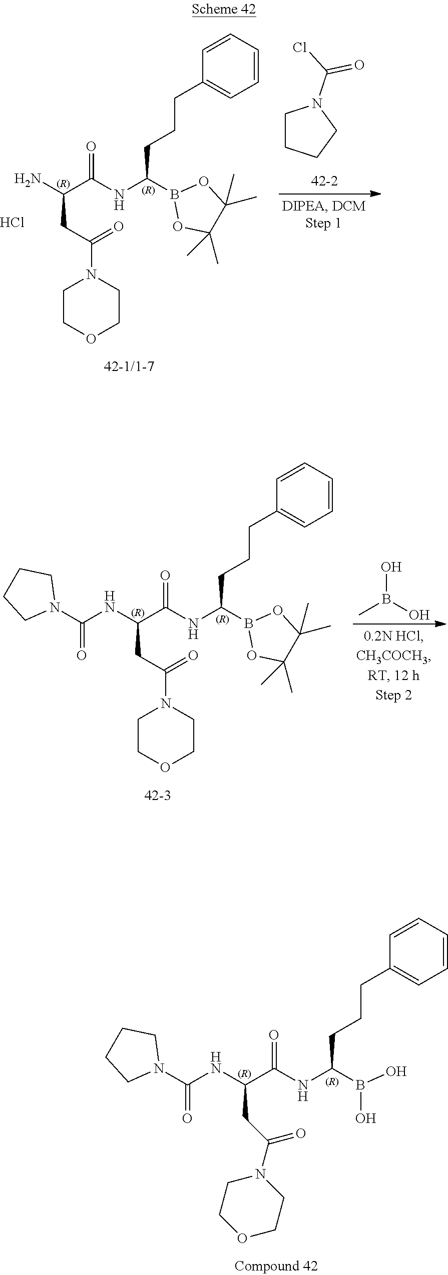

Synthesis of N—((R)-4-morpholino-1,4-dioxo-1-(((R)-4-phenyl-1-(4,4,5,5-tetramethyl-1,3,2-dioxaborolan-2-yl)butyl)amino)butan-2-yl)pyrrolidine-1-carboxamide [Step 1]: To a stirred solution of (R)-2-amino-4-morpholino-4-oxo-N—((R)-4-phenyl-1-(4,4,5,5-tetramethyl-1,3,2-dioxaborolan-2-yl)butyl)butanamide hydrochloride (42-1/1-7, 220 mg, 0.4 mmol) in DCM (5 mL) was added DIPEA (0.3 mL, 2 mmol) at 0° C. followed by pyrrolidine-1-carbonyl chloride (42-2, 0.1 mL, 0.7 mmol) and stirred for 3 h. The reaction was diluted with DCM and washed with water and brine, dried over anhydrous Na₂SO₄ and evaporated under reduced pressure. The product was purified by prep HPLC purification and lyophilized to afford N—((R)-4-morpholino-1,4-dioxo-1-(((R)-4-phenyl-1-(4,4,5,5-tetramethyl-1,3,2-dioxaborolan-2-yl)butyl)amino)butan-2-yl) pyrrolidine-1-carboxamide (42-3, 70 mg). [M−H]⁻=555.5, [M-83]⁻=473.3.

Synthesis of ((R)-1-((R)-4-morpholino-4-oxo-2-(pyrrolidine-1-carboxamido)butanamido)-4-phenylbutyl)boronic acid [Step 2]: To a stirred solution of N—((R)-4-morpholino-1,4-dioxo-1-(((R)-4-phenyl-1-(4,4,5,5-tetramethyl-1,3,2-dioxaborolan-2-yl)butyl)amino)butan-2-yl)pyrrolidine-1-carboxamide (42-3, 70 mg, 0.1 mmol) and methyl boronic acid (90 mg, 1.5 mmol) in acetone (4 mL) was added 0.2N HCl in water (4 mL) and the reaction mixture was stirred at ambient temperature for 16 h. The volatiles were evaporated under reduced pressure and the product was purified by prep HPLC purification and lyophilized to afford ((R)-1-((R)-4-morpholino-4-oxo-2-(pyrrolidine-1-carboxamido)butanamido)-4-phenylbutyl)boronic acid (Compound 42, 32 mg). [M−H]⁻=473.5. 1H NMR (400 MHz, MeOD) δ 7.22-7.16 (m, 4H), 7.12 (d, 1H), 4.94 (brs, 1H), 3.65-3.59 (m, 4H), 3.50 (brs, 4H), 3.34-3.30 (m, 1H), 3.04 (dd, 1H), 2.88 (dd, 1H), 2.61-2.59 (m, 4H), 1.94-1.90 (m, 6H), 1.67 (brs, 2H), 1.56-1.49 (m, 2H).

### Example 43

### Synthesis of ((R)-1-((R)-2-(3-methylureido)-4-morpholino-4-oxobutanamido)-4-phenyl butyl)boronic acid

In another embodiment, Compound 43 can be produced from compound (43-1/31-2) by the method shown in Scheme 43.

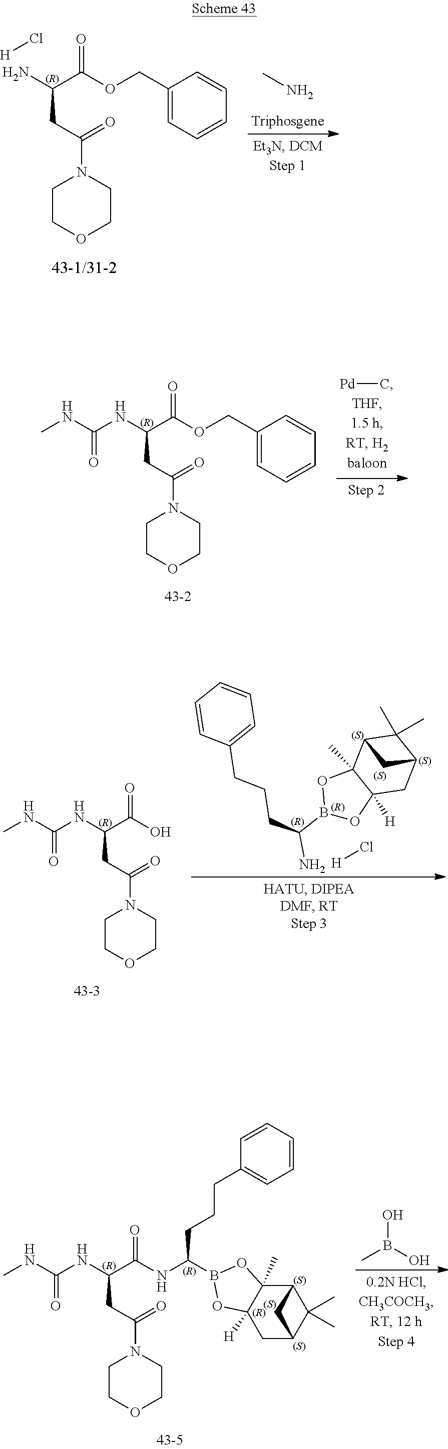

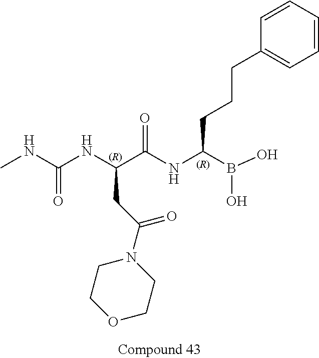

Synthesis of benzyl (R)-2-(3-methylureido)-4-morpholino-4-oxobutanoate [Step 1]: To a stirred solution of triphosgene (200 mg, 0.67 mmol) in DCM (5 mL) was added an ice cold solution of benzyl (R)-2-amino-4-morpholino-4-oxobutanoate hydrochloride (43-1, 600 mg, 1.82 mmol) in DCM (7 ml) and DIPEA (0.82 mL, 6.02 mmol) followed by Methylamine (2M in THF) (2.8 mL, 5.47 mmol) at −15° C. The reaction mixture was stirred at −15° C. for 1 h. The reaction mixture was diluted with DCM and washed with water. Organic layer was dried over anhydrous Na₂SO₄ and concentrated under reduce pressure. The product was purified by column chromatography to afford benzyl (R)-2-(3-methylureido)-4-morpholino-4-oxobutanoate (43-2, 580 mg). [M+H]⁺=350.4.

Synthesis of (R)-2-(3-methylureido)-4-morpholino-4-oxobutanoic acid [Step 2]: Benzyl (R)-2-(3-methylureido)-4-morpholino-4-oxobutanoate (43-2, 580 mg, 1.7 mmol)) was dissolved in THF (20 mL). 10% Pd—C (205 mg) was added to it and the reaction mixture was hydrogenated under hydrogen balloon pressure at ambient temperature for 3 h. The reaction mixture was filtered through a celite bed and washed with ethyl acetate (twice). Combined filtrate was concentrated under reduced pressure to afford ((R)-2-(3-methylureido)-4-morpholino-4-oxobutanoic acid (43-3, 420 mg). [M+H]⁺=260.1.

Synthesis of (R)-2-(3-methylureido)-4-morpholino-4-oxo-N—((R)-4-phenyl-1-((3aS,4S,6S,7aR)-3a,5,5-trimethylhexahydro-4,6-methanobenzo[d][1,3,2]dioxaborol-2-yl)butyl)butanamide [Step 3]: To a stirred solution of (R)-4-phenyl-1-((3aS,4S,6S,7aR)-3a,5,5-trimethylhexahydro-4,6-methanobenzo[d][1,3,2]dioxaborol-2-yl)butan-1-amine hydrochloride (43-4, 250 mg, 0.69 mmol) and ((R)-2-(3-methylureido)-4-morpholino-4-oxobutanoic acid (43-3, 196 mg, 0.76 mmol) in DMF (5 mL) was added DIPEA (0.62 mL, 3.44 mmol) followed by HATU (392 mg, 1.0 mmol) at 0° C. The reaction mixture was stirred at ambient temperature for 2 h. The product was purified by prep HPLC purification and lyophilized to afford (R)-2-(3-methylureido)-4-morpholino-4-oxo-N—((R)-4-phenyl-1-((3aS,4S,6S,7aR)-3a,5,5-trimethylhexahydro-4,6-methanobenzo[d][1,3,2]dioxaborol-2-yl)butyl)butanamide (43-5, 140 mg). [M−H]⁻=567.5 and [M-135]⁻=433.3.

Synthesis of ((R)-1-((R)-2-(3-methylureido)-4-morpholino-4-oxobutanamido)-4-phenyl butyl)boronic acid [Step 4]: To a stirred solution of (R)-2-(3-methylureido)-4-morpholino-4-oxo-N—((R)-4-phenyl-1-((3aS,4S,6S,7aR)-3a,5,5-trimethylhexahydro-4,6-methanobenzo[d][1,3,2]dioxaborol-2-yl)butyl)butanamide (43-5, 145 mg, 0.25 mmol) and methylboronic acid (153 mg, 2.5 mmol) in acetone (4 mL) was added 0.2 N HCl (4.0 mL). The reaction mixture was stirred at ambient temperature for 16 h. Volatiles were removed under reduced pressure. The product was purified by prep HPLC purification and lyophilized to afford ((R)-1-((R)-2-(3-methylureido)-4-morpholino-4-oxobutanamido)-4-phenylbutyl)boronic acid (Compound 43, 90 mg). [M−H]⁻=433.4. ¹H NMR (400 MHz, MeOD) δ 7.24-7.10 (m, 5H), 4.89 (brs, 1H), 3.65-3.60 (m, 4H), 3.50 (brs, 4H), 3.10-3.04 (m, 1H), 2.84-2.79 (m, 1H), 2.69 (s, 3H), 2.61-2.56 (m, 3H), 1.63 (brs, 2H), 1.56-1.50 (m, 2H).

### Example 44

### Synthesis of ((R)-1-((R)-2-((R)-3-hydroxypyrrolidine-1-carboxamido)-4-morpholino-4-oxobutanamido)-4-phenylbutyl)boronic acid

In another embodiment, Compound 44 can be produced from compound (44-1/31-2) by the method shown in Scheme 44.

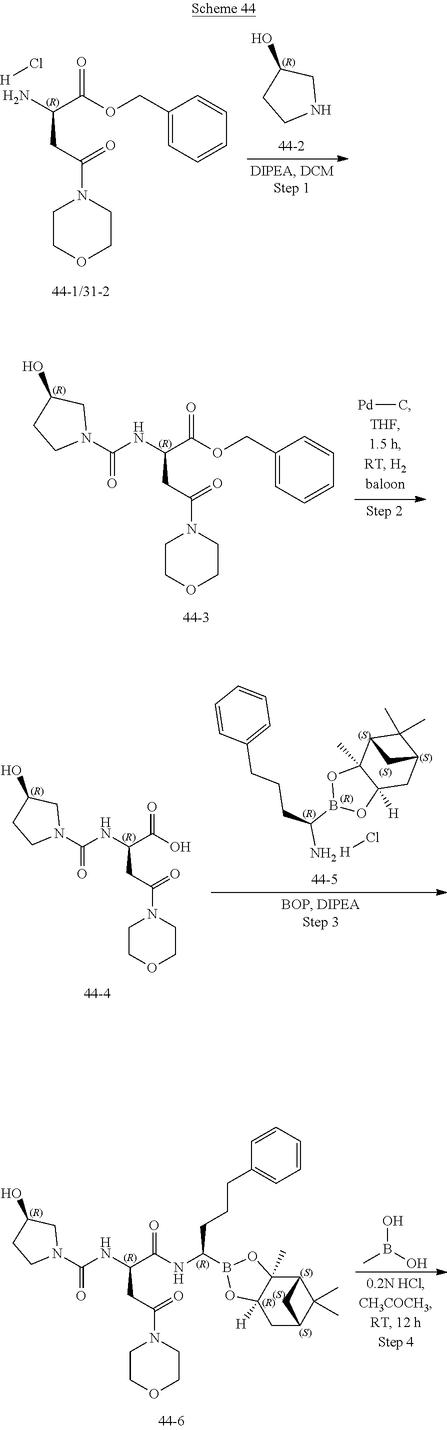

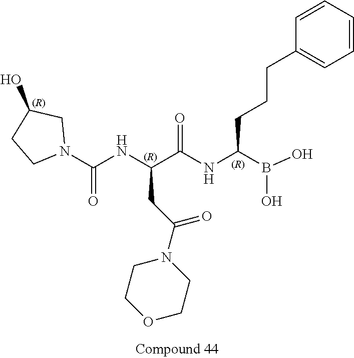

Synthesis of benzyl (R)-2-((R)-3-hydroxypyrrolidine-1-carboxamido)-4-morpholino-4-oxobutanoate [Step 1]: To a stirred solution of triphosgene (234 mg, 0.79 mmol) in DCM (5 mL) was added an ice cold solution of benzyl (R)-2-amino-4-morpholino-4-oxobutanoate hydrochloride (44-1/43-1, 700 mg, 2.13 mmol) in DCM (10 mL) and DIPEA (0.96 mL, 7.03 mmol) followed by a solution of (3R)-pyrrolidin-3-ol (44-2, 185 mg, 2.13 mmol) in DCM (5 mL) at −15° C. The reaction mixture was stirred at same temperature for 1 h. The reaction mixture was diluted with DCM and washed with water. Organic layer was dried over anhydrous Na₂SO₄ and concentrated under reduce pressure. The product was purified by column chromatography to afford benzyl (R)-2-((R)-3-hydroxypyrrolidine-1-carboxamido)-4-morpholino-4-oxobutanoate (44-3, 700 mg). [M+H]⁺=406.1.

Synthesis of (R)-2-((R)-3-hydroxypyrrolidine-1-carboxamido)-4-morpholino-4-oxobutanoic acid [Step 2]: To a solution of benzyl (R)-2-((R)-3-hydroxypyrrolidine-1-carboxamido)-4-morpholino-4-oxobutanoate (44-3, 350 mg, 0.86 mmol) in THF (20 mL) was added 10% Pd—C (106 mg). The reaction mixture was hydrogenated under hydrogen balloon pressure for 3 h at ambient temperature. The reaction mixture was filtered through a celite bed and washed with ethyl acetate (twice). The filtrate was concentrated under reduced pressure to afford (R)-2-((R)-3-hydroxypyrrolidine-1-carboxamido)-4-morpholino-4-oxobutanoic acid (44-4, 272 mg). [M+H]⁺=316.19.

Synthesis of (R)-3-hydroxy-N—((R)-4-morpholino-1,4-dioxo-1-(((R)-4-phenyl-1-((3aS,4S,6S,7aR)-3a,5,5-trimethylhexahydro-4,6-methanobenzo[d][1,3,2]dioxaborol-2-yl) butyl)amino)butan-2-yl)pyrrolidine-1-carboxamide [Step 3]: To a stirred solution of (R)-4-phenyl-1-((3aS,4S,6S,7aR)-3a,5,5-trimethylhexahydro-4,6-methanobenzo[d][1,3,2]dioxaborol-2-yl)butan-1-amine hydrochloride (44-5, 200 mg, 0.55 mmol) and (R)-2-((R)-3-hydroxypyrrolidine-1-carboxamido)-4-morpholino-4-oxobutanoic acid (44-4, 191 mg, 0.61 mmol) in DMF (4 mL) was added DIPEA (0.49 mL, 2.75 mmol) followed by HATU (314 mg, 0.825 mmol) at 0° C. The reaction mixture was stirred at ambient temperature for 2 h. The reaction mixture was filtered and the filtrate was purified by prep HPLC purification and lyophilized to afford (R)-3-hydroxy-N—((R)-4-morpholino-1,4-dioxo-1-(((R)-4-phenyl-1-((3aS,4S,6S,7aR)-3a,5,5-trimethylhexahydro-4,6-methanobenzo[d][1,3,2]dioxaborol-2-yl)butyl)amino)butan-2-yl)pyrrolidine-1-carboxamide (44-6, 110 mg). [M−H]⁻=623.6.

Synthesis of ((R)-1-((R)-2-((R)-3-hydroxypyrrolidine-1-carboxamido)-4-morpholino-4-oxobutanamido)-4-phenylbutyl)boronic acid [Step 4]: To a stirred solution (R)-3-hydroxy-N—((R)-4-morpholino-1,4-dioxo-1-(((R)-4-phenyl-1-((3aS,4S,6S,7aR)-3a,5,5-trimethylhexahydro-4,6-methanobenzo[d][1,3,2]dioxaborol-2-yl)butyl)amino)butan-2-yl)pyrrolidine-1-carboxamide (44-6, 110 mg, 0.18 mmol) and methylboronic acid (105 mg, 1.76 mmol) in acetone (4 mL) was added 0.2N HCl (4.0 mL). The reaction mixture was stirred at ambient temperature for 16 h. The volatiles were removed under reduced pressure. The product was purified by prep HPLC purification and lyophilized to afford ((R)-1-((R)-2-((R)-3-hydroxypyrrolidine-1-carboxamido)-4-morpholino-4-oxobutanamido)-4-phenylbutyl)boronic acid (Compound 44, 55 mg). [M−H]⁻=489.3. ¹H NMR (400 MHz, MeOD) δ 7.24-7.11 (m, 5H), 4.95-4.94 (m, 1H), 3.40 (brs, 1H), 3.66-3.41 (m, 12H), 3.09-3.04 (m, 1H), 2.92-2.86 (m, 1H), 2.60-2.57 (m, 3H), 2.0-1.94 (m, 2H), 1.70-1.66 (m, 2H), 1.56-1.48 (m, 2H).

### Example 45

### Synthesis of ((R)-1-((R)-2-((S)-3-hydroxypyrrolidine-1-carboxamido)-4-morpholino-4-oxobutanamido)-4-phenylbutyl)boronic acid

In another embodiment, Compound 45 can be produced from compound (45-1/31-2) by the method shown in Scheme 45.

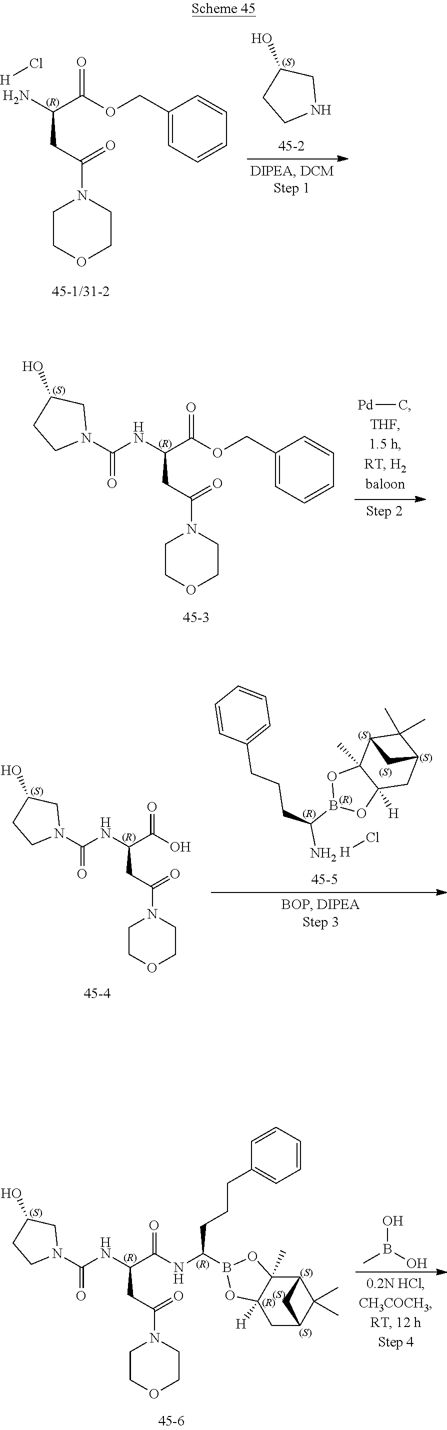

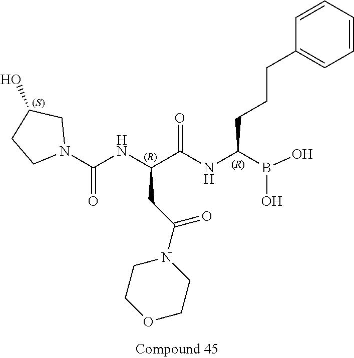

Synthesis of benzyl (R)-2-((S)-3-hydroxypyrrolidine-1-carboxamido)-4-morpholino-4-oxobutanoate [Step 1]: To a stirred solution of triphosgene (234 mg, 0.79 mmol) in DCM (5 mL) was added an ice cold solution of benzyl (R)-2-amino-4-morpholino-4-oxobutanoate hydrochloride (45-1/41-2, 700 mg, 2.13 mmol) in DCM (10 mL) and DIPEA (0.96 mL, 7.03 mmol) followed by a solution of (3S)-pyrrolidin-3-ol (45-2, 185 mg, 2.13 mmol) in DCM (5 mL) at −15° C. The reaction mixture was allowed to stir at same temperature for 1 h. The reaction mixture was diluted with DCM and washed with water. Organic layer was dried over anhydrous Na₂SO₄ and concentrated under reduce pressure. The product was purified by column chromatography to afford benzyl (R)-2-((S)-3-hydroxypyrrolidine-1-carboxamido)-4-morpholino-4-oxobutanoate (45-3, 680 mg). [M+H]⁺=406.2.

Synthesis of (R)-2-((S)-3-hydroxypyrrolidine-1-carboxamido)-4-morpholino-4-oxobutanoic acid [Step 2]: To a solution of benzyl (R)-2-((S)-3-hydroxypyrrolidine-1-carboxamido)-4-morpholino-4-oxobutanoate (45-3, 420 mg, 1.04 mmol) in THF (20 mL) was added 10% Pd—C (130 mg). The reaction mixture was hydrogenated under hydrogen balloon pressure at ambient temperature for 3 h. The reaction mixture was filtered through a celite bed and washed with ethyl acetate (twice). The combined filtrate was concentrated under reduced pressure to afford (R)-2—((S)-3-hydroxypyrrolidine-1-carboxamido)-4-morpholino-4-oxobutanoic acid (45-4, 310 mg). [M+H]⁺=316.2.

Synthesis of (S)-3-hydroxy-N—((R)-4-morpholino-1,4-dioxo-1-(((R)-4-phenyl-1-((3aS,4S,6S,7aR)-3a,5,5-trimethylhexahydro-4,6-methanobenzo[d][1,3,2]dioxaborol-2-yl)butyl)amino)butan-2-yl)pyrrolidine-1-carboxamide [Step 3]: To a stirred solution of (R)-4-phenyl-1-((3aS,4S,6S,7aR)-3a,5,5-trimethylhexahydro-4,6-methanobenzo[d][1,3,2]dioxaborol-2-yl)butan-1-amine hydrochloride (45-5, 200 mg, 0.55 mmol) and (R)-2-((S)-3-hydroxypyrrolidine-1-carboxamido)-4-morpholino-4-oxobutanoic acid (45-4, 191 mg, 0.61 mmol) in DMF (5 mL) was added DIPEA (0.49 mL, 2.75 mmol) followed by HATU (314 mg, 0.82 mmol) at 0° C. The reaction mixture was stirred at ambient temperature for 2 h. The reaction mixture was filtered and the filtrate was purified by prep HPLC purification and lyophilized to afford (S)-3-hydroxy-N—((R)-4-morpholino-1,4-dioxo-1-(((R)-4-phenyl-1-((3aS,4S,6S,7aR)-3a,5,5-trimethylhexahydro-4,6-methanobenzo[d][1,3,2]dioxaborol-2-yl)butyl)amino)butan-2-yl)pyrrolidine-1-carboxamide (45-6, 180 mg). [M−H]⁻=623.4.

Synthesis of ((R)-1-((R)-2-((S)-3-hydroxypyrrolidine-1-carboxamido)-4-morpholino-4-oxobutanamido)-4-phenylbutyl)boronic acid [Step 4]: To a stirred solution (S)-3-hydroxy-N—((R)-4-morpholino-1,4-dioxo-1-(((R)-4-phenyl-1-((3aS,4S,6S,7aR)-3a,5,5-trimethylhexahydro-4,6-methanobenzo[d][1,3,2]dioxaborol-2-yl)butyl)amino)butan-2-yl)pyrrolidine-1-carboxamide (45-6, 100 mg, 0.16 mmol) and methylboronic acid (96 mg, 1.6 mmol) in acetone (4 mL) was added 0.2 N HCl (4.0 mL). The reaction mixture was stirred at ambient temperature for 16 h. The volatiles were evaporated under reduced pressure. The product was purified by prep HPLC purification and lyophilized to afford ((R)-1-((R)-2-((S)-3-hydroxypyrrolidine-1-carboxamido)-4-morpholino-4-oxobutanamido)-4-phenylbutyl)boronic acid (Compound 45, 45 mg). [M−H]⁻=489.4. ¹H NMR (400 MHz, MeOD) δ 7.24-7.11 (m, 5H), 4.96-4.94 (m, 1H), 3.40 (brs, 1H), 3.65-3.34 (m, 12H), 3.09-3.07 (m, 1H), 2.92-2.86 (m, 1H), 2.62-2.57 (m, 3H), 2.02-1.92 (m, 2H), 1.68-1.66 (m, 2H), 1.56-1.53 (m, 2H).

### Example 46

### Synthesis of (R)-4-morpholino-4-oxo-N—((R)-4-phenyl-1-((3aS,4S,6S,7aR)-3a,5,5-trimethyl hexahydro-4,6-methanobenzo[d][1,3,2]dioxaborol-2-yl)butyl)-2-(phenylamino)butanamide

In another embodiment, Compound 46 can be produced from compound (46-1) by the method shown in Scheme 46.

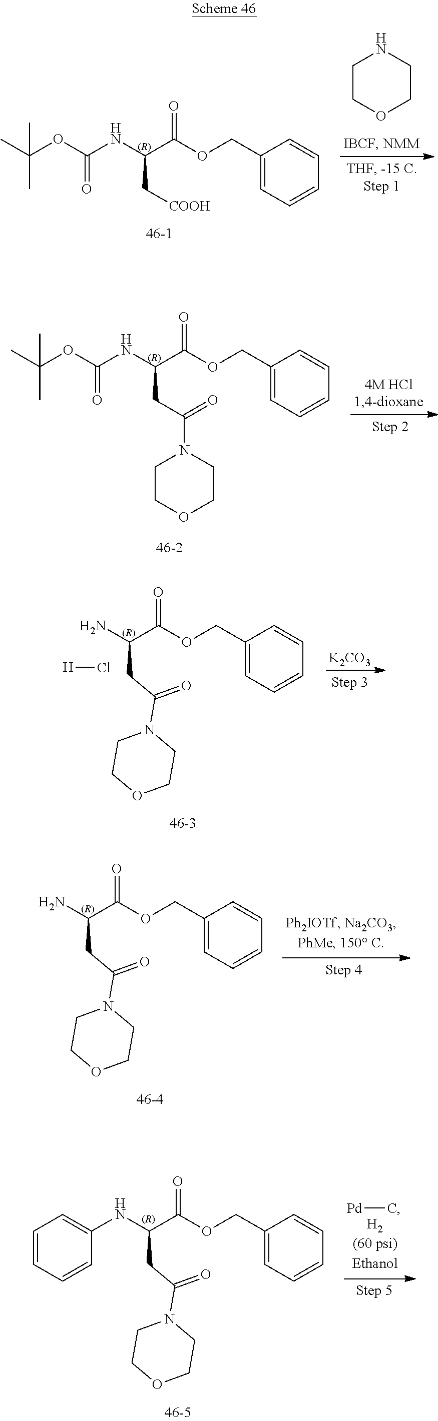

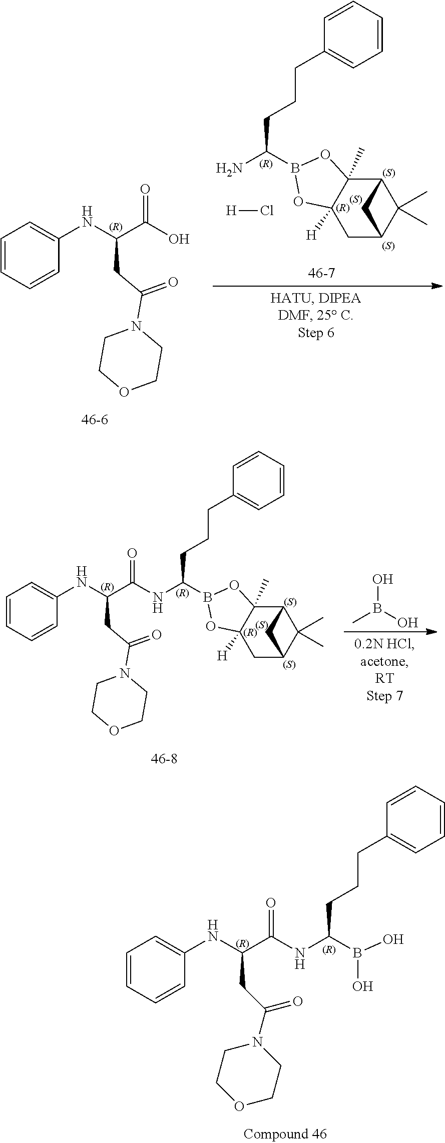

Synthesis of benzyl (R)-2-((tert-butoxycarbonyl)amino)-4-morpholino-4-oxobutanoate [Step 1]: To a stirred solution of (R)-4-(benzyloxy)-3-((tert-butoxycarbonyl)amino)-4-oxobutanoic acid (46-1, 4.1 g, 12.6 mmol) in THF (30 mL) was added IBCF (1.7 mL, 12.6 mmol) followed by NMM (1.7 mL, 12.6 mmol) at −15° C. After 45 min, a solution of morpholine (1.0 mL, 11.5 mmol) in DMF (2 mL) was added dropwise followed by NMM (1.6 mL, 11.5 mmol). After stirring for 1 h at the same temperature, the reaction was diluted with EtOAc, and washed successively with 0.1 N aqueous HCl (twice), 5% aqueous K₂CO₃, water and brine. The organic phase was dried over anhydrous Na₂SO₄, filtered and concentrated under reduced pressure. The compound was purified by column chromatography to afford benzyl (R)-2-((tert-butoxycarbonyl)amino)-4-morpholino-4-oxobutanoate (46-2, 3.75 g). [M+H]⁺=392.8.

Synthesis of benzyl (R)-2-amino-4-morpholino-4-oxobutanoate hydrochloride [Step 2]: To an ice-cold solution of benzyl (R)-2-((tert-butoxycarbonyl)amino)-4-morpholino-4-oxobutanoate (46-2, 1.7 g, 4.3 mmol) in 1,4-dioxane (10 mL) was added HCl (4M dioxane) (11 mL, 43.3 mmol), and the reaction mixture was stirred at 25° C. After 16 h, the reaction mixture was concentrated under reduced pressure to afford crude benzyl (R)-2-amino-4-morpholino-4-oxobutanoate hydrochloride (46-3, 1.20 g), which was used in the next step without further purification. [M+H]⁺=293.2.

Synthesis of benzyl (R)-2-amino-4-morpholino-4-oxobutanoate [Step 3]: To a solution of benzyl (R)-2-amino-4-morpholino-4-oxobutanoate hydrochloride (46-3, 1.1 g, 3.4 mmol) in DCM (10 mL) was added K₂CO₃ (1.1 g, 7.9 mmol) followed by water (5 mL). The reaction mixture was stirred vigorously for at 25° C. After 30 min, the reaction mixture was diluted with DCM, and washed with water. The organic phase was dried over anhydrous Na₂SO₄, and concentrated under reduced pressure to obtain crude benzyl (R)-2-amino-4-morpholino-4-oxobutanoate (46-4, 700 mg), which was used in the next step without further purification. [M+H]⁺=292.9.

Synthesis of benzyl (R)-4-morpholino-4-oxo-2-(phenylamino)butanoate [Step 4]: A sealed tube was charged with crude benzyl (R)-2-amino-4-morpholino-4-oxobutanoate (46-4, 700 mg, 2.4 mmol), diphenyliodonium triflate (661 mg, 4.8 mmol) and Na₂CO₃ (254 mg, 2.4 mmol) under argon. Degassed toluene (10 mL) was added, and the reaction mixture was stirred at 150° C. for 16 h. The reaction mixture was concentrated under reduced pressure, and purified by column chromatography to afford benzyl (R)-4-morpholino-4-oxo-2-(phenylamino)butanoate (46-5, 120 mg). [M+H]⁺=368.4.

Synthesis of (R)-4-morpholino-4-oxo-2-(phenylamino)butanoic acid [Step 5]: To a solution of benzyl (R)-4-morpholino-4-oxo-2-(phenylamino)butanoate (46-5, 200 mg, 0.5 mmol) in ethanol (2 mL) was added 10% Pd/C (58 mg) under N₂. The reaction vessel was evacuated and backfilled with H₂ (twice), and then kept under a positive pressure of H₂. The reaction mixture was stirred at ambient temperature. After 16 h, the reaction mixture was filtered through a pad of celite, and the filtrate was concentrated under reduced pressure to afford crude @-4-morpholino-4-oxo-2-(phenylamino)butanoic acid (46-6, 100 mg), which was used for the next step without further purification. [M+H]⁺=279.1.

Synthesis of (R)-4-morpholino-4-oxo-N—((R)-4-phenyl-1-((3aS,4S,6S,7aR)-3a,5,5-trimethyl hexahydro-4,6-methanobenzo[d][1,3,2]dioxaborol-2-yl)butyl)-2-(phenylamino)butanamide [Step 6]: To an ice-cold solution of (R)-4-morpholino-4-oxo-2-(phenylamino)butanoic acid (46-6, 100 mg, 0.4 mmol), (R)-4-phenyl-1-((3aS,4S,6S,7aR)-3a,5,5-trimethylhexahydro-4,6-methano benzo[d][1,3,2]dioxaborol-2-yl)butan-1-amine hydrochloride (46-7, 120 mg, 0.3 mmol) and HATU (138 mg, 0.4 mmol) in DMF (3 mL) was added DIPEA (0.1 mL, 0.5 mmol), and the reaction mixture was stirred at 25° C. for 2 h. The reaction was quenched with cold water, and extracted with EtOAc (twice). Combined organic phase was washed with brine, dried over anhydrous Na₂SO₄, filtered and concentrated under reduced pressure afford crude (R)-4-morpholino-4-oxo-N—((R)-4-phenyl-1-((3aS,4S,6S,7aR)-3a,5,5-trimethylhexahydro-4,6-methano benzo[d][1,3,2]dioxaborol-2-yl)butyl)-2-(phenylamino)butanamide (46-8, 200 mg), which was used in the next step without further purification. [M+H]⁺=588.0.

Synthesis of (R)-4-morpholino-4-oxo-N—((R)-4-phenyl-1-((3aS,4S,6S,7aR)-3a,5,5-trimethyl hexahydro-4,6-methanobenzo[d][1,3,2]dioxaborol-2-yl)butyl)-2-(phenylamino)butanamide [Step 7]: To an ice-cold solution of crude (R)-4-morpholino-4-oxo-N—((R)-4-phenyl-1-((3aS,4S,6S,7aR)-3a,5,5-trimethylhexahydro-4,6-methanobenzo[d][1,3,2]dioxaborol-2-yl)butyl)-2-(phenylamino)butanamide (46-8, 200 mg, 0.3 mmol) and methylboronic acid (306 mg, 5.1 mmol) in acetone (10 mL) was added 0.2N aqueous HCl (10 mL), and the reaction mixture was stirred at 25° C. After 16 h, the reaction mixture was concentrated under reduced pressure, and then lyophilized. The compound was purified by prep HPLC (RP), and lyophilized to afford (R)-4-morpholino-4-oxo-N—((R)-4-phenyl-1-((3aS,4S,6S,7aR)-3a,5,5-trimethylhexahydro-4,6-methano benzo[d][1,3,2]dioxaborol-2-yl)butyl)-2-(phenylamino)butanamide (Compound 46, 12 mg). [M−H]⁻=452.5. ¹H NMR (400 MHz, MeOD): δ 7.21-7.19 (m, 2H), 7.15-7.11 (m, 5H), 6.70-6.68 (m, 3H), 4.65 (m, 1H), 3.62-3.58 (m, 4H), 3.52-3.49 (m, 4H), 2.98-2.94 (m, 2H), 2.57-2.55 (m, 3H), 1.62-1.60 (m, 2H), 1.50-1.48 (m, 1H), 1.41-1.39 (m, 1H).

### Example 47

### Synthesis of (R)-4-morpholino-4-oxo-N—((R)-4-phenyl-1-((3aS,4S,6S,7aR)-3a,5,5-trimethyl hexahydro-4,6-methanobenzo[d][1,3,2]dioxaborol-2-yl)butyl)-2-(phenylamino)butanamide

In another embodiment, Compound 47 can be produced from compound (47-1/31-2) by the method shown in Scheme 47.

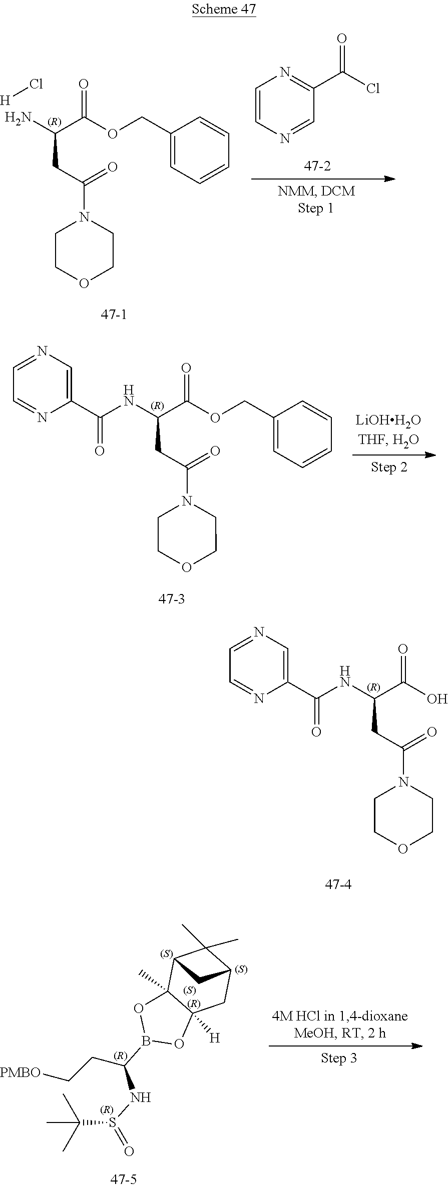

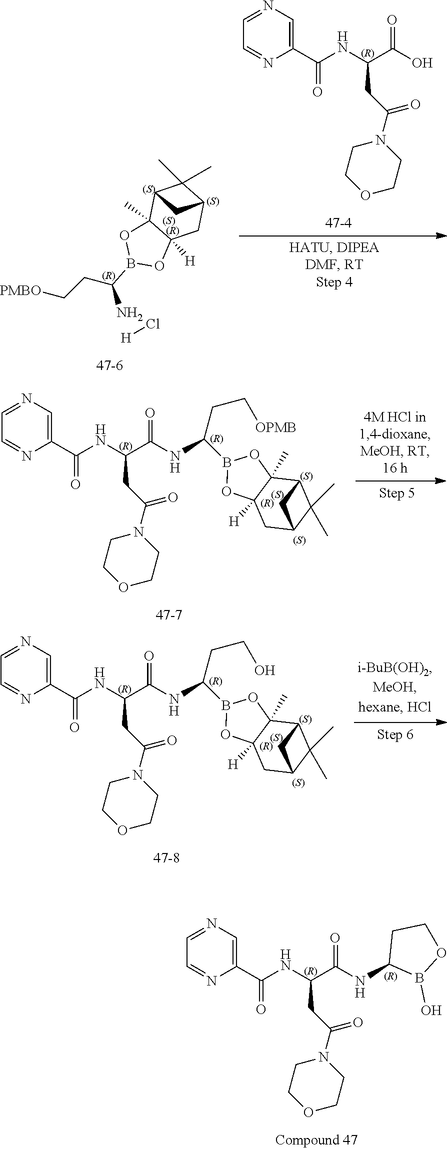

Synthesis of benzyl (R)-4-morpholino-4-oxo-2-(pyrazine-2-carboxamido)butanoate [Step 1]: To a stirred solution of benzyl (R)-2-amino-4-morpholino-4-oxobutanoate hydrochloride (47-1, 400 mg, 1.37 mmol) in DCM (8 mL) was added NMM (0.3 mL, 2.74 mmol) at −15° C. followed by the addition of pyrazine-2-carbonyl chloride (47-2, 234 mg, 1.64 mmol). The reaction mixture was stirred at ambient temperature for 2 h. The reaction mixture was diluted with DCM and washed with water and brine, dried over anhydrous sodium sulfate and concentrated under reduced pressure. The product was purified by flash chromatography to afford benzyl (R)-4-morpholino-4-oxo-2-(pyrazine-2-carboxamido)butanoate (47-3, 320 mg). LCMS (ESI) [M+H]⁺=399.2.

Synthesis of (R)-4-morpholino-4-oxo-2-(pyrazine-2-carboxamido)butanoic acid [Step 2]: To a stirred solution of benzyl (R)-4-morpholino-4-oxo-2-(pyrazine-2-carboxamido)butanoate (47-3, 225 mg, 0.56 mmol) in THF (3 mL) was added LiOH·H2O (47 mg, 1.13 mmol) in water (1.5 mL) at 0° C. The reaction mixture was stirred at ambient temperature for 2 h. The reaction mixture was neutralized with 1N HCl (pH: 5-6) and lyophilized to afford (R)-4-morpholino-4-oxo-2-(pyrazine-2-carboxamido)butanoic acid (47-4, 174 mg). [M+H]⁺=309.1.

Synthesis of (R)-3-((4-methoxybenzyl)oxy)-1-((3aS,4S,6S,7aR)-3a,5,5-trimethylhexahydro-4,6-methanobenzo[d][1,3,2]dioxaborol-2-yl)propan-1-amine hydrochloride [Step 3]: To a solution of (R)—N—((R)-3-((4-methoxybenzyl)oxy)-1-((3aS,4S,6S,7aR)-3a,5,5-trimethylhexahydro-4,6-methanobenzo[d][1,3,2]dioxaborol-2-yl)propyl)-2-methylpropane-2-sulfinamide (47-5, 408 mg, 0.85 mmol) in 1,4-dioxane (5 mL) and methanol (0.34 mL) was added 4M HCl in 1,4-dioxane (0.2 mL, 0.855 mmol) and stirred at −15° C. for 2 h. Volatiles were removed under reduced pressure. The residue was washed with diethyl ether and lyophilized to afford ((R)-3-((4-methoxybenzyl)oxy)-1-((3aS,4S,6S,7aR)-3a,5,5-trimethylhexahydro-4,6-methanobenzo[d][1,3,2]dioxaborol-2-yl)propan-1-amine hydrochloride (47-6, 350 mg). Analytical data of this compound was not clear. Used the compound directly for forwarding step.

Synthesis of N—((R)-1-(((R)-3-((4-methoxybenzyl)oxy)-1-((3aS,4S,6S,7aR)-3a,5,5-trimethyl hexahydro-4,6-methanobenzo[d][1,3,2]dioxaborol-2-yl)propyl)amino)-4-morpholino-1,4-dioxobutan-2-yl)pyrazine-2-carboxamide [Step 4]: To a stirred solution of ((R)-3-((4-methoxybenzyl)oxy)-1-((3aS,4S,6S,7aR)-3a,5,5-trimethylhexahydro-4,6-methanobenzo[d][1,3,2]dioxaborol-2-yl)propan-1-amine hydrochloride (47-6, 350 mg, 0.85 mmol) and (R)-4-morpholino-4-oxo-2-(pyrazine-2-carboxamido)butanoic acid (47-4, 290 mg, 0.94 mmol) in DMF (10 mL) was added DIPEA (0.37 mL, 2.14 mmol) followed by HATU (389 mg, 1.0 mmol) at 0° C. and stirred at ambient temperature for 2 h. The reaction mixture was diluted with ethyl acetate and washed with water and brine, dried over anhydrous Na₂SO₄ and concentrated under reduced pressure to afford N—((R)-1-(((R)-3-((4-methoxybenzyl)oxy)-1-((3aS,4S,6S,7aR)-3a,5,5-trimethylhexahydro-4,6-methanobenzo[d][1,3,2]dioxaborol-2-yl)propyl)amino)-4-morpholino-1,4-dioxobutan-2-yl)pyrazine-2-carboxamide (47-7, 450 mg). [M−H]⁻=662.6.

Synthesis of N—((R)-1-(((R)-3-hydroxy-1-((3aS,4S,6S,7aR)-3a,5,5-trimethylhexahydro-4,6-methanobenzo[d][1,3,2]dioxaborol-2-yl)propyl)amino)-4-morpholino-1,4-dioxobutan-2-yl)pyrazine-2-carboxamide [Step 5]: To a solution of N—((R)-1-(((R)-3-((4-methoxybenzyl)oxy)-1-((3aS,4S,6S,7aR)-3a,5,5-trimethylhexahydro-4,6-methanobenzo[d][1,3,2]dioxaborol-2-yl) propyl)amino)-4-morpholino-1,4-dioxobutan-2-yl)pyrazine-2-carboxamide (47-7, 480 mg, 0.72 mmol) in 1,4-dioxane (3 mL) and methanol (0.29 mL, 7.2 mmol) was added 4M HCl in 1,4-dioxane (0.90 mL, 3.6 mmol) and stirred for 2 h. Volatiles were removed under reduced pressure. The residue was triturated with diethyl ether and lyophilized to afford N—((R)-1-(((R)-3-hydroxy-1-((3aS,4S,6S,7aR)-3a,5,5-trimethylhexahydro-4,6-methanobenzo[d][1,3,2]dioxaborol-2-yl) propyl)amino)-4-morpholino-1,4-dioxobutan-2-yl)pyrazine-2-carboxamide (47-8, 390 mg). [M−H]⁻=542.3.

Synthesis of N—((R)-1-(((R)-2-hydroxy-1,2-oxaborolan-3-yl)amino)-4-morpholino-1,4-dioxobutan-2-yl)pyrazine-2-carboxamide [Step 6]: To a solution of N—((R)-1-(((R)-3-hydroxy-1-((3aS,4S,6S,7aR)-3a,5,5-trimethylhexahydro-4,6-methanobenzo[d][1,3,2]dioxaborol-2-yl) propyl)amino)-4-morpholino-1,4-dioxobutan-2-yl)pyrazine-2-carboxamide (47-8, 225 mg, 0.41 mmol) in hexane (5 mL) and MeOH (5 mL) were added isobutyl boronic acid (127 mg, 1.24 mmol) and 4M HCl in 1,4-Dioxane (1.0 mL, 4.1 mmol) and stirred at ambient temperature for 16 h. Volatiles were removed under reduced pressure and purified by prep HPLC purification and lyophilized to afford N—((R)-1-(((R)-2-hydroxy-1,2-oxaborolan-3-yl)amino)-4-morpholino-1,4-dioxobutan-2-yl)pyrazine-2-carboxamide (Compound 47, 10 mg). [M−H]⁻=390.2. ¹H NMR (400 MHz, CD₃OD), δ 9.27 (s, 1H), 8.81 (s, 1H), 8.69 (s, 1H), 5.32 (brs, 1H), 3.76-3.65 (m, 5H), 3.57-3.47 (m, 5H), 3.40-3.36 (m, 1H), 3.01-2.95 (m, 1H), 2.85-2.83 (m, 1H), 1.84-1.74 (m, 2H).

### Example 48

### Synthesis of ((R)-1-((R)-4-morpholino-4-oxo-2-((R)-tetrahydro-2H-pyran-2-carboxamido) butanamido)-4-phenylbutyl)boronic acid

In another embodiment, Compound 48 can be produced from compound (48-1/1-7) by the method shown in Scheme 48.

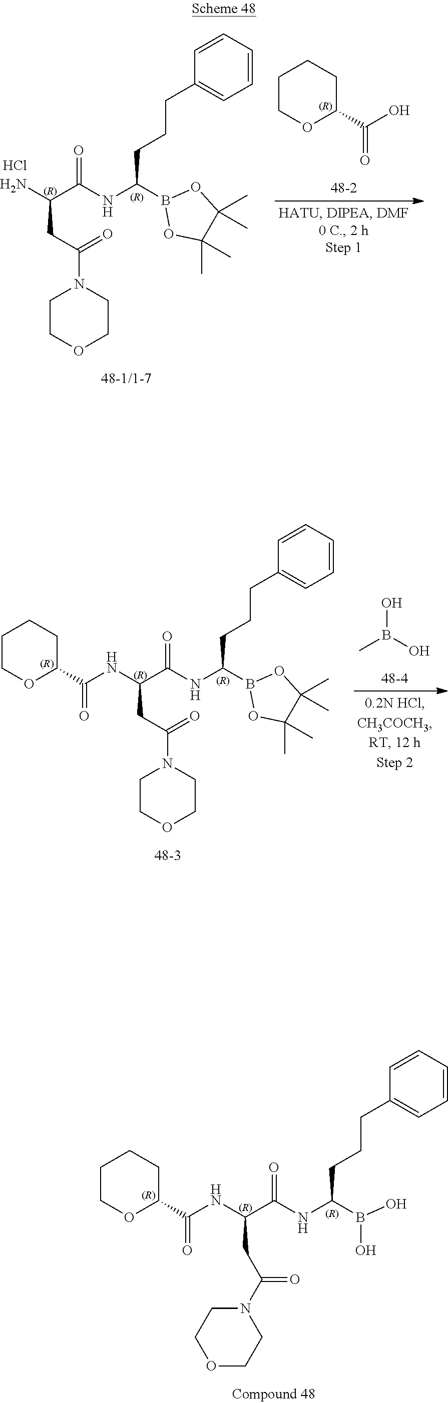

Synthesis of (R)—N—((R)-4-morpholino-1,4-dioxo-1-(((R)-4-phenyl-1-(4,4,5,5-tetramethyl-1,3,2-dioxaborolan-2-yl)butyl)amino)butan-2-yl)tetrahydro-2H-pyran-2-carboxamide [Step 1]: (R)-2-amino-4-morpholino-4-oxo-N—((R)-4-phenyl-1-(4,4,5,5-tetramethyl-1,3,2-dioxaborolan-2-yl)butyl)butanamide hydrochloride (48-1/1-7, 293 mg, 0.6 mmol), (R)-tetrahydro-2H-pyran-2-carboxylic acid (48-2, 70 mg, 0.5 mmol) and O-(7-azabenzotriazol-1-yl)-N,N,N′,N′-tetramethyluronium hexafluorophosphate, HATU (245 mg, 0.6 mmol) were suspended in dry DMF (2 mL) and cooled to 0° C. N,N-diisopropylethylamine (0.3 mL, 1.6 mmol) was added to the stirred solution and stirred at ambient temperature for 2 h. The reaction mixture was diluted with ethyl acetate and washed with water (thrice) and brine. The organic extract was dried over anhydrous Na₂SO₄ and concentrated under reduced pressure. The crude product was purified by prep HPLC purification to afford (R)—N—((R)-4-morpholino-1,4-dioxo-1-(((R)-4-phenyl-1-(4,4,5,5-tetramethyl-1,3,2-dioxaborolan-2-yl)butyl)amino)butan-2-yl)tetrahydro-2H-pyran-2-carboxamide (48-3, 45 mg). [M−H]⁻: 570.4, [M-83]⁻=488.5.

Synthesis of ((R)-1-((R)-4-morpholino-4-oxo-2-((R)-tetrahydro-2H-pyran-2-carboxamido) butanamido)-4-phenylbutyl)boronic acid [Step 2]: To a stirred solution of (R)—N—((R)-4-morpholino-1,4-dioxo-1-(((R)-4-phenyl-1-(4,4,5,5-tetramethyl-1,3,2-dioxaborolan-2-yl)butyl) amino)butan-2-yl)tetrahydro-2H-pyran-2-carboxamide (48-3, 50 mg, 0.1 mmol) and methylboronic acid (48-4, 52 mg, 0.9 mmol) in acetone (4 mL) was added 0.2N HCl (4.0 mL) at ice cold condition and stirred at ambient temperature for 16 h. Volatiles were evaporated under reduced pressure and crude product was purified by prep HPLC purification and lyophilized to afford ((R)-1-((R)-4-morpholino-4-oxo-2-((R)-tetrahydro-2H-pyran-2-carboxamido)butanamido)-4-phenylbutyl)boronic acid (Compound 48, 25 mg). [M−H]⁻: 488.3; ¹H NMR (400 MHz, CD3OD) δ_{H}: 7.24-7.10 (m, 5H), 5.05-5.03 (m, 1H), 4.04 (d, 1H), 3.87-3.84 (m, 1H), 3.66-3.57 (m, 4H), 3.52-3.46 (m, 4H), 3.15-3.09 (m, 1H), 2.90-2.84 (m, 2H), 2.65-2.57 (m, 3H), 2.00-1.89 (m, 2H), 1.67-1.35 (m, 8H).

### Example 49

### Synthesis of ((R)-1-((R)-4-morpholino-4-oxo-2-((S)-tetrahydro-2H-pyran-2-carboxamido) butanamido)-4-phenylbutyl)boronic acid

In another embodiment, Compound 49 can be produced from compound (49-1/1-7) by the method shown in Scheme 49.

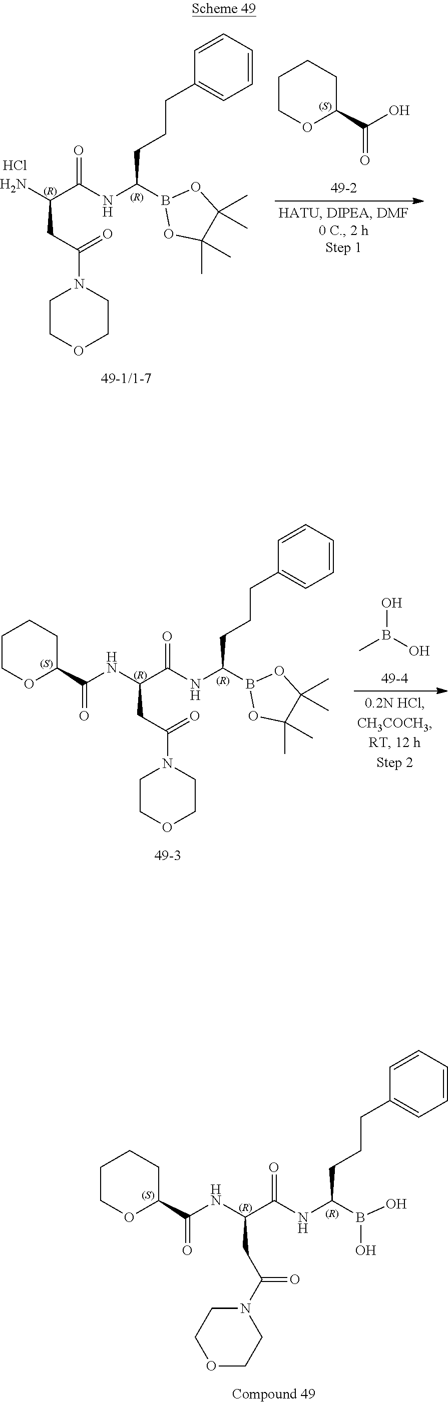

Synthesis of (S)—N—((R)-4-morpholino-1,4-dioxo-1-(((R)-4-phenyl-1-(4,4,5,5-tetramethyl-1,3,2-dioxaborolan-2-yl)butyl)amino)butan-2-yl)tetrahydro-2H-pyran-2-carboxamide [Step 1]: (S)-tetrahydro-2H-pyran-2-carboxylic acid (49-2, 92 mg, 0.7 mmol), (R)-2-amino-4-morpholino-4-oxo-N—((R)-4-phenyl-1-(4,4,5,5-tetramethyl-1,3,2-dioxaborolan-2-yl)butyl) butanamide hydrochloride (49-1/1-7, 386 mg, 0.8 mmol) and O-(7-azabenzotriazol-1-yl)-N,N,N′,N′-tetramethyluronium hexafluorophosphate, HATU (70 mg, 0.2 mmol) were suspended in DMF (2 mL) and cooled to 0° C. N,N-Diisopropylethylamine (0.1 mL, 0.5 mmol) was added to the stirred solution and stirred at ambient temperature for 2 h. The reaction mixture was diluted with ethyl acetate (100 mL), washed with water (thrice) and brine. The organic extract was dried over anhydrous Na₂SO₄ and concentrated under reduced pressure. The crude product was purified by prep HPLC purification to afford (S)—N—((R)-4-morpholino-1,4-dioxo-1-(((R)-4-phenyl-1-(4,4,5,5-tetramethyl-1,3,2-dioxaborolan-2-yl)butyl)amino)butan-2-yl)tetrahydro-2H-pyran-2-carboxamide (49-3, 75 mg). [M+H]⁺: 572.4.

Synthesis of ((R)-1-((R)-4-morpholino-4-oxo-2-((S)-tetrahydro-2H-pyran-2-carboxamido) butanamido)-4-phenylbutyl)boronic acid [Step 2]: To a stirred solution of (S)—N—((R)-4-morpholino-1,4-dioxo-1-(((R)-4-phenyl-1-(4,4,5,5-tetramethyl-1,3,2-dioxaborolan-2-yl)butyl) amino)butan-2-yl)tetrahydro-2H-pyran-2-carboxamide (49-3, 75 mg, 0.13 mmol) and methylboronic acid (49-4, 79 mg, 1.3 mmol) in acetone (6 mL) was added 0.2N HCl (6 mL) at ice cold condition and stirred at ambient temperature for 16 h. Volatiles were evaporated under reduced pressure and the crude product was purified by prep HPLC purification and lyophilized to afford ((R)-1-((R)-4-morpholino-4-oxo-2-((S)-tetrahydro-2H-pyran-2-carboxamido)butanamido)-4-phenylbutyl)boronic acid (Compound 49, 25 mg). [M−H]⁻: 488.6; ¹H NMR (400 MHz, CD3OD) δ_{H}: 7.24-7.10 (m, 5H), 5.03 (t, 1H), 4.04 (d, 1H), 3.82-3.79 (m, 1H), 3.66-3.59 (m, 4H), 3.50-3.46 (m, 4H), 3.12-3.02 (m, 1H), 2.93-2.87 (m, 1H), 2.65-2.58 (m, 3H), 1.94-1.88 (m, 2H), 1.69-1.65 (m, 2H), 1.64-1.46 (m, 6H).

### Example 50

### General Procedures for the Preparation of Compounds:

The following general procedures are provided to prepare compounds that are prepared using similar reaction conditions.

General Procedure A: Amide Formation from an Amine and a Carboxylic Acid

To a stirred solution of a carboxylic acid containing compound in tetrahydrofuran (THF) was added isobutyl chloroformate (IBCF, 1 equivalent) and 4-methylmorpholine (NMM, 1 equivalent) at −15° C. The reaction mixture was stirred at the same temperature for about 30 minutes. The corresponding amine (0.9-1.1 equivalents) in dimethylformamide (DMF) was added followed by NMM (0.9-1.1 equivalents) at −15° C. The reaction mixture was gradually warmed to 0° C. and stirred for about 2 hours. The resulting product was neutralized with aqueous 0.1 N HCl solution and extracted several times with ethyl acetate. The organic layers were combined and washed with a solution of 5% potassium carbonate, water, brine, and dried over anhydrous sodium sulfate.

The mixture was filtered, concentrated under reduced pressure, and purified by silica gel column chromatography to afford the corresponding amide product.

The following compounds described in the above reactions schemes were prepared using General Procedure A for amide formation using an amine and a carboxylic acid: 1-3, 1-9, 2-2, 2-8, 3-6, 4-6, 5-6, 6-6, 7-3, 7-9, 8-3, 8-9, 10-3, 10-9, 15-3, 15-9, 18-6, 21-6, 26-3, 26-9, 27-6.

Compound (3-6): N—((R)-1-(((R)-3-methyl-1-((3aS,4S,6S,7aR)-3a,5,5-trimethylhexahydro-4,6-methanobenzo[d][1,3,2]dioxaborol-2-yl)butyl)amino)-4-morpholino-1,4-dioxobutan-2-yl)pyrazine-2-carboxamide: [M−H]⁻=554.5.

Compound (4-6): N—((R)-4-morpholino-1,4-dioxo-1-(((R)-3-phenyl-1-(4,4,5,5-tetramethyl-1,3,2-dioxaborolan-2-yl)propyl)amino)butan-2-yl)pyrazine-2-carboxamide: [M−H]⁻=550.4.

Compound (5-6): N—((R)-4-morpholino-1,4-dioxo-1-(((R)-1-((3aS,4S,6S,7aR)-3a,5,5-trimethyl hexahydro-4,6-methanobenzo[d][1,3,2]dioxaborol-2-yl)butyl)amino)butan-2-yl)pyrazine-2-carboxamide: [M−H]⁻=540.4.

Compound (6-6): 2,4-dimethyl-N—((R)-1-(((R)-3-methyl-1-((3aS,4S,6S,7aR)-3a,5,5-trimethyl hexahydro-4,6-methanobenzo[d][1,3,2]dioxaborol-2-yl)butyl)amino)-4-morpholino-1,4-dioxobutan-2-yl)oxazole-5-carboxamide: [M−H]⁺=571.4.

Compound (7-3): Benzyl N²-(tert-butoxycarbonyl)-N⁴,N⁴-dimethyl-D-asparaginate: [M+H]⁺350.8. ¹H NMR (400 MHz, DMSO-d₆) (400 MHz, DMSO-d₆) δ 7.52-7.17 (m, 5H), 5.10 (s, 2H), 4.56-4.36 (m, 1H), 2.91 (s, 3H), 2.80 (s, 3H), 2.79-2.64 (m, 2H), 1.36 (s, 9H), 1.32-1.26 (m, 1H).

Compound (7-9): (R)—N⁴,N⁴-dimethyl-N¹—((R)-4-phenyl-1-(4,4,5,5-tetramethyl-1,3,2-dioxaborolan-2-yl)butyl)-2-(pyrazine-2-carboxamido)succinimide: [M−H]⁺=522.5.

Compound (8-3): Benzyl N²-(tert-butoxycarbonyl)-N⁴-ethyl-D-asparaginate: [M+H]⁺=350.8. 1H NMR (400 MHz, DMSO-d₆) δ 7.86 (s, 1H), 7.34 (s, 5H), 7.14 (d, 1H), 5.09 (s, 2H), 4.40 (d, 1H), 3.15-2.92 (m, 2H), 2.65-2.51 (m, 1H), 2.48-2.34 (m, 1H), 1.45-1.22 (m, 9H), 0.97 (t, 3H).

Compound (8-9): (R)—N⁴-ethyl-N¹—((R)-4-phenyl-1-(4,4,5,5-tetramethyl-1,3,2-dioxaborolan-2-yl)butyl)-2-(pyrazine-2-carboxamido)succinimide: [M−H]⁺=522.6.

Compound (10-3): Benzyl (R)-2-((tert-butoxycarbonyl)amino)-4-oxo-4-(piperidin-1-yl)butanoate: [M+H]⁺=390.8.

Compound (10-9): N—((R)-1,4-dioxo-1-(((R)-4-phenyl-1-(4,4,5,5-tetramethyl-1,3,2-dioxaborolan-2-yl)butyl)amino)-4-(piperidin-1-yl)butan-2-yl)pyrazine-2-carboxamide: [M−H]⁺=562.6.

Compound (15-3): (R)-2-((tert-butoxycarbonyl)amino)-5-morpholino-5-oxopentanoate: 1H NMR (400 MHz, DMSO-D₆) δ 7.36-7.31 (m, 6H), 5.17-5.05 (m, 2H), 4.06-4.01 (m, 1H), 3.51-3.50 (m, 4H), 3.41-3.39 (m, 2H), 2.39-2.28 (m, 2H), 1.95-1.89 (m, 1H), 1.84-1.77 (m, 1H), 1.37 (s, 9H).

Compound (15-9): N—((R)-5-morpholino-1,5-dioxo-1-(((R)-4-phenyl-1-(4,4,5,5-tetramethyl-1,3,2-dioxaborolan-2-yl)butyl)amino)pentan-2-yl)pyrazine-2-carboxamide: [M−H]⁺=578.

Compound (18-6): N—((R)-4-morpholino-1,4-dioxo-1-(((R)-1-(4,4,5,5-tetramethyl-1,3,2-dioxaborolan-2-yl)pentyl) amino)butan-2-yl)pyrazine-2-carboxamide: [M−H]⁻=502.4.

Compound (21-6): N—((R)-4-morpholino-1,4-dioxo-1-(((S)-4-phenyl-1-(4,4,5,5-tetramethyl-1,3,2-dioxaborolan-2-yl)butyl)amino)butan-2-yl)pyrazine-2-carboxamide: [M−H]⁺=564.4.

Compound (26-3): Benzyl (S)-2-((tert-butoxycarbonyl)amino)-4-morpholino-4-oxobutanoate: [M+H]⁺=393.0.

Compound (26-9): N—((S)-4-morpholino-1,4-dioxo-1-(((R)-4-phenyl-1-(4,4,5,5-tetramethyl-1,3,2-dioxaborolan-2-yl)butyl)amino)butan-2-yl)pyrazine-2-carboxamide: [M−H]⁺=564.4.

Compound (27-6): N—((S)-4-morpholino-1,4-dioxo-1-(((S)-4-phenyl-1-(4,4,5,5-tetramethyl-1,3,2-dioxaborolan-2-yl)butyl)amino)butan-2-yl)pyrazine-2-carboxamide: [M−H]⁺=564.3.

General Procedure B: Amide Formation from an Amine and an Acid Chloride, Anhydride, or Sulfonyl Chloride

To a stirred solution of the amine compound 0.6 mmol) and acetic anhydride (1.1 equivalents) in dichloromethane was added an ice cooled solution of diisopropylethylamine (DIPEA) (5 equivalents) and the reaction mixture was stirred at room temperature for about 2 hours. Thin layer chromatography showed complete disappearance of the starting material. The reaction mixture was diluted with dichloromethane (DCM) and washed with water and brine solution. The organic phase was dried over anhydrous sodium sulfate, filtered, and concentrated under reduced pressure. The product was purified by reverse-phase prep-HPLC and lyophilized to afford the desired amide product.

In General Procedure B, acetic anhydride can be replaced with an acyl chloride (e.g., morpholine-4-carbonyl chloride) or a sulfonyl chloride (e.g., benzenesulfonyl chloride), and the DIPEA can be replaced with another base (e.g., N-methylmorpholine, NMM).

The following compounds described in the above reactions schemes were prepared using General Procedure B for amide formation using an amine and an acid chloride, anhydride, or sulfonyl chloride: 11-3, 12-3, 19-6, 20-3, 22-3, 23-3, 24-3, 25-3.

Compound (11-3): (R)-2-acetamido-4-morpholino-4-oxo-N—((R)-4-phenyl-1-(4,4,5,5-tetramethyl-1,3,2-dioxaborolan-2-yl)butyl)butanamide: [M−H]⁺=500.1.

Compound (12-3): N—((R)-4-morpholino-1,4-dioxo-1-(((R)-4-phenyl-1-(4,4,5,5-tetramethyl-1,3,2-dioxaborolan-2-yl)butyl)amino)butan-2-yl)morpholine-4-carboxamide: [M−H]⁺=571.5. ¹H NMR (400 MHz, MeOD) δ 7.24-7.09 (m, 5H), 4.96-4.92 (m, 1H), 3.65-3.59 (m, 8H), 3.51-3.49 (m, 4H), 3.40-3.32 (m, 4H), 3.01-2.87 (m, 2H), 2.62-2.57 (m, 3H), 1.70-1.48 (m, 4H), 1.19-1.14 (m, 5H).

Compound (19-6): N—((R)-4-morpholino-1,4-dioxo-1-(((R)-1-(4,4,5,5-tetramethyl-1,3,2-dioxaborolan-2-yl)propyl)amino)butan-2-yl)pyrazine-2-carboxamide: [M−H]⁻=474.3.

Compound (20-3): (R)-2-(3,3-dimethylureido)-4-morpholino-4-oxo-N—((R)-4-phenyl-1-(4,4,5,5-tetramethyl-1,3,2-dioxaborolan-2-yl)butyl)butanamide: [M−H]⁺=529.5.

Compound (22-3): N—((R)-4-morpholino-1,4-dioxo-1-(((R)-4-phenyl-1-(4,4,5,5-tetramethyl-1,3,2-dioxaborolan-2-yl)butyl)amino)butan-2-yl)cyclohexanecarboxamide: [M−H]⁺=568.7.

Compound (23-3): N—((R)-4-morpholino-1,4-dioxo-1-(((R)-4-phenyl-1-(4,4,5,5-tetramethyl-1,3,2-dioxaborolan-2-yl)butyl)amino)butan-2-yl)tetrahydro-2H-pyran-4-carboxamide: [M−H]⁺=570.4.

Compound (24-3): (R)-4-morpholino-4-oxo-N—((R)-4-phenyl-1-(4,4,5,5-tetramethyl-1,3,2-dioxaborolan-2-yl)butyl)-2-(phenylsulfonamido)butanamide: [M−H]⁺=598.

Compound (25-3): N—((R)-4-morpholino-1,4-dioxo-1-(((R)-4-phenyl-1-(4,4,5,5-tetramethyl-1,3,2-dioxaborolan-2-yl)butyl)amino)butan-2-yl)tetrahydro-2H-pyran-2-carboxamide: [M−H]⁺=570.5.

### General Procedure C: Hydrogenolysis of Benzyl Esters

To a stirred solution of the benzyl ester containing compound in tetrahydrofuran (THF) was added nitrogen gas for 10 minutes. Then 10% Pd—C (0.7 equivalents) was added and the reaction mixture was hydrogenated under a hydrogen balloon from 3 to 12 hours and monitored by thin layer chromatography. The reaction mixture was filtered over celite using excess ethyl acetate. The solvents were removed by concentration under reduced pressure to afford the corresponding carboxylic acid product.

The following compounds described in the above reaction schemes were prepared using General Procedure C for hydrogenolysis of benzyl esters: 1-4, 2-3, 3-1, 5-1, 6-1, 7-4, 8-4, 10-4, 15-4, 18-1, 19-1, 26-4, 27-1.

Compound (7-4): N²-(tert-butoxycarbonyl)-N⁴,N⁴-dimethyl-D-asparagine: [M+H]⁺=260.8. ¹H NMR (400 MHz, DMSO-d₆) δ 12.48 (s, 1H), 6.70 (d, 1H), 4.31 (s, 1H), 3.97-3.69 (m, 2H), 2.93 (s, 3H), 2.80 (s, 3H), 1.38 (s, 9H).

Compound (8-4): N²-(tert-butoxycarbonyl)-N⁴-ethyl-D-asparagine: [M+H]⁺=260.8. ¹H NMR (400 MHz, DMSO-d₆) δ 12.52 (s, 1H), 7.89-7.76 (m, 1H), 6.89 (d, 1H), 4.26 (q, 1H), 3.10-2.98 (m, 2H), 2.50-2.35 (m, 2H), 1.37 (s, 9H), 0.99 (t, 3H).

Compound (10-4): (R)-2-((tert-butoxycarbonyl)amino)-4-oxo-4-(piperidin-1-yl)butanoic acid: [M+H]⁺=301.3.

Compound (15-4): (R)-2-((tert-butoxycarbonyl)amino)-5-morpholino-5-oxopentanoic acid: ¹H NMR (400 MHz, DMSO-Ds) δ 12.5 (br s, 1H), 7.08 (d, 1H), 3.93-3.88 (m, 1H), 3.55-3.51 (m, 4H), 3.41 (d, 4H), 2.40-2.28 (m, 2H), 1.99-1.88 (m, 1H), 1.80-1.73 (m, 1H), 1.38 (s, 9H).

Compound (26-4): 47-4: (S)-2-((tert-butoxycarbonyl)amino)-4-morpholino-4-oxobutanoic acid: [M+H]⁺=301.2.

General Procedure D: Amide Formation with a Protected Boronic Acid

To a stirred solution of a carboxylic acid containing compound in tetrahydrofuran was added isobutyl chloroformate (IBCF) (1 equivalent) and N-methylmorpholine (NMM) (1 equivalent) at −15° C. The reaction mixture was stirred at the same temperature for about 30 minutes. Then the protected boronic acid compound bearing an amine group (1 equivalent) in dimethylformamide was added to the reaction mixture followed by NMM (1 equivalent) at −15° C. The reaction mixture was gradually warmed to 0° C. and stirred for about 2 hours. LCMS of the reaction mass confirmed the formation of the desired product, and the reaction mixture was neutralized with an aqueous solution of 0.1 N HCl and extracted with ethyl acetate. The organic layers were combined and washed with 5% potassium carbonate solution, water, brine, dried over sodium sulfate, filtered, and concentrated under reduced pressure to afford the coupled product.

The following compounds described in the above reaction schemes were prepared using General Procedure D for amide formation with a protected boronic acid: 1-6, 2-5, 3-3, 4-3, 5-3, 6-3, 7-6, 8-6, 10-6, 13-1, 15-6, 18-3, 19-3, 21-3, 26-6, 27-3.

Compound (3-3): Tert-butyl ((R)-1-(((R)-3-methyl-1-((3aS,4S,6S,7aR)-3a,5,5-trimethyl hexahydro-4,6-methanobenzo[d][1,3,2]dioxaborol-2-yl)butyl)amino)-4-morpholino-1,4-dioxobutan-2-yl)carbamate: [M+H]⁺=550.2.

Compound (4-3): ((R)-4-morpholino-1,4-dioxo-1-(((R)-3-phenyl-1-(4,4,5,5-tetramethyl-1,3,2-dioxaborolan-2-yl)propyl)amino)butan-2-yl)carbamate: [M−H]⁻=544.4.

Compound (5-3): Tert-butyl ((R)-4-morpholino-1,4-dioxo-1-(((R)-1-((3aS,4S,6S,7aR)-3a,5,5-trimethylhexahydro-4,6-methanobenzo[d][1,3,2]dioxaborol-2-yl)butyl)amino)butan-2-yl) carbamate: [M−H]⁻=534.5.

Compound (6-3): Tert-butyl ((R)-1-(((R)-3-methyl-1-((3aS,4S,6S,7aR)-3a,5,5-trimethyl hexahydro-4,6-methanobenzo[d][1,3,2]dioxaborol-2-yl)butyl)amino)-4-morpholino-1,4-dioxobutan-2-yl)carbamate: [M−H]⁺: 548.

Compound (7-6): Tert-butyl ((R)-4-(dimethylamino)-1,4-dioxo-1-(((R)-4-phenyl-1-(4,4,5,5-tetramethyl-1,3,2-dioxaborolan-2-yl)butyl)amino)butan-2-yl)carbamate: [M+H]⁺=518.1.

Compound (8-6): Tert-butyl ((R)-4-(ethylamino)-1,4-dioxo-1-(((R)-4-phenyl-1-(4,4,5,5-tetramethyl-1,3,2-dioxaborolan-2-yl)butyl)amino)butan-2-yl)carbamate: [M+H]: 518.1.

Compound (10-6): Tert-butyl ((R)-1,4-dioxo-1-(((R)-4-phenyl-1-(4,4,5,5-tetramethyl-1,3,2-dioxaborolan-2-yl)butyl)amino)-4-(piperidin-1-yl)butan-2-yl)carbamate: [M+H]⁺=558.0.

Compound (15-6): Tert-butyl ((R)-5-morpholino-1,5-dioxo-1-(((R)-4-phenyl-1-(4,4,5,5-tetramethyl-1,3,2-dioxaborolan-2-yl)butyl)amino)pentan-2-yl)carbamate: [M−H]⁺=572.

Compound (18-3): Tert-butyl ((R)-4-morpholino-1,4-dioxo-1-(((R)-1-(4,4,5,5-tetramethyl-1,3,2-dioxaborolan-2-yl)pentyl)amino)butan-2-yl)carbamate: [M+H]⁺=498.1.

Compound (19-3): Tert-butyl ((R)-4-morpholino-1,4-dioxo-1-(((R)-1-(4,4,5,5-tetramethyl-1, 3,2-dioxaborolan-2-yl)propyl)amino)butan-2-yl)carbamate: [M−H]⁻=468.4.

Compound (21-3): Tert-butyl ((R)-4-morpholino-1,4-dioxo-1-(((S)-4-phenyl-1-(4,4,5,5-tetramethyl-1,3,2-dioxaborolan-2-yl)butyl)amino)butan-2-yl)carbamate: [M+H]⁺: 560.1.

Compound (26-6): Tert-butyl ((S)-4-morpholino-1,4-dioxo-1-(((R)-4-phenyl-1-(4,4,5,5-tetramethyl-1,3,2-dioxaborolan-2-yl)butyl)amino)butan-2-yl)carbamate: [M+H]⁺=558.4.

Compound (27-3): Tert-butyl ((S)-4-morpholino-1,4-dioxo-1-(((S)-4-phenyl-1-(4,4,5,5-tetramethyl-1,3,2-dioxaborolan-2-yl)butyl)amino)butan-2-yl)carbamate: [M−H]⁺=558.5.

### General Procedure E. Removal of a BOC Protection Group

To a solution of a BOC-protected compound was added 4 M HCl in dioxane (10 equivalents) at 0° C. The reaction mixture was gradually warmed to ambient temperature and stirred for about 16 hours. Thin layer chromatography showed complete consumption of the starting material and the reaction mixture was concentrated under reduced pressure to obtain the desired product as the hydrochloride salt. The product was used without purification.

The following compounds described in the above reaction schemes were prepared using General Procedure E for removal of a BOC protection group: 1-7, 2-6, 3-4, 4-4, 5-4, 6-4, 7-7, 8-7, 10-7, 11-1, 12-1, 15-7, 18-4, 19-4, 21-4, 26-7, 27-4.

Compound (3-4): (R)-2-amino-N—((R)-3-methyl-1-((3aS,4S,6S,7aR)-3a,5,5-trimethylhexahydro-4,6-methanobenzo[d][1,3,2]dioxaborol-2-yl)butyl)-4-morpholino-4-oxobutanamide hydrochloride: [M+H]⁺=450.2.

Compound (4-4): (R)-2-amino-4-morpholino-4-oxo-N—((R)-3-phenyl-1-(4,4,5,5-tetramethyl-1,3,2-dioxaborolan-2-yl)propyl)butanamide hydrochloride: [M−H]⁻=444.4.

Compound (5-4): (R)-2-amino-4-morpholino-4-oxo-N—((R)-1-((3aS,4S,6S,7aR)-3a,5,5-trimethyl hexahydro-4,6-methanobenzo[d][1,3,2]dioxaborol-2-yl)butyl)butanamide hydrochloride: [M−H]⁻=434.3.

Compound (6-4): (R)-2-amino-N—((R)-3-methyl-1-((3aS,4S,6S,7aR)-3a,5,5-trimethylhexahydro-4,6-methanobenzo[d][1,3,2]dioxaborol-2-yl)butyl)-4-morpholino-4-oxobutanamide: [M−H]⁺=448.6.

Compound (7-7): (R)-2-amino-N⁴,N⁴-dimethyl-N¹—((R)-4-phenyl-1-(4,4,5,5-tetramethyl-1,3,2-dioxaborolan-2-yl)butyl)succinamide hydrochloride: The product was used directly in the next step without further purification or characterization.

Compound (8-7): (R)-2-amino-N⁴-ethyl-N¹—((R)-4-phenyl-1-(4,4,5,5-tetramethyl-1,3,2-dioxaborolan-2-yl)butyl)succinamide hydrochloride: The product was used directly in next step without purification or characterization.

Compound (10-7): (R)-2-amino-4-oxo-N—((R)-4-phenyl-1-(4,4,5,5-tetramethyl-1,3,2-dioxaborolan-2-yl)butyl)-4-(piperidin-1-yl)butanamide hydrochloride: The product was used directly in next step without purification or characterization.

Compound (15-7): (R)-2-amino-5-morpholino-5-oxo-N—((R)-4-phenyl-1-(4,4,5,5-tetramethyl-1,3,2-dioxaborolan-2-yl)butyl)pentanamide: [M−H]⁺=472.

Compound (18-4): (R)-2-amino-4-morpholino-4-oxo-N—((R)-1-(4,4,5,5-tetramethyl-1,3,2-dioxaborolan-2-yl)pentyl)butanamide hydrochloride: [M−H]⁻=396.4.

Compound (19-4): (R)-2-amino-4-morpholino-4-oxo-N—((R)-1-(4,4,5,5-tetramethyl-1,3,2-dioxaborolan-2-yl)propyl)butanamide hydrochloride: [M+H]⁺=370.3 Compound (21-4): (R)-2-amino-4-morpholino-4-oxo-N—((S)-4-phenyl-1-(4,4,5,5-tetramethyl-1,3,2-dioxaborolan-2-yl)butyl)butanamide hydrochloride: The product was directly used in the next step without purification or characterization.

Compound (26-7): (S)-2-amino-4-morpholino-4-oxo-N—((R)-4-phenyl-1-(4,4,5,5-tetramethyl-1,3,2-dioxaborolan-2-yl)butyl)butanamide hydrochloride: [M+H]⁺=458.4.

Compound (27-4): (S)-2-amino-4-morpholino-4-oxo-N—((S)-4-phenyl-1-(4,4,5,5-tetramethyl-1,3,2-dioxaborolan-2-yl)butyl)butanamide hydrochloride: [M+H]⁺=458.4.

### General Procedure F: Hydrolysis of a Boronate Ester

To a stirred solution of the boronate ester and methylboronic acid (8 equivalents) in acetone was added an equivalent volume of 0.2 N HCl and the reaction mixture was stirred at ambient temperature overnight. Thin layer chromatography showed the complete disappearance of the starting material and the reaction mixture was concentrated under reduced pressure. The product was redissolved in a mixture of acetone and deionized water, and lyophilized to obtain the boronic acid product.

### General Procedure G: Oxidative Removal of a Boronate Ester

To a stirred solution of a boronate ester in a mixture of (1:1) acetone and water was added ammonium acetate (1 equivalent) and the reaction mixture was stirred for 5 minutes. Sodium periodate (NaIO₄) (1 equivalent) was added portion-wise and reaction mixture was stirred for 3 hours. The reaction mixture was concentrated under reduced pressure and partitioned between ethyl acetate and water. The organic layer was collected and the aqueous layer was further extracted with ethyl acetate (twice). The combined organic layers were dried over anhydrous sodium sulfate and concentrated under reduced pressure. The product was purified through reverse-phase prep-HPLC to afford the desired boronic acid product.

### Example 51—Biological/Biochemical Evaluation

### General Protocol for In Vitro Analysis of Compounds:

The inhibitory activity of the compounds of the present invention against LONP1, 20S proteasome and other proteases are determined by assays known to persons of ordinary skill in the art (see, e.g., Fishovitz, J. et al. “Active-Site-Directed Chemical Tools for Profiling Mitochondrial Lon Protease” ACS Chem. Biol. 6, 781-788 (2011)).

In this Example, LONP1 (NM_004793.4) activity was measured by a FRET-based assay for protease activity using a fluorogenic peptide DabcylYRGIT(2Abu)SGRQK(5-FAM) (Cambridge Research Biochemicals) as substrate. LONP1 activity is followed by an increase in fluorescence signal due to the degradation of the peptide. Inhibition of LONP1 protease activity by an inhibitor compound of the disclosure elicits a decrease in the fluorescent signal.

The assay is performed in a 384-well plate (Greiner, cat. #781076) using the following reagents and conditions: substrate (3 μM) was incubated for 1 hour at 37° C. in the presence of LONP1 (15 nM as monomer), 25 mM Tris pH 8.0, 10 mM MgCl₂, 0.03 mg/mL BSA, 0.5 mM DTT, 0.0003% Tween-20, 10 mM NaCl, 0.06 mM ATP and 0.5 mM EGTA in a 15 μL final volume. The LONP1-containing mix (10 μL) was incubated with the test compound for 15 min at 37° C. before adding the peptide-containing mix (5 μL). Solutions were dispensed using a small cassette-Multidrop Combi (Thermo Scientific). Fluorescence was measured using a PheraStar plate reader (BMG Labtech) FI-FRET EX 485 nm Em 520 nm.

The IC₅₀ values for binding to LONP1 are summarized in Table 2 below. Each value is based on an average of a minimum of two repeats.

TABLE 2
IC50 assay data for compounds of
the disclosure binding to LONP1;

| Compound No. | Activity IC₅₀ (μM) |
|---|---|
| 1 | A |
| 2 | A |
| 3 | B |
| 4 | B |
| 5 | B |
| 6 | B |
| 7 | A |
| 8 | A |
| 9 | A |
| 10 | A |
| 11 | B |
| 12 | A |
| 13 | A |
| 14 | B |
| 15 | A |
| 16 | — |
| 17 | — |
| 18 | B |
| 19 | B |
| 20 | B |
| 21 | C |
| 22 | A |
| 23 | A |
| 24 | A |
| 25 | A |
| 26 | A |
| 27 | B |
| 28 | C |
| 29 | A |
| 30 | B |
| 31 | B |
| 32 | A |
| 33 | A |
| 34 | C |
| 35 | A |
| 36 | A |
| 37 | A |
| 38 | B |
| 39 | B |
| 40 | B |
| 41 | C |
| 42 | B |
| 43 | B |
| 44 | B |
| 45 | B |
| 46 | B |
| 47 | D |
| 48 | B |
| 49 | B |

A: <0.05 μM;
B: 0.05-0.5 μM;
C: 0.5-5 μM;
D >5 μM

In one embodiment, beneficial compounds of this disclosure have IC₅₀ values of less than 5 μM. In another embodiment, beneficial compounds of this disclosure have IC₅₀ less than 2.5 μM. In another embodiment, beneficial compounds of this disclosure have IC₅₀ less than 1 μM. In another embodiment, beneficial compounds of this disclosure have IC₅₀ less than 0.5 μM. In another embodiment, beneficial compounds of this disclosure have IC₅₀ less than 0.1 μM. In another embodiment, beneficial compounds of this disclosure have IC₅₀ less than 0.05 μM. In another embodiment, beneficial compounds of this disclosure have IC₅₀ less than 0.01 μM.

### Cell Viability Assay:

Materials and kits:
Cell Proliferation Kit I (MTT), Merck, Cat #11465007001
DMEM GlutaMax, Thermo Fisher Scientific, Cat #31966021—for expansion and assay
DMEM GlutaMax, low glucose, Thermo Fisher Scientific, Cat #21885025—for expansion for cell viability assay
FBS, Gibco, Cat #A3840402

### Assay Procedure:

One day before treatment, 3,000-5,000/mL of 143b cells are placed in aliquots of 100 μL per well in flat bottom ThermoFisher 96 well plate. The starting seeding number is optimised in relation to the batch of cells and medium. The assay lasts for 8 days from seeding to MTT assay, and so the seeding number must be selected to avoid over-confluency at the last day of the assay.

On day 0, 100 μl of medium (Cat #21885025) is transferred to compound/DMSO plate, then the compound/DMSO-containing medium is transferred to the plates with pre-seeded cells.

Incubate for 7 days at 37° C., 5% CO₂ incubator.

On day 7, the medium is discarded. 100 μl of MTT labelling reagent mixed 1:10 in culture medium (Cat #21885025) is added and incubated for 4 hours at 37° C., 5% CO₂ incubator. 100 μl of MTT solubilization solution is added, mixed well and incubated overnight at 37° C.

Absorbance is measured at 570 nm on a plate reader.

### Compound Plate Setup:

The compounds are dispensed in a 96-well Greiner plate (cat no. 651201).
Volume of each compound solution: 200 nL
Final conc of DMSO: 0.1% in all wells
Starting concentration: 10 mM (final concentration on assay plate: 10 μM). In total, 8 doses and three replicates per dose per compound.
Dilution factor: 3.162

The compounds are dissolved in DMSO and dispensed according to the concentration titrations and experimental design (indicated above).

Two plates of the same compounds are dispensed and the remaining plates are retained as a backup.

Compounds can be dispensed in an Echo dispenser and sealed immediately so that they are not exposed to air and contamination. The protocol is performed under the LAF bench.

On the first day of treatment (Day 0), compound plates are opened under the LAF bench. 100 μl of assay medium (Cat #21885025) is added to each well and 100.2 μl of medium+compound/DMSO is transferred to the assay plates containing pre-seeded cells.

1. A compound of structural Formula 1: 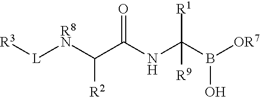
or a pharmaceutically acceptable salt, solvate, stereoisomer or mixture of stereoisomers, tautomer, isotopic form, pharmaceutically active metabolite thereof, or combinations thereof, wherein:
R¹ is selected from the group consisting of: deuterium, C1-C4 alkyl, C1-C4 alkoxyl, C₁-C₄ oxoalkyl, C1-C5 alkyl-alkoxyl, wherein each alkyl, oxoalkyl or alkoxyl is optionally substituted with C3-C6 cycloalkyl, phenyl, phenoxy, or a 5- or 6-membered heteroaryl, wherein said phenyl, phenoxy, or heteroaryl are each optionally substituted with one or more substituent selected from deuterium, halogen, hydroxyl, CN, CO2H, CO2R¹⁰, CONR¹⁰R¹¹, NR¹⁰R¹¹, SR¹⁰, SO2NR¹⁰R¹¹, C1-C4 alkyl, C1-C4 haloalkyl, C1-C4 alkoxy, phenyl, or a 5- or 6-membered heteroaryl;
R² is (C₁-C₄ alkyl)-C(O)NR⁴R⁵;
R⁴ and R⁵ are each independently selected from hydrogen, deuterium, C1-C4 alkyl; C1-C4 haloalkyl, C1-C5 alkyl-alkoxyl or R⁴ and R⁵ together with the N to which they are attached form 5 or 6 membered heterocyclic ring optionally having one or more additional heteroatoms selected from N, O and S, wherein the 5 or 6 membered heterocyclic ring is optionally substituted with one or more substituent selected from deuterium, halogen, hydroxyl, oxo, CN, C1-C4 alkyl, C1-C4 haloalkyl or C1-C4 alkoxyl;
L is C(O), C(O)O, C(O)NR⁶, S(O)₂, or a bond;
R³ is C₁-C₄ alkyl optionally substituted with one or more substituents each independently selected from the group consisting of deuterium, halogen, cyano, hydroxyl, C₁-C₄ alkoxyl, 5 or 6 membered aryl (e.g. phenyl) or 5 or 6 membered heteroaryl; or
R³ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, oxo, C₁-C₄ alkoxyl, or C₁-C₄ alkyl that is optionally substituted with one to three substituents selected from deuterium, halogen, cyano, hydroxyl, or C₁-C₄ alkoxyl; or
R³ is aryl or heteroaryl having one or more heteroatoms selected from N, O and S, wherein aryl or heteroaryl is optionally substituted with one or more substituents selected from deuterium, halogen, cyano, hydroxyl, OR, CO2H, CO2R¹⁰, CONR¹⁰R¹¹, NR¹⁰R¹¹, SR¹⁰, SO2NR¹⁰R¹¹, C₁-C₄ alkoxyl, or C₁-C₄ alkyl that is optionally substituted with one to three substituents selected from deuterium, halogen, cyano, hydroxyl, or C₁-C₄ alkoxyl;
R⁶ is hydrogen or C₁-C₄ alkyl optionally substituted with one or more of halogen, hydroxyl and phenyl, wherein phenyl is optionally substituted with one or more substituent selected from halogen, hydroxyl and C₁-C₂ alkyl;
R⁷ is hydrogen, or R⁷ and R¹, together with the boron atom to which R⁷ is attached form a 5-membered heteroalkyl ring;
R⁸ is selected from hydrogen, deuterium or C₁-C₂ alkyl optionally substituted with one or more substituents each independently selected from the group consisting of deuterium, halogen, hydroxyl, cyano, methoxyl and phenyl;
R⁹ is selected from hydrogen, deuterium or C₁-C₂ alkyl; and
R¹⁰ and R¹¹ are each independently selected from hydrogen, deuterium, C1-C4 alkyl; C1-C4 haloalkyl, C1-C5 alkyl-alkoxyl, C3-C7 cycloalkyl, or R¹⁰ and R¹¹ together with the N to which they are attached form 3 to 7 membered heterocyclic ring optionally having one or more additional heteroatoms selected from N, O and S, wherein the C3-C7 cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl.

2. A compound according to claim 1, wherein R¹ is selected from methyl, ethyl, n-propyl, i-propyl, n-butyl or tert-butyl, each optionally substituted with a phenyl ring.

3. The compound according to claim 1, wherein R¹ is selected from methyl, n-propyl, n-butyl or tert-butyl.

4. The compound according to claim 1, wherein R¹ is selected from phenyl-(CH₂)— or phenyl-(CH₂)₃—.

5. The compound according to claim 1, wherein R² is —CH₂—C(O)NR⁴R⁵.

6. The compound according to claim 1, wherein one of R⁴ and R⁵ is C1-C2 alkyl, or wherein both of R⁴ and R⁵ are C1-C2 alkyl.

7. The compound according to claim 1, wherein one of R⁴ and R⁵ is methyl, or wherein both of R⁴ and R⁵ are methyl.

8. The compound according to claim 1, wherein one of R⁴ and R⁵ is hydrogen; or wherein both of R⁴ and R⁵ are hydrogen.

9. The compound according to claim 1, wherein one of R⁴ and R⁵ is hydrogen; the other of R⁴ and R⁵ is ethyl.

10. The compound according to claim 1, wherein NR⁴R⁵ is selected from a 5- or 6-membered heterocycloalkyl group having one or two heteroatoms.

11. The compound according to claim 10, wherein NR⁴R⁵ is selected from N-pyrrolidinyl, N-morpholinyl and N-piperidinyl.

12. The compound according to claim 1, wherein L is selected from C(O), C(O)O, C(O)NH, C(O)N(CH₃), SO2.

13. The compound according to claim 1, wherein L is selected from C(O), C(O)O and C(O)NH.

14. The compound according to claim 1, wherein L is C(O).

15. The compound according to claim 1, wherein R³ is C₁-C₄ alkyl, a 5- or 6-membered heteroaryl, C6 aryl, a 5- or 6-membered heterocycloalkyl or C6 cycloalkyl, and wherein R³ is optionally substituted.

16. The compound according to claim 1, wherein R³ is methyl, ethyl, n-propyl, i-propyl, n-butyl or tert-butyl, each optionally substituted with a phenyl ring.

17. The compound according to claim 16, wherein R³ is selected from methyl, i-propyl and tert-butyl.

18. The compound according to claim 16, wherein R³ is selected from phenyl, phenyl-(CH₂)— and phenyl-(CH₂)₂—, wherein the phenyl group is optionally substituted.

19. The compound according to claim 15, wherein R³ is selected from an aryl, heteroaryl, cycloalkyl or heterocycloalkyl selected from tetrahydropyranyl, pyrazinyl, tetrahydropyrrolyl, tetrahydrofuranyl, tetrahydropyranyl, cyclohexanyl, oxazolyl and morpholinyl, wherein said aryl, heteroaryl, cycloalkyl or heterocycloalkyl is optionally substituted.

20. The compound according to claim 19, wherein R³ is selected from n-tetrahydropyrrolyl, n-and morpholinyl.

21. The compound according to claim 18, wherein said substituent is selected from one to three of halogen, hydroxyl and C1-C2 alkyl.

22. The compound according to claim 18, wherein said substituent is selected from one or two of C1, hydroxyl and methyl.

23. The compound according to claim 18, wherein R³ is selected from 2-chlorophenyl, 3-chlorophenyl, 2,5-dichlorophenyl, 2,4-dimethyloxazolyl, and 3-hydroxy n-tetrahydropyrrolyl.

24. The compound according to claim 1, wherein R³ is an 8, 9 or 10 membered bicyclic heteroaryl, aryl, saturated or unsaturated heterocycloalkyl or saturated or unsaturated cycloalkyl, and wherein R³ is optionally substituted.

25. The compound according to claim 24, wherein R³ is selected from indolyl, isoindolyl, indolinyl, isoindolinyl, indolizinyl, benzimidazolyl, purinyl, quinolinyl, isoquinolinyl, quinazolinyl, or pteridinyl.

26. The compound according to claim 24, wherein R³ is isoindolinyl or N-isoindolinyl.

27. The compound according to claim 1, wherein R⁶ is hydrogen or methyl.

28. The compound according to claim 1, wherein R⁷ is hydrogen.

29. The compound according to claim 1, wherein R⁸ is selected from hydrogen, phenyl-(CH₂)— or phenyl-(CH₂)₂—.

30. The compound according to claim 1, wherein R⁹ is hydrogen or C1-C2 alkyl.

31. The compound according to claim 1, wherein R⁹ is hydrogen.

32. The compound according to claim 1, wherein R¹⁰ and R¹¹ are each independently selected from hydrogen, deuterium, C1-C2 alkyl; C1-C2 haloalkyl, C1-C2 alkyl-alkoxyl or C3-C7 cycloalkyl, wherein C3-C7 cycloalkyl is optionally substituted with one or more substituent selected from deuterium, F, C1, hydroxyl, oxo, CN, C1-C2 alkyl, C1-C2 haloalkyl or C1-C2 alkoxyl.

33. The compound according to claim 1, wherein R¹⁰ and R¹¹ together with the N to which they are attached form 3 to 7 membered heterocyclic ring optionally having one or two additional heteroatoms selected from N, O and S, which is optionally substituted with one or more substituent selected from deuterium, F, C1, hydroxyl, oxo, CN, C1-C2 alkyl, C1-C2 haloalkyl or C1-C2 alkoxyl.

34. The compound according to claim 1, wherein halogen is selected from fluoro or chloro.

35. The compound according to claim 1, wherein halogen is chloro.

36. The compound according to claim 1, which is selected from any one of:
((R)-1-((R)-4-morpholino-4-oxo-2-(pyrazine-2-carboxamido)butanamido)-4-phenyl butyl)boronic acid;
((R)-1-((R)-4-oxo-2-(pyrazine-2-carboxamido)-4-(pyrrolidin-1-yl)butanamido)-4-phenyl butyl)boronic acid;
((R)-3-methyl-1-((R)-4-morpholino-4-oxo-2-(pyrazine-2-carboxamido)butanamido)butyl) boronic acid;
((R)-1-((R)-4-morpholino-4-oxo-2-(pyrazine-2-carboxamido)butanamido)-3-phenylpropyl) boronic acid;
((R)-1-((R)-4-morpholino-4-oxo-2-(pyrazine-2-carboxamido)butanamido)pentyl)boronic acid;
((R)-1-((R)-2-(2,4-dimethyloxazole-5-carboxamido)-4-morpholino-4-oxobutanamido)-3-methylbutyl)boronic acid;
((R)-1-((R)-4-(dimethylamino)-4-oxo-2-(pyrazine-2-carboxamido)butanamido)-4-phenyl butyl)boronic acid;
((R)-1-((R)-4-(ethylamino)-4-oxo-2-(pyrazine-2-carboxamido)butanamido)-4-phenylbutyl) boronic acid;
((R)-1-((R)-4-amino-4-oxo-2-(pyrazine-2-carboxamido)butanamido)-4-phenylbutyl) boronic acid;
((R)-1-((R)-4-oxo-4-(piperidin-1-yl)-2-(pyrazine-2-carboxamido)butanamido)-4-phenyl butyl)boronic acid;
((R)-1-((R)-2-acetamido-4-morpholino-4-oxobutanamido)-4-phenylbutyl)boronic acid;
((R)-1-((R)-2-((tert-butoxycarbonyl)amino)-4-morpholino-4-oxobutanamido)-4-phenyl butyl)boronic acid;
((R)-1-((R)-2-(morpholine-4-carboxamido)-4-morpholino-4-oxobutanamido)-4-phenyl butyl)boronic acid;
((R)-1-((R)-2-(3-(tert-butyl)ureido)-4-morpholino-4-oxobutanamido)-4-phenylbutyl)boronic acid;
((R)-1-((R)-5-morpholino-5-oxo-2-(pyrazine-2-carboxamido)pentanamido)-4-phenylbutyl) boronic acid;
((R)-1-((R)-4-morpholino-2-(oxazol-2-ylamino)-4-oxobutanamido)-4-phenylbutyl)boronic acid;
((R)-1-((R)-4-morpholino-4-oxo-2-(trifluoromethylsulfonamido)butanamido)-4-phenyl butyl)boronic acid;
((R)-1-((R)-4-morpholino-4-oxo-2-(pyrazine-2-carboxamido)butanamido)butyl)boronic acid;
((R)-1-((R)-4-morpholino-4-oxo-2-(pyrazine-2-carboxamido)butanamido)propyl)boronic acid;
((R)-1-((R)-2-(3,3-dimethylureido)-4-morpholino-4-oxobutanamido)-4-phenylbutyl)boronic acid;
((S)-1-((R)-4-morpholino-4-oxo-2-(pyrazine-2-carboxamido)butanamido)-4-phenylbutyl) boronic acid;
((R)-1-((R)-2-(cyclohexanecarboxamido)-4-morpholino-4-oxobutanamido)-4-phenylbutyl)boronic acid;
((R)-1-((R)-4-morpholino-4-oxo-2-(tetrahydro-2H-pyran-4-carboxamido)butanamido)-4-phenylbutyl)boronic acid;
((R)-1-((R)-4-morpholino-4-oxo-2-(phenylsulfonamido)butanamido)-4-phenylbutyl) boronic acid;
((1R)-1-((2R)-4-morpholino-4-oxo-2-(tetrahydro-2H-pyran-2-carboxamido)butanamido)-4-phenylbutyl)boronic acid;
((R)-1-((S)-4-morpholino-4-oxo-2-(pyrazine-2-carboxamido)butanamido)-4-phenylbutyl) boronic acid;
((S)-1-((S)-4-morpholino-4-oxo-2-(pyrazine-2-carboxamido)butanamido)-4-phenylbutyl) boronic acid;
((R)-1-((R)-4-morpholino-4-oxo-2-(pyrazine-2-carboxamido)butanamido)-2-phenylethyl) acid;
((R)-1-((R)-2-((3-chlorophenyl)sulfonamido)-4-morpholino-4-oxobutanamido)-4-phenyl butyl)boronic acid;
((R)-1-((R)-2-((2-chlorophenyl)sulfonamido)-4-morpholino-4-oxobutanamido)-4-phenyl butyl)boronic acid;
(R)-1-((R)-2-(benzylamino)-4-morpholino-4-oxobutanamido)-4-phenylbutyl) boronic acid;
((R)-1-((R)-4-morpholino-4-oxo-2-(((((S)tetrahydrofuran-3-yl)oxy)carbonyl)amino) butanamido)-4-phenylbutyl)boronic acid;
((R)-1-((S)-4-morpholino-4-oxo-2-(phenylsulfonamido)butanamido)-4-phenylbutyl) boronic acid;
((R)-1-((R)-2-(benzyl(tert-butoxycarbonyl)amino)-4-morpholino-4-oxobutanamido)-4-phenylbutyl)boronic acid;
((R)-1-((R)-2-(2,5-dichlorobenzamido)-4-morpholino-4-oxobutanamido)-4-phenylbutyl) boronic acid;
((R)-1-((R)-4-morpholino-4-oxo-2-(((((R)-tetrahydrofuran-3-yl)oxy)carbonyl)amino) butanamido)-4-phenylbutyl)boronic acid;
((R)-1-((R)-2-((2,5-dichlorophenyl)sulfonamido)-4-morpholino-4-oxobutanamido)-4-phenylbutyl)boronic acid;
((R)-1-((R)-2-(3-isopropylureido)-4-morpholino-4-oxobutanamido)-4-phenylbutyl)boronic acid;
((R)-1-((R)-4-morpholino-4-oxo-2-(pyrazine-2-sulfonamido)butanamido)-4-phenylbutyl) boronic acid;
((R)-1-((R)-4-morpholino-4-oxo-2-(phenethylamino)butanamido)-4-phenylbutyl)boronic acid;
((R)-1-((R)-2-((tert-butoxycarbonyl)(phenethyl)amino)-4-morpholino-4-oxobutanamido)-4-phenylbutyl)boronic acid;
((R)-1-((R)-4-morpholino-4-oxo-2-(pyrrolidine-1-carboxamido)butanamido)-4-phenyl butyl)boronic acid;
((R)-1-((R)-2-(3-methylureido)-4-morpholino-4-oxobutanamido)-4-phenylbutyl)boronic acid;
((R)-1-((R)-2-((R)-3-hydroxypyrrolidine-1-carboxamido)-4-morpholino-4-oxobutanamido)-4-phenylbutyl)boronic acid;
((R)-1-((R)-2-((S)-3-hydroxypyrrolidine-1-carboxamido)-4-morpholino-4-oxobutanamido)-4-phenylbutyl)boronic acid
(R)-4-morpholino-4-oxo-N—((R)-4-phenyl-1-((3aS,4S,6S,7aR)-3a,5,5-trimethyl hexahydro-4,6-methanobenzo[d][1,3,2]dioxaborol-2-yl)butyl)-2-(phenylamino)butanamide;
(R)-4-morpholino-4-oxo-N—((R)-4-phenyl-1-((3aS,4S,6S,7aR)-3a,5,5-trimethyl hexahydro-4,6-methanobenzo[d][1,3,2]dioxaborol-2-yl)butyl)-2-(phenylamino)butanamide;
((R)-1-((R)-4-morpholino-4-oxo-2-((R)-tetrahydro-2H-pyran-2-carboxamido) butanamido)-4-phenylbutyl)boronic acid;
((R)-1-((R)-4-morpholino-4-oxo-2-((S)-tetrahydro-2H-pyran-2-carboxamido) butanamido)-4-phenylbutyl)boronic acid;
((R)-1-((R)-4-(4-ethylpiperazin-1-yl)-4-oxo-2-(pyrazine-2-carboxamido)butanamido)-4-phenylbutyl)boronic acid;
((R)-1-((R)-4-oxo-4-(4-propionylpiperazin-1-yl)-2-(pyrazine-2-carboxamido)butanamido)-4-phenylbutyl)boronic acid;
((R)-1-((R)-4-(((1H-pyrazol-4-yl)methyl)amino)-4-oxo-2-(pyrazine-2-carboxamido) butanamido)-4-phenylbutyl)boronic acid;
((R)-1-((R)-4-oxo-2-(pyrazine-2-carboxamido)-4-((((S)-tetrahydrofuran-3-yl)methyl) amino)butanamido)-4-phenylbutyl)boronic acid;
((R)-1-((R)-4-oxo-2-(pyrazine-2-carboxamido)-4-((((R)-tetrahydrofuran-3-yl)methyl) amino)butanamido)-4-phenylbutyl)boronic acid;
((R)-1-((R)-4-((6-ethylpyridin-3-yl)amino)-4-oxo-2-(pyrazine-2-carboxamido)butanamido)-4-phenylbutyl)boronic acid;
((R)-1-((R)-4-((1-methyl-1H-pyrazol-4-yl)amino)-4-oxo-2-(pyrazine-2-carboxamido) butanamido)-4-phenylbutyl)boronic acid;
((R)-1-((R)-4-(1,1-dioxidothiomorpholino)-4-oxo-2-(pyrazine-2-carboxamido)butanamido)-4-phenylbutyl)boronic acid;
((R)-1-((R)-4-(isoindolin-2-yl)-4-oxo-2-(pyrazine-2-carboxamido)butanamido)-4-phenyl butyl)boronic acid; and
((R)-1-((R)-4-oxo-2-(pyrazine-2-carboxamido)-4-((pyridin-3-ylmethyl)amino)butanamido)-4-phenylbutyl)boronic acid.

37. The compound according to claim 1, which is selected from any one of structures 1 to 59.

38. The compound according to claim 1, which is selected from a compound of the group consisting of:
(i) structure 1, 2, 3, 4, 5, 6, 7, 8, 9, 10, 11, 12, 13, 14, 15, 18, 19, 20, 22, 23, 24, 25, 26, 27, 29, 30, 31, 32, 33, 35, 36, 37, 38, 39, 40, 42, 43, 44, 45 and 46;
(ii) structure 1, 2, 7, 8, 9, 10, 12, 13, 15, 22, 23, 24, 25, 26, 29, 32, 33, 35, 36 and 37;
(iii) structure 3, 4, 5, 6, 11, 14, 18, 19, 20, 27, 30, 31, 38, 39, 40, 42, 43, 44, 45, 46, 48 and 49;
(iv) structure 16, 17, 50, 51, 52, 53, 54, 55, 56, 57, 58 and 59.

39. The compound according to claim 1 to 39, wherein the compound is an inhibitor of LONP1.

40. A pharmaceutical composition comprising one or more compounds according to claim 1 or pharmaceutically acceptable salt, solvate, stereoisomer or mixture of stereoisomers, tautomer, isotopic form, or pharmaceutically active metabolite thereof, or combinations thereof, and one or more pharmaceutically acceptable carrier.

41. A pharmaceutical composition comprising a compound according to Formula 1, 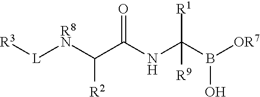
or a pharmaceutically acceptable salt, solvate, stereoisomer or mixture of stereoisomers, tautomer, isotopic form, or pharmaceutically active metabolite thereof, or combinations thereof, and one or more pharmaceutically acceptable carrier, wherein:
R¹ is selected from the group consisting of: deuterium, C1-C4 alkyl, C1-C4 alkoxyl, C₁-C₄ oxoalkyl, C1-C5 alkyl-alkoxyl, wherein each alkyl, oxoalkyl or alkoxyl is optionally substituted with C3-C6 cycloalkyl, phenyl, phenoxy, or a 5- or 6-membered heteroaryl, wherein said phenyl, phenoxy, or heteroaryl are each optionally substituted with one or more substituent selected from deuterium, halogen, hydroxyl, CN, CO2H, CO2R¹⁰, CONR¹⁰R¹¹, NR¹⁰R¹¹, SR¹⁰, SO2NR¹⁰R¹¹, C1-C4 alkyl, C1-C4 haloalkyl, C1-C4 alkoxy, phenyl, or a 5- or 6-membered heteroaryl;
R² is (C1-C4 alkyl)-C(O)NR⁴R⁵;
R⁴ and R⁵ are each independently selected from hydrogen, deuterium, C1-C4 alkyl; C1-C4 haloalkyl, C1-C5 alkyl-alkoxyl or R⁴ and R⁵ together with the N to which they are attached form 5 or 6 membered heterocyclic ring optionally having one or more additional heteroatoms selected from N, O and S, wherein the 5 or 6 membered heterocyclic ring is optionally substituted with one or more substituent selected from deuterium, halogen, hydroxyl, oxo, CN, C1-C4 alkyl, C1-C4 haloalkyl or C1-C4 alkoxyl;
L is C(O), C(O)O, C(O)NR⁶, S(O)₂, or a bond;
R³ is C1-C4 alkyl optionally substituted with one or more substituents each independently selected from the group consisting of deuterium, halogen, cyano, hydroxyl, C1-C4 alkoxyl, 5 or 6 membered aryl (e.g. phenyl) or 5 or 6 membered heteroaryl; or
R³ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, oxo, C1-C4 alkoxyl, or C1-C4 alkyl that is optionally substituted with one to three substituents selected from deuterium, halogen, cyano, hydroxyl, or C1-C4 alkoxyl; or
R³ is aryl or heteroaryl having one or more heteroatoms selected from N, O and S, wherein aryl or heteroaryl is optionally substituted with one or more substituents selected from deuterium, halogen, cyano, hydroxyl, OR, CO2H, CO2R¹⁰, CONR¹⁰R¹¹, NR¹⁰R¹¹, SR¹⁰, SO2NR¹⁰R¹¹, C1-C4 alkoxyl, or C1-C4 alkyl that is optionally substituted with one to three substituents selected from deuterium, halogen, cyano, hydroxyl, or C1-C4 alkoxyl;
R⁶ is hydrogen or C1-C4 alkyl optionally substituted with one or more of halogen, hydroxyl and phenyl, wherein phenyl is optionally substituted with one or more substituent selected from halogen, hydroxyl and C1-C2 alkyl;
R⁷ is hydrogen, or R⁷ and R¹, together with the boron atom to which R⁷ is attached form a 5-membered heteroalkyl ring;
R⁸ is selected from hydrogen, deuterium or C1-C2 alkyl optionally substituted with one or more substituents each independently selected from the group consisting of deuterium, halogen, hydroxyl, cyano, methoxyl and phenyl;
R⁹ is selected from hydrogen, deuterium or C1-C2 alkyl; and
R¹⁰ and R¹¹ are each independently selected from hydrogen, deuterium, C1-C4 alkyl; C1-C4 haloalkyl, C1-C5 alkyl-alkoxyl, C3-C7 cycloalkyl, or R¹⁰ and R¹¹ together with the N to which they are attached form 3 to 7 membered heterocyclic ring optionally having one or more additional heteroatoms selected from N, O and S, wherein the C3-C7 cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl.

42. The pharmaceutical composition of claim 41, wherein the compound of Formula 1 is defined according to claim 2.

43. The compound according to claim 1 for use in the treatment of a disease or disorder or disease.

44. The compound for use according to claim 43, wherein the disease or disorder is characterized by mitochondrial dysfunction, such as mitochondrial disorders, including a neurodegenerative disorder, a metabolic disorder and a disease associated with the aging process.

45. The compound for use according to claim 43, wherein the disease or disorder is an oncologic disease or disorder, such as a cancer and/or a proliferative disease or disorder.

46. The compound for use according to claim 43, wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma colon cancer, colorectal cancer, cancer of connective tissue, esophageal cancer, embryonal carcinoma, fibrosarcoma, glioblastomas, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), liposarcoma, liver cancer, lung cancer, lymphoid cancers (e.g., Hodgkin's and non-Hodgkin's lymphomas, mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML).

47. The compound for use according to claim 43, wherein the use comprises administering the compound orally; topically; by inhalation; by intranasal administration; by intracerebroventricular; or systemically by intravenous, intraperitoneal, subcutaneous, or intramuscular injection.

48. The compound for use according to claim 43, wherein the use comprises administering to a subject one or more compounds according to claim 1, optionally in combination with one or more additional therapeutic agent.

49. The compound for use according to claim 48, wherein the administering comprises administering the one or more compounds according to claim 1 simultaneously, sequentially or separately from the one or more additional therapeutic agent.

50. A method for treating or preventing a disease or disorder in a subject where inhibition of LONP1 may be beneficial, wherein said method comprises administering to the subject one or more compounds according to claim 1.

51. The method according to claim 50, wherein the disease or disorder is characterized by mitochondrial dysfunction, such as mitochondrial disorders, including a neurodegenerative disorder, a metabolic disorder and a disease associated with the aging process.

52. The method according to claim 50, wherein the disease or disorder is an oncologic disease or disorder, such as a cancer and/or a proliferative disease or disorder.

53. The method according to claim 52, wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma colon cancer, colorectal cancer, cancer of connective tissue, esophageal cancer, embryonal carcinoma, fibrosarcoma, glioblastomas, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), liposarcoma, liver cancer, lung cancer, lymphoid cancers (e.g., Hodgkin's and non-Hodgkin's lymphomas, mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML).

54. The method according to claim 50, wherein one or more compounds according to claim 1 is administered in combination with one or more additional therapeutic agent.

55. The method according to claim 54, wherein the administering comprises administering the one or more compounds according to claim 1 simultaneously, sequentially or separately from the one or more additional therapeutic agent.

56. The method according to claim 50, wherein the method comprises administering the compound orally; topically; by inhalation; by intranasal administration; by intracerebroventricular; or systemically by intravenous, intraperitoneal, subcutaneous, or intramuscular injection.
